SEQUENCE LISTING (113 sequences)

SEQ ID NO: 1 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 2 - protein, 629 aa, Homo sapiens
MPRYGASLRQSCPRSGREQGQDGTAGAPGLLWMGLVLALALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSADSPALCRHLMPDGSLPEAQSLWPRPLFC

SEQ ID NO: 3 - protein, 529 aa, Homo sapiens
MTSSRLWFSLLLAAAFAGRATALWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 4 - protein, 540 aa, Homo sapiens
MTSSRLWFSLLLAAAFAGRATALWPWPQNFQTSDQRYVLYPNNFQFQYDVSSAAQPGCSVLDEAFQRYRDLLFGSGSWPRPYLTGWPHQAYPVFLGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 5 - protein, 580 aa, Homo sapiens
MTAPAGPRGSETERLLTPNPGYGTQAGPSPAPPTPPEEEDLRRRLKYFFMSPCDKFRAKGRKPCKLMLQVVKILVVTVQLILFGLSNQLAVTFREENTIAFRHLFLLGYSDGADDTFAAYTREQLYQAIFHAVDQYLALPDVSLGRYAYVRGGGDPWTNGSGLALCQRYYHRGHVDPANDTFDIDPMVVTDCIQVDPPERPPPPPSDDLTLLESSSSYKNLTLKFHKLVNVTIHFRLKTINLQSLINNEIPDCYTFSVLITFDNKAHSGRIPISLETQAHIQECKHPSVFQHGDNSFRLLFDVVVILTCSLSFLLCARSLLRGFLLQNEFVGFMWRQRGRVISLWERLEFVNGWYILLVTSDVLTISGTIMKIGIEAKNLASYDVCSILLGTSTLLVWVGVIRYLTFFHNYNILIATLRVALPSVMRFCCCVAVIYLGYCFCGWIVLGPYHVKFRSLSMVSECLFSLINGDDMFVTFAAMQAQQGRSSLVWLFSQLYLYSFISLFIYMVLSLFIALITGAYDTIKHPGGAGAEESELQAYIAQCQDSPTSGKFRRGSGSACSLLCCCGRDPSEEHSLLVN

SEQ ID NO: 6 - DNA, 10415 bp, Homo sapiens
tgactgggctcaaacattttctcctttaagagctgccatttttcctggctggtgccataggaatcatctgggtgcctgggcacacccgctgctgctttaaggcttccgccctgatgctgacactgctgctccacgggcccagttctgcatctccaggaaagacaaacagtctccagttttgggcccagctttcctagtctcttcttttccttaccctcagccctgatcttgtgtttgtacggacagtgagctcaccctaggcctggacccaggcccagtttccaaagcaagcagcacacagcgcatgttcacataagcatgggggctggggggacactggggcttactgatctttttctaggggcctccagcccctggcaccacctagaggggaaagtgagtcacccaaaccattgcccctgggcttacgtcgctgtaagctcacactggccctgctgtgccctctttagtcacagacagcgtgtgagctgactctgtccctttaatgcccaggctgagcccagtgcctccttgagtatctgctccatcactggcgacgccacaggtaggtgtgaatggagtagccaggtgagattgtctccaggaagcccacagcaggatccttgatggtaagaggcacatccttagaggagctagggagcagggaggagaagctgagagtgtgatcctgccaaggcccccaacgctgtcttcagcccacttcccagacctcaccattgccctcaccggtttagcacgaccacaacagcagagccatcgggatgcatcagtgccactgcgtccaggtcgttcttctgactggcaaccagccccactctctgggagccctcaggaatgaacttgctgaatgtgggaacagatggtcagagtccctcggggtacctcccatgaaaccctcatctaagaagtcacccacccacggacccaccccataactcctgcagaggctctgccctggctctctaggcctggagccatgctgctgggcactgaccctgcttttctgcatcgcagtccagcctcaggcattggggttttctgttgctacctagtcacttcctgcctccatggtgcaaaaggggatgggtgtgcctcttccgaggttccaccctgaacaccttcctgctccctcgtggtgtagagtgatgtaagccatccgatgtaggagatgataggcctggtatggaatgggggtgcccgccctccactcacctgaagtggccaaggtggtagaacatgggctgtttgtaaaacgtgtccttggtgatgtctacaatgatgggactgtcgacaaagttacgcacccaattgggtcctccttcggggttcagggcaaggttccagtcggtccagccgaccacatggtacaggaggttctagggtaaggacaaaggcaaagagacaaaggcgcaacactgggggtccccagagagtgtaggtaagggtcacatgtgggagaggcagctgtgggtaggtcagccctgtgaggggcacattccttagtagctaaggagttgggggtgtgaagatccaggcatctcaaggggagctgagaagtctgaggcagctgcaagtgcctcagtagttgcaaaaggggcaatgaggtgtgcagacctgtgaaggaaagggaagatagggaatcatggttccccagagttgctcaaaagggcaggctagctggggaaagctggacaggaagggcttctgtcagtctttggtgaaactagtaagaggtctgaggtctgctttgcaggaagggagactggggtggcttaccgtgatgatgctgtggctgtactgcatccctcgatcccaggagcctagccgcacactctgctcccagaacttggagcccacacaggcctctgaggcaaagagcatggtgttggggaacaggcggtgtgtctcccctagggtggctttggctggagccagaaagtccaggtaccaatgtacagcaatgccatgaacatatttagctgcttctgggtctgtcagtacctgcaaaggaagagcaactgatcctggaccttgcacacaggcttctggaacttctagttcctgttgtaggaatcctggagttgggtgacgggaagaatgcaactagagaggtttggggagatttttttttgtttttgagacaggatgtcactctgtcactcgggctggagtacagtggcgcaatcacgactcactgcagccttgacttcctagggtcaactgatcctcccacattagcctcctgagtagctgggactacacgggtgccacacccagctaatttgtgtgtgtatgtgtgtgtatgtatgtgtgtgtgtgtatatatatatacatataaacacatatatatgtatatacacatacatacatgaaccaccacccccagcctagatagttttgttttgttttgtttcgagatggagtctcgctcagcctcccaggctggagtgcagtggcgcgatctcggctcaccgcaaccaccatctcccgggttcaactgattctcccttctcagcctcctgagtagctgggattacagacacccaccatcatgcccagataattttttttttttgtattttagtagacacagggtttcaacacgttggccagggtggtcttaaactcctgacctcaggtgatcacccgcctcggcttcccaaagtgctgggtttgcatgagtgagccacctcgcccagcccctagaaaggtttcaagcgacaactgtgggatccatggcaccctggaggtccaggggaatggtgctctaggaatccatagttgggtagagaaatcgctctaagtttgggagccagtcatttggatgctggatttgaaggtcactggagcaccatggaggtccaggccttaccacctttgcccagtggggcagcagcaagcgttggtcatccagcatgagtaggcggacattgtggtgagtactgttggcgagggtaggacctaggtcacgggcaatgaagtctcgctgatgttcaggggtgaagcccaggcactggaaggggtatccactcaacagcccagcagaaggctcattttcagctgtcactgcccagaactgtaacttgtgctcagcataggcatccaggaacctggcaagagaaaggtcatgaatgatccggccaagaaagtggaccagaccagctgggtgtggtggctcacacctgtaatcccagcactttgggaagccgaggcaggtggatcacttgagttcaggagttcgagaacagcctggcgaaaccccgtctctactaaaaatagaaaaatcagctgggcctggtggcaggcgcctataatcccagctacttggaaggctgaggcaggagaattgcttgaactcaggaggcagaggttacagtgagtgaagatggcgccacagcactccagcctgggtgacagagagagagactccttctcaaaaaaaaaaaaaagaagaaaaataaaaagaaagtgggccagaccgagagaacaggaagcctgatggagtgggcaagattgacaggcccaaggctgaaaggcccagaaggtagaaaggtgagctgaggacaggcagatctggaagtggaactaggttgagggttgggacacagatcagcatggctaaatgggaggccagtcctgatcccacatccttgctgatcccttacttcacaaagtatctggcccaggtctggtggtagatgtctccgggctgtcccttgagtgaccccttcccattcaccgctccattggtcttgagccaagtgggtgatgtccaggggctggcaaggagtgaaacgggacgctgggccaactgcagggctcggtgaatcaggggtatctagagacaaaggtagtgaagagagaagcacccagagttggaacacatactagcccaaccagtgcatccggttcagccattagcctccaccctcccacccccaggacaaaacagcaggggacaaaatgtctgtacaagcagacctaccctacagtttctcaacccccagacatcagggccctcagggcctgaaaaagctagaatgcctaccttgagcttggtatcttcctctgggaggctgaagttgtgcaactggaaatcatcaggggtgtctgcataggtgtaggtgcggatggagaagtcacagctggccatgggtacccggatgatgttatatccgattcctacagaaaaggatgatcaagatatggtagtccgagtcaataggagagtatgggactctgcttatcacttgccagtcctaatagtgtctgagtcagggccaaagggaacttgggctcctgggttggaacctgtggaggctggcacctgggtgaagcgcaggcctttctgagcctgagtccgtagcagttagcagatgataggcggtgaaatcttatttcacagggcattaaaacaggaaccaaatgtcagggatgggcagaagtcagggtccaaagaaagggcaaagaaaagtgtcagtggctcacgcatgtaatcccagcactttgggaggccgatgtgggcagatcacgaggtcaggagttcgcaatcatcctggccaacatagtgaaaccctgtctctactaaaaatacaaaaaattagccgggcgtggtggcaggcacctgtaatcccaggtactcgagaggctgagacaggagaatcacctgaacccgggaggtggaggttgcagtgagctgagattgtgccactgcactccagcctgggtgacagtgcgagactctgtctcaaaaaaaaaaaaagaaaaaagaaaagtgtctgctgggctcggtggctcacacttgtaattccagcacttcgggaggccaaggcaggtatatcatttgaggtcaggagtttaagactagcctggtcaacatgatgaaaccctgtctctactaaaaatacaaaaattagccaggtggtagtggcgcacgcctataattccagctactcaggaggctgaggcaggagaatcactatagcctgggaggcagaggttgctgtgaggggagatcacaccactgcactcctgtctcgccgacagaatgggcagagtgagattctgcctcaaaaaaattttaaaaaaagaaaagaaaaacgaaaagtttcaatggctctatgtcatcttgtccccttcctcctcaccttcttcagagaagtacgatttaagtagcaaattttgggcagggggtgacagggcaaggatgttgagagcagcagcatctgtcatggcccctccaaatcccttcactttctggaacttctgttctggctgcagggtcagtagcaggcctgaggacatccacagggaataagggtatcagtacccagcgggaaactccatggtgatcactgacaccatttacctctaggaggacccagcctggcccagggggtgaggggtgtaatggttacctgtgcccgtgtgattagcctggatgggccccatactcagctccatccgtcgcccactgcgtgtactctcatagcggctgaaggtaccaagggcaggaaaggtcggggggtcaaaggagtcacagtatgtggcattgcagacacacaccaccgagctgtagccgaagcttttagggatgcaggggcgggcacctgggagggagggagtacaagcagagtgaggtctgatgaagacatggagaatggacacatctgctaggagagactgaacacggtttcaaaattcctcaccccttggccgggcgcaggggctcacacctgtaatcctagcactttaggaggccgaggtgggcggatcacctgaggtgaggagtttgagacctgccttgccagcatggtgaaaccccgtctctactaaaaatacaaaaattagctgggcgtagtggtggccgcctgtaatcccagctacctgggaagctgaagcaagagaatcgcttgaatctgggaggcagaggttggaatgagccaaaattgcaccactgcactccagcccaggcaacagagtaagactctgtttcaaaagaaaaaaaacaaaatcctcaccccaaagttggtctcagtcactcaaaaggatttattgagcacctactaaaagtctaccaccagcttactggaaggctaccaaaggactatgaggcagaagggaggctctgtgctacctccccactgccttgactcactcacctgatgcccacgacactgcctgaagtagaagcaatcctgtgaggctgccagccatgatgcttaccctactcaaaggcttgggacattcctgaggacagaatgaggaatgactgaaaagcaagcccctctccacccctcaactactctcctgggcagggcttagctgcctttgggtgcccatggcccggtctcccacattcattaggacaaggcccacagaaacctgggtgcagctctccctgcaacccttctgatgacaactctctgcccaccccaaatcaggatgctccccaccactcttcccacccatttcaacttcgacccctccctccatctgtgccttgctcaaagagccatgatggccctggattcaaagagagtctgtcattcattaaattccagtgccaggattccagaagcactgtgacaatgctgattgggagctctctctcttacctctctggaaggacttgaaaactccatcccctcagggtcattagatgaagagaagaccacaggggttccagagtctctgaaggatagaggatccactaaacaaaaacaaggatgcaggtacctgccttagctataggcactaggttagccctgcaagtaattccggcttcccgatgtggatgggtcatgtgatgactaggagcgtcacatgacacaggaagtgaggcaatcacagccatatttctaaagggcaattggcttcctctcatctgttacagattatatgccctataaaactctggagggcatgtatgggtgacaactttaggaagagcctagaacacagatttgcatggagaatggggtagaggctcctaaatcccagaggatgggaaccacagcaggcacacttcttttttgatagaaatgtcaaaaaggtacaaataaagctgatataatttaaaaaaaaaaacttcactgtgcaaaatactgacactaccaagatgtttaagacagtctttgccctatagaggtgtgtgaggcatggaagtcagacacacagatatttacagataaagacagaaacggtaacaggtgtgcatggagagctctctgagatgaggagggaccattcgtgggtggggaattatccaggatggcttcacagaggaaaaagtgaggggggttattagccagtgaagtgcagggcacaagagggtgggacactggcagtgggatgacaggactggagggagggagtggtcagtgcggctcctctgcagcgtcccttgtttcatcatcagatgcagatggtaataactgttcttccttcctcacaagacagggaggtttgtaaagtcgttcaaaaaccaaagtgttgtacaaagccatatcctcagtggacacaaggaggaagctgtccatggtgtggcctcatgaaccacatcaaatgagatttagcgggagtggcacacacagtcatgacctgactaatcccagctctcagcccatttccttgcctggaaaatggaggcaatgccacaacctcaaagggtggttactgcagtcagtgaggtaagtgcagtgcctgaccacttggtaggcacctgggaactacttgtctcttgtttgtattttttgttgttgttgttttttgagacagagtctcactctgttgcccaggctggagtgcagtggcgtgatcttggctcactgcaacctccacctcctgggttcgagtgattctcctgcctcagcctcccaagtagctgggattacaggtgccctccaacatgctcagctaatttttttatttttagtagagatggggtttcactatgttggccaggctggtctgaaactctggacctcaggtgatctgctgccttggtatccgaaagtgctgggattacaggcatgagccactgcgctggaccccagccatcctttttgttctttaaatgatctcagtgaagtctttctagatacactccaagcaaaattgatcatttcttcttctaggttcctccagtaatttttttttttttggttttgagacagagtcttgccctgttgcccaggctggagtgcagtgtgatctcggctcaagcaattgccctgcctcagcctcctgagtagctgggattacaggagcccaccaccatgcccagctaatttttgtatttttagtagagatggggtttcaccatgttggccaggctggtctcgaacccctgacctcaggtgatccaccagcctcagcctcccaaggtgttgggattatagcaatgagccaccacacccggtccctccagtaattaagtacagtctcagtgagacagcaagtttggaatcctggctacaccatttactggctgtttgactttcaataaatcaatcactctaagcctctgtttcatctataaaaggggagtgataactcctacctcacagaattgttgtgaggtttgagtgtgataatgtgtctctagtacactgcttgacactaaacattgcaacatgtcaggcctctgttcctggagttccttgaaggaatgtcttatgcattctaagtatcctcgtagatagcctggcacaggggtagctgtcaagttgtagaattgaactagttgcttcactatgtcagtagccaccccttccagacttctcacttttcaaggaaacattgcacaggatttgttctgggtagggcaggtaatatctagtaccttacttccctcaagttcattcatctcacagatatttcctgagcacattctacatttgcctctcctgctctattgagtttagaagtccaaacatctccttccacttcccctctgcatagtgagcctcttcttttttggccaggtactgagcaattttttgtttgtttgtttgagacagggtctccgtatgttgccctggatggagtgtagtagcttgatttcggctcagtgcaacctctgcctccagaataagcaatcctcccacctcagcctccagaatagctgggactataggcgcacaccaccacacctggctaatttctgtattttttgtagagacggggtcttactgtgttgcgcaggttggtctcaaactcctaaactcaaaggatcctcccgccttggctttgcaaagtgctgggattacagggtgaggcactgcgcccagtccgcttgttttttgttttgtttttttttttgagacggagtatctctgtcacccaggctggagtgcagtggcacgatctcggttcactgcaacctccgcctcctaggttcaagcggagggtagggaccagtccatcctggcacccatctgcagctccagtgcgaatcccaaccccgacgctcgtcgccgggctccgtgaatgtttgtcacatgtctgaagaacgtatgaattacataaccttcttcccactccacccctcaaaaagcaagtggatataaagacttgaagattttataatctcttcttcattagtaaaatctgaccatccttaccgttaaaaataataatgatggttggccgggcgcggtggctcacgcctataatcacagctgtttggcaggccgaggcgggcggatcacgaggtcaggaattggagaccagcctgaccaacatggtgaaaccccgtctctactaaaagatacaaaaaattagccgtgcgtggtggcgggtgcctgtaatcccacgtacttgggaggctgaggtcggagaatcgcctgaacccgggaggcggaggttgcagtgagccaagaaggcgccattacactccagcctgggcgacagggcgagactccctcaaaaaataataataataacaataataataataatggtgaaaaaggttaagtgcgaacgcagggaggggacagctaagatccaaaggtcgaaatattcattacgcctaccgtcggcgaagagaaacagcagccccaaccggaggcaaaacgaaatcccaccgcagcctgcaaaggcgcctgggcgggactggagactggggccccgcgcagtaagactctgaaggcaggatgcagcccgaccacccgcagccgcgagaaaagcagccctggggagtcggggcgggacctggattggaaaagagacggtcactcatgcagaggcgggactcagagcccttcctcaagtctcattggtcaagttgaacgaacaagtgtcgctggcgagccggagagagagagagagcgagagcgagagagcgagcgagagagagcgagagagcgagagagcgagcgagagagagagag

SEQ ID NO: 7 - DNA, 4585 bp, Homo sapiens
agctgtcagagatcagaggaagaggaaggggcggagctgctttgcggccggccgcggagcagtcagccgactacagagaagggtaatcgggtgtccccggcgccgcccggggccctgagggctggctagggtccaggccgggggggacgggacagacgaaccagccccgtgtaggaagcgcgacaatgccccgctacggagcgtcactccgccagagctgccccaggtccggccgggagcagggacaagacgggaccgccggagcccccggactcctttggatgggcctggtgctggcgctggcgctggcgctggcgctggcgctggctctgtctgactctcgggttctctgggctccggcagaggctcaccctctttctccccaaggccatcctgccaggttacatcgcatagtgccccggctccgagatgtctttgggtgggggaacctcacctgcccaatctgcaaaggtctattcaccgccatcaacctcgggctgaaggtgagcactgaaggggctgcagtggaggaggccgaaaggagtgctggggctgggggctggggctgatgctggtgcgctgggctcagaatgcatccctgatggagagggtggcatctacaatccatcactgagtttgctcccctttggggacacccatggctacatgccaccatcaccccattgtgacctttgtgaagtaagaaaataatgcagacagtgcctgaggaagtcagcttgccaagcaaaggcctcatgccacaggccgctgagctaaagaagaagcgatggcctggtgctgcctgagttacagggcaatatctggaaggcaaaggtgtgcactgagcttggtgcactgagtcctgcccagccccagtttggaaatggaggcccaaggggtggtggccaggggttggcctggttcctctgctctgcctctgatttctcaccatgcgctcctcccactgcagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcaggtacttatcgtccgtggaaacccaggaagggaaaagaaaggtgaatgaaagtgaagggagaagggaacctggggcattgtctctgattgctctagcatgagtccttagtgctcttcatttggctcccctaatctgactcctccttccctttctactgttttgccgcaccaggctttttttttttttttttttttagtttagtttttgtagagacaagatcttgctatgttgcccaggctggtctcaaacacctaacctcaagcaatcctcccgcctcggcctcccaaaatgctgggaccacaggcatcagctactgctcctggccctccctttttttttttttttttttttttttttttgagatggaatcttgctctgttgcccaggctggagtgcagtggcaccatctcagctcactacagcctccacctcctgggttcaagcaattctgcctcagcctcccaagtacctgggactacaggtgcacgccaccacacccagctaatttttgtatttttagtagagatggggtttcaccatgttggccaagatggtcttgatctcctgacctcatgatctgcccacctcggcctcccaaagtgctgggattacaggcatgaaccactgcacccagctttccagccctccctttctactcttatctccagccaccctccttcaaaggtctggcagcataacctctctatgccccagctgtgtctttgctcatgttggccctctggaaatgatttcccccttttttttaagtgctccagtttttcccaccttatccatcccatgtcatcttccctctgtgtggtccttgcttcccattctagctaactcttatccctcccccatactcctggagccctctgccctcagagtcttttgtgtcacacagacccaataattagaactgtttggtctctggctagactgtgagctccttgcaggtggggaagatgtcatgtatgcttttaccctccacccaaatgcccagcacaggaggaccaggattggaacaagtgttgacctctcatgtttactttgtttcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgagggccagtagtgggaacacggtggtgctgggggacaagcaggctcctgttgagctggagcacctctgggcacagaagttttattttcctggcattcccaacaagtgttccctggggattcagctcatggtcactgttgaaagccttcattcagtccccctttctctagccagggctgcctggacccctggatgccctgattaccatccttaattctccctactaggtgcatataattggccacattcccccagggcactgtctgaagagctggagctggaattattaccgaattgtagccaggtaggacggagatgagggtgggaatagggacagggtgagtgtctgaaggctgaaaattcccttgagcatctcaccatccctgttgtcccatggagtggggaggctcctcactagaacaggttggagaaagagggcatcctatctccccagatgtcttcctacccctccctagaatcttctgaatgtagtaccttctggccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggtgagtgaggcagaagggagcctcccttatcctggagttggtgggataggggaaggaggttggagccagagcctgcaaagcatgggcaggatgtgtggcccctccctggagttacccttgctccttgcccctccagtcagccccacatccttgcaggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctagggccccagggcccacatttgggaaagttcttgatgtaggaaagggtgaaaaagcccaaatgctgctgtggttcaaccaggcaagatcatccggtgaaagaaccagtccctgggccccaaggatgccggggaaacaggaccttctcctttcctggagctggtttagctggatatgggagggggtttggctgcctgtgcccaggagctagactgccttgaggctgctgtcctttcacagccatggagtagaggcctaagttgacactgccctgggcagacaagacaggagctgtcgccccaggcctgtgctgcccagccaggaaccctgtactgctgctgcgacctgatgctgccagtctgttaaaataaagataagagacttggactccaga

SEQ ID NO: 8 - DNA, 33039 bp, Homo sapiens
cctagagaacaatatgtgtttaataatatttaaataatggttgtataaaattgaagcagcaagaaacccaaaggagaatagctctaggggagggaggtggatgagtatgcatgggggagaggctcttctgtgaccaggttgggtctggagccctccccactgtccagaacacctccaagcccctacatctttttccatataccaacgccttggagatataatgcagaagtgaagtgagcaggctgaggattagggcaggtgtctggaatatggtcaggagtgggaggggagtgacatagctcacaggcaaggcagaacagcacaaaggctataggtttcattcccagccctcaacttaaaagacctcaggggcagacactgactccagcctggctgtgcccttaccctaacgccattcacacttttttttttaaacacaggtaatccatgtttattatagaaaaatgccacattactctttattgaatgcgagcgccagcaccggcccctttctctccaagcacaggggcacgcaggcaaggggcacgaaggcaaggggctccgtcccctggccaggatgcagtggaagcctggctccactaccattcacctacagccagcaccctcctcggtgcctggggctcaggtctgttcaaactcctgctcacagaagcctacattgaggggttgggcctggacacctcgcctgcaagaggacatgaagaaatggcaagataagcccctcagaaggggccccagcaacacttttcacaccagtcaacagtctctccttccccatttcggtgactctcaaactgtcactgtacaccaaatgcacctgggaatctcaattccagagattctcacctggggtggggcaaagtattggcaagaatctgcaagctgaacaggtgcctcaggtgattctgatacaggtcttggagatccagaaacgttggcgacatctatttatatcctgcccatctctgtaccctaaccccaaataactcaatccctttagaatatagatggatgtctgaaaggcagccaatgctcttggtctacttagccctctgttcctgactctaaaatgctgtaaatatggtggcactgggggacttaggtgctgtcctgtccatgaacatggatgcgaaatcctgtccttgggtctcagtctataacctgaccaatctccttaggggacctctaggcatgcttcccttatcatccaagtaagcaacatgcttagagaaccagctggaagttacccagtgaatgacataattgaggcttagacaccatcatgcttgactctcagcccaggactctgctctgcactaggctatgtcccccaagtactcaagacctagccaacgaaggaatcagggaaatgactatcttacacaggcacatccctgacagaggcaaaggtgtcatgacatcctgtaagtcagcctggttcaaatcctgcttcttagtgactttgggcaaggtcctgaactctgtgcttcagtgtctatctctaaaatgcacctgtctcatggggctgtggtgaagactaaatgagacaatatttgtaacaaacttagcacagtgtttgccacaaaatacagttgtccctcagtatccacaggggatttgtttcaggacccccgtggataccaaaatccaaggatgctcaagtcccttatataaaaatgacatagtatttgcatataaccaatgtacaacttcccatatacttgaaatgatcttagattacttatacctaatacaatataaatgctatgtaaatagttgttatacagtattttttatttgtaccattttttgttgtattttttttttcctgaatactttttatcgcagttggttgaatccgtggatgagggctgactataagcctctgtaagtgttagctattgttaattattgtcttcctctctctaaggggttccccagccctggatctggcctgctccgccttgcgaaggccccacagcttgcttacctcagcaattcacagcggaagtgtgacaaacgttcataggcaaatgtcaggtcagatgtcaacttgttgctccacagcctttcggcaacagcccctgctctgggcctggaggaaaaggggcatgtgccagattgggccctgtattccctgcaaaggtgctggacatccacaggctgctacctcttgtctaggcaccctggactcactcaggccaaaggaagtgatcaataccttgttatgagccacagccaggttggatggctcccagagagttctacggctcagctccaccttcattaaaatgtgggtagcaatcccactctcttcctctctcaaagatatgggaataagaataaaccttaaagccatcattaaccacaatgacagtggcagcagcagaggtccttaaaatgtaaactttgcacacccaaccatacttggtaaggctgcagtgaaaacagcagagggaagccttattacaggaatcatggatgcttgtgctgggtcttgagcaactcacctatgttctccaggcctcattatctctctgggaagtaaggttaacaatccctatcccacataccaaagggcagctggagggatatagacggaagtcatgtggagagtgaatattgcacacaaatcttcagaaggctcgttgcacggcctgctctctctcaggcctgaaagaagggtactagtccagaggtggctagatgggattgggtctctaagggagaactcctgctctcaggcccaaccctccacctcccccccgaaaacccttaccagagcctggggaccaggtttgtgttgtccacatattctccccacatacaagcctctccaccaatcaccagagccttctgctcaggggtacctgagggaaaacaagcaacaacagtctggtgatggtggggtaactccagggtccctctcaaccaccttcccaatgtggccctcaccccagctaagttgtttcatttactaactggaaatgtctggcccagaccttcctgcctcccatcctgtgccccaacccagcctcctttggttagcaaggagagctctctgctttcaccttcaaatgccaggggttccactatgtagaaatccttccagtcagggccataggatatacggttcaggtaccagggggcagagagaagggcccggaagccggccttggtgaccagttccagctccttcatatagttcactggaatatcctctcgccacacctgtatgattgtgtctggctgaatctgttataaaaggtcaaatggcagtaaggacacaaagctgaggagattcctgggccttattcatacacaggcaacatgggacaacaggtatgtgctccctctgttccccagcagcaaagtatgtctctgtgactcctgaccattgagctcacacctcaggacagaggccaaggaatccagggccctaagaggcctccctgcagctctcctccccgtccctctgcagcctccctgtccacacagagctagtaataccagctgtgagctgagggaggcaagcagtgggacccaggagctgccatgtccaaaagtctccagatgtttctggggacgggacaaggttcctgaagagtccttgcatgtgacaggagactgggatcttactgatcaacaataaaggagaaggtaaccttggtcccacagacccagaagaccctcatctatgatttaggaagaaaaatccagcctatccaagtaacctaacagcagaaatagttcttaattaagtttgtagtgagggaagatagaaactctgacacataaacccagggctcataccacagcggcctttggtttttttgaggcagggtctcactctgttgcccagggttgagtgcagaggcacatcttggctcacagcaacctctgcctcctgggctcaagcagtaccccaacctcagcctcctgatttttgtattttttgtagagatgagggttttgctatgttggccaggctggtcttgaactcctaggctcaagcaatccccctgccttggcctaccaaagtgcttggattacaggcgtgagccactgcacccagcactctgtggcctttctagagagaaaaggagagtgctccgaccattaaagcccaatccaaaccaggaggatcagtctctgtagaggcagggaggagctggggagaccagagggaggcactgctggtggcttcttctcttctctgccccggctcacctttactttattatcaaacacctcctgccacaccacatagcccttgccataagaagagacgatgtccagcagcctggagaggagaggagtgtctagtaagtgtctgcttagctcagatgggttctagactgtttgtgccagtgctctaggggttgagcctggccaggggcctattcctcattaagcagtgtctttcccctgggctgaaaaccaaccactccaaatctgaaaggatgggacaaaggacaagggaaccctgcagggaccagacagtggccaagcagggcctgactcggtatggaaagggaggaccccacaggaggacccccaagggaccccacccaccctccttccttcctcacgtctggatgtagaaggactccagctgcttgaagtcctcaccgaagcctttcttcctcataaagtcctggatctctgggttggacttcctgaatcccaagagaaaatgaagattaatctttcaacatcctgaaagcctaatgcctggggattagtcacctggccccctataacttcctcttttcacctgaaaaatcaaataagcaaggatctcagaagccctacatgtggaaagtgtggccaagcaaagttatgtgaagcaggtgggctgttgacacaggctgagaaaggctgagtctggacctgaagagttttaagttcaaactcccaaagagaaaacatattaatcctctctggactgcaacatgagaatccaaggtcttaacttgaagctccagcacactggatttcggctttcctatctgctcttccagttggatgacaagccttgctgtctaacacctgctgcagatcccttgcgttatgacgtccacattttgctcacattgttctccttctggaatattttcccctgctcacatctccaaaaagccaaattgatttccccagattcagctctagcgccctcttctcaaagaagcccctcagcctcttttgagcttactagaaccaagggtttggactttacaactgaatactaattagacattgtcacctattagtcttgaattctgtgtcttgactcttcaactagacaattttaagccttcataaacagggattatttttctctatcccgaacttttccaagacaattctgtgcccagggctgttttttctatcaatacctcaaggatatgagagtaggaaggaagaggttcacagtgaaagccttgccaaaatcaggtaggatctctacgtagatgggcagaggaaggcctaagacctgagcaatgtgagcccatacagacccctgagagcagcagctgagctaagcagcccctcgggtgctaacttctattctgagtaagcaactgatcaggccacagtgggaagatcaaagggctcataccagcaggtgaaatcaacctcatctcctccaagatgaagataaaaatctgggaagacagagctgacttctaagaagaatgtgctcatgaactcataggtattattgagactgggattcactggtccaaaggtgccagagggctcagacccagagtagcaaggagtcagtaatccagggatacctaagccaagagaaaaccccatatgagtgtcacaaatacataaacccccacgcacagtcctacacgtaaggacacgagtcacacaaagcaagacaagtgtattcataaacatcacacacatttaggtaggcacacttaggcactcacagacactggtagtcaccaccatttagtcacagtaaaaagatacaaatatatccacaatcataggcagaggctaagttccagccagacaactgtcagtgcgcacactcttgcctggacacactgcttctagcaggtgctacccttgctcccaaggctgctctcctcacaggccttgaggcagagagagagcctaatccagccaaactggcaaaatctttcttctctatctcctcctgtaggtggggtcaaccatcacccagaatctttttacatttgctaagggatatctcccttggcccagagactacttcctgacgctgaggtcacaagatatcttcagggtttttttttggttgttgttgtttgtttgtttttgagacagagtcttgctctgtcgcccaggctggagtacagtggcatgatcttggctcactgcaacctctgcctcctgggttcaagcgattctcctgcctcagcctcccgagtagctgggattacaggcgcctgccaccacgcctggctaatttttgtatttttagtagagatggagtttcaccatgttggccaggctggtcttgaactcctgacctcaggtgatccacccgccttggcctccaaaagtggtgggattacagacatgagccaccacgcccggccaatatcttcagttttctatcaggccagtagcccttttcaactgtcatttaacctgtgttgggggctcacactggagggctgagggtatacagaagaaagggagcaggggtactgactgactctgacctctgcaggctgcccttgggcttctttctttcagattctaccctggtggtcagattcctcagcatccactctaggcacaactacctgcaacggcatctgagaaaaatgtgcccttacatagtctaacagtacatattttaatagtaagaagcagcctccattgtccaaagtgagttcttcctatctaaattcccaggtggaagaagtcgatggaaaacattcttctaaggaccaaggctgggatatgccacttccatgagccagtgccctgaagcttcactctgagcataacaagcagagtccctctggtcccagacatcattcttacctggtccccaggacaaagtgtggccaggagtgtcaaactctgcaagcacacggataccccggagccgtgcgtattcaatgacctccttcacatcctgtgctgtgtagatgtgggtgacagggttgtaggacccctgaaaggcacaagacacccttcaggttcacacttcctgaaagctagcagagtagaagatactcaaaatgcccacaagactccccagatatcagaaaacctgcccatagccctttggtgtcagggactatcttcaaaaaacttgatcataatttcccagaagttatcacatctgttttatctgagtatcataatgccagtgagataatcatggtaggtattaatactatgtccatttaacagaatataaatacataaaaagggaataaggccagaagagattctctcctgaaggtcacaaggcaaattatcggcaaagttttggacttgaactcaagtctcctgactccaaatccagtgtccttcccctatattggtctaaaactggctggttaggatgagagaccctgttcttgccagcagggccacagccagattcagacattgacccataaacttggtctgagtgaaacgggaacatacctttctcatgagctctggaaaagtgaagctctcatatgggaaggaaggatcatctaccagatgccagtggaacacgttcaatttattgtacgccatgacatcctgtaggttaaagtgcacactgtgaacccatcacagtctctccggtttcagcctcaaacttgcgatgttgggcgagctctcaggccgctccacacacccctacaggcttgacctgcctcagctctcaattaagtatttatggggtctatcaaaccttcccatcagggagggatggcatggagggaaggcccagcacacttctacttttcccagaacacatccaaagatggatgatagaagtggtctttcctcttcttgaaataaattctggccacattaagaggagggctgcagctactgtggtagcctggaatcttccaaaagcctgaagatcaatacttcctcttgccatttgtgttcggttgtctgactgatgttaagcccaactgtgagacctcctgggaacttagtagcctcttaactcataatctcagaagcaaaggctggcagatgtgtggcctcctttggttccgtcacaggagcaaaggaaaaggcagacacaggaactggattgggaactgtcagataagactgcacattaaactcaagagagttaggaacttccccaggaaaggctttttttttttttttttttttttttgagacagagtcttgctgtgtcacccaggctggagtgcagtggcacaatctcagctcactgcaacctccacctcccaggttcaagtgcttctcatgcctcagcctccttccttccttcacttatttatttatttatttatttttgagacagggtctctgttgtccaggctggagtgcagtggtgcaatcacagttcatgcatccttgatctccccaggctcaggtgattctcccatcacagactcctgagtagctgggactacaggtttgcgccatcgcgcctggctaattttttgtattttcagtagagatggggtttcaccgtcttggccaggttagtctcaaactcctgagctcgggcaatccacccgccttggcctcccagagtgctaggattacaggtgtgagccactgcacctggccccaagttaaggctttgaacgtctctctcctatgaccttgttctaactaaagttagctctgccggccaggtgtagtgactcatgcctgtaattctaccactctggcaggccaaggtgggcagattgcttgagcccaggagttcaagaccagcctgggtaacatgacgaaaccccagctctacaaaaaatacaaaaaaaaaaaaaaaaaattgagacgctgtctcaaaaacaaatatagttagctctgccaatgaagccaattcccctaaacatttgtatccctggaaacagcctgaacattgatctacagtagcctgtaaggtagatttaagtcactctgttattttttgagtcttggagcagctcatcttctacatttttcccaggagctcatagaattacatacaatttttttttttttttttgagacagcgtctcactctttcacccaggctggagtgcagtggcgtgatctccacccactgcaacctctgcctcctgggttcatgcgattctcctgcctcagcctcccaggtagctgggattacaagcatgcaccaccatgcccagctaatttttgtatttttagtagagacagagtttcaccatgttggccaggctggccttgaacttctgacttcaggtgatccgcccacctcggcctcccaaagtgttgggattacaggcatgagccaccacacccagtccatacattccctctctaactctttaagaatttggaacttggtctgtccgttgctccatcaccctagaactcttaagtgtgaagaaggccttaaggcctggttaccagagtgtccaggatgctagagagtggcaggtaatggcgagatgtatccaacagcaagccccggtgaggaaagcggggaaagtcctcaatctcagtcttgttgataaagaactgtgcagaacaaacattgaacatgtcagtttcaaaggaagcttactatgggggcacagggagatgaagacaactctcccaggattcttaaaaagccaatctgtgactgttctcagggtcgctctctccctaaatgctccccacctctataaatgaccataagcagccaccttcccagagagcaattgtccaacgccagcctaatacaactgtcccaaacaccactcagcctacaaattctggattttcctgttaacatagtttattttcttcaaagcagtgtggcctcaaggtcattactaaatgatgactcagggggaccaaattacagtgattccaaacagaccaaatctaccaaaccagttttccactcacatctcctcttccatttctactggggctatctcaagccaccaggatcctaaggcaaaacaagatttttctaggattctcaatattgggatccaaccccagagatgaaaaaggagccctttttgagggtccacacttactgtgccctcagcagatttccaaacaagctggctaaaagtctccagacctaggaagatgtagagaggcagagtaaagactcagggagtggaaaaagagatgtctctatttggaaggttctcaatgtagcagagcataacatttgggtacacagggaaaatggatgtagtgtaggcttgacgatttttaacactgatgaaaaggggatattaggaagccactatccatctttacttctgtatttcattttgggagatgtcagagtaagggttttagtgcccttccttacaacatactaaccttcaaaacccacatacctcttatctatatttcctgtgccttcacaagaatattgagaagagaggccaagacatatactcatgaggacagacacagagacaactcagcctgcaagagttggacttttcatggataacccctatcaggagctaagagagctgagaaaaggggtcccagaataaattgtgagaaagtgatcataagcttgggtttccaaggagcctgggttcatggctggacctagaagataaaaaaaatcatccactcttcctcttcagagtctaagaagagacccagctatagggagctgaggtgtggtggacagcaactgccccagaaggactcccctagtccactgacactgtctcctagagtctagcaaggcccacacacaggtatgttgaacccagagtattgtatgttctcaacagctggggcccctcttctggaaaccctgcagaagccccatgaaaattatcagagtccccagtttcaaagtgaaagaatccatgagacctcagcagtcactcagcacctgatttgagcagactgctagtgtactgttttggcttctccccactctgactctgacacccagcattctggagttccaggtcctcccttacctctagcctgactcaccatcagcacctgcatgaaaggtacaagagggcaggaaggggaggcagcatgctgttgctccaagcatagaagtggagcaagggaatgaagacaaactaaagcaaggtgtggaattccgagagagtttcccacatcattcaccagcagagggtttaccaaaagtgggagagatatttggctaagtctgtatctaggcaccaaatgccttctcagtaaacccagaagaagggcaagggccaatagtttagccagtaaactcattttctccagaaataatgtagttctaaatatttcactactatttatctttttttttgcatgtatctatttcttttgttatagttttccttttaaaaaaacctttccaattacatgtctttagcaagagctggacagacagtgtctgtggtagcatcattccccctgcccccagctacctgtcccctgctggcacctagaacatcagcccacagactctcctactgcttcccagagcccctgactagggcagcagcagttgctgatgctgcacagggtaaaacctgtcaacttccagggacaattccacagagacgggaaaacacgccaattgttcccataaaaaaagcttcctggctgggcgcggtggcacacacctgtaatcccagaactttgggaggccgagatgggtggatcacttgaactcaggagttcaagactggcctgggcaatatggtgaaatcccatctctactaaaaatacaagaattagctgggcatggtggcaggcacctgtaatcccagctacttgggaggctaaagagggaagattgcttgagcctaggaggcagaggttgcagtgagcagggactgggccactgcactccaccaacaccaaccttcccacatcatcctttctctctctcttttaatcagccccaatttgttacctcggagagctccccagacagtctcagagaggagtaaacactggtcatcatttatggtcagggtatctgaaatgacagaaatgaactcatttagttggttaaggttttctattctcagattatagaccagatattctatataaatcactggactaaaaaaaaaaaacagtaagaccatatattttaaaaaatctttcaatgaggagatgtccccagagcaagacagctgtaggatggcctggtctggggccagagtgggagggtggtcaccacagtgactctgtagaccctggatgatgatgtacagcaagagtcaggctggagtggaggtctgtacaaagcaccacctacctcatgggtcagtttccacagcagaaagatgaaagaagtcttctcctggcaaagggaacagggatgcctgcaggggtgagtgcagcacgcgatgctcccaatgcctttgtaatcaccctcctttccccttcctctatgcttcagctacttcctccaagaatgtgaggaaggagccaaactcaactgttcactttggagactgagaagtccccaaatgctgctcagggaagacaggaggcagtcactggactaatgacttaacttcatctcagtataagtgaccagaacccctgctttccccagtgtagggtctattagagaaaagtagcaaagtatacactttcctaaaatagaaatgtattcagtacaccaggtagaagtcctgtgggactcagtgaagagccagtgtgatgttccacagccaacctttagcactggttaagtttctgcataggaaaaactcatcttccatggctctcagcacttggtgaccctcggtccccagggtcaagggggactccaggccgagcatcagcagtttaggccaggccatccagagttacagcttcagacaagtgtttgctcttctaagacagggaacaggatggtacttacaattctccactgactccaaagtaggaagctggttacatccaggtgtgactacagagacaaccaacacattcttctccagtgtatgccgtttccctaggaagacagggtaagcttggtgcggccaacctgccattagaagcaacaggcaaaaccaagaccctagctctagtccctcagctcacacgcctgtggtaaaggaaagggaggacatggcatttacccttggcataggcagtgcctgatcatctgttcagctgtctcactctcgttgtgcccctcttctcctcagtagctgccctgacccatgtctgaatgctctgggtgacagctcttcttaaccagggctctatatttctgactgcccttggcctgtaccaaacaaacaactccatgacctcaacaactgcttctgaatgaaaggcagctctagagcactgctactccaagtatggtctgcagattagctgccaatttgcaaactgtgttactggctggtcactatatagaaaacttatatgaagcctgaatgtaaatcaattatgtcagagtaaacacactgtctagttctaacattttttcatacaagattttcttgatgaagagatcagtcttacattctggtacatatgcttcttatctcattgaagattggcatattaagtagtactgctcttgagggctccagatgaagggtttccagttttgcaaaagcctaggagctaatggagcagatgacagaaggatcaggcagaggaggcctgagatgatgcaactgatacctcagagaagatcagggtcacccacccatcttgcttccttccttcctcttcccaaacaggttctctatccaaggtagattttaccctaatagtggagggttgaaccaacatagtatctaatatagctttacagacccttgtctatgcagaaaaaatgccaaagccaaaatatgaatacactgtctcttctcaccctcctgctggttcttccctgtgattttcagggtggctctggtgttatagccccaagccttaacttaatgaacccctctctattctattctttcctggagccatattcatcccactggcatcacaacatccatgacttcttatggcactacaatgtcagggataggatggcctggcagaaggcaatgagagaattcagaaccaaactggaatctcaaatgttacccatctccaggcagaaggaatccaagcgctcagatcatctcacggaagcctgctcagacctcttgagcactaccacatggctgtatctcagctctttggtctgccaagaagcaagccaataagggggcctttcctaccatcatgaacaacacattctttcccttaccagtaaataccctggatccccacaccaatacctgcctacattctgcaaagaggagggaatctgaggcaactctgggccaggtcaattcacagctccatacttgaaccctaaaactacattgttacaacacaaaaggacaagcagaccttcaaggcaaagaccacactcaaggcagaatctgcagggccatccaagcagacaggctacaaacccatctccacaggagactctacagggagaccaatgaggacttaacctaggagcaagggtgaggaaaaactataccccatcctctgacttcttgcccccactcccaaccccacacatcatggagccccaatatcattgtgtggagaattcatctgcatatctacccaggcagccatccaaaagacagccgaagtcctgagctttagttctaatcctctggggctcagcctgccacataaggagaaatcacaactaatccatatgcaactaccatggctcgctgccctcatgacaaaataagagaggccacacaaacaccaggtttatctgaactattacttggaagacagaaaaatacctaatctttcgatttttaaaaaatttatttttctaaaagacacctgcactgtactccctaaatctgtagtcttttggagagtcacagactcctttgcagatctgctgaataggaaaacactttttttcccagaacaatgcccctaatttaagggctgtttctccagaattacaattcttccactccctgccatcaatctcatggtgaagaatccctgtccgaagttctctcagtgactcatctgggtttgggctgaaagaagtagatacgggggcatggctgctgtttaaacaaattatgagagggatgtttgggggaggcagcaacatgagagctggcaaaacaggagggacactaggtcattgtaaaggatgtaaatagggtatcaccactgtggttttccgcctccaagttgctactttgcctggctatgaagagggcaggaaataggcaggggacagtagtacagccgcctctacacacacccgcccacaccctgaacactgagaagcaccaacgtgtgggaagcacagtatggcaaacagcccggtcagccagcaatgtcatgagcacatgtgccatgagtctggctggaacagcaggaaattgagaagcctccagggcttaggaggtagcatgaaaaacagaccagagggctgggaaatcaggacaggttgcttctggttctggttttgctgctgccaagctgagcaagtctcagggctcccatttccttccctgtaaacaatggcagggtgggaccagattccagctactcctcttcagatctaagaccttgtgattccatagacagataatacactccagggtcccattccaggaccacagcctgctacctctgcagctcactcatctccctgctttcatgtaaacctactcttctctttgggttcactgaaaactttgtatctttttttaaaatcaaaacagttgtataggcctggtgcggtggctcaggtccatgatctcagcactttgggaggctgaggcaggcggatcacttaaggtcgggagttagtgaccagcctggccaacacggtgagaccctgtctctactaaaaatacaaacaaattagctggtcatggtggtgggccctgtaatcccagctactcaggaggctgaggcaggagaatcacttgaacccaagagacggaggctacagtgagcagagactgcaccactgcaccccagcctgggcaatagagtgaaactgtctcaaaaaaaaaaaaaaaaaaaaaaaaaagagtagtacatactggttaaaaaaaaaatcaaacagtactaccaaacagtagaaaatatttacgattaaaaaaaaaaagtatctttggtacccttccttagtcccctcagtaaagagacaaacatcactggtagttttctacccctgtagtaccttatagagtaattttgccttattcatcacagacaggtcacctctggacatggaattctaataacaaggacgctctatcaccaaagtcctactgttgaaccttctccagactccagtactatgacccctccatttcccttctgttctcagtcttctctcattcttgcttccttccctgctggcctacatgtagtgattcatcacctcaaacgcaatctagccaatactcccaacgatctgtctcttttcttctgtcacaaccaatgggcaaaactttcaaccctggatcaatctaacatctgcctgctgtgcccctgaccccggctactgtaccctactggagggaagggagaaaaatcacaaaacagcaggactggaccactgcaaaataatggtctccatcttggctgggcccttgatgctaccctgaaaatcttctatctccatcattgcttcattctattctctacaccttcctcaagtctacttgatcaccacctcccttatgctcagcagacaatcagaagagaactttcaactatccctaccctcttcccctctcctcgctgacaaaaatatctgcaacacacacctgcctgtaccctctcctggacctccaccttgatggaaggggcttccttttacctgtgctctggagctcttctcagtctcctcttgtataaattatttccccttttcccaaagccttcaaattatttctttcaactctcatcagcattaaaatgtttgcaatcttcctggcctcacttactctttcttttgccttcctgccacaaagactccctagaggaaatgtagcctgggcagctaaggacctgaactccaaaggaagacagatctggattcaaatcttggttctaacactttctagctgcaggccttctctaggcaagttatttaccctctctaagctgcagtttcctcaactgtaaaatagggatcataatacctatataattgggctgctgggaaggttaaatgagttaatacatgtaaagtgcttagctcagtgcctggtacctcctatgtgctcatgatgttggtcatacttagtattttgtagctgcttccttacaacctatctattcctcaatccactataatctggcttccttcaccaccacttctctcaaaatggcttcaccaaagtcctgtggttctgtaaccaaattcaagggactcttttcaattcattcattcattcaacagtaatttactgaatgcttactatgtgccagggtgttgtaagtgctggggatacagcagtggtcaaaacagacaaaaatcccagctgtcagagagctaatagaaaccactcactccttctgggacctcttgacagcatcagacatctctgaccttcttgaaactaaactcccttagcttaggggacattgctctcttggttttccttctaactccttgaatatgcttcttcattcaacttctcctttaccaatcccaatgtactaatgttacccagggtatccaaggttccaaacttctctttttcccttcgcttacactctccctagacaatgtcatccactcccacagcaggccaatgactcccaaatctatctttccaagccaactcttcttctggagctctagacttgtacacctccactcaaatgtctcaaaagccacttgaatcttaaacataaaactaatctaactcttcattcctcctcctggctgatcctctctctcactttgataacagccccatctattcacctataccagaaatctccttcttctacaccttccatctaactgagtagatcacaagttcatcaactcaaaataacatttacagactgtctgatatctcaaacattgtgttaagcactgagattacaacagtaaaaatactttttaaaagtcttacaattacacaatccttataatctcttaacacatcgccattttcccagccccactgggccaaagccttaattcagcctcttgtaatttctcacctggatgactataaaaaatctcccaggcagtcttcccctgcctccaggaatacccatgtcaatctactatcctcagagatattagaatatgaagataagagcatgacagtctccgcttaaaatcttccaatggctcccatagctttcagaatacagtccagaattcactgggtaatcataagaaaatgccttgtctttttctgctcctgtgcaggaactctcacaatgaacaatgaactcttaacagaaccctgaaaatgccatattttctccatatttttctttgcacatattgcttcctccaattgaaacaaactttgccttcccgccacatctcccctaattaagaaacagctctattttccttttaggaaacttttctgttattccccccctctttcctaagaaaccctcctatgtgatcatgcagcaatgtatacatacatttttcaaaacacttttcttaagatatgctgtttatttgtctatctcctcacctaggttgtgagttcctcaacaggagggaccaatctcagtgtctttgtattcccagtgcctgacacacagcagtcattcagtacatgtttgctgaaaaaatgaatgatgttggaattcaaaaaatgcatattttatgattgtgctatgtgataatcaggcataaatagcaagtagttaatgtagagattacttttatatccccaaacctagtatttctgaaatcctgagactggggtgggcgatgctaagaagacgcccagacttacacaaagtgccttttcagacactgctctgaagacctctggatccataaattctaagatttaggtctttccacttctctgagaggaagaactcgttgctgtattagggtcactaataactattagggtaacctggaaactatcaatctttacctcagtgcacaatagcctttccctctaccacccgttccctcctgaatcaaaacacttccatcgtatcaatgtgtttgattcacagaatcccagcactatggaaaaaaaacacattggtttcggttataaagtggtagctgattttgcttctggattgaaaactaagaaaggcaacccagactttgccaccttcccactctttaatgtctactctgctcccatatcccctggtaccctcctaggctggtgccaaagtttgttcccatgcccactttcttctcttccccctcccacctttatacacatcatatccaggtttcatctcccccatattctgcctaataaaagcccattatatcattcatttcccactcatttcttcattccaaccaagatatattgtgcatttaatgtgcatcaggaactgtgctaggtgctggggcagttaaaaagaaaaacaaaccctggtccctgcccttgaggagatcacagtctagcaggagagatgaacacaaacaataattacaaacattgtgataagggctatggtagatttaatagccacgtccaggaaaggtggaaggcttgacgttgaaggggacctctgagtgggatcttgaaccctgaattggtgttcaccaagctaccaagaggagaaaaggtattccaagcctgggaagtgaattggtgtagctaggagagaagtcaaggccaaccttgtcaaacttcttatcccagtaaagtgggagtctgtgaagccgactgccctgagctggggccttctccttagtgctgctgaatttagtctagatcatggtacctcctcctgtaagttccaagattcttcaccctccacagactattaatacctacttggaagtatcactctctccagcccacagatgaatcctgaatgttcttccttcctctcattcccatcccatattttttgggtaaaagttgtacccatctttttttgggaaaagctgtagttaaaaattttctattttcccctacttttaaaactgatctgtccctcccaaccctatccccactggaaagagaacatctccctaaaattctagatctcaagggataactttagttataacattttgcacaattttccaatttttttgtaaatacataaatttattaatttttaaaataaaaatgggcccaggctaggtggctcatacctgcaaccccagcactttgggaggctgaggcaggtggatcacttgagttcaagagttcgagcccagcgaacgacttcaaggttcgccagtcaacatggcgaaaccttgtctctactaaaaatacaaaaattagctgggcgtggtagcaggcgcctgtaaacccagctacttgggaggctgcggcaggagaatcacttgaacctgggaggtggcagctgcagtgagccaagcgaccacgccactgcactctagccttggtgacagagcgagactgtctcaaaaaaaccccaaaacaaaaaacaaaaacagaaaaaaaaaatgggatcctaatactattagtttgtaacatttttccccttaccaaaatgagctgtagatctgttaatgtcaatatatatagatttacatccttcttgtaaatgttgaatataattccaatgatgagatatacattaacttacctaacttctcttctgtttgtagacattcggtaatgtttataagtttttagtattataaacaacactctagtaaacaaatattcatatatatgtatataagcaattgtgaaagatatctgtaggataaattcttggaggtggaaatgctaggccacaggtaagaaaagttttggttttgatactgccaaattcctcttccaagttactgtactgacttacacccaaatccacatatatgggcccatctctccacatctttccaacactagaacatctcaattctttttcaccattgccagctggatttaaaaaaacacatacattgtttcaatttcaatttgtatttctttttaagttttaatttaattttttttttttttttttttagagacagggtcttgctctgtcacccaggctggagtgcagtggcacaatcatagctcactgcagcctcaacctcctaggctcaagtgatcctcctgcctcagcctcccaaacagctaagactacaagcgtgcaccagcatgtcaggctaatttttatttttattttttgtagagacagggtctcactatgctgcccaggctggtcttgaactcttggcctcaagcatttctttgcatttactagccaaattttttttactcttttttttgagacggagtctcgctctgtcgcccaggctggagtgcaatggcgcgatctcggctcaccgcaagctccgcctcccaggttcatgccattctcctgcctcagcctcctgagtagctgggaccacaggcgcacgccaccgtgcccggctaattttttattattattattatttttagtagagacgatgtttcaccatgttagccaggatggtctagatctcctgacctcatgatccacccgccttggcctcccaaagtgctgggactacaggcgtgagccacggcacccagccattctttttactttttaaagttacttttcttttattgacttgttgcaaatatctgcctcattttatcccttattttttaaccttatgctttcttttgctttaatttttatgtagtcaaagctatgactcttttcctttacggtctctttatttgcttacttagaaaggcctcctctacccctaaaattataataatagtttccttcatttctgtctaacacttcaatggtcttttatcttcagatctttaatctccctggaatttacttttgagtttgatatgaggtaaggatttatttttttcccaattggataaccagtcatccttaggtatttattgactaatacatcttttcacatttgatcagaaatacttctttgatcatatattaaattcccacatatatacacatgcttgcttgtctctggaatctattttcttggtaatcttattgtcagggaaatccgccatgtcatatctctaccatattttgtcactttgcccaatatataaccccctttgctcacttctggtcctgcacttctatttttatacatgttgcttcctcttctccaccagaccacctgtgtactacatatcattcaccttgtaaaccctctgagatcttgttgtgtagttacttaattactcagcctcttctacttcctccctaactgaagcctggctctccccaggaataccacacaccacttcccctacaacatgctctgagaggcagccacttcccacatcatagggttgagatatggggctgcttctgggtcatccattccaccaccaccaagtaataaaacacctttcttctgtggttcatgtcatccagctgttaaatcttcagctctccttgttacaacactctgtagcttcctgatcacttctttattcacttaatttttttttttttttgagatggagtcctcctctgttgcccaggctggggtgcagtggcgcgatctcagctcactgcaatctccgcctcccaggctcaagtgatcctcctacctcagcctcctaagtagctgggattacaggtgtgcaccaccacacctggctaatttttctatttttacaaattagacggggtttcaccacattagccaagctggtctcgaactcctgacctcaagtgatcccacagcctcagcctcccaaagtgctgggattacaggtgtgagccaccatgcccaaccttatttacttaatactttggatcctgttgcccagtattcctctccacctcaaggctggctattatcctagatgattttcacacccaagaggaagaactaacccatgttctggcttagcagtcatccaacaacatcctcctcctcctcctcaaccaccctcctgtggttgccttttggaccttcttctcctcggaagtgttcaaactcattctcccaagctctagtactacatagctaactgtttgcttgatacctcatagtcaacacgtcaaaatcaaatttacttttcatgattctcactctacctctcaacctcttaatttcactgttttttttttgttttgttttttgagatggagtttcactcctgttgcccaggctggagtgcagtggcgcactctcggctcactgcaacctccacctcctggattcaagcgattctgctgcctcagcctccagagtagctgggattacaggaatgcaccactatgcccagctaattttgtattttcagtagagatggggtttctccatgttggtcagactggtctcgaactctcgacctcaggtaatccacctgcctcagcctcccaaagtgctgggattacaggtgtgagccaccacgaccaccaatttagctctttttactccttcccttcctctcctactattttccctatctttgttaatggcagcacgagtgaatgagagccaacattcaaactgctaagtcagaaatctgggagtcatcctcagcaccctttccctccaacctaactcccacttccaagcagccacctcctaaatctctcttcagtctttctagttttctctgtccttacagccatcaacccaacttcagccactactctctcttatctagaccactgttggcagccttttaactgggtttcctgcctctaatcccactctacagcaagccccaatgtagctagagtttagtcacatcacttatcctgttgactggactttactggccttctttgaccttaggataaagctccattccttaccagggcttattaggccatttcatgacctttccatctatcacctctttagcttcagaacttgctgatccaccctagagttgtgtacttcagctagacaccttgagttccttgaatatggcatgaaggcttctcgcctgtaggccttttccacgcgcagttctttctatgcggaatactatcttcacccccactaacttctacttttagatctccttcaacagctctctaggaaagccttttcaatgtgcactttctccttttccccccatacacttaggtcaggtgttcctcttctgcactcccaaagtaccttgtgcttcagttatcatagaaaaatatcacactctgtctgtatccccacttccagaaactgtaaattctttaaggacggggaagttcaatttaatattccttggacctagtactattgacaattaaagttacttaataaatactacctgtactaagaatagatgggaggaaggaagaaatgctactggaatctggtaacaatgggccagggatcaaaatattcataacctgagtgcttatgagtaactagatgttagaagttactacctatttgaagtaattaacactaagatgtgattagagcttaatcctgccagaggccagtagttttccaaattgctaactggactgacaaagacgtgtatgagtatattacaagactgtacagtgacaaaagcaaaatttatgcatgactggaatttctgctccatccctacaagctgtgtaaccctgggcatgttacttagcctctatgtctccattctcaaagattgggataacactgacctcacagagctgtttatgaagattaaagggcaataacatacatcaagtgccagacaataaatgctagtttccttccgtgttctacttcccttttcccaataagacagttttgccccagcaaacctcgactctctcatttggccagaggagtcctggggcagggtctcctatggcaaagccacactctaggagcagcttccctgaagggtttttcatgagcctcttataggagactgaaggcttcactactgtgagatgccattctgtatatgaacaggctgacacttcaaaatacaagagggacatggccttagaactctgaatctcccccaacctaggactgcagctccaggcaaaacaacaaagccactaatgtggaatcctagttacaacaaggacaaggttgaacaaacagtaagtagttaaacttttcatgcataacagtgtacctagtaaggcatctctcttaccccctcaatcccaagagctttacaaaagaaggggaaggctgcttaatgacagctgacaaacacccatacgtcctgtatttttgagtatacgcttccacagaaaggagctctacaccacacccaagcctaaccagcctcaagatgtggacacaacaatagcatacagcagttggctggggatcagggctgagtgtgtggccaggaagcttaagcaaatgccacagcttgaaacaggaacaaaggacaagaagagagaagactcaagttcctgacccaataatgggttttattcattacagccaggaatacagaagaaaatataatccatttctgtattacaatggactgtaaacatgtaaaccttgagacagctcacaccacagaaaactcttcacacatacacctctctgacccctacagacccaacctcctttcttcagctgactttgttgtcagtggctccaaaaaggtcatggaacctcttgaggatgatgcaaattgactctattttatcacttattttcaggaccagtagaagaaatttaggaaactgcagaaacataagagtgatccctttcttttgtttttgtttctaggtttttgtttgtttgtttgtttgtttgagacaaggtctggctctgttgtccaggctggagtgcaatgacacgatctcagctaactgcaacctccacctcctgagctcaagccatcctcccacctcagcctctggagtagctgggactataggcatgagacaccatgcccagctttatttcttaaaagaagtactggtaaggctgtaaaactcctttccctctatataacaacaggaaagggattgaggcttatgccttgaacaggctaagaaacagacaacatgccctagcatgagacggttaaaaacaaacaacaacaaaacgacaaggtatccattgtcaaaaaccttccaatgctatgcttctcgctcctgtttggaggtaaggaactggggtagaaggaaatggaaggaaaggcatctctggggcttggacttttaatttttattgttttatttttaaaatcaccaagttcttcccactattataccctcccttaaagactgaaaaaacattcataactattttcttgttatcctcggaaatgtctggaaggcaggcagagttaaaaaaaaaaaaaaaaaaaaggaagtagcagcaacagcttggaaattaggcaattagtctgaaggcacagagttaaggggttaataaacactttctcagctgactcctgaatgaatcatgttccacatcctgagtcctccaatgtatactgtgttcaaacagaatatccaaagcaggggaacacctacagctcacagaaataaaactcaaatttttcataaaccaggcccaggctacccacatacaccttgatcaagtttagcttcttggactcagtactatacagggatactagaaaagtatcatgctttaggtaataaatggtggctcacacctgtaatctcagcactcggggaggccgagacaagaggatcgcttgaagccaggggttcaagaccagcctgggcaacacagtaagacccccatctctacaaaatgcaaaaaataaaaaaattagctgggtgcggtggcatgcgtctgtagtcctagctactcatcagtgaggcgggaagactgcttgagtcctggagttcgaggttgctgtaagccgtgatcacaccactgcactccagtctgaatgacagagaaagacctgtttcttaaaaaaaaaaagaaagaaaagaaaagaaaaaaacagggctgtgagaagagaggctggtggaagactgcaggccaagctaatctaggccctgggattcaaaatgaaatatgacacttattttgcccttcaactaaaaactgtgcttctgccatcaccttctttctaaagacaatttattcatcactgtatttctcctatgtatccctcgttctccagaaaccccttcatgagaaaaagtaattctactacaaaaccttcttcccgtgttgttttcttcctctttgcttttaaaacaataaacttagtggcaatcatctttggcaggaatatgccttgtctttgccgtgggaaaggttaaagtatagttagccgctagcccaattaaagtggagagaactaatcggccttattctttctccccagcccagctgcttgaccttatctgtttccctcctagatgcaatcccaatgcatctctcactgcatgctgagttaagagcagcacgagttgggatcacacaataagcagcatagtgtgattatcctttgcctggaatcacacacttacactccaaattggtaacctgaaaggaaaggtaatcctcaatgaaaacctgcaaatcatatctttttgatgatcttctcagacctgtaatcccaacactttgggtggctgaagcaggcagagtggttgagcccaggggttcgagaccagctggggcaacatggtggtactccatctctacaaaaattagctatgcacctgtagtcccagctactcaagaggctgagatgggaggacagcttgagcctcggaggtcgaggctgcagtgggctgtgtttgtgccactgcactccagactgggtgacaaaataagaccctgtctctaaaagaaaaaaaaaaaaaaaaaaaaaagaaaacaaaacccaaacccataaagccttcttacaaatgtttacacaaagtagctctttaacgttaatatattttacaaataattactacatatctacaacgttccagatacagaagaaatggccaaagaaggtattttaggagatccagattataatgaactatgccatttattaatttagtcacttctttaatatctgaggatgagcaaatcaaatactgataatggctgcagaaggacacaggctcttatataataatcttacttccattccagaataaaagggctggagaaggggggcaggattattctgttgaaggacaatggcatcctcagcaactcacagggatgtgcaggtgaacatcagaccaggatgggaaccttataatatctatgatatactatggcttctatcaaccagtttcaggaaaagcaaagaagaatgcagtcatggccaggtgcagtggctcacacctgtaatcttgaggtgggcagatcacatgaggccaggagttcgagaccagtctggccaacgtggtgaaaccccatcgctactaaaccccgtctctactaaaaaataaaaaattagccaggcatggtggtgggtgcctgaattcccagctacttgggaggctgaggcaggataatcacttgaacccaggaggtggaggttgcagtgagtcaaaatcacaccactgcattccagcctgggcaacagagcaagactccatctcaaaaaaaaaaaaaaaatgcagtctaatggcagaattagacttagccactttacttggaaaaatttggactttatcagctccatcaagcaccatgctccacaccatgcttattaaatgtctggagcaacattagatgggtgccattccaaattccaaataatcacatctttgctttgcaaacaggatgtggcttaataatcaaacagggaactagattcattcaagtatctagttcctaaaataagtctatttaacaggttcaacaacatgcaacacacaccctccaattggtagaaacacctctcaatgacaaagaaaggtggcaaatactaaacaccttcacagaccatttagcaactggtgggactaacccttgaaggtgaattcaaaatagaactgaacctcaataccagacttctttttcagaaaagggagtatgaattcttagtttcaggttcagacaaaaaccagaagttgtattgcattaggaaaaacactgggctggtcaggtggctcattccaccctgtaatcccaggactttggtaggccaaagcagggggatcacttgagctcaggaattggagaccagcttgggcaacatggcgaaaccccatctacagaaaatacaaaaattagccaggtgtggtggcatgctcctgtggtcccagctactcaggaggctgaggtgggagaattgcttgagaccgggaggcagaggttgcagtgagccaagattgtgccactgaactccagcctaggtgactaagcaagattctgtctcaaaaaaacaaaaacaaacaaacaaacaaaacctactggcccagagcctacaaataagttgtttggaggaagaggatggaaatctgttagttaagatttaacctgcatatgttgccttctttactaaaatcagactcataaagaaatgtattctggcttcagccttgttggtattgctctacctctatttgtttcccatgctattgcttcatccttgtttctgcagaactaggattttagctcaaagtcacctaaaagcctccacaggaaactacattccaaagggaagactccatgggaagagcaatagagaaagaagttacttttaagtctttggtaatgttcacccaacccagttttcattttctctcaatggacaagaaccctcaactaataaagctaatgaacttacaggaaggtaatatatggaaataaacatcttattgaatttatctggctctaaatctcttttgttagggaatttatagtattccattatcttacaatctctccatttctatctacttgattaaactatggtgctatagcaatggaggtgaaggcctaatttagaagtataggaattaggtatggcgcggtggctcatgcctgtaatctcagcactttgggaggctgaggtgggtggatcacttgaggccaggagtttgagaccagcctggccaacatggtgaaaccccatctctactaaaaatacaaaaattagctgggcgtggtggcaagcacgtgtagtcccacctactcgggaggctgaggcaggagaatcgcttgaacccaggaggcggaggctgcagtgagccaagatcgtatcactgcactcagcctcagtgtaggctgagtgataaagcgagactccgtctcaaaaaaaaaaaaaaaaaggaaaggaattattctcatgtatacaatagtaggaaacaacctataaagcttttgagaatcttataattcactgtgtacctccctctgtttcatattttcgcaattgaactatagagcctaggcctaggtcttaagacttttcttgtaaccttcagagtgcccagcactgggattgacatataccagttagacggatttttttttccataaaccaggctcaaactcttctaaaccaaaatcaacatattctgagtgccaatcttgggtaaggcactctattatttgtcatgaaggattcagaggaaaatgtgatctaccacactcaacagcaggcctcttatcctctccagcttaatgcattttaccagttaaggactaccctgatgctgagattgtgtttctttccccaaatcaaatgtcactctctaagggtcatctgtataattaaatgcttttaattctatatatcaaatgataacttttgtgaaagtaatttaaacacacttgaaacatcatataaaatattactacaagacttaaccgtaaatgtggctcccacccccataacatctatacaatataaccaaagagctagattccagattatcaagaaacttctaaattttgaacccatgcagcagaattgtattcctttacattgttggctcgacttacaaatgtgctgtggaataattatcctcaaatacaattttcagtactgattacatagctgcctaaaagaaaactgttagccccttccattttatgtgaaaccaatgtctaaagcgctcttgactcttcaaccccacattttccccaccctcatccctcctcaatccccagcccccactcatcacatccactagacctgaactttgaactcaatcataattctttgtgcctctgctcaagcccttcttcctgcctggaataccctcttttcagtcccggctaattacatctaccatcccattccaagtggccgctggaaactcacttccttatgttctatgatttctttcacaatttacactttttttgttttgttttgttttgtttgggggggggggttgtttttctgagagggagtcttgctctgttgcccaggctggagtgcagtggcgcgatctcagctcaccacaacctccacctcatgggttcaaccgattctcctgcctcagcatcccgagtagctgggactacaggcgcgcgccgccacgcctggctaattttttgtgtttttggtagagacggggtttcactatgttggccgggctggtctcgaactcctgacctcgtgatccgcccgccaaacattctatgtgccagaaacttgtatgtaactttgcatagttcttgtattatgtctccccaattttgagtgttttctctggtctctactcagcccttgctcacagtctcactaggaccctggctagcttgaacacggtcagaaggatcaccttagatttctgctggcacagtattttccataaattaatctcctgattgaaccgtagtcctatagtagttatctttggatgaaaacctaatgcagctcgaggaggaagtggagtgcctgtgatcagagggctggacaaaagcccatagggcgtctctggagccctgggggaactgtccccaggcaggcactctcagggcccaggaacagggcgggacaagtccgactcacctgtgaggtaaggacggggccaagacccggaaccgaaaagcaggtcacgatagcgctggaaggcctcgtcgaggactgagcagccgggctgcgcggccgagctgacatcgtactggaattgaaagttgttcgggtaaaggacgtagcgctggtcggaggtttggaagttctgaggccagggccagagggccgtcgcccgtcctgcgaacgctgccgccagcagcagcgaaaaccaaagcctggagcttgtcatggcccgctggtctcccctctcggagggggctggccacgtgagaccctggtcaggtgagcggcgcccccgtgacttatcggcgaatcacgtgaccagaggcggctctgcgcctgcgaccccaggaggacccacaccctgccctttcacttccggcctggacctgaggctcctgctacgtgcagcggattgtgccgggcgtcggcaactgctcggctcgccagactgttcacgggcccagtagagcagtaaataaccggccacccctgcggccgtgccggccgtgatggaactagacagctcacccgactcgagaagaacggggtggggaggcgaggacgcagggcgacctggagaagaaagtttcccagatggctctagggtgaatcttgaatgttgtgatgcagctcgccaggcctacggggaggtaaaggacttgccaggctgaagtaacttcagaagcagaaaaacggaggattaaaaccacaagtcgttgtgatgtga

SEQ ID NO: 9 - DNA, 11400 bp, Homo sapiens
cacgtgaccgaggcacagatcagctgatgccggagggtttgaagccgcgccgcgagggagcgaggtcgcagtgacagcggcgggcgatcggacccaggctgccccgccgtacccgcctgcgtcccgcgctcccgccccagcatgacagccccggcgggtccgcgcggctcaggtgagggcgcgggcggcaccgtggggccccgaactcaggcgggcgggctgtgtctcccacctggggcggcggagctcctagtctctttttttctaagctccagcgctgacttttcacggtggagaaaagggcagacggctcctagaacttgggcggcgggtgggcaccagcctctccaattcttcctcctgaacccaggctctgctgggttcccaaactcaggcagggatcgcgccgggccgccagcttctccctctggggcggcgaggttcctgggattcccactgggagcctaggttccgattgctcaacttcgtctggaactcagacagcgggcaccagcttctccaacccgcacgtgagactcccaggcttcccctcctgattccaggggacaaatgctcagcttccctaagctcaagcctggagagctggagggattgcccccaggcgattaactcagttttagctttccaaaccgctggaagcgcagccttcttaaattcgggcttctagccaattctgatgccacccctcctcggggaggctggaggaagaccccttgtgttagcttccccttctggagctagctggggacccctacctgatagatgtcccggtgtcccagctagtagggtctggggtgggttagctgtaatctcagctctgtaagcgggccctgccctctggctttgtcgtaaacagccacagcagcatctcattgcaaagggaggggccgggaacttgtccctctctgcaagggaggttctgacagtgcacacatttatcctgactgctttgctaggcaggaggccaggccctagaaagcagcacggggccaggccctagaaagcacatccccatgggggtgtgacagggacagttttgggctactgtgactggttttgactccagcagttgctgaaagcttagatctaaccattaggctggaaaaaaataaacagtgattagaacagcttgtgtttgctgaagaggtctttatctgctgtgtctcactgaattctcagagcagcttcaggatctcaacctcaaggctcagggagagggtggactttttttttttttaataaacttttttttgttgcccaggctgtagtgcagtggcataatcctagctcattgtaacatcgaactcctgggctcaagtgatcctcccaactcagcctcccgggtagatggggtcccagctactaactacgggcatgagccgtcacacctgactatttaaaaaaaatgttttttttttgtagacagggaggtctcgctgtattacctaggctggatcctcccaccttggcctcccaaagccgttgggataacaggcatgagccactgagcccagccaaggggtcgcctttttaaaatttccactcttcagatgaggagatggaggctcagggaggtacctggagtcaacctactgtaaagtggcaggtctgggatttgatgctagggctgcatgatttctaggagctggtgcttttcagggagataaaatgagtctttagcgaatgtgttccattattattacttatgttgtcaattacctcttctccaggtccttggcttctgagagtgtcagctgatgggccaggttataatgaacccagaggtcatcttttgggtatttgtccagacaaacctagaatacaggctgagttctatgctcatgtctggaagctggagttgggataagcccagcaggcttgaacgcccagtgaaaagccagtgggagcagttcattctctccccactgatcaataacgggaacattgatgaaatgttctgacattcaccatggaccagcccctgtgatcaatgcttcataagcatccagtccttagcgttcccatgagacatattattgccccatttcgcagatgaggaaactgaggctcagagagctggtgagcaggaggggcaggaatcagcccaggccctgtacctcccaaacccaaactcataacctctgagcaggacgggtgcatagatacctacaatgtcacaggttttctggttttctttagacctctcagagctcttccttggcaggagcatggggacatgaagatagggcgtgtgctgccttcctggttggagaaaggggaaaaggggagttgcccaggcctcaccccagtgccctctcctattcccacagagaccgagcggcttctgacccccaaccccgggtatgggacccaggcggggccttcaccggcccctccgacacccccagaagaggaagaccttcgccgtcgtctcaaatactttttcatgagtccctgcgacaagtttcgagccaagggccgcaagccctgcaagctgatgctgcaagtggtcaagatcctggtggtcacggtgcaggtgaggccagccaagcaggggccccagctgaaggccacctgtggctgctgtgctccttgaagagagtcttaaagcagcactttggaaggccgaggccggtggatcgcttgaggctgggagttcaagaccagtctggccagcatggtgaaaccccatctctactaaaaatacaaaaaaattagccgtgcgtggtggcgggtgcctgtaatcccagctacttggcaggctgaggcaggagaatcgcttgaattgggaggcggaggttgccgtgagctgaaatcatgccactgcactccagcctgggcaacagagcaagactgtctcaaaaaaaaaaagaagccgactctgaggctcagagaggttaggagacttgcccaaagtcacacagcaatagaacattgggagctgggatttgaacccaggcagtctgacaccatgttgacccaatggctgcacagatagttctccctcccccatgccagaccctgtgctgggctctgggaaccccaagatgaatcagacccagccactgccctaagtgcttacttcatgttttgggctgactttagcatgtcaccatgcctctaattttccctctgaaaagggacccaattgtccaggcatggtggctcatgcctgtaatgccagcactttgggaggctgagttgggtggatcatttgaggccaggagtttgagaccagcctggccaacattgcaaaaccccgtctctactaaaaatacaaaaattagctgggtttggtggcaggtacctgtaactcagctactcaggaggctgagacaggagaattgcttgaacccagggggtggaggttgtagtgagctgagatcataccatggcactccaacttgggcaacagagtgagactctgtctcaaaaaagaaaagaaaagggacccagtcatggtacttaccctgaaagtttgggtttaacacagaatcggacatccagtaaacatttaatgaacgttagtccctgcagtgagatagatgagtccccaccctgtgttgtacgggggaggacacagtggtgggcgtggcatggagcttatgccaggaggtggggtgaaattaatcaaagcaaagaaatgcacaagtgaaatccgtgtttgtggcccaagttagcagggccctgccccaccccagtggacatctgcagggccctccctgtcctcttccagggcctgtgccctgagggagatacaccccaacccccatcctagccatgccaacctctactaccctctccccagctcatcctgtttgggctcagtaatcagctggctgtgacattccgggaagagaacaccatcgccttccgacacctcttcctgctgggctactcggacggagcggatgacaccttcgcagcctacacgcgggagcagctgtaccaggccatcttccatgctgtggaccaggtgctggtgggcgggcaggtgctggtgggcaggcaggtgcaggtgggcgggcaggtgcagttgggcgggcaggtgctggtgggcgggcaggtgcaggtgggtgggctgcagagagcgggccggactcacaggccctccccttctctgcccacagtacctggcgttgcctgacgtgtcactgggccggtatgcgtatgtccgtggtgggggtgacccttggaccaatggctcagggcttgctctctgccagcggtactaccaccgaggccacgtggacccggccaacgacacatttgacattgatccgatggtggttactggtgagtgggcaggacgaggcttcactgttgggagcctgagctgctgggattaaaatcaacagctgtggctgggcacggtggctcacgcctataataccagcactttgggaggctgaggaggaaggattgcttgaggccagaagtttgagaccagcctgggccacgtaggaagaccttgtctctacgcacaaacaaattagctgggcgtggtggcgtgcccctgtggtcccagctactcaggaggctgaggcaggaggatcgcttgagtccgggaggttgaggctgcagtaagctatgaccacgctgctgcactccaccctgggtgacagagtgagaccctgtctcaaaaaaaaaaaaaaaaaaaaaaaacaagtatgcttagtgtgagtgtgactcttgccacgtagaaagcaccagatgttatattttaatatggctcattcagtaaaacatccgcaggcccagagagtgccaggcctgtaggaatgacccaaccctggggaagcacagggaagaaggccactggggactctggggagaccagcctggcctccccggccccctgaggcccttccctgactccctgtccttagactgcatccaggtggatccccccgagcggccccctccgccccccagcgacgatctcaccctcttggaaagcagctccagttacaagaacctcacgctcaaattccacaagtactgcctgctcactcgaggggggcccagggtgggggaggcagcacactaggcactctcaccccagcaactacttccctaaggtggggacagggcccccccgcccgcgctggtgcctgctgggtgagcacttcccctgccagctgcagagtcagcacgtggcaggggacgctggcacttggggccggaagggacccgaagacgcccctgaccctcacccgagcctcctgcctaggctggtcaatgtcaccatccacttccggctgaagaccattaacctccagagcctcatcaataatgagatcccggactgctataccttcagcgtcctggtgaggccccccgggaacccacagggctcctgagttccagggcagggacctggtcagggagtgtcttgggagcactggccaagggcaagcgtgcgggtgatgagggagggagcccggggtctgtcaggccacctgtcatgtggaccttggggcttggggctgccaaggtttactctgcccccaactggcccccacagatcacgtttgacaacaaagcacacagtgggcggatccccatcagcctggagacccaggcccacatccaggagtgtaagcaccccagtgtcttccagcacggtgagcccctgagccccagaccagcactgaccaggggccctggcctgtcctgggattccccaagccccagatcagcgctgcctgggggccgtgacctccccaggaatccgctgagcctcagatcagcacagaccagggaccccgtcctgtgctgagatcccccaagccccagaccagcactgaccggggttcttgactcaccccaagcaagccctgagcccactgaccaaccaaaaccagccgtgcagccccctaggtctccagcctggcctggcaccaatgctagcctcccaaggctccatgccatccttggccctacccgctctgccctccccgcaggagacaacagcttccggctcctgtttgacgtggtggtcatcctcacctgctccctgtccttcctcctctgcgcccgctcactccttcgaggcttcctgctgcagaacgtgaggcttctgcgtcatgtgtgctggtgtcctccccgcctggccctggggcgataaaagccagggctttgagggtcctgtgcctggtcaggccctcaccccgcctgccttctgcaggagtttgtggggttcatgtggcggcagcggggacgggtcatcagcctgtgggagcggctggaatttgtcaatggctggtacatcctgctcgtcaccagcgatgtgctcaccatctcgggcaccatcatgaagatcggcatcgaggccaaggtgcgtcctgccaacaccctgggccccaggtcccatccctgctgtcagtgcctatccggggccatatcctcccccaggccccccaaaggaagggctgggccagataggttgacgcagctcccacccgcagaacttggcgagctacgacgtctgcagcatcctcctgggcacctcgacgctgctggtgtgggtgggcgtgatccgctacctgaccttcttccacaactacaatgtgagttttgcacatgcagctgggccttccacatggttactccacaccctccaaataaatccctacacacgcagccctcaccagccccggccaatggccccttgcaagcctcctcctcctacctgcccacaccagatatatctgtcactgcacctgcgcggggccccgggagcctgctcctttgtgcccacccagctgagtctagccgtgcgttgccctcggaccccctcagacgtggccacgccccctctaggcacccactggctcccatgaccacaccggctgtgccctcggcaaggccccgcccctcccacccccatctgggtgcccacagctgacctgagttgtggccacaccctcaacgaggctccctctgccccaacccagatcctcatcgccacactgcgggtggccctgcccagcgtcatgcgcttctgctgctgcgtggctgtcatctacctgggctactgcttctgtggctggatcgtgctggggccctatcatgtgaaggtacatctaacccctgatgtccctgacattgaccctgtgaccttgtcattgacactgtgacccccagatgaccccttggtgactgctgggagtctgtccactgtcccctgtggtccttggtgaccctgacactgaccctgtgccattattgttgtcacagttgttgatgaccctatttcgacctgaattactcccctcctgctctatctacccagaccctaggtcggccctgtggccctgtcattgacccgtggtcccggccattcacatgggaccccagcctgggacctggccattcacatagtgaccccagcctgggacccggccattcacgtgggaccccagcctgggtcccggccattcacgtgggaccccagcctgggacccggccattcacaggggccctagcctggaacccgaccattcacatggtgaccgcagcccgggacccggccattcatgtggggccccagccaccagctcctagccatttgcatgggaccccagcctgaccccagcccccggttcctggccatgccttggctccctctgaccccgccgcccctctggcagttccgctcactctccatggtgtctgagtgcctgttctcgctcatcaatggggacgacatgtttgtgacgttcgccgccatgcaggcgcagcagggccgcagcagcctggtgtggctcttctcccagctctacctttactccttcatcagcctcttcatctacatggtgctcagcctcttcatcgcgctcatcaccggcgcctacgacaccatcaaggtcagccgcatgcacccagccctgagctcgggctctgggtgccctggagtctgccatgagggggtcttggggacaccgcagggtgaacagagaagacccaggagagaatatgggagactctatgaaaccaaaaagagggtggttcagaactggggggcgcagggggatgtcaaggtgggcttgggccaggagggggcctgagtcagtctttgccaacagggcaaccgagtcatagagtttatttatttatttgtttatttgagacggagtcttgctctgtcacccagggtggagtgcagtggtgcgatcttgactcactgcaacctccacctcccgggttcaagcaattctgtctcagcctcctgagtagctgggactacaggcacacgccaccacgtccagctaatttttgtatttttagtagagatggcatttcaccgcattggtcaggctggtctcaaactcctggcctcaggagatctactgccttggcctcccaaagtgttgggattacaggcgtgagccaccacgcccggcctattttattttattattaaagtattgttctttattttattagagacaagggtctcactgtgttacccaggctggtttcaaactcctgaggtcaagtgatcctcccactttggcctcccaaagtgctgggattacaggcgtaagccaccacacccagcctattattattattttttttttgaaatggaatcttaccctgtggcccaggctggagtgcaatggcatgatctcggctcactgcaacctccaccttctgagttgaagcgatccttgtgcctcagcctcctgagtagctgggattacttgcacgtgccaccacacctggctaatttttgtatttttactagagatggggtttcaccacgttggccaggctggtctcgaactcctgacctcaggtgatccacctgccttggcctcccaaggtgctgggatttcaggcatgagccactgaacccagctaagtcatacagtttcaatgaccttgtcattgaccctgggacgttgccattaacatggtgatcctcagctggccccattcctatggcggacctctaaaaacccaaccctgaccccagcccccagccatgcccccgactccctctgaccctgcccaaggttagcttctttatttatttattttttttgagacggagtctcgctgtgtcacccaggctggagtgcagtggtgcaatctcggctcactgcaacctctgcctcccgggttcaagcgattctcctgcctcagcctactgagtagctgggattataggcacacgccaccatgcctggctaatttttgtatttttagtagagatggggtttcaccatgttgaccaggctggtctcaaactcctgaccttgtgatccgcccacctcaggctcccaaagtgctgggattacgggcgtgagccactgtgcccggcccaggttagcttctgagcagtaaaactgggctcaacccagggctgtctgattccagaagccgtgctcctaacccctctgtcctcagtttagtagggtggctgggaacagtggtttccctgcaagctgcaagggtcaggggacagagcaggatgcggaagtggcaggtagataggattctttcagcagatatatctaagggccaagatctgtgctgggttctgggcatggaggaaaatcaggtgtgcatgatccgtccaaggcctgtgggcaaggatggcacaggaacagacatcccatgaccaatgacctacttgtaacaggtatgaaggaagagtggaaggttgcagagggacccctgctttagattggtatgacaggagatccaggagagcttctagaatgttctatccatcactagtctctagccctatgcagctatttaaattttgattttaattccggctgggcacggtgactcacgcctgtaatcccagccctttgggaggccgagggggggtgggggtggatcacctgaggttaggagttcgagaccagcctggccaacatggcaaaaccccatctctactaaaaatacaaaaaattagcggacgtggtggcaggcacctgtaatcccagctactccggaggctgaggcagtagaattgcttgaacctgggacgtagaggttgcagtgagccgagatcaagccactgcactccaacgtgggcgagagaccgagactctgcttcaaagacaaaacaacaattttttaaaaattttaattcaaatgaagtacaattgcaaatttagcctctgacttgcaccatcctgtatccagtgctcaaaagccagtgtggctggtggctgccatattggacagcatagatattgaatacttccatcgcctctagactgaagagatgggagcccaggggcagtgcaccgaggggaaggaatagctaaagcaaaggtctagtagcctgaaaaaacttggagaaaagatggcccctccatgaggccgagtgagaggaaggaagccttggctgggaccctgccacatccaatgtcaccggcagatgggtacacccccttttccccatgcatggattcagctgtcccacagacacattgactcaggcccttggaactacttcctgtcttgccttagcacgtagacatcacacacatgcatccactcaggtgggcagtctcaggccctgctcccactgctgtgctcagcgtgcatccagctcactcaatagatggtttctgagcatcgaggtcatgtcagccctggctctaggtctgtaggtgctggacctacagcagaaggcaaagacacagactggcaaagacacagcttgtatccaggttcaggggtcagggaaggtccctctgtgcaagcaaactgtggaacaacgggtggagcaggcccagcaagtgcaaaggcccggaggtgggaagcgatgcagatatggctggaggggagggcggacttcaagggccttggaggttgggagccactttcaggctgagcctcccggcttctctccccagcatcccggcggcgcaggcgcagaggagagcgagctgcaggcctacatcgcacagtgccaggacagccccacctccggcaagttccgccgcgggagcggctcggcctgcagccttctctgctgctgcggaaggttcgagtcccgggtctggcacattcagattggaggttagggaatggggaaaggggagcgagccagagaaaactgacgcccctcttccctgcttccttcctccagggacccctcggaggagcattcgctgctggtgaattgattcgacctgactgccgttggaccgtaggccctggactgcagagacccccgcccccgaccccgcttatttatttgtagggtttgcttttaaggatcggctccctgtcgcgcccgaggagggcctggacctttcgtgtcggacccttgggggcggggagactgggtggggagggtgttgaataaaagggaaaataaatgtgtcgttttcattttta

SEQ ID NO: 10 - DNA, 20 bp, Artificial Sequence
  Description of Artificial Sequence Synthetic primer
gatcagaggaagaggaaggg

SEQ ID NO: 11 - DNA, 20 bp, Artificial Sequence
  Description of Artificial Sequence Synthetic primer
atggattgtagatgccaccc

SEQ ID NO: 12 - DNA, 21 bp, Artificial Sequence
  Description of Artificial Sequence Synthetic primer
tcaccccattgtgacctttgt

SEQ ID NO: 13 - DNA, 19 bp, Artificial Sequence
  Description of Artificial Sequence Synthetic primer
acaagtgctgtgacggtgg

SEQ ID NO: 14 - DNA, 20 bp, Artificial Sequence
  Description of Artificial Sequence Synthetic primer
catgactacctggagggcac

SEQ ID NO: 15 - DNA, 22 bp, Artificial Sequence
  Description of Artificial Sequence Synthetic primer
gagccaaatgaagagcactaag

SEQ ID NO: 16 - DNA, 17 bp, Artificial Sequence
  Description of Artificial Sequence Synthetic primer
tcctggagccctctgcc

SEQ ID NO: 17 - DNA, 20 bp, Artificial Sequence
  Description of Artificial Sequence Synthetic primer
gatgccctctttctccaacc

SEQ ID NO: 18 - DNA, 20 bp, Artificial Sequence
  Description of Artificial Sequence Synthetic primer
cttgagcatctcaccatccc

SEQ ID NO: 19 - DNA, 20 bp, Artificial Sequence
  Description of Artificial Sequence Synthetic primer
ggtaacctgcaaggatgtgg

SEQ ID NO: 20 - DNA, 20 bp, Artificial Sequence
  Description of Artificial Sequence Synthetic primer
tcctggagttggtgggatag

SEQ ID NO: 21 - DNA, 20 bp, Artificial Sequence
  Description of Artificial Sequence Synthetic primer
agctccaggaaaggagaagg

SEQ ID NO: 22 - DNA, 16 bp, Artificial Sequence
  Description of Artificial Sequence Synthetic probe
tccgcagcgttgacag

SEQ ID NO: 23 - DNA, 24 bp, Artificial Sequence
  Description of Artificial Sequence Synthetic probe
gtccccggcgccgcccggggccct

SEQ ID NO: 24 - DNA, 112 bp, Homo sapiens
ccgcccgccaccgagagatcagctgtcagagatcagaggaagaggaaggggcggagctgctttgcggccggccgcggagcagtcagccgactacagagaagggtaatcgggt

SEQ ID NO: 25 - DNA, 17 bp, Artificial Sequence
  Description of Artificial Sequence Synthetic probe
cagctctggcggagtga

SEQ ID NO: 26 - DNA, 23 bp, Artificial Sequence
  Description of Artificial Sequence Synthetic probe
cccgattacccttctctgtagtc

SEQ ID NO: 27 - DNA, 112 bp, Homo sapiens
cgctccgtagcggggcattgtcgcgcttcctacacggggctggttcgtctgtcccgtcccccccggcctggaccctagccagccctcagggccccgggcggcgccggggaca

SEQ ID NO: 28 - DNA, 16 bp, Artificial Sequence
  Description of Artificial Sequence Synthetic probe
tgccccgctacggagc

SEQ ID NO: 29 - DNA, 24 bp, Artificial Sequence
  Description of Artificial Sequence Synthetic probe
gcgctggcgctggctctgtctgac

SEQ ID NO: 30 - DNA, 112 bp, Homo sapiens
gtcactccgccagagctgccccaggtccggccgggagcagggacaagacgggaccgccggagcccccggactcctttggatgggcctggtgctggcgctggcgctggcgctg

SEQ ID NO: 31 - DNA, 18 bp, Artificial Sequence
  Description of Artificial Sequence Synthetic probe
ggggcactatgcgatgta

SEQ ID NO: 32 - DNA, 22 bp, Artificial Sequence
  Description of Artificial Sequence Synthetic probe
caggcccatccaaaggagtccg

SEQ ID NO: 33 - DNA, 112 bp, Homo sapiens
acctggcaggatggccttggggagaaagagggtgagcctctgccggagcccagagaacccgagagtcagacagagccagcgccagcgccagcgccagcgccagcgccagcac

SEQ ID NO: 34 - DNA, 17 bp, Artificial Sequence
  Description of Artificial Sequence Synthetic probe
ctcgggttctctgggct

SEQ ID NO: 35 - DNA, 23 bp, Artificial Sequence
  Description of Artificial Sequence Synthetic probe
aaggtctattcaccgccatcaac

SEQ ID NO: 36 - DNA, 112 bp, Homo sapiens
ccggcagaggctcaccctctttctccccaaggccatcctgccaggttacatcgcatagtgccccggctccgagatgtctttgggtgggggaacctcacctgcccaatctgca

SEQ ID NO: 37 - DNA, 18 bp, Artificial Sequence
  Description of Artificial Sequence Synthetic probe
atgcattctgagcccagc

SEQ ID NO: 38 - DNA, 22 bp, Artificial Sequence
  Description of Artificial Sequence Synthetic probe
cagattgggcaggtgaggttcc

SEQ ID NO: 39 - DNA, 112 bp, Homo sapiens
gcaccagcatcagccccagcccccagccccagcactcctttcggcctcctccactgcagccccttcagtgctcaccttcagcccgaggttgatggcggtgaatagacctttg

SEQ ID NO: 40 - DNA, 19 bp, Artificial Sequence
  Description of Artificial Sequence Synthetic probe
ctgcctctgatttctcacc

SEQ ID NO: 41 - DNA, 21 bp, Artificial Sequence
  Description of Artificial Sequence Synthetic probe
ccacctctttgaggatgacat

SEQ ID NO: 42 - DNA, 112 bp, Homo sapiens
atgcgctcctcccactgcagaaggaacccaatgtggctcgcgtgggctccgtggccatcaagctgtgcaatctgctgaagatagcaccacctgccgtgtgccaatccattgt

SEQ ID NO: 43 - DNA, 19 bp, Artificial Sequence
  Description of Artificial Sequence Synthetic probe
caaagagatgttccaagat

SEQ ID NO: 44 - DNA, 21 bp, Artificial Sequence
  Description of Artificial Sequence Synthetic probe
caatggattggcacacggcag

SEQ ID NO: 45 - DNA, 112 bp, Homo sapiens
gagaaaatgtcccagtgcccacaggtggagcccaggagcaggccacaggcctcagatgggctcagcactgagcgtctccacacctccaccatgtcatcctcaaagaggtgga

SEQ ID NO: 46 - DNA, 16 bp, Artificial Sequence
  Description of Artificial Sequence Synthetic probe
ggcctgctcctgggct

SEQ ID NO: 47 - DNA, 24 bp, Artificial Sequence
  Description of Artificial Sequence Synthetic probe
catcctcttcctcactgacctgca

SEQ ID NO: 48 - DNA, 112 bp, Homo sapiens
ccacctgtgggcactgggacattttctcatcttggaacatctctttgcctactgtgccgaagccgccccccaaaccccctagccccccagccccaggtgcccctgtcagccg

SEQ ID NO: 49 - DNA, 19 bp, Artificial Sequence
  Description of Artificial Sequence Synthetic probe
gtatccggcacctggccgg

SEQ ID NO: 50 - DNA, 21 bp, Artificial Sequence
  Description of Artificial Sequence Synthetic probe
ggctgacaggggcacctgggg

SEQ ID NO: 51 - DNA, 112 bp, Homo sapiens
gatgcgggcggcaggccagaaccccggcggcagcacagtgggtctgcacagtcagggtccgtgccctccaggtagtcatgatcccagtgcaggtcagtgaggaagaggatgc

SEQ ID NO: 52 - DNA, 16 bp, Artificial Sequence
  Description of Artificial Sequence Synthetic probe
ccggggttctggcctg

SEQ ID NO: 53 - DNA, 24 bp, Artificial Sequence
  Description of Artificial Sequence Synthetic probe
atatggtgtactggacaggagaca

SEQ ID NO: 54 - DNA, 112 bp, Homo sapiens
ccgcccgcatcccggccaggtgccggatactggggcgaatacagcaagtgtgacctgcccctgaggaccctggagagcctgttgagtgggctgggcccagccggcccttttg

SEQ ID NO: 55 - DNA, 19 bp, Artificial Sequence
  Description of Artificial Sequence Synthetic probe
cccaggaacttcctcacaa

SEQ ID NO: 56 - DNA, 21 bp, Artificial Sequence
  Description of Artificial Sequence Synthetic probe
cagcccactcaacaggctctc

SEQ ID NO: 57 - DNA, 112 bp, Homo sapiens
gtgctgtgacggtggtcagggcccgcagttggtcctgacgagtctggtgccagacatcatgtgcggggatgtctcctgtccagtacaccatatcaaaagggccggctgggcc

SEQ ID NO: 58 - DNA, 18 bp, Artificial Sequence
  Description of Artificial Sequence Synthetic probe
atgtctggcaccagactc

SEQ ID NO: 59 - DNA, 22 bp, Artificial Sequence
  Description of Artificial Sequence Synthetic probe
cttccctccccccttcattgag

SEQ ID NO: 60 - DNA, 112 bp, Homo sapiens
gtcaggaccaactgcgggccctgaccaccgtcacagcacttgtgaggaagttcctggggccagtgccagtgtaccctgctgtgggtaaccatgaaagcacacctgtcaatag

SEQ ID NO: 61 - DNA, 18 bp, Artificial Sequence
  Description of Artificial Sequence Synthetic probe
ccacggacgataagtacc

SEQ ID NO: 62 - DNA, 22 bp, Artificial Sequence
  Description of Artificial Sequence Synthetic probe
gacaggtgtgctttcatggtta

SEQ ID NO: 63 - DNA, 112 bp, Homo sapiens
tgagggtgcgcagggcttcggcaggcagccagggctcccaagccttggccatcgcttcatagagccagcgggaggagtggttgccctcaatgaaggggggagggaagctatt

SEQ ID NO: 64 - DNA, 16 bp, Artificial Sequence
  Description of Artificial Sequence Synthetic probe
cttgggagccctggct

SEQ ID NO: 65 - DNA, 24 bp, Artificial Sequence
  Description of Artificial Sequence Synthetic probe
attgctctagcatgagtccttagt

SEQ ID NO: 66 - DNA, 112 bp, Homo sapiens
gcctgccgaagccctgcgcaccctcaggtacttatcgtccgtggaaacccaggaagggaaaagaaaggtgaatgaaagtgaagggagaagggaacctggggcattgtctctg

SEQ ID NO: 67 - DNA, 19 bp, Artificial Sequence
  Description of Artificial Sequence Synthetic probe
aggattggaacaagtgttg

SEQ ID NO: 68 - DNA, 21 bp, Artificial Sequence
  Description of Artificial Sequence Synthetic probe
gctcttgatcaactccacgga

SEQ ID NO: 69 - DNA, 112 bp, Homo sapiens
acctctcatgtttactttgtttcagaattggggggttctatgctctttccccataccccggtctccgcctcatctctctcaatatgaatttttgttcccgtgagaacttctg

SEQ ID NO: 70 - DNA, 17 bp, Artificial Sequence
  Description of Artificial Sequence Synthetic probe
cttgtcccccagcacca

SEQ ID NO: 71 - DNA, 23 bp, Artificial Sequence
  Description of Artificial Sequence Synthetic probe
gaagttctcacgggaacaaaaat

SEQ ID NO: 72 - DNA, 112 bp, Homo sapiens
ccgtgttcccactactggccctcactttgtctcctcgatcctcagcagcctgaagctcccccaccagccactggagctgtcctgcgggatccgtggagttgatcaagagcca

SEQ ID NO: 73 - DNA, 19 bp, Artificial Sequence
  Description of Artificial Sequence Synthetic probe
ctggatgccctgattacca

SEQ ID NO: 74 - DNA, 21 bp, Artificial Sequence
  Description of Artificial Sequence Synthetic probe
gggtgggaatagggacagggt

SEQ ID NO: 75 - DNA, 112 bp, Homo sapiens
tccttaattctccctactaggtgcatataattggccacattcccccagggcactgtctgaagagctggagctggaattattaccgaattgtagccaggtaggacggagatga

SEQ ID NO: 76 - DNA, 19 bp, Artificial Sequence
  Description of Artificial Sequence Synthetic probe
gagataggatgccctcttt

SEQ ID NO: 77 - DNA, 21 bp, Artificial Sequence
  Description of Artificial Sequence Synthetic probe
catctccgtcctacctggcta

SEQ ID NO: 78 - DNA, 112 bp, Homo sapiens
ctccaacctgttctagtgaggagcctccccactccatgggacaacagggatggtgagatgctcaagggaattttcagccttcagacactcaccctgtccctattcccaccct

SEQ ID NO: 79 - DNA, 20 bp, Artificial Sequence
  Description of Artificial Sequence Synthetic probe
tacccctccctagaatcttc

SEQ ID NO: 80 - DNA, 20 bp, Artificial Sequence
  Description of Artificial Sequence Synthetic probe
cgctggctgtagccttcctg

SEQ ID NO: 81 - DNA, 112 bp, Homo sapiens
tgaatgtagtaccttctggccaggtatgagaacaccctggctgctcagttctttggccacactcatgtggatgaatttgaggtcttctatgatgaagagactctgagccggc

SEQ ID NO: 82 - DNA, 17 bp, Artificial Sequence
  Description of Artificial Sequence Synthetic probe
tctggctccaacctcct

SEQ ID NO: 83 - DNA, 23 bp, Artificial Sequence
  Description of Artificial Sequence Synthetic probe
cggctcagagtctcttcatcata

SEQ ID NO: 84 - DNA, 112 bp, Homo sapiens
tcccctatcccaccaactccaggataagggaggctcccttctgcctcactcaccaggattaaggccgatgtaggtagttgcactgggtgccaggaaggctacagccagcggc

SEQ ID NO: 85 - DNA, 18 bp, Artificial Sequence
  Description of Artificial Sequence Synthetic probe
tccctggagttacccttg

SEQ ID NO: 86 - DNA, 22 bp, Artificial Sequence
  Description of Artificial Sequence Synthetic probe
gaatctgacccaggcaaacata

SEQ ID NO: 87 - DNA, 112 bp, Homo sapiens
ctccttgcccctccagtcagccccacatccttgcaggttaccgtgtgtaccaaatagatggaaactactccgggagctctcacgtggtcctggaccatgagacctacatcct

SEQ ID NO: 88 - DNA, 17 bp, Artificial Sequence
  Description of Artificial Sequence Synthetic probe
cccgcatgcgatatacc

SEQ ID NO: 89 - DNA, 23 bp, Artificial Sequence
  Description of Artificial Sequence Synthetic probe
ggatgtaggtctcatggtccagg

SEQ ID NO: 90 - DNA, 112 bp, Homo sapiens
aggttgtgccaggcggtaggcagtgtgttgggcagcccataggtttctcgagccctgtagagaagctgccagtgcggtatggctcccggtatgtttgcctgggtcagattca

SEQ ID NO: 91 - DNA, 18 bp, Artificial Sequence
  Description of Artificial Sequence Synthetic probe
cccaacacactgcctacc

SEQ ID NO: 92 - DNA, 22 bp, Artificial Sequence
  Description of Artificial Sequence Synthetic probe
gtctggctactctttgtgccca

SEQ ID NO: 93 - DNA, 112 bp, Homo sapiens
gcctggcacaacctggtatatcgcatgcggggcgacatgcaacttttccagaccttctggtttctctaccataagggccacccaccctcggagccctgtggcacgccctgcc

SEQ ID NO: 94 - DNA, 16 bp, Artificial Sequence
  Description of Artificial Sequence Synthetic probe
ccacaggctctgggcc

SEQ ID NO: 95 - DNA, 24 bp, Artificial Sequence
  Description of Artificial Sequence Synthetic probe
agggtgggtggcccttatggtaga

SEQ ID NO: 96 - DNA, 112 bp, Homo sapiens
tctgggaggctcccatctggcatcaggtggcggcacagagcagggctgtcagcacgggcagagagctgggcacaaagagtagccagacggcagggcgtgccacagggctccg

SEQ ID NO: 97 - DNA, 16 bp, Artificial Sequence
  Description of Artificial Sequence Synthetic probe
tgctctgtgccgccac

SEQ ID NO: 98 - DNA, 24 bp, Artificial Sequence
  Description of Artificial Sequence Synthetic probe
aaagcccaaatgctgctgtggttc

SEQ ID NO: 99 - DNA, 112 bp, Homo sapiens
ctgatgccagatgggagcctcccagaggcccagagcctgtggccaaggccactgttttgctagggccccagggcccacatttgggaaagttcttgatgtaggaaagggtgaa

SEQ ID NO: 100 - DNA, 17 bp, Artificial Sequence
  Description of Artificial Sequence Synthetic probe
cagctaaaccagctcca

SEQ ID NO: 101 - DNA, 23 bp, Artificial Sequence
  Description of Artificial Sequence Synthetic probe
cctacatcaagaactttcccaaa

SEQ ID NO: 102 - DNA, 112 bp, Homo sapiens
ggaaaggagaaggtcctgtttccccggcatccttggggcccagggactggttctttcaccggatgatcttgcctggttgaaccacagcagcatttgggctttttcacccttt

SEQ ID NO: 103 - DNA, 20 bp, Artificial Sequence
  Description of Artificial Sequence Synthetic probe
aaacaggaccttctcctttc

SEQ ID NO: 104 - DNA, 20 bp, Artificial Sequence
  Description of Artificial Sequence Synthetic probe
actgccctgggcagacaaga

SEQ ID NO: 105 - DNA, 112 bp, Homo sapiens
ctggagctggtttagctggatatgggagggggtttggctgcctgtgcccaggagctagactgccttgaggctgctgtcctttcacagccatggagtagaggcctaagttgac

SEQ ID NO: 106 - DNA, 18 bp, Artificial Sequence
  Description of Artificial Sequence Synthetic probe
ctggagtccaagtctctt

SEQ ID NO: 107 - DNA, 22 bp, Artificial Sequence
  Description of Artificial Sequence Synthetic probe
caacttaggcctctactccatg

SEQ ID NO: 108 - DNA, 112 bp, Homo sapiens
atctttattttaacagactggcagcatcaggtcgcagcagcagtacagggttcctggctgggcagcacaggcctggggcgacagctcctgtcttgtctgcccagggcagtgt

SEQ ID NO: 109 - DNA, 20 bp, Artificial Sequence
  Description of Artificial Sequence Synthetic probe
gccagtctgttaaaataaag

SEQ ID NO: 110 - DNA, 20 bp, Artificial Sequence
  Description of Artificial Sequence Synthetic probe
gttgatggtgtggtacaggc

SEQ ID NO: 111 - DNA, 112 bp, Homo sapiens
ataagagacttggactccagacccctgtgtgactgtcccaatttcttctttccaggcaagcagggcaaggagatctttggagcaagatcataactgaggcacaggctaatgg

SEQ ID NO: 112 - DNA, 21 bp, Artificial Sequence
  Description of Artificial Sequence Synthetic oligonucleotide
  Description of Combined DNA/RNA Molecule Synthetic oligonucleotide
ucacagcacuugugaggaatt

SEQ ID NO: 113 - DNA, 21 bp, Artificial Sequence
  Description of Artificial Sequence Synthetic oligonucleotide
ctgctgtgggtaacctagaaa

(19) United States
(12) Patent Application Publication
(10) Pub. No.: US 2018/0051337 A1
(43) Pub. Date: Feb. 22, 2018

(54) PREDICTIVE VALUE OF COMBINED GENETIC ENZYMATIC AND LIPIDOMIC DATA IN DISEASE RISK FOR LEWY BODY DISEASE

(71) Applicant: THE TRUSTEES OF COLUMBIA UNIVERSITY IN THE CITY OF NEW YORK, NEW YORK, NY (US)

(72) Inventor: Lorraine Clark, Tenafly, NJ (US)

(73) Assignee: THE TRUSTEES OF COLUMBIA UNIVERSITY IN THE CITY OF NEW YORK, NEW YORK, NY (US)

(21) Appl. No.: 15/726,059

(22) Filed: Oct. 5, 2017

Related U.S. Application Data
(63) Continuation of application No. PCT/US2016/026238, filed on Apr. 6, 2016.
(60) Provisional application No. 62/143,749, filed on Apr. 6, 2015.

(51) Int. Cl. C12Q 1/68; G01N 33/68; G01N 33/53; G01N 33/573

(52) U.S. Cl. CPC C12Q 1/6883; C12Q 1/6841; G01N 33/6896; G01N 33/53; G01N 2800/2814; C12Q 2600/156; C12Q 2600/118; G01N 2800/50; G01N 33/573

(57) ABSTRACT

This present invention relates to genetic mutations that may be used to evaluate the risk of Lewy body disease in a subject. This invention is based, at least in part, on the discovery that genetic mutations in the genes GBA, SMPD1, HEXA and MCOLN1 are associated with Lewy body disease. As such, these mutations may be used in methods of diagnosing and treating Lewy body disease patients.

### CROSS-REFERENCE TO RELATED APPLICATION

The present application is a continuation of International Application No. PCT/US2016/026238 filed Apr. 6, 2016, which claims priority to U.S. Provisional Application No. 62/143,749 filed Apr. 6, 2015, the contents of each of which are incorporated by reference in their entireties herein.

### GRANT INFORMATION

This invention was made with government support under grant numbers NS060113, P50AG08702, K02NS080915, and UL1 TR000040 awarded by the National Institute of Health. The government has certain rights in the invention.

### SEQUENCE LISTING

The specification further incorporates by reference the Sequence Listing submitted herewith via EFS on Oct. 5, 2017. Pursuant to 37 C.F.R. §1.52(e)(5), the Sequence Listing text file, identified as “070050_6001_SL.txt,” is 125,744 bytes and was created on Oct. 5, 2017. The Sequence Listing, electronically filed herewith, does not extend beyond the scope of the specification and thus does not contain new matter.

### 1. INTRODUCTION

This present invention relates to genetic variants that may be used to evaluate the risk of Lewy body disease in a subject. As such, these variants may be used in methods of diagnosing and treating Lewy body disease patients.

### 2. BACKGROUND OF THE INVENTION

Lewy body disorders, which include Parkinson's Disease (PD) and Dementia with Lewy bodies (DLB), are characterized by neuronal loss in the substantia nigra (SN) and the presence of neuronal cytoplasmic inclusions composed predominantly of α-synuclein termed Lewy Bodies (LBs)[1-3]. α-synuclein immunoreactivity, including LB, have been described as features seen in the neuropathology of several lysosomal storage disorders including notably Gaucher disease (GD), but also Sandhoff disease, Tay Sachs disease, and Sanfilippo syndrome[4-8]. Heterozygosity for mutations in the gene encoding glucocerebrosidase (GBA), which cause Gaucher disease (GD), has been identified as a risk factor for both PD and DLB. In sporadic and familial PD, GBA mutations are associated with early-onset PD and may modify age at onset of PD[9,10], and that in brain autopsies GBA mutation status was significantly associated with the presence of cortical LB (OR=6.48, 95% CI, 2.45-17.16, p<0.001) and a neuropathological diagnosis of DLB after adjusting for sex, age at death, and presence of APOE- 4[11]. A recent study that assessed the association of specific founder mutations in each of the lysosomal storage disorder genes HEXA, SMPD1 and MCOLN1 in 938 Ashkenazi Jewish (AJ) PD patients and 282 matched AJ controls, reported SMPD1 L302P as a risk factor for PD in the AJ population[12]. Despite these advances in the field, there remains a need for biomarker and therapeutic strategy development, such as those described in the instant application.

### 3. SUMMARY OF THE INVENTION

This present disclosure relates to genetic mutations that may be used to evaluate the risk of Lewy body disease (LBD) in a subject. This invention is based, at least in part, on the discovery that genetic variants (i.e., mutations) in the genes GBA, SMPD1, HEXA, and/or MCOLN1 are associated with Lewy body disease. As such, these mutations may be used in methods of diagnosing and treating Lewy body disease patients. Accordingly, in non-limiting embodiments, the present disclosure provides for assay methods and kits for determining whether such a variant is present in a sample from a subject, wherein the presence of said variant indicates that the subject is at risk of having Lewy body disease.

In certain non-limiting embodiment, the method of determining whether a subject is at risk of having Lewy body disease comprises obtaining a biological sample from a subject and determining the presence of one or more variants in a gene selected from the group consisting of GBA, SMPD1, HEXA, MCOLN1, and combinations thereof in the biological sample, wherein the presence of the one or more variants indicates that the subject is at risk of having Lewy body disease.

In certain embodiments, the gene is GBA. In a non-limiting embodiment, the one or more variants is a rs114099990 single nucleotide polymorphisms in GBA.

In certain embodiments, the GBA variant comprises a mutation in the GBA nucleic acid sequence selected from the group consisting of g.1864A>G, c.38A>G, g.7549A>C, c.1584A>C, g.3940C>T, c.474C>T, g.5026C>T, c.795C>T, g.7314G>A, c.1443G>A, g. 7366G>C, c.1495G>C, g.3942G>A, c.476G>A, g.1367C>T, and combinations thereof (wherein “g.” describes the position comprising the nucleotide variation in the GBA gene, and “c.” describes the position comprising the nucleotide variation in the GBA cDNA).

In certain embodiments, the GBA variant comprises a mutation in the GBA amino acid sequence selected from the group consisting of p.Lys13Arg, p.Asp482Asn, p.Va1499Leu, p.Arg159Gln, and combinations thereof

In certain embodiments, the gene is SMPD1. In certain embodiments, the variant of SMPD1 comprises a single nucleotide polymorphism (SNP) selected from the group consisting of rs144465428, rs1050228, rs71056748, rs7951904, rs1050239, rs8164, rs72896268, rs2723669, rs142178073, rs144873307, rs142787001 and combinations thereof.

In certain embodiments, the SMPD1 variant comprises a mutation in the SMPD1 nucleic acid sequence selected from the group consisting of a IVS3+2T>C in the SMPD1 genomic nucleic acid, c.1829delCGG mutation in SMPD1 cDNA, and combinations thereof.

In certain embodiments, the SMPD1 variant comprises a mutation in the SMPD1 amino acid sequence selected from the group consisting of p.Q19R, p.V36A, p.Leu49_Ser50insAL, p.Leu49_Ser50insALAL, p.D212D, p.E358K, p.G508R, p.R542L, p.V301I, p.M33I, p.G492S, p.E517V, p.R418Q, p.R291H, p.A487V, p.P331A, p.R378H, p.R498L, p.G530A, p.R610H, and combinations thereof.

In certain embodiments, the gene is HEXA. In certain embodiments, the variant of HEXA comprises a single nucleotide polymorphism (SNP) selected from the group consisting of rs2302449, rs387906309, rs73440586, rs117513345, rs1800428, rs121907970, rs117160567, rs2288259, rs1800431, rs121907954, rs117160567, rs10220917, and combinations thereof.

In certain embodiments, the HEXA variant comprises a 1277 1278insTATC mutation in the HEXA genomic nucleic acid sequence, a c.672+30T>G mutation in the HEXA cDNA sequence, or combinations thereof.

In certain embodiments, the HEXA variant comprises a mutation in the HEXA amino acid sequence selected from the group consisting of p.Y427I, p.V253V, p.S3S, p.R247W, p.I436V, p.G269S, p.V192I, and combinations thereof.

In certain embodiments, the gene is MCOLN1. In certain embodiments, the variant of MCOLN1 comprises a single nucleotide polymorphism (SNP) selected from the group consisting of rs45513896, rs145706318, rs73003348, rs2305889, rs139922988, rs145386883, rs686796, rs113261161, rs61736600, rs612862, rs142259322, rs147754092, and combinations thereof.

In certain embodiments, the MCOLN1 variant comprises a mutation in the MCOLN1 amino acid sequence selected from the group consisting of p.P197S, p.T261M, p.C386C, p.G528G, p.S257R, p.R322R, p.N328N, p.A138V, S424S, and combinations thereof.

In certain non-limiting embodiment, at least two variants are present, wherein one of the at least two variants is in GBA, and the other variant is in SMPD1.

In another non-limiting embodiment, at least three variants are present, wherein a first variant is in GBA, a second variant is in SMPD1, and a third variant is in MCOLN1.

In certain embodiments, the one or more variants comprise a variant in GBA, wherein GCase activity is decreased in the subject compared to a subject without LBD, or a reference level determined from one or more subjects without LBD. In another embodiment, the subject has a decreased β-glucocerebrosidase: α-hexosaminidase ratio.

In certain embodiments, the one or more variants comprise a variant in SMPD1, wherein ASMase (Acid sphingomyelinase) activity is decreased in the subject compared to a subject without LBD, or a reference level determined from one or more subjects without LBD.

In certain embodiments, the subject comprising the one or more variant further comprises an altered lipid profile. In one embodiment, the level of phosphatidylcholine, sphingolipid sphingomyelin, and/or phosphatidylethanolamine is decreased in the subject compared to a subject without LBD, or a reference level determined from one or more subjects without LBD.

In certain embodiments, the level of phosphatidylserine, dihydrosphingomyelin, ceramide, glycosphingolipid, and/or galactosylceramide is increased in the subject comprising the one or more variant compared to a subject without LBD, or a reference level determined from one or more subjects without LBD.

In certain embodiments, the Minor Allele Frequency (MAF) of the one or more variants are less than 0.05.

In certain embodiments, the subject is human.

In certain embodiments, the biological sample is selected from the group consisting of a tissue sample, for example, a brain sample, a blood sample, and a cerebral spinal fluid sample.

In certain embodiments, the presence of the variant is detected by in situ hybridization.

The present disclosure further provides for a method of preventing or treating Lewy body disease in a subject, comprising: (a) determining the presence of one or more variants in a biological sample obtained from a subject, wherein the one or more variants are in a gene selected from the group consisting of GBA, SMPD1, HEXA, MCOLN1, and combinations thereof; and (b) if the one or more variants are present in the biological sample, treating the subject with a Lewy body disease therapy.

In certain embodiments, the Lewy body disease therapy is gene therapy. In one non-limiting embodiment, the gene therapy comprises administering a nucleic acid encoding a protein with GBA, SMPD1, HEXA, and/or MCOLN1 protein activity. In certain embodiments, the therapy comprises administering protein replacement therapy, wherein a protein with GBA, SMPD1, HEXA, and/or MCOLN1 protein activity is administered to the subject.

The present disclosure further provides for a kit for determining whether a subject is at risk of having Lewy body disease, comprising reagents for detecting the presence of one or more variants in a gene selected from the group consisting of GBA, SMPD1, HEXA, MCOLN1, and combinations thereof, in a biological sample from a subject.

In certain embodiments, the reagent comprises a plurality of nucleic acid probes that specifically hybridize to a nucleic acid comprising the one or more variants.

In certain embodiments, the reagent comprises an antibody or antigen-binding fragment thereof, that specifically binds to a protein encoded by a nucleic acid comprising the one or more variants.

The foregoing has outlined rather broadly the features and technical advantages of the present application in order that the detailed description that follows may be better understood. Additional features and advantages of the application will be described hereinafter which form the subject of the claims of the application. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of the application, both as to its organization and method of operation, together with further objects and advantages will be better understood from the following description.

### 4. BRIEF DESCRIPTION OF FIGURES

FIG. 1A-B. GCase and HexA activity in autopsy brain tissue. A) GCase activity was significantly reduced in LBD cases carrying GBA mutations (n=16) compared to LBD non-GBA carriers (n=18) and controls (n=30). Differences in activity for HEXA were not significant in any group. B) GCase was significantly reduced in LBD cases with mutations classified as ‘severe’ Gaucher disease type (L444P, 84insGG etc.) compared to controls, and to LBD cases with mutations producing ‘mild’ Gaucher phenotypes (N370S) or variants of unknown phenotypic effect (E326K, T369M). Differences in activity for HEXA were not significant. * p<0.05, ** p<0.01, *** p<0.001

FIG. 2A-B. Heat Maps showing significant changes in lipid classes. A) Heat map showing statistically significant changes in major lipid subclasses in LBD GBA mutation carriers compared to LBD wildtype, AD cases and controls. B) Heat map showing statistically significant changes in lipid classes in LBD GBA mutation carriers compared to LBD wildtype, AD cases and controls. The heat map columns reflect all significant lipid changes (q<0.05) in a diseased compared to control patients. The color bar represents the log 2 value of the ratio of each lipid species. Statistical analysis for the AD and LBD Mutation samples was based on the one way analysis of variance followed by post hoc Fisher's least significant difference test while the LBD (wildtype GBA) samples was based on Student's T-test. A false discovery rate control was used to correct for multiple comparisons.

FIG. 3. Comparative lipid profile of post-mortem brain tissue obtained from patients diagnosed with various neurological conditions. The individual lipid subclasses of each group of patients was expressed as relative to control group levels for 2 separate sets of experiments (i.e. AD and LBD GBA mutation carrier relative to Control S1, LBD non carrier (wildtype GBA) relative to Control S2). Statistical analysis for the AD and LBD Mutation samples was based on the one way analysis of variance followed by post hoc Fisher's least significant difference test while the LBD non carrier (wildtype GBA) samples was based on Student's T-test. A false discovery rate control was used to correct for multiple comparisons. * q<0.05, ** q<0.01, *** q<0.001. PC, phosphatidylcholine; ePC, ether phosphatidylcholine; PE, phosphatidylethanolamine; pPE, plasmalogen phosphatidylethanolamine; PS, phosphatidylserine; PI, phosphatidylinositol; PA, phosphatidic acid; PG, phosphatidylglycerol; LBPA, lysobisphosphatidic acid; Cer, ceramide; SM, sphingomyelin; dhSM, dihydrosphingomyelin; GalCer, galactosylceramide; GluCer, glucosylceramide; Sulf, sulfatide; Sulf-h, hydroxylated sulfatide; GM3, monosialodihexosylganglioside.

FIG. 4A-B. Principle Component Analysis to examine AJ ancestry. A) All autopsies with genome-wide association study (GWAS) data (n=62); B) White autopsies with GWAS data (n=49).

FIG. 5. Demographic and neuropathological characteristics of white autopsy subjects described by Example 1.

FIG. 6A-B. Gene-wise association of SKAT analysis with AJ controls, as described by Example 1. (A) Gene-wise association with SKAT analysis with AJ controls only (n=128). (B) Gene-wise association SKAT analysis with brain controls (n=33) and AJ controls (n-128).

FIG. 7. Characteristics of autopsy subjects who had lipidomic analysis, as described by Example 1.

FIG. 8. Listing of post-mortem intervals (PMI) of subject brains for cold and frozen autopsies.

FIG. 9. Forest plot of meta-analysis of SMPD1 mutations in synucleinopathies. The forest plot depicts the odds ratios (ORs) from five previously published studies and Example 5, using the fixed-effect model. The combined OR was estimated with the Cochran-Mantel-Haenszel Test: CMH=65.0480, df=1, p<0.0001. Tarone's Test for Heterogeneity: χ2=9.4178, df=5, p=0.0935.

FIG. 10A-C. Knockdown of SMPD1 leads to α-synuclein accumulation in cellular models. siRNA knockdown efficiency for SMPD1 and its effect in the level of α-synuclein is demonstrated by Western blot analysis. Expression of SMPD1 and α-synuclein was analyzed by Western blot in cell lysates from HeLa cells (a,b) and M17 cells (c) transfected with ASM/1 (a,c) and ASM/2 (b) siRNAs targeting SMPD1 at 96h after transfection. Densitometry analysis shows the band density ratios of SMPD1 and alphα-synuclein to actin as indicated in the panels next to each blot. The quantifications are average of two independent experiments.

FIG. 11. Amino acid sequence of human GBA (SEQ ID NO:1).

FIG. 12. Amino acid sequence of human SMPD1 (SEQ ID NO:2).

FIG. 13A-B. Amino acid sequence of human HEXA (SEQ ID NO:3 and 4).

FIG. 14. Amino acid sequence of human MCOLN1 (SEQ ID NO:5).

FIG. 15. Genomic nucleotide sequence of human GBA (SEQ ID NO:6).

FIG. 16. Genomic nucleotide sequence of human SMPD1 (SEQ ID NO:7).

FIG. 17. Genomic nucleotide sequence of human HEXA (SEQ ID NO:8)

FIG. 18. Genomic nucleotide sequence of human MCOLN1 (SEQ ID NO:9).

FIG. 19. Primers used for PCR amplification and sequencing of SMPD1, as described by Example 5.

FIG. 20. Molecular inversion probes (MIPs) used for next generation sequencing of the coding region of SMPD1, as described by Example 5.

### 5. DETAILED DESCRIPTION

For clarity and not by way of limitation the detailed description of the invention is divided into the following subsections:

5.1 Definitions;

5.2 Lysosomal Storage Genes;

5.3 Methods of Diagnosis;

5.4 Methods of Treatment; and

5.5 Kits.

### 5.1 Definitions

As used herein, the use of the word “a” or “an” when used in conjunction with the term “comprising” in the claims and/or the specification can mean “one,” but it is also consistent with the meaning of “one or more,” “at least one,” and “one or more than one.” Some embodiments of the invention can consist of, or consist essentially of, one or more elements, method steps, and/or methods of the invention. It is contemplated that any method or composition described herein can be implemented with respect to any other method or composition described herein.

The term “biomarker” as used herein, includes nucleic acids and proteins that are related to the activity level of the genes GBA, SMPD1, HEXA and MCOLN1, as described herein.

As used herein, the term “variant” refers to a mutation that is a change in genomic DNA, messenger RNA (mRNA), and/or protein that differs from the general population, for example, the general population of individuals that do not have LBD. It includes, but is not limited to, single-nucleotide polymorphisms (SNPs), and copy-number variations (CNV). A SNP is a variation in a single nucleotide at a specific position in the genome, which is present to some appreciable degree within a population. SNPs can lead to differences in susceptibility to disease and response to treatments. In certain embodiments, a mutation is a permanent alteration of the nucleotide sequence in the genome. A mutation can result from DNA damage, errors during replication, insertion or deletion of segments of DNA, etc. A CNV is a deletion or duplication of certain segment in the genome which can lead a deletion or duplication of one or more genes.

As used herein, the term “Minor Allele Frequency”, or “MAF” refers to the frequency at which an allele of a gene occurs in a given population. Said alleles can be, for example, the variants described herein.

As used herein, the term “biological sample” refers to a sample of biological material obtained from a subject, preferably a human subject, including tissue, a tissue sample, a cell sample, a tumor sample, a stool sample and a biological fluid, e.g., blood, urine, lymphatic fluid, ascites, pancreatic cyst fluid and a nipple aspirate. In one non-limiting embodiment, the presence of one or more variants described herein is determined in a peripheral blood sample obtained from a subject.

The term “patient” or “subject,” as used interchangeably herein, refers to any warm-blooded animal, preferably a human. Non-limiting examples of non-human subjects include non-human primates, dogs, cats, mice, rats, guinea pigs, rabbits, fowl, pigs, horses, cows, goats, sheep, etc.

The term “Lewy body disease”, as used herein, refers to disorders characterized by aggregates of protein comprising alphα-synuclein that develop inside nerve cells. The alphα-synuclein protein aggregates can further comprise other proteins, such as ubiquitin, neurofilament protein, alpha B crystalline, and/or tau proteins. In certain embodiments, subjects with Lewy body disease exhibit dementia. Dementia is the loss of mental functions severe enough to affect normal activities and relationships. The symptoms of the LBD include, but are not limited to, changes in alertness and attention, hallucinations, problems with movement and posture, muscle stiffness, confusion, and loss of memory. In certain embodiments, LBD includes Parkinson's disease and/or Lewy body with dementia.

### 5.2 Lysosomal Storage Genes

A lysosome is a membrane-bound cell organelle found in most animal cells, containing hydrolytic enzymes capable of breaking down biomolecules, including but not limited to proteins, nucleic acids, carbohydrates, lipids, and cellular debris. Lysosomes contain more than fifty different enzymes and channel proteins, dysfunction of which may result in lysosomal storage disease. Lysosomal storage genes include, but are not limited to, GBA, SMPD1, HEXA and MCOLN1.

Glucosylceramidase beta (official symbol: GBA, GenBank ID:2629, also known as β-Glucocerebrosidase, acid β-glucosidase, D-glucosyl-N-acyl sphingosine glucohydrolase, or GCase) is a gene encoding a lysosomal membrane protein that cleaves the beta-glucosidic linkage of glycosylceramide, an intermediate in glycolipid metabolism. Mutations in this gene are known to cause Gaucher disease, a lysosomal storage disease characterized by an accumulation of glucocerebrosides. In certain embodiments, the GBA is a human GBA encoded by a nucleic acid sequence described by NCBI GenBank Accession Nos. NG_009783.1, NM_000157.3, NM_001005741.2, NM_001005742.2, NM_001171811.1, NM_001171812.1, NC_000001.11, XM_011509407.1, XM_006711270.1, NW_003315906.1, XM_011546930.1, XM_006726211.1, and/or NC_018912.2. In certain embodiments, the GBA for use in the presently disclosed subject matter can include a nucleotide sequence having at least 85%, at least 90%, at least 91%, at least 92%, at least 93%, at least 94%, at least 95%, at least 96%, at least 97%, at least 98% or at least 99% identity to a GBA nucleic acid sequence described herein.

In certain embodiments, the GBA is a human GBA comprising an amino acid sequence described by NCBI GenBank Accession Nos. NP_000148.2, NP_001005741.1, NP_001005742.1, NP_001165282.1, NP_001165283.1,) XP_011507709.1,XP_006711333.1,XP_011545232.1, and/or XP_0067262741 In certain embodiments, the GBA for use in the presently disclosed subject matter can include an amino acid sequence having at least 85%, at least 90%, at least 91%, at least 92%, at least 93%, at least 94%, at least 95%, at least 96%, at least 97%, at least 98% or at least 99% identity to a GBA amino acid sequence described herein.

In certain embodiments, the GBA is a human GBA comprising an amino acid sequence described by SEQ ID NO:1, or a nucleic acid encoding said amino acid sequence.

Sphingomyelin phosphodiesterase 1 (official symbol: SMPD1, GenBank ID:6609, also known as acid sphingomyelinase) is a gene encoding a lysosomal acid sphingomyelinase that converts sphingomyelin to ceramide. The encoded protein also has phospholipase C activity. Defects in this gene can be a cause of Niemann-Pick disease type A (NPA) and Niemann-Pick disease type B (NPB). In certain embodiments, the SMPD1 is a human SMPD1 encoded by a nucleic acid sequence described by NCBI GenBank Accession Nos. NG_011780.1, NM_000543.4, NM_001007593.2, NM_001318087.1, NM_001318088.1, NR_027400.2, NR_134502.1, NC_000011.10, XM_011520303.1, XM_005253075.3, XM_011520304.1, XR_930886.1, and/or NC_018922.2. In certain embodiments, the SMPD1 for use in the presently disclosed subject matter can include a nucleotide sequence having at least 85%, at least 90%, at least 91%, at least 92%, at least 93%, at least 94%, at least 95%, at least 96%, at least 97%, at least 98% or at least 99% identity to a SMPD1 nucleic acid sequence described herein.

In certain embodiments, the SMPD1 is a human SMPD1 comprising an amino acid sequence described by NCBI GenBank Accession Nos. NP_000534.3, NP_001007594.2, NP_001305016.1, NP_001305017.1, XP_011518605.1, XP_005253132.1, and/or XP_011518606.1. In certain embodiments, the SMPD1 for use in the presently disclosed subject matter can include an amino acid sequence having at least 85%, at least 90%, at least 91%, at least 92%, at least 93%, at least 94%, at least 95%, at least 96%, at least 97%, at least 98% or at least 99% identity to a SMPD1 amino acid sequence described herein.

In certain embodiments, the SMPD1 is a human SMPD1 comprising an amino acid sequence described by SEQ ID NO:2, or a nucleic acid encoding said amino acid sequence.

Hexosaminidase subunit alpha (official symbol: HEXA, GenBank ID: 3073, also known as Hexosaminidase A) is a gene encoding a member of the glycosyl hydrolase 20 family of proteins. The encoded preproprotein is proteolytically processed to generate the alpha subunit of the lysosomal enzyme betα-hexosaminidase. This enzyme, together with the cofactor GM2 activator protein, catalyzes the degradation of the ganglioside GM2, and other molecules containing terminal N-acetyl hexosamines. Mutations in this gene lead to an accumulation of GM2 ganglioside in neurons, the underlying cause of neurodegenerative disorders termed the GM2 gangliosidoses, including Tay-Sachs disease (GM2-gangliosidosis type I). Alternative splicing results in multiple transcript variants, at least one of which encodes a preproprotein that is proteolytically processed. In certain embodiments, the HEXA is a human HEXA encoded by a nucleic acid sequence described by NCBI GenBank Accession Nos. NG_009017.1, NM_000520.5, NM_001318825.1, NR_134869.1, NC_000015.10, and/or NC_018926.2. In certain embodiments, the HEXA for use in the presently disclosed subject matter can include a nucleotide sequence having at least 85%, at least 90%, at least 91%, at least 92%, at least 93%, at least 94%, at least 95%, at least 96%, at least 97%, at least 98% or at least 99% identity to a HEXA nucleic acid sequence described herein.

In certain embodiments, the HEXA is a human HEXA comprising an amino acid sequence described by NCBI GenBank Accession Nos. NP_000511.2, and/or NP_001305754.1. In certain embodiments, the HEXA for use in the presently disclosed subject matter can include an amino acid sequence having at least 85%, at least 90%, at least 91%, at least 92%, at least 93%, at least 94%, at least 95%, at least 96%, at least 97%, at least 98% or at least 99% identity to a HEXA amino acid sequence described herein.

In certain embodiments, the HEXA is a human HEXA comprising an amino acid sequence described by SEQ ID NO:3 or 4, or a nucleic acid encoding said amino acid sequence.

Mucolipin 1 (official symbol: MCOLN1, GenBank ID:57192, also known as transient receptor potential cation channel, mucolipin subfamily, member 1 or TRPML1) is a gene encoding a member of the transient receptor potential (TRP) cation channel gene family. The transmembrane protein localizes to intracellular vesicular membranes including lysosomes, and functions in the late endocytic pathway and in the regulation of lysosomal exocytosis. The channel is permeable to Ca(2+), Fe(2+), Na(+), K(+), and H(+), and is modulated by changes in Ca(2+) concentration. Mutations in this gene result in mucolipidosis type IV. In certain embodiments, the MCOLN1 is a human MCOLN1 encoded by a nucleic acid sequence described by NCBI GenBank Accession Nos. NG_015806.1, NM_020533.2, NC_000019.10, and/or NC_018930.2. In certain embodiments, the MCOLN1 for use in the presently disclosed subject matter can include a nucleotide sequence having at least 85%, at least 90%, at least 91%, at least 92%, at least 93%, at least 94%, at least 95%, at least 96%, at least 97%, at least 98% or at least 99% identity to a MCOLN1 nucleic acid sequence described herein.

In certain embodiments, the MCOLN1 is a human MCOLN1 comprising an amino acid sequence described by NCBI GenBank Accession No. NP_065394.1. In certain embodiments, the MCOLN1 for use in the presently disclosed subject matter can include an amino acid sequence having at least 85%, at least 90%, at least 91%, at least 92%, at least 93%, at least 94%, at least 95%, at least 96%, at least 97%, at least 98% or at least 99% identity to a MCOLN1 amino acid sequence described herein.

In certain embodiments, the MCOLN1 is a human MCOLN1 comprising an amino acid sequence described by SEQ ID NO:5, or a nucleic acid encoding said amino acid sequence.

Pathogenic mutations in patients with associated lysosomal storage disorder are shown in Table 2. Other lysosomal storage genes, dysfunction of which may lead to lysosomal storage disorders, include but not limit to, ceramidase, alpha-galactosidase A, alpha-galactosidase B, sphingomyelinases, and battenin.

### 5.3 Methods of Diagnosis

Embodiments of the present disclosure relate to methods for determine the risk of Lewy body disease in a subject. In certain embodiments, the method comprises obtaining a biological sample from the subject and determining if the biological sample from the subject comprises one or more variants of one or more genes selected from the group consisting of GBA, SMPD1, HEXA and MCOLN1, or combinations thereof, wherein the presence of the one or more variants is an indication that the subject is at risk of having or developing Lewy body disease.

In certain embodiments, a method for determine the risk of Lewy body disease in the subject includes obtaining at least one biological sample from the subject, wherein the sample can comprise, but is not limited to, blood (including plasma or serum); tissue sample, for example, a brain biopsy; and/or cerebral spinal fluid. The step of collecting a biological sample can be carried out either directly or indirectly by any suitable technique. For example, a blood sample from a subject can be carried out by phlebotomy or any other suitable technique, with the blood sample processed further to provide a serum sample or other suitable blood fraction.

In certain embodiments, the method comprises obtaining a biological sample from the subject and determining if the biological sample from the subject comprises one or more variants of GBA, wherein the variant of GBA comprises a single nucleotide polymorphism (SNP) selected from the group consisting of rs80356773, rs80356771, rs421016, rs1064651, rs76763715, rs75548401, rs2230288, rs367968666, rs61748906, rs114099990, rs387906315, and combinations thereof.

In certain embodiments, the GBA variant comprises the SNP rs114099990.

In certain embodiments, the GBA variant comprises a mutation in the GBA nucleic acid sequence selected from the group consisting of g.1864A>G, c.38A>G, g.7549A>C, c.1584A>C, g.6728A>G, c.1226A>G, g.6195G>A, c.1093G>A, g.7319T>C, c.1448T>C, g.3940C>T, c.474C>T, g.4343T>C, c.667T>C, g.5026C>T, c.795C>T, g.7314G>A, c.1443G>A, g.7375C>T, c.1504C>T, g.7366G>C, c.1495G>C, g.3942G>A, c.476G>A, g.1367C>T, 84insGG, and combinations thereof (wherein “g.” describes the position comprising the nucleotide variation in the GBA gene, and “c.” describes the position comprising the nucleotide variation in the GBA cDNA).

In certain embodiments, the GBA variant comprises a mutation in the GBA amino acid sequence selected from the group consisting of p.R535H, p.R502C, p.L483P, p.D448H, p.N409S, p.T408M, p.E365L, p.H294Q, p.W223R, p.Leu29AlafsX188, p.E388K, p.N188R, p.S196P, p.V191G, and combinations thereof.

In certain embodiments, the GBA variant comprises a mutation in the GBA amino acid sequence selected from the group consisting of p.Lys13Arg, p.Asp482Asn, p.Va1499Leu, p.Arg159Gln, and combinations thereof.

In certain embodiments, the method comprises obtaining a biological sample from the subject and determining if the biological sample from the subject comprises one or more variants of SMPD1, wherein the variant of SMPD1 comprises a single nucleotide polymorphism (SNP) selected from the group consisting of rs144465428, rs1050228, rs71056748, rs7951904, rs1050239, rs8164, rs72896268, rs2723669, rs142178073, rs144873307, rs142787001 and combinations thereof.

In certain embodiments, the SMPD1 variant comprises a mutation in the SMPD1 nucleic acid sequence selected from the group consisting of a IVS3+2T>C in the SMPD1 genomic nucleic acid, c.1829delCGG mutation in SMPD1 cDNA, and combinations thereof.

In certain embodiments, the SMPD1 variant comprises a mutation in the SMPD1 amino acid sequence selected from the group consisting of p.Q19R, p.V36A, p.Leu49_Ser50insAL, p.Leu49_Ser50insALAL, p.D212D, p.E358K, p.G508R, p.R542L, p.V301I, p.M33I, p.G492S, p.E517V, p.R418Q, p.R291H, p.A487V, p.P331A, p.R378H, p.R498L, p.G530A, p.R610H, and combinations thereof.

In certain embodiments, the SMPD1 variant comprises a mutation in the SMPD1 amino acid sequence selected from the group consisting of p.E108K, p.V114M, p.I127V, p.E141D, p.C159R, p.D225Y, p.C228R, p.G247S, p.K251R, p.D253A, p.Q289X, p.R296Q, p.P325A, p.R341H, p.L379F, p.K435R, p.W437C, p.V462M, p.G504X, p.E543X, p.V5591, p.M566T, and combinations thereof.

In certain embodiments, the method comprises obtaining a biological sample from the subject and determining if the biological sample from the subject comprises one or more variants of HEXA, wherein the variant of HEXA comprises a single nucleotide polymorphism (SNP) selected from the group consisting of rs2302449, rs387906309, rs73440586, rs117513345, rs1800428, rs121907970, rs117160567, rs2288259, rs1800431, rs121907954, rs117160567, rs10220917, and combinations thereof.

In certain embodiments, the HEXA variant comprises a 1277_1278insTATC mutation in the HEXA genomic nucleic acid sequence, a c.672+30T>G mutation in the HEXA cDNA sequence, or combinations thereof.

In certain embodiments, the HEXA variant comprises a mutation in the HEXA amino acid sequence selected from the group consisting of p.Y427I, p.R247W, p.I436V, p.G269S, p.V192I, and combinations thereof.

In certain embodiments, the method comprises obtaining a biological sample from the subject and determining if the biological sample from the subject contains one or more variants of MCOLN1, wherein the variant of MCOLN1 comprises a single nucleotide polymorphism (SNP) selected from the group consisting of rs45513896, rs145706318, rs73003348, rs2305889, rs139922988, rs145386883, rs686796, rs113261161, rs61736600, rs612862, rs142259322, rs147754092, and combinations thereof.

In certain embodiments, the MCOLN1 variant comprises a mutation in the MCOLN1 amino acid sequence selected from the group consisting of p.P197S, p.T261M, p.S257R, p.A138V, and combinations thereof.

In certain embodiments, the GBA, HEXA, SMPD1, and MCOLN1 are as described by tables 2, 6, 8, 12, and 13.

In certain embodiments, a subject at risk for having LBD further exhibits decreased GCase activity in a sample from the subject compared to the GCase activity in a sample from a subject who does not have LBD, or compared to a reference level determined from one or more subjects that do not have LBD or do not have a GBA mutation(s).

In certain embodiments, a subject at risk for having LBD further exhibits a decreased β-glucocerebrosidase: α-hexosaminidase protein activity ratio in a sample from the subject compared to the protein activity ratio in a sample from a subject who does not have LBD, or compared to a reference level determined from one or more subjects that do not have LBD or do not have a GBA mutation(s).

In certain embodiments, a subject at risk for having LBD further exhibits an altered lipid profile, including changes in concentrations of phospholipid subclasses (such as phosphatidylcholine (PC), phosphatidylethanolamine (PE) and phosphatidylserine (PS)), sphingolipid composition (such as sphingolipid sphingomyelin (SM), dihydrosphingomyelin (dhSM) species, and ceramide (Cer)), GCase substrate (such as GluCer), complex glycosphingolipid (such as GM3), galactosylceramide (GalCer) and its biosynthetic derivative sulfatides containing hydroxy fatty.

In one non-limiting embodiment, the amount of phosphatidylcholine, phosphatidylethanolamine, and/or sphingolipid sphingomyelin is decreased in a sample from the subject compared to a sample from a subject who does not have LBD, or compared to a reference level determined from one or more subjects that do not have LBD or do not have a GBA mutation(s).

In another non-limiting embodiment, the level of phosphatidylserine, dihydrosphingomyelin, ceramide, glycosphingolipid, and/or galactosylceramide (or biosynthetic derivative sulfatides thereof) is increased in a sample from the subject compared to a sample from a subject who does not have LBD, or compared to a reference level determined from one or more subjects that do not have LBD or do not have a GBA mutation(s).

In certain non-limiting embodiments, a subject at risk for having LBD further exhibits decreased ASMase (Acid sphingomyelinase) activity in a sample from the subject compared to the ASMase activity in a sample from a subject who does not have LBD, or compared to a reference level determined from one or more subjects that do not have LBD or do not have an SMPD1 mutation(s). In certain embodiments, a decreased level of ASMase activity indicates a risk of early onset LBD, for example, early onset Parkinson's Disease.

In certain embodiments, the Minor Allele Frequency (MAF) of the one or more mutations are below a predetermined threshold. In one embodiment, the MAF is less than 0.05. In other embodiments, the MAF is less than 0.5, 0.4, 0.3, 0.2, 0.1, 0.04, 0.03, 0.02, 0.01 or 0.001.

In certain embodiments, one or more variants that are expressed with a higher frequency in a control subject population that do not have LBD, or are not at risk for LBD, are protective variants. In certain embodiments, the presence of one or more of such protective variants in a sample is indicative of a reduced risk for developing LBD.

A variant used in the methods of the disclosure can be identified in a biological sample using any method known in the art. Determining the presence of a variant, protein or degradation product thereof, the presence of mRNA or pre-mRNA, or the presence of any biological molecule or product that is indicative of the presence of the variant, or degradation product thereof, can be carried out for use in the methods of the disclosure by any method described herein or known in the art.

### 5.3.1 DNA Detection Techniques

Any method for detecting the presence of a nucleic acid can be used to identify the presence of a variant in a GBA, SMPD1, HEXA, and/or MCOLN1 gene, as described herein. Detection of a DNA variant can be achieved, for example, by southern blotting, wherein a preparation of DNA is run on an agarose gel, and transferred to a suitable support, such as activated cellulose, nitrocellulose or glass or nylon membranes. Radiolabeled DNA probes are then hybridized to the preparation, washed and analyzed by autoradiography.

Detection of DNA can further be accomplished using amplification methods. For example, it is within the scope of the present disclosure to use primers designed for detecting a variant via polymerase chain reaction (PCR) or a variantion thereof for genotyping. Variations of PCR include but not limited to, multiplex PCR (multiple selected target regions in a sample are amplified simultaneously using different pairs of primers), nested PCR (includes two successive PCRs to amplify templates in low copy numbers in specimens), amplification refractory mutation system (ARMS) PCR (genotype of a sample could be determined using two complementary reactions: one containing a specific primer for the amplification of normal DNA sequence at a given locus and the other containing a mutant specific primer for amplification of mutant DNA), and real time PCR (amplified DNA is detected as the PCR progresses).

Fluorescence in situ hybridization (FISH) can also be employed to show specific localization of a variant in chromosomes. Rapid diagnosis can be achieved by using specific probes. Usually a denatured probe is added to a metaphase chromosome spread and incubated overnight to allow sequence-specific hybridization. After washing off the unbound probe, the bound probe is visualized by its fluorescence when exposed to an energy wavelength that induces the probe to fluoresce. The site of the variant can be visualized in situ.

Alternatively, DNA microarray can be used to test for multiple variants. In this technology, single DNA strands including sequences of different targets are fixed to a solid support in an array format. The sample DNA or cDNA labeled with fluorescent dyes is hybridized to the chip. The variants and their quantities in the sample are determined by the fluorescence under a laser system.

Any DNA sequencing methods can be used to detect the one or more variants. For example, a Sanger sequencing can be used to detect a variant in one of the genes consist of GBA, SMPD1, HEXA, MCOLN1 or combinations thereof. Double stranded DNA is denatured into single stranded DNA. A Sanger reaction comprises a single strand DNA, primer, a mixture of a particular ddNTP with normal dNTPs. A fluorescent dye molecule is covalently attached to the dideoxynucleotide. ddNTPs cannot form a phosphodiester bond with the next deoxynucleotide so that they terminate DNA chain elongation.

In certain embodiments, next generation sequencing (NGS) is used to detect the one or more variants. NGS systems provide several sequencing approaches including whole-genome sequencing (WGS), whole exome sequencing (WES), transcriptome sequencing, methylome, etc. Representative technologies include but not limited to, Illumina (Solexa) sequencing (Mardis ER (2008). “Next-generation DNA sequencing methods”. Annu Rev Genomics Hum Genet 9: 387-402. doi:10.1146/annurev.genom.9.081307.164359. PMID 18576944. Incorporated herein), Roche 454 sequencing (Schuster S C (January 2008). “Next-generation sequencing transforms today's biology”. Nat. Methods 5 (1):16-8. doi:10.1038/nmeth1156. PMID 18165802. Incorporated herein), ion torrent: Proton/PGM sequencing (Rusk N (2011). “Torrents of sequence”. Nat Meth 8 (1):44-44. doi:10.1038/nmeth.f330. Incorporated herein), and SOLiD sequencing (Huang Y F, Chen S C, Chiang Y S, Chen T H, Chiu K P (2012). “Palindromic sequence impedes sequencing-by-ligation mechanism”. BMC Systems Biology. 6 Suppl 2: S10. doi:10.1186/1752-0509-6-S2-S10. PMID 23281822. Incorporated herein).

### 5.3.2 RNA Detection Techniques

Any method for detecting the presence of a nucleic acid can be used to identify the presence of a variant in a GBA, SMPD1, HEXA, and/or MCOLN1 gene, as described herein. Detection of RNA transcripts can be achieved, for example, by Northern blotting, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present disclosure to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994).

In one embodiment, quantitative real-time polymerase chain reaction (qRT-PCR) is used to evaluate mRNA levels of a variant. The levels of a variant or RNA product thereof and a control mRNA can be quantitated in cancer tissue or cells and adjacent benign tissues. In one specific embodiment, the levels of one or more variant or RNA product thereof can be quantitated in a biological sample.

Other known amplification methods which can be utilized herein include but are not limited to the so-called “NASBA” or “3 SR” technique described in PNAS USA 87: 1874-1878 (1990) and also described in Nature 350 (No. 6313):91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO9322461.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with haematoxylin to demonstrate the histological composition of the sample, and dark field imaging with a suitable light filter shows the developed emulsion. Non-radioactive labels such as digoxigenin can also be used.

Another method for evaluation of a variant or RNA product thereof is by fluorescent in situ hybridization (FISH). FISH is a technique that can directly identify a specific region of RNA in a cell and therefore enables to visual determination of the biomarker expression in tissue samples. The FISH method has the advantages of a more objective scoring system and the presence of a built-in internal control consisting of the variant gene signals present in all non-neoplastic cells in the same sample. Fluorescence in situ hybridization is a direct in situ technique that is relatively rapid and sensitive. FISH test also can be automated.

Alternatively, mRNA product of a variant can be detected on a DNA array, chip or a microarray. Oligonucleotides corresponding to the genetic variant(s) are immobilized on a chip which is then hybridized with labeled nucleic acids of a test sample obtained from a subject. Positive hybridization signal is obtained with the sample containing variant DNA or transcripts. Methods of preparing DNA arrays and their use are well known in the art. (See, for example, U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

Types of probes for detection of RNA include cDNA, riboprobes, synthetic oligonucleotides and genomic probes. The type of probe used will generally be dictated by the particular situation, such as riboprobes for in situ hybridization, and cDNA for Northern blotting, for example. Most preferably, the probe is directed to nucleotide regions unique to the particular variant or RNA product thereof. The probes can be as short as is required to differentially recognize the particular variant or RNA product thereof, and can be as short as, for example, 15 bases; however, probes of at least 17 bases, more preferably 18 bases and still more preferably 20 bases are preferred. Preferably, the primers and probes hybridize specifically under stringent conditions to a nucleic acid fragment having the nucleotide sequence corresponding to the target gene. As herein used, the term “stringent conditions” means hybridization will occur only if there is at least 95% and preferably at least 97% identity between the sequences.

The form of labeling of the probes can be any that is appropriate, such as the use of radioisotopes, for example, 32P and 35S. Labeling with radioisotopes can be achieved, whether the probe is synthesized chemically or biologically, by the use of suitably labeled bases.

Alternatively, mRNA product of a variant can be detected by RNA-seq (RNA sequencing). RNA-seq uses next-generation sequencing (NGS) to reveal the presence and quantity of RNA in a biological sample. RNA-Seq can analyze the continually changing cellular transcriptome. RNA-Seq can also analyze at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

### 5.3.3 Protein Detection Techniques

Methods for the detection of protein biomarkers are well known to those skilled in the art, and include but are not limited to mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry. These methods use antibodies, or antibody equivalents, to detect protein, or use biophysical techniques. Antibody arrays or protein chips can also be employed, see for example U.S. Patent Application Nos: 20030013208A1; 20020155493A1, 20030017515 and U.S. Pat. Nos. 6,329,209 and 6,365,418, herein incorporated by reference in their entirety.

ELISA and RIA procedures can be conducted such that a variant or protein product thereof is labeled (with a radioisotope such as 125I or 35S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the variant or protein product thereof in the sample is allowed to react with the corresponding immobilized antibody, radioisotope or enzyme-labeled anti-biomarker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting the mixture with labeled antibody. Other conventional methods can also be employed as suitable.

In one embodiment, a method for detecting the one or more variants includes the steps of: contacting a biological sample, e.g., blood, with an antibody or equivalent (e.g., fragment) thereof which selectively binds the variant or protein product thereof, and detecting whether the antibody or equivalent thereof is bound to the sample. A method can further include contacting the sample with a second antibody, e.g., a labeled antibody. The method can further include one or more steps of washing, e.g., to remove one or more reagents.

It can be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene can provide a suitable support.

Enzymes employable for labeling are not particularly limited, but can be selected, for example, from the members of the oxidase group. These catalyze production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect a variant or protein product thereof according to a practitioner's preference based upon the present disclosure. One such technique that can be used for detecting and quantitating a variant's protein levels is Western blotting (Towbin et al., Proc. Nat. Acad. Sci. 76:4350 (1979)). Cells can be frozen, homogenized in lysis buffer, and the lysates subjected to SDS-PAGE and blotting to a membrane, such as a nitrocellulose filter. Antibodies (unlabeled) are then brought into contact with the membrane and assayed by a secondary immunological reagent, such as labeled protein A or anti-immunoglobulin (suitable labels including 125I, horseradish peroxidase and alkaline phosphatase). Chromatographic detection can also be used. In some embodiments, immunodetection can be performed with antibody to a variant or protein product thereof using the enhanced chemiluminescence system (e.g., from PerkinElmer Life Sciences, Boston, Mass.). The membrane can then be stripped and re-blotted with a control antibody, e.g., anti-actin (A-2066) polyclonal antibody from Sigma (St. Louis, Mo.).

Immunohistochemistry can be used to detect the expression and/ presence of a variant or protein product thereof, e.g., in a biopsy sample. A suitable antibody is brought into contact with, for example, a thin layer of cells, followed by washing to remove unbound antibody, and then contacted with a second, labeled, antibody. Labeling can be by fluorescent markers, enzymes, such as peroxidase, avidin or radiolabeling. The assay is scored visually, using microscopy and the results can be quantitated.

Other machine or autoimaging systems can also be used to measure immunostaining results for the variant or protein product thereof. As used herein, “quantitative” immunohistochemistry refers to an automated method of scanning and scoring samples that have undergone immunohistochemistry, to identify and quantitate the presence of a specified variant or protein product thereof, such as an antigen or other protein. The score given to the sample is a numerical representation of the intensity of the immunohistochemical staining of the sample, and represents the amount of target present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Antibodies against a variant or protein product thereof can also be used for imaging purposes, for example, to detect the presence of a variant or protein product thereof in cells of a subject. Suitable labels include radioisotopes, iodine (125I, 121I), carbon (14C), sulphur (35S), tritium (3H), indium (112In), and technetium (99mTc), fluorescent labels, such as fluorescein and rhodamine and biotin. Immunoenzymatic interactions can be visualized using different enzymes such as peroxidase, alkaline phosphatase, or different chromogens such as DAB, AEC or Fast Red.

For in vivo imaging purposes, antibodies are not detectable, as such, from outside the body, and so must be labeled, or otherwise modified, to permit detection. Markers for this purpose can be any that do not substantially interfere with the antibody binding, but which allow external detection. Suitable markers can include those that can be detected by X-radiography, NMR or MRI. For X-radiographic techniques, suitable markers include any radioisotope that emits detectable radiation but that is not overtly harmful to the subject, such as barium or caesium, for example. Suitable markers for NMR and MRI generally include those with a detectable characteristic spin, such as deuterium, which can be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99 m.

The labeled antibody or antibody fragment will then preferentially accumulate at the location of cells which contain a variant or protein product thereof. The labeled antibody or variant thereof, e.g., antibody fragment, can then be detected using known techniques. Antibodies include any antibody, whether natural or synthetic, full length or a fragment thereof, monoclonal or polyclonal, that binds sufficiently strongly and specifically to the variant or protein product thereof to be detected. In certain embodiments, an antibody can have a Kd of about 10⁻⁶M, 10⁻⁷M, 10⁻⁸M, 10⁻⁹M, 10⁻¹⁰M, 10⁻¹¹M, 10⁻¹²M, or less. The phrase “specifically binds” refers to binding of, for example, an antibody to an epitope or antigen or antigenic determinant in such a manner that binding can be displaced or competed with a second preparation of identical or similar epitope, antigen or antigenic determinant.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof, including, but not limited to Fv, Fab, Fab′ and F(ab′)2 fragments can be used. Such fragments can be produced by enzymatic cleavage or by recombinant techniques. For example, papain or pepsin cleavage can generate Fab or F(ab′)2 fragments, respectively. Other proteases with the requisite substrate specificity can also be used to generate Fab or F(ab′)2 fragments. Antibodies can also be produced in a variety of truncated forms using antibody genes in which one or more stop codons have been introduced upstream of the natural stop site. For example, a chimeric gene encoding a F(ab′)2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

### 5.4 Methods of Treatment

In certain embodiments, the present disclosure provides methods for prophylactic or therapeutic treatment (for example, preventative Lewy body disease treatment or treatment of diagnosed Lewy body disease) of subjects identified as being at risk for having LBD according to the methods of the present application. In certain embodiments, a method of preventing or treating Lewy body disease in a subject, comprises: (a) determining the presence of one or more variants in a biological sample obtained from the subject, wherein the one or more variants comprise one or more mutations in a gene selected from the group consisting of GBA, SMPD1, HEXA, MCOLN1, and combinations thereof; and (b) if the one or more mutations are present in the biological sample, treating the subject with a Lewy body disease therapy.

In certain embodiments, the Lewy body disease therapy is gene therapy. Gene therapy is a technique that uses genes or products thereof to treat or prevent disease. Common forms of gene therapy include, but are not limited to, inserting a normal gene to compete with an abnormal/mutated gene, inserting a normal gene to replace an abnormal/mutated gene, inactivating an abnormal/mutated gene, repairing an abnormal/mutated gene, altering the degree to which a gene is expressed (e.g., RNA interference), and introducing a new gene into the body to help fight a disease. Theraputic agents include but not limited to, single strand DNA or any modification or derivatives thereof, double strand DNA or any modification or derivatives thereof, single strand RNA or any modification or derivatives thereof, double strand RNA or any modification or derivatives thereof, peptide/protein or any modification or derivatives thereof, and biological products of said DNA, RNA or protein, or any modification or derivative thereof. Non-limiting examples of the agents includes viral vectors (e.g., retroviruses, lentiviruses adenoviruses, adeno-associated viruses), plasmids, BACs, YACs, peptides, proteins, oligonucleotides, microRNAs, siRNAs, dsRNAs, shRNAs. Therapeutic agents can be active at the genome level, RNA transcripts level, and/or protein level. The effect of a gene therapy can be permanent or temporary. Different agents, approaches can be combined to achieve an optimal outcome.

Methods for delivering the agents can also vary depending on the need. Common delivery methods include but not limited to, electroporation, microinjection, gene gun, impalefection, hydrostatic pressure, continuous infusion, sonication, magnetofection, viral vectors (e.g., retroviruses, lentiviruses adenoviruses, adeno-associated viruses, envelope protein pseudotyping of viral vectors, replication-competent vectors cis and trans-acting elements, herpes simplex virus) and chemical vehicles (e.g., oligonucleotides, lipoplexes, polymersomes, polyplexes, dendrimers, inorganic Nanoparticles, and cell-penetrating peptides).

In certain embodiments, the gene therapy comprises replacing the mutant variant with a functional variant of the gene. In one embodiment, the CRISPR system is used to replace the variant. Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

In certain embodiments, the gene therapy comprises delivering to the subject a functional product of the gene through a gene delivery vehicle. In one embodiment, the gene delivery vehicle is selected from the group consisting of viral vectors, plasmids, BACs, YACs, peptides and modified lipids. The another embodiment the gene delivery vehicle is an adenovirus vector.

In certain embodiments, the methods of treatment comprise administering a therapeutically effective amount of a GBA, SMPD1, HEXA, and/or MCOLN1 nucleic acid to a subject determined to be at risk for having Lewy body disease according to the methods described herein.

In certain embodiments, the methods of treatment comprise protein replacement therapy, which comprises administering a therapeutically effective amount of a protein that exhibits GBA, SMPD1, HEXA, and/or MCOLN1 protein activity. In certain embodiments, the method comprises administering a protein encoded by a GBA, SMPD1, HEXA, and/or MCOLN1 nucleic acid to a subject determined to be at risk for having Lewy body disease according to the methods described herein.

A “therapeutically effective amount” refers to an amount that is able to achieve one or more of a reduction in clinical symptom or signs of Lewy body disease, for example, dementia, decline in cognitive abilities (e.g., thinking, memory, language), Parkinsonian motor deficiencies (e.g., impairment in walking, muscle stiffness), and/or visual hallucinations. In certain embodiments, a “therapeutically effective amount” results in a prolongation of survival.

In certain embodiments, the Lewy body disease therapy comprises administration of a cholinesterase inhibitor (e.g., an acetylcholinesterase inhibitor) for treating cognitive symptoms; a DOPA Decarboxylase or DDC inhibitor such as carbidopa and L-DOPA (e.g., levodopa) for treating motor deficiencies; an antipsychotic medication (e.g., Olanzapine) for treating hallucinations; and/or antidepressants (e.g., fluoxetine).

### 5.5 Kits

In non-limiting embodiments, the present disclosure provides for a kit for determining whether a subject is at risk of having Lewy body disease, comprising reagents for detecting the presence of one or more variants in a gene selected from the group consisting of GBA, SMPD1, HEXA, MCOLN1, and combinations thereof, in a biological sample from a subject, wherein said variants comprise on or more of the mutations described herein.

Types of kits include, but are not limited to, packaged probe and primer sets (e.g. TaqMan probe/primer sets), arrays/microarrays, variant-specific antibodies and beads, which further contain one or more probes, primers or other detection reagents for detecting one or more variants of the present disclosure.

In a specific, non-limiting embodiment, a kit can comprise a pair of oligonucleotide primers suitable for polymerase chain reaction (PCR) or nucleic acid sequencing, for detecting one or more variants to be identified. A pair of primers can comprise nucleotide sequences complementary to a variant described herein, and be of sufficient length to selectively hybridize with said variant. Alternatively, the complementary nucleotides may selectively hybridize to a specific region in close enough proximity 5′ and/or 3′ to the variant position to perform PCR and/or sequencing. Multiple variant-specific primers can be included in the kit to simultaneously assay large number of variants. The kit can also comprise one or more polymerases, reverse transcriptase and nucleotide bases, wherein the nucleotide bases can be further detectably labeled.

In non-limiting embodiments, a primer can be at least about 10 nucleotides or at least about 15 nucleotides or at least about 20 nucleotides in length and/or up to about 200 nucleotides or up to about 150 nucleotides or up to about 100 nucleotides or up to about 75 nucleotides or up to about 50 nucleotides in length.

In a further non-limiting embodiment, the oligonucleotide primers can be immobilized on a solid surface or support, for example, on a nucleic acid microarray, wherein the position of each oligonucleotide primer bound to the solid surface or support is known and identifiable.

In a specific, non-limiting embodiment, a kit can comprise at least one nucleic acid probe, suitable for in situ hybridization or fluorescent in situ hybridization, for detecting the biomarker(s) to be identified. Such kits will generally comprise one or more oligonucleotide probes that have specificity for a plurality of variants.

In certain embodiments, the kits may comprise containers (including microliter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP, or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase).

In other non-limiting embodiments, a kit can comprise at least one antibody for immunodetection of the variant or protein product thereof to be identified. Antibodies, both polyclonal and monoclonal, specific for a the variant or protein product thereof, can be prepared using conventional immunization techniques, as will be generally known to those of skill in the art. The immunodetection reagents of the kit can include detectable labels that are associated with, or linked to, the given antibody or antigen itself. Such detectable labels include, for example, chemiluminescent or fluorescent molecules (rhodamine, fluorescein, green fluorescent protein, luciferase, Cy3, Cy5, or ROX), radiolabels (3H, 35S, 32P, 14C, 131I) or enzymes (alkaline phosphatase, horseradish peroxidase).

In a further non-limiting embodiment, the variant-specific antibody can be provided bound to a solid support, such as a column matrix, an array, or well of a microtiter plate. Alternatively, the support can be provided as a separate element of the kit.

In certain non-limiting embodiments, a kit can comprise one or more primers, probes, microarrays, or antibodies suitable for detecting one or more variants set forth in Table 2, or combinations thereof.

A kit may further contain means for comparing a variant's expression level (e.g., mRNA and/or protein expression level) in a subject sample and the expression level of the variant in a reference control sample. For example, but not by way of limitation, a kit of the present disclosure may contain one or more probes, primers, antibodies or other detection reagents for detecting a reference protein or mRNA, which can be used to normalize the expression levels of the one or more variants from the samples to allow comparison. Non-limiting examples of a reference protein, e.g., a housekeeping protein, include alpha- or beta-tubulin, actin, cofilin, vinculin and GADPH.

In certain non-limiting embodiments, a kit can further include instructions for using the kit to detect the one or more variants.

### 6. EXAMPLES

The following Example is offered to more fully illustrate the disclosure, but is not to be construed as limiting the scope thereof

### 6.1. Example 1:

### Gene-Wise Association of Variants in Four Lysosomal Storage Disorder Genes in Neuropathologically Confirmed Lewy Body Disease.

To determine whether variants in other lysosomal storage disease genes, in the same pathway as GBA, are associated with LBs, the inventor conducted an independent genetic study of the lysosomal storage disorder genes GBA, HEXA,

SMPD1, and MCOLN1 in 231 brain autopsies from the New York Brain Bank at Columbia University. Brain autopsies included neuropathologically defined LBD without AD changes (n=59), AD without significant LB pathology (n=71), ADLBV (n=68), and control brains without LB or AD neuropathology (n=33). The functional effect of GBA mutations was also determined by performing a biochemical analysis of GBA in a subset of brains.

### Methods

### Clinical Material

Brain tissue samples were obtained from the New York Brain Bank at Columbia University including cases obtained through the Alzheimer's Disease Research Center (ADRC) and the Center for Parkinson's Disease and Other Movement Disorders. Brain autopsies included neuropathologically defined LBD without AD changes (n=59), AD without significant LB pathology (n=71), ADLBV (n=68), and control brains without LB or AD neuropathology (n=33). (Table 1 and FIG. 5). LB and Alzheimer plaque and tangle pathology was assessed according to published guidelines as described previously[13-15]. Clinical information on dementia was available for 208 brain autopsy samples. While the primary analysis was of autopsy proven cases, an additional group of living controls (128 Ashkenazi Jewish (AJ) healthy individuals were used in a secondary analysis to supplement the limited number of brain autopsy controls. Columbia University Institutional Review Board approved the protocols and consent procedures. Written informed consent was obtained from all participants in the study.

### Brain Material

### Neuropathological Evaluation

LB pathology was assessed according to the Third Report of the DLB consortium, and utilized α-synuclein immunohistochemistry, with LB presence characterized as brainstem-predominant, or “cortical” (limbic or neocortical)[38]. Alzheimer's plaque and tangle pathology was detected using H&E and Bielschowsky stains, and β-amyloid and AT-8 immunohistochemistry, and rated using Braak & Braak, CERAD, and NIA-Reagan Institute (NIA-RI) criteria[39]. All cases with Braak stage III, IV, V, or VI neurofibrillary pathology, and/or plaque-based CERAD possible, probable, or definite AD, were rated as having “any Alzheimer's pathology”. Neuropathological findings were described as per the National Alzheimer's Coordinating Center (NACC) Neuropathology Manual V8.0[40]. Only cases that met NIA-RI criteria for intermediate or high likelihood of AD were deemed to have an “AD pathological diagnosis”. Cases with cortical LB by consortium criteria, were termed DLB if they did not have concomitant AD pathological diagnosis, and were termed Lewy Body Variant of AD (ADLBV) if they did.

### Clinical Evaluation of Dementia

Dementia was determined by consensus conference using DSM-IV-TR criteria; AD was determined using NINDS-ADRDA criteria, and LBD using McKeith criteria. For cases not seen in proximity to death, clinical history was re-obtained. As such, “onset of dementia” age was generally obtained prospectively during evaluations, but occasionally retrospectively in cases for whom there was more than a year between last clinical evaluation and death/autopsy.

### Population Stratification and Ashkenazi Jewish Ancestry

Since a founder effect for LSD gene mutations have been reported in the

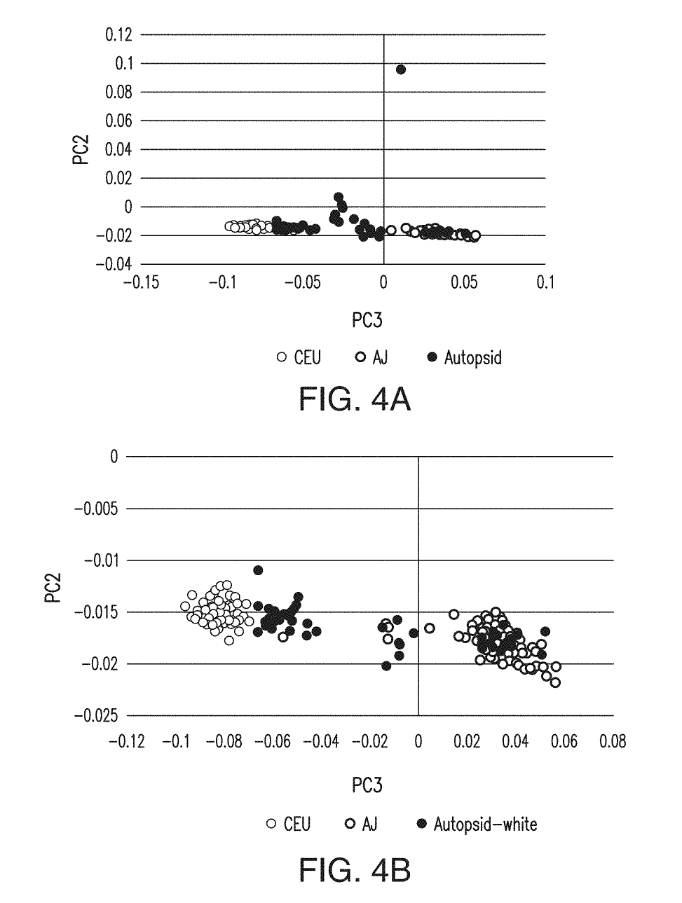

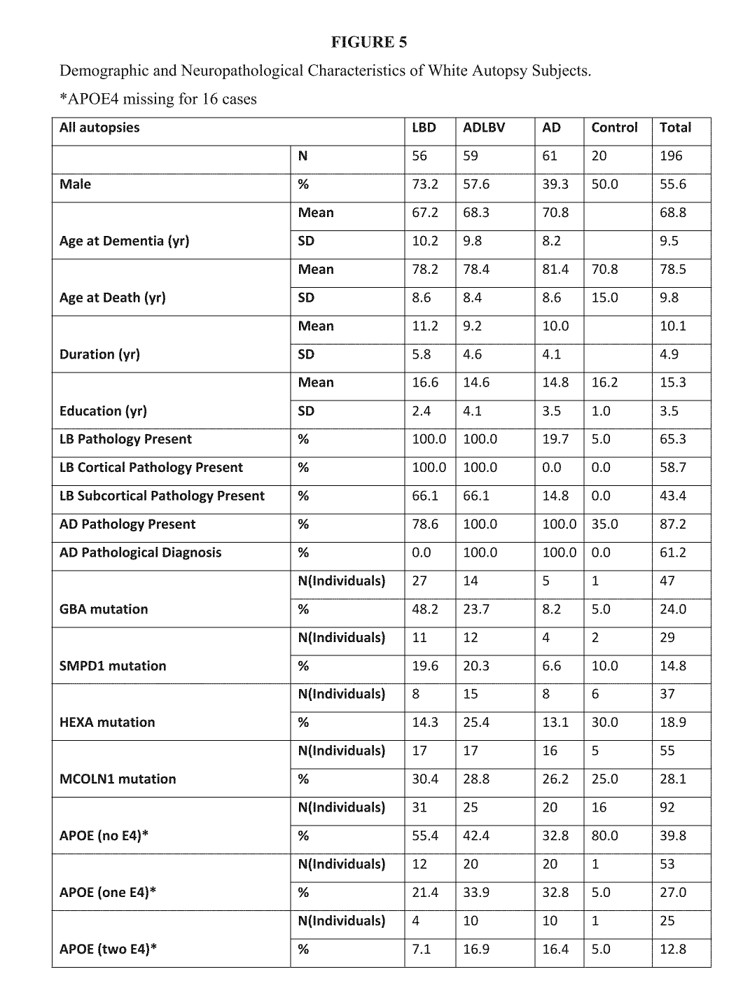

AJ population the inventor also determined AJ ancestry in brain autopsy samples. Information about AJ ancestry was not available for brain autopsies. The inventor used two methods to examine AJ ancestry and underlying population structure in brain autopsies. In the first method, Multidimensional scaling (MDS) as implemented in the program PLINK (Version 1.07) for detecting population outliers and adjusting for population stratification was used. Briefly, the inventor used 288,963 autosomal SNPs for brain autopsies (n=62), augmented with 252 AJ samples with subjects from the HapMap website (http:www.hapmap.org/), which included 90 CEU, 90 Yorubans and 90 Asians. The best fitting model assumed two underlying populations with overlap of 27 white brain autopsies with the AJ cluster and the remainder of the white brain autopsies with the white CEU cluster. In the second method, principle component analysis (PCA) as implemented in the GCTA package [16] was used to examine ancestry and admixture in white brain autopsies, AJ samples together with subjects from HapMap. Projection of all the sample genotypes along the two principle components (PC2 and PC3) is shown in FIG. 4. As in the MDS analysis performed in PLINK, there is tight clustering of 27 brain autopsies with AJ sample cluster and the remainder of the white brain autopsies cluster with CEU samples.

### AJ Control Samples

A total of 128 Ashkenazi Jewish (AJ) healthy controls (a first group of 74 subjects and a second group of 54 subjects) were used in a secondary analysis to supplement autopsy controls. (Table A1)

The inventor include data for 128 AJ population healthy controls. High depth whole genome sequencing was performed in 128 healthy controls and an AJ reference panel developed. Compared to a European reference panel, the AJ panel is 47% richer in novel variants and 8-fold more effective at filtering benign variants, which is necessary for interpreting AJ clinical genomes. The demographic and medical characteristics of the 128 sequenced individuals is summarized below. Further details of the cohort including sample selection is described in Carmi et al (2014)[41] .

TABLE A1
Demographic and medical characteristics of the AJ samples.

| | Mean ± | | Mean ± |
|---|---|---|---|
| | Standard | | Standard |
| Trait (Group 1) | Deviation | Trait (Group 2) | Deviation |
| All (n) | 74 | All (n) | 54 |
| Female (n) | 45 | Female (n) | 33 |
| Male (n) | 29 | Male (n) | 21 |
| Age (years) | 68.8 ± 7.7 | Age (years) | 68.7 ± 10.4 |
| | (range 49-85) | | (range 39-88) |
| Cholesterol (mg/dL) | 200 ± 42.1 | Intellectual impairment (n) | 1 |
| Triglycerides (mg/dL) | 132 ± 71.4 | Thought disorder (n) | 1 |
| HDL (mg/dL) | 65.4 ± 17.2 | Depression (n) | 2 |
| LDL (mg/dL) | 108 ± 34.4 | Family history of PD in | 2 |
| | | first degree relatives | |
| | | (conservative) (n) | |
| Glucose (mg/dL) | 81.9 ± 14.6 | Family history of AD in | 3 |
| | | first degree relatives | |
| | | (conservative) (n) | |
| Waist circumference (inch) | 35.1 ± 6.9 | Total mMMS Score | 56.2 ± 1.4 |
| Body Mass Index (kg/m²) | 26.4 ± 5.1 | Total UPDRS part II score | 0.25 ± 0.85 |
| Systolic Blood pressure (mm Hg) | 139 ± 20.6 | Total UPDRS part III | 1.81 ± 3.20 |
| | | score | |

| | |
|---|---|
| Diastolic Blood pressure (mm Hg) | 79.6 ± 11.2 |

For description of the cohorts, see Carmi et al (2014). Except the gender and the mMMS score, all traits in the Group 2 cohort were computed over 53 samples. PD: Parkinson's disease. AD: Alzheimer's disease. The mMMS score is calculated from a modification of the modified Mini-Mental State Examination, with a maximum score of 57 (computed over 15 samples). The Unified Parkinson's Disease Rating Scale (UPDRS) parts II and III contain 44 questions each measured on a 5-point scale (0-4).

### Molecular Genetic Analysis

Frozen cerebellar tissue was used to extract DNA. Sequencing of all GBA exons was performed as described previously[9]. Sequencing of all exons of HEXA, SMPD1 and MCOLN1 was also performed. APOE genotyping was performed by MALDI-TOF mass spectrometry on the Sequenom platform as described previously[11].

### Analysis of Functional Effect of Variants

The National Center for Biotechnology information (NCBI), ClinVar, the NHLBI Exome Sequencing project (ESP) exome variant server in addition to in silico prediction was used to assess the deleterious effect of variants.

Condel uses a consensus deleteriousness score that combines various tools (SIFT, Polyphen2, MAPP LogR Pfam E-value and Mutation assessor). The scores of different methods are weighted using the complementary cumulative distributions produced by the five methods on a dataset of approximately 20000 missense SNPs, both deleterious and neutral. The probability that a predicted deleterious mutation is not a false positive of the method and the probability that a predicted neutral mutation is not a false negative are employed as weights[42].

### Enzyme Activity Measurements

For enzyme activity measurements, a subset (n=64) of the total autopsy sample (n=231) for which frozen brain tissue was available was selected based on neuropathological diagnosis and GBA mutation carrier status (GBA mutation carriers (n=16), LBD brains without GBA mutations (n=18) and control brains (n=30)). Brain autopsy tissue (Cerebellum, BA4 and BA9 and ScxV) samples were homogenized in water (10% wt./vol.) using a Misonix Sonic Dismembrator and centrifuge at 30,000 Xg for 20 min. Protein concentration was determined using the Lowry method. The reaction mixture for β-glucocerebrosidase determination consisted of 50 ug of protein, 50 ul of 20mM 4-methylumbelliferyl-β-D-glucopyranoside, 10 ul of 1M Citrate-Phosphate pH 5.0 and 10 ul of 2% Sodium Tauro Deoxycholate. The reaction mixture was incubated at 37° C. for 2 Hours and thensubsequently stopped with 2 ml 0.2 M glycine buffer, pH 10.3. The Hexosamindase A enzymatic reaction mixture consisted of bug of protein and 100u1 of 3mM 4-methylumbelliferyl-2-acetoamido-2-deoxy-b-D-glucopyranoside in Citrate-Phosphate buffer pH4.0. Samples were incubated at 37° C. for 10 min and 0.2M glycine buffer was also used to stop the reaction. Fluorescence was determined in fluorescence spectrophotometer (Hitachi F-2500) at an excitation wavelength of 365 nm and emission wavelength of 448 nm. Samples were compared against a 4-methylumbelliferone (4-MU) standard curve prepared in 0.2 M glycine buffer. Enzyme activities were calculated in nmoles of 4-MU hydrolyzed/mg protein/hr. LBD brains did not carry variants in any of the other LSD genes analyzed. Frozen post-mortem interval (PMI) was available for all autopsy tissue and PMI did not appear to influence GCase activity.

### Lipid Profiling

For lipid profiling, a convenience subset (n=67) of the total autopsy sample (n=231) was selected based on neuropathological diagnosis and GBA mutation carrier status that included LBD brains from GBA mutation carriers (n=13), LBD brains without GBA mutations (n=33), AD brains (n=4) and control brains (n=17). Lipid extracts were prepared using a modified Bligh/Dyer extraction procedure, spiked with appropriate internal standards. The samples were analysed using an Agilent 1260 HPLC system coupled to an Agilent 6490 Triple Quadrupole mass spectrometer. The lipidomic profiles generated for each sample were obtained through a combination of HPLC separation and mass spectrometry in multiple reactions monitoring mode which allows for the unambiguous identification of lipids as described previously [17,18].

Lipid extracts were prepared using a modified Bligh/Dyer extraction procedure, spiked with appropriate internal standards including PC (14:0/14:0), PE (14:0/14:0), PS (14:0/14:0), PA (14:0/14:0), PG (15:0/15:0), LBPA (14:0/14:0), Cer (d18:1/17:0), GalCer (d18:1/12:0), GluCer (d18:1/12:0), Sulf (d18:1/12:0) and SM (d18:1/12:0) obtained from Avanti Polar Lipids (Alabaster, Ala.) and PI (16:0/16:0) obtained from Echelon Biosciences (Salt Lake City, Utah)[45]. The samples were analyzed using an Agilent 1260 HPLC system coupled to an Agilent 6490 Triple Quadrupole mass spectrometer. Separation of individual phospholipid and sphingolipid subclasses by normal phase HPLC was carried out using a Phenomenex Luna Si column (i.d. 2.0×100 mm, 3 μm) or Agilent Rx-Sil column (i.d. 2.1×100mm, 1.8 μm) with the following conditions: mobile phases A (chloroform: methanol: ammonium hydroxide, 89.8:10:0.2) and B (chloroform: methanol: ammonium hydroxide: water, 55:39:0.2:5.8); flow rate of 0.3 ml/min; 5% B for 2 min, then linearly changed to 70% B over 18 min and maintained for 3 min. The column was re-equilibrated for the next sample by changing the gradient back to 5% B over 2 min and maintained for 6 min for column re-equilibration. Multiple reaction monitoring transitions were set up for quantitative analysis of various lipid species and referenced to the spiked internal standard concentrations as done previously[45,46]. The lipid levels for each sample was calculated by summing up the total number of moles of all lipid species measured, and then normalizing that total to give mol %. The final data are presented as mean mol % with error bars showing standard error of means.

### Statistical Analysis

T tests and chi square tests were used to compare continuous and categorical variables respectively. To determine whether a gene, represented by multiple sequenced variants, is associated with affection status or not, the inventor applied the sequence kernal association test (SKAT) algorithm [19]. As above, age and sex were included in one model as covariates, and permutation based p-value was computed. To determine whether multiple variants in the lysosomal disease genes are associated with LB pathology in an additive manner, after correcting for age and sex as covariates, the inventor applied multiple logistic regression. Said additive model is one way to gain insight into a set of functional (i.e., nonsynonymous) variants in the common disease pathway. For lipidomics data, Statistical analysis for the AD and LBD mutation samples was based on the one way analysis of variance followed by post hoc Fisher's least significant difference test while the LBD wild type samples was based on Student's T-test. In all cases, *, p<0.05; **, p<0.01; ***, p<0.001.

### Data Access

All deidentified genotype data and related meta data underlying the reported findings are available at the public repository Dryad (datadryad.org). The doi is: doi:10.5061/dryad.61c8t. Genotype data from 128 Ashkenazi Jewish (AJ) healthy individuals that were used in a secondary analysis to supplement the limited number of brain autopsy controls is available upon request at the European Genome-Phenome Archive (dataset accession: EGAD00001000781).

### Results

### Demographic and Neuropathological Characteristics of Autopsy Samples

The basic demographic and neuropathologic information of autopsy samples analysed is shown in Table 1 (all autopsies, N=231) and FIG. 5 (white non-Hispanic ethnicity only, N=196). Overall, the proportion of men (71.2%) was higher in the LBD and Alzheimer disease and lewy body variant (ADLBV) group, compared with that in the AD group (36.6%). Overall, LBD patients had a significantly earlier age at onset of dementia (66.58+10.28 years vs. 70.71+8.46; p=0.05), earlier age at death (78.26+8.66 years vs. 81.64+8.60; p=0.04), and had more years of education (16.61+2.33 years vs. 13.84+4.34; p=0.02) compared to patients with AD. APOE4 allele frequencies did not differ from reported population frequencies in non-AD groups.

TABLE 1
Characteristics of Autopsy Subjects

| All autopsies | | LBD | ADLBV | AD | Control | Total |
|---|---|---|---|---|---|---|
| | N | 59 | 68 | 71 | 33 | 231 |
| Male | % | 71.2 | 52.9 | 36.6 | 54.5 | 52.8 |
| Age at Dementia (yr) | Mean | 66.6 | 67.6 | 70.7 | | 68.4 |
| | SD | 10.3 | 9.6 | 8.5 | | 9.5 |
| Age at Death (yr) | Mean | 78.3 | 79.1 | 81.6 | 70.3 | 78.4 |
| | SD | 8.7 | 8.5 | 8.6 | 14.2 | 10.1 |
| Duration (yr) | Mean | 11.6 | 10.3 | 10.0 | | 10.6 |
| | SD | 6.1 | 6.1 | 4.3 | | 5.5 |
| Education (yr) | Mean | 16.6 | 14.1 | 13.8 | 14.2 | 14.5 |
| | SD | 2.3 | 4.1 | 4.3 | 3.4 | 4.0 |
| Ethnicity | % White | 94.9 | 86.8 | 85.9 | 60.6 | 84.8 |
| % AJ ancestry | N (individuals) | 3 | 7 | 15 | 2 | 27 |
| | % AJ (n/total | 100 | 35 | 41.67 | 66.67 | 43.5** |
| | samples with | (3/3) | (7/20) | (15/36) | (2/3) | (27/62) |
| | GWAS) | | | | | |
| LB Pathology Present | % | 100.0 | 100.0 | 16.9 | 3.0 | 60.6 |
| LB Cortical Pathology | % | 100.0 | 100.0 | 0.0 | 0.0 | 55.0 |
| Present | | | | | | |
| LB Subcortical Pathology | % | 64.4 | 69.1 | 12.7 | 0.0 | 40.7 |
| Present | | | | | | |
| AD Pathology Present | % | 79.7 | 100.0 | 100.0 | 33.3 | 853 |
| AD Pathological | % | 0.0 | 100.0 | 100.0 | 0.0 | 60.2 |
| Diagnosis | | | | | | |
| GBA mutation | N(Individuals) | 28 | 16 | 6 | 1 | 51 |
| | % | 47.5 | 23.5 | 8.5 | 3.0 | 22.1 |
| SMPD1 mutation | N(Individuals) | 12 | 14 | 4 | 3 | 33 |
| | % | 20.3 | 20.6 | 5.6 | 9.1 | 14.3 |
| HEXA mutation | N(Individuals) | 8 | 17 | 8 | 6 | 39 |
| | % | 13.6 | 25.0 | 11.3 | 18.2 | 16.9 |
| MCOLN1 mutation | N(Individuals) | 17 | 20 | 17 | 8 | 62 |
| | % | 28.8 | 29.4 | 23.9 | 24.2 | 26.8 |
| APOE (no E4)* | N(Individuals) | 33 | 26 | 26 | 24 | 109 |
| | % | 66.0 | 41.9 | 42.6 | 77.4 | 47.2 |
| APOE (one E4)* | N(Individuals) | 13 | 26 | 25 | 6 | 70 |
| | % | 26.0 | 41.9 | 41.0 | 19.4 | 30.3 |
| APOE (two E4)* | N(Individuals) | 4 | 10 | 10 | 1 | 25 |
| | % | 8.0 | 16.1 | 16.4 | 3.2 | 10.8 |

*APOE missing in 27 cases,
**% AJ in brain autopsy sample with GWAS data available (n = 62)

### Sequencing and Association Analysis

### Variants Identified and Predicted Impact on Function.

Overall, the inventor identified 51 (22.1%) subjects with a GBA variant, 39 (16.9%) with a HEXA variant, 33 (14.3%) with an SMPD1 variant and 62 (26.8%) with an MCOLN1 variant (Table 1). Many of the variants that the inventor identified have been reported previously as pathogenic mutations in patients with the associated lysosomal storage disorder (Table 2). LSD variants that were significantly associated in brain autopsies with a neuropathological diagnosis of LBD are shown in Table 2.

TABLE 2
Variants identified in brain autopsy samples

| | Chr, genomic | Protein | | MAF (1000 | Clinical |
|---|---|---|---|---|---|
| Gene | coordinates* | (allele name) | dbSNP | genomes) | significance** |
| GBA | 1:155235002 | p.R535H (p.R496H) | rs80356773 | NA (rare) | Pathogenic[20] |
| | 1:155235196 | p.R502C (p.R463C) | rs80356771 | NA (rare) | Pathogenic[20] |
| | 1:155235252 | p.L483P (p.L444P) | rs421016 | 0.0034 | Pathogenic[20] |
| | 1:155235727 | p.D448H (D409H) | rs1064651 | NA (rare) | Pathogenic[20] |
| | 1:155235843 | p.N409S (N370S) | rs76763715 | 0.0006 | Pathogenic[20] |
| | 1:155236246 | p.T408M (T369M) | rs75548401 | 0.0018 | Uncertain |
| | | | | | significance |
| | 1:155236376 | p.E365L (p.E326K) | rs2230288 | 0.0050 | Pathogenic[20] |
| | 1:155237458 | p.H294Q (p.H255Q) | rs367968666 | NA (rare) | Pathogenic[20] |
| | 1:155238228 | p.W223R (p.W184R) | rs61748906 | NA (rare) | Pathogenic[20] |
| | 1:155238392 | — | rs114099990 | NA (rare) | Unknown |
| | 1:155240660- | p.Leu29AlafsX188 (84GG) | rs387906315 | NA (rare) | Pathogenic[20] |
| | 155240661 | | | | |
| | 1:155236304 | p.E388K | — | NA (rare) | Unknown |
| SMPD1 | 11:6390654 | p.Q19R | rs144465428 | NA (rare) | unknown |
| | 11:6390705 | p.V36A | rs1050228 | 0.4387 | Benign/likely |
| | | | | | benign |
| | 11:6390741- | p.Leu49_Ser50insAL | rs71056748 | NA (rare) | Unknown |
| | 6390742 | p.Leu49_Ser50insALAL | | | |
| | 11:6391701 | p.D212D | rs7951904 | 0.1282 | Benign/likely |
| | | | | | benign |
| | 11:6392137 | p.E358K | — | NA (rare) | Unknown |
| | 11:6394233 | p.G508R | rs1050239 | 0.15 | Benign/likely |
| | | | | | benign |
| | 11:6394336 | p.R542L | — | NA (rare) | Unknown |
| | 11:6394652 | — | rs8164 | 0.1484 | Unknown |
| | 11:6392136 | p.A357A | rs72896268 | 0.0034 | Benign/likely |
| | | | | | benign |
| | 11:6391966 | p.V301I | rs2723669 | 0.0032 | Unknown |
| | 11:6390697 | p.M33I | rs142178073 | 0.0038 | Unknown |
| | 11:6394029 | p.G492S | rs144873307 | 0.0014 | Likely |
| | | | | | Pathogenic[21] |
| | 11:6394261 | p.E517V | rs142787001 | 0.0014 | Likely |
| | | | | | Pathogenic[22] |
| | 11:6333377 | p.R418Q | — | NA (rare) | Unknown |
| HEXA | 15:72347852 | — | rs2302449 | 0.0759 | Unknown |
| | 15:72346579- | p.Y427I | rs387906309 | NA (rare) | Pathogenic |
| | 72346580 | (1277_1278insTATC) | | | |
| | 15:72349307 | — | rs73440586 | 0.0721 | Unknown |
| | 15:72350564 | p.V253V | rs117513345 | 0.0016 | Unknown |
| | 15:72375964 | p.S3S | rs1800428 | 0.0441 | Unknown |
| | 15:72350584 | p.R247W | rs121907970 | 0.0004 | Pathogenic |
| | 15:72351103 | — | rs117160567 | 0.0144 | Unknown |
| | 15:72345619 | — | rs2288259 | NA (rare) | Unknown |
| | 15:72346551 | p.I436V | rs1800431 | NA (rare) | Benign/likely |
| | | | | | benign |
| | 15:72350518 | p.G269S | rs121907954 | NA (rare) | Pathogenic |
| | 15:72351103 | c.672 + 30 T > G | rs117160567 | 0.0144 | Unknown[23] |
| | 15:72351231 | p.V192I | — | NA (rare) | Unknown |
| | 15:72355693 | — | rs10220917 | 0.0875 | Unknown |
| MCOLN1 | 19:7526723 | — | rs45513896 | 0.0222 | Unknown |
| | 19:7527537 | p.P197S | rs145706318 | NA (rare) | Unknown |
| | 19:7528162 | p.T261M | rs73003348 | 0.0026 | Unknown |
| | 19:7528283 | — | rs2305889 | 0.2821 | Unknown |
| | 19:7529124 | p.C386C | rs139922988 | 0.0004 | Unknown |
| | 19:7533531 | p.G528G | rs145386883 | 0.0006 | Unknown |
| | 19:7533693 | — | rs686796 | 0.0122 | Unknown |
| | 19:7527954 | p.S257R | rs113261161 | 0.0088 | Unknown |
| | 19:7528685 | p.R322R | rs61736600 | 0.0375 | Unknown |
| | 19:7528703 | p.N328N | rs612862 | 0.2556 | Unknown |
| | 19:7526768 | p.A138V | rs142259322 | 0.0008 | Unknown |
| | 19:7529625 | p.S424S | rs147754092 | 0.0012 | Unknown |

All mutations are described as recommended at www.hgvs.org/mutnomen
*Chr and genomic coordinates as based on assembly GRCh38 and genome build 106.
**Clinical Significance was assessed based on citations (published articles and URLs) documenting the clinical significance or based on pathogenic status repotted in dbSNP or ClinVar

Multiple Variants in GBA, SMPD1 and MCOLN1 are Associated with a Neuropathological Diagnosis of LBD

### Single Gene Wise Association

SNP-set (Sequence) Kernal Association Test (SKAT) was used to evaluate association of variants in GBA, HEXA, SMPD1 and MCOLN1 (Table 3). When evaluating all variants, strongest association was observed for GBA variants in LBD (p=2.95 ×10−5) and ADLBV (p=3.59 ×10−2) (Table 3). Risk variants in GBA, SMPD1 and MCOLN1 were also significantly associated with LBD (p range=0.03−4.14×10−5) and ADLBV (p range=0.02−0.01) pathology but not AD (Table 3). The inventor also evaluated association of protective variants and observed association of variants in SMPD1 in LBD (p=0.03) and ADLBV (p=0.02), but not AD, and MCOLN1 variants in LBD (p=0.02), ADLBV (p=0.005) but not AD (Table 3).

TABLE 3
Gene wise association SKAT analysis* in all samples

| | LBD vs. CTRL | | ADLBV vs. CTRL | | AD vs. CTRL | |
|---|---|---|---|---|---|---|
| | (n = 59 vs. 33) | | (n = 68 vs. 33) | | (n=71 vs. 33) | |
| Sample size | | Marker | | Marker | | Marker |
| Gene | P value | (n)** | P value | (n)** | P value | (n)** |

All variants

| | | | | | | |
|---|---|---|---|---|---|---|
| GBA | 2.95 × 10⁻⁵ | 11 | 3.59 × 10⁻² | 7 | 0.363 | 4 |
| SMPD1 | 0.114 | 12 | 0.259 | 12 | 0.347 | 9 |
| HEXA | 0.885 | 9 | 0.450 | 13 | 0.638 | 7 |
| MCOLN1 | 3.25 × 10⁻² | 6 | 8.31 × 10⁻² | 11 | 0.368 | 9 |
| GBA + SMPD1 | 2.89 × 10⁻⁴ | 23 | 3.94 × 10⁻² | 19 | 0.563 | 13 |
| GBA + SMPD1 + MCOLN1 | 1.29 × 10⁻³ | 29 | 5.09 × 10⁻² | 30 | 0.492 | 22 |

Risk variants¹

| | | | | | | |
|---|---|---|---|---|---|---|
| GBA | 4.14 × −10⁻⁵ | 11 | 1.27 × 10⁻² | 6 | 0.356 | 4 |
| SMPD1 | 1.93 × 10⁻² | 10 | 6.50 × 10⁻² | 10 | 7.68 × 10⁻² | 5 |
| HEXA | 0.124 | 5 | 0.105 | 10 | 0.254 | 3 |
| MCOLN1 | 3.33 × 10⁻² | 4 | 2.50 × 10⁻² | 9 | 8.78 × 10⁻² | 7 |
| GBA + SMPD1 | 3.87 × 10⁻⁵ | 21 | 8.35 × 10⁻³ | 16 | 5.85 × 10⁻² | 9 |
| GBA + SMFD1 + MCOLN1 | 1.11 × 10⁻⁴ | 25 | 4.35 × 10⁻³ | 25 | 3.39 × 10⁻² | 16 |

Protective variants¹

| | | | | | | |
|---|---|---|---|---|---|---|
| GBA | —² | | 1 | 1 | —² | |
| SMPD1 | 2.80 × 10⁻² | 2 | 1.68 × 10⁻² | 2 | 0.152 | 4 |
| HEXA | 0.360 | 4 | 0.553 | 3 | 0.166 | 4 |
| MCOLN1 | 2.18 × 10⁻² | 2 | 4.89 × 10⁻³ | 2 | 8.24 × 10⁻² | 2 |
| GBA + SMPD1 | 2.76 × 10⁻² | 2 | 0.152 | 3 | 0.152 | 4 |
| GBA + SMPD1 + MCOLN1 | 1.79 × 10⁻³ | 4 | 2.38 × 10⁻³ | 5 | 4.29 × 10⁻² | 6 |

*Corrected for covariates.
**Indicates number or markers included in the test.
¹Risk variants are variants more frequent among cases than controls; whereas, variants are considered protective when they are more frequent in controls than cases.
²No protective variants were observed.

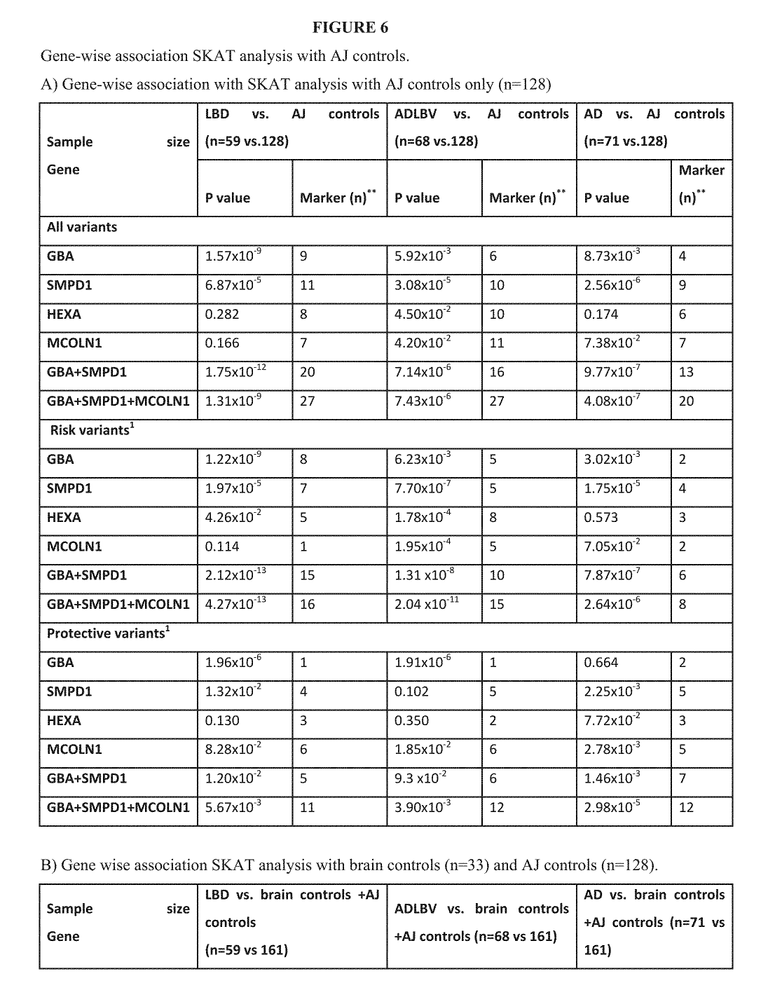

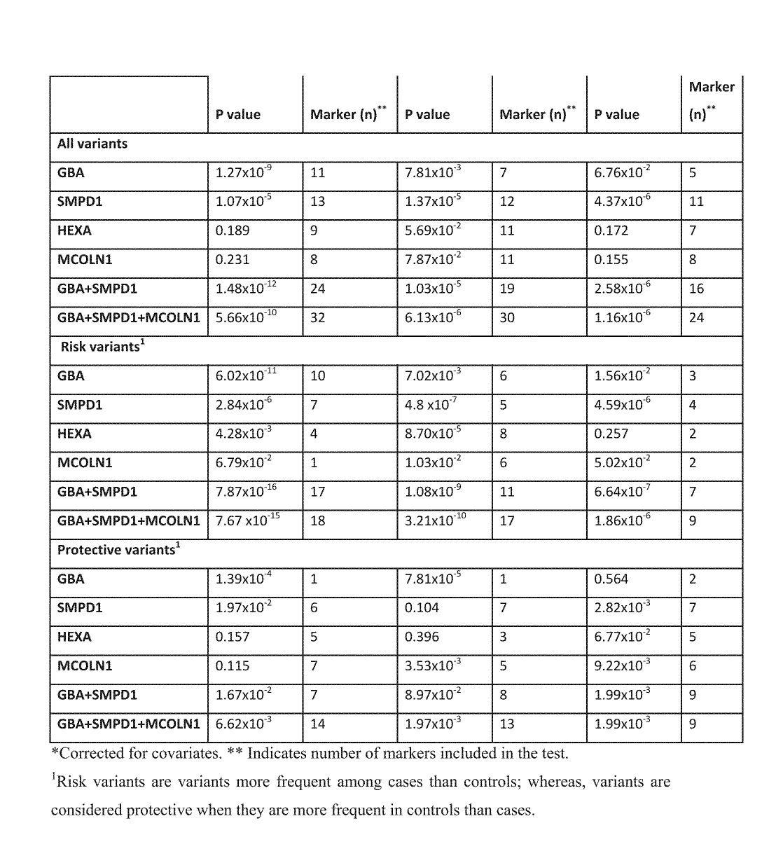

The following secondary analyses of the same SKAT models was also performed:1) SKAT analysis of LSD variants with MAF<5% in all samples (n=231) (Table 4), 2) SKAT analysis of LSD variants with MAF<5% in ‘white’ subjects only (n=196) (Table 5) and 3) SKAT analysis of LSD variants in all samples (n=231) using a larger control group which included the brain autopsy controls (n=33) and the AJ controls (n=128) (FIG. 6). When the inventor restricted the analysis to variants with MAF<5% (Table 4), strongest association was observed for GBA variants in LBD (p=1.37×10−4). Risk variants in GBA, SMPD1 and MCOLN1 remained significantly associated with LBD (p range=0.04−1.77×10−4) and ADLBV (p range=0.04−0.02) pathology but not AD (Table 4). When the inventor restricted the analysis to whites only with variants with MAF<5% (Table 5) strongest association was observed for GBA variants in LBD (p=0.0118). Risk variants in SMPD1 in ADLBV were also significant (p=0.0274). Although not significant, there was a trend towards significance for MCOLN1 risk variants in LBD (p=0.189) and ADLBV (p=0.072). However, the small sample size of the ‘white’ controls (n=20) in this stratified analysis may be a confounding factor and the results should be interpreted with caution. SKAT analysis using the larger control group replicated the findings observed using the brain autopsy controls (n=33) alone (FIG. 6).

TABLE 4
Gene wise SKAT analysis of LSD variants with MAF <5% in all samples

| | LBD vs. CTRL | | ADLBV vs. CTRL | | AD vs. CTRL | |
|---|---|---|---|---|---|---|
| | (n = 59 vs. 33) | | (n = 68 vs. 33) | | (n = 71 vs. 33) | |
| Sample size | | Marker | | Marker | | Marker |
| Gene | P value | (n)** | P value | (n)** | P value | (n)** |

All variants

| | | | | | | |
|---|---|---|---|---|---|---|
| GBA | 1.37 × 10⁻⁴ | 11 | 9.90 × 10⁻² | 7 | 0.404 | 4 |
| SMPD1 | 0.200 | 11 | 0.305 | 11 | 0.571 | 8 |
| HEXA | 0.663 | 7 | 0.470 | 10 | 0.233 | 5 |
| MCOLN1 | 6.64 × 10⁻² | 5 | 5.45 × 10⁻² | 9 | 0.110 | 6 |
| GBA + SMPD1 | 4.67 × 10⁻⁴ | 22 | 8.18 × 10⁻² | 18 | 0.644 | 12 |
| GBA + SMPD1 + MCOLN1 | 6.27 × 10⁻⁴ | 27 | 2.94 × 10⁻² | 27 | 0.344 | 18 |

Risk variants¹

| | | | | | | |
|---|---|---|---|---|---|---|
| GBA | 1.77 × 10⁻⁴ | 11 | 3.80 × 10⁻² | 6 | 0.397 | 4 |
| SMPD1 | 2.48 × 10⁻² | 9 | 8.36 × 10⁻² | 9 | 0.039 | 4 |
| HEXA | 6.82 × 10⁻² | 4 | 6.27 × 10⁻² | 8 | 0.522 | 2 |
| MCOLN1 | 3.91 × 10⁻² | 4 | 2.31 × 10⁻² | 8 | 4.10 × 10⁻² | 5 |
| GBA + SMPD1 | 2.94 × 10⁻⁵ | 20 | 1.68 × 10⁻² | 15 | 3.46 × 10⁻² | 8 |
| GBA + SMPD1 + MCOLN1 | 9.07 × 10⁻⁵ | 24 | 7.60 × 10⁻³ | 23 | 1.78 × 10⁻² | 13 |

Protective variants¹

| | | | | | | |
|---|---|---|---|---|---|---|
| GBA | —² | | 0.824 | 1 | —² | |
| SMPD1 | 0.253 | 2 | 5.73 × 10⁻² | 2 | 0.615 | 4 |
| HEXA | 0.580 | 3 | 0.598 | 2 | 2.54 × 10⁻² | 3 |
| MCOLN1 | 0.373 | 1 | 0.810 | 1 | 0.448 | 1 |
| GBA + SMPD1 | 0.253 | 2 | 0.262 | 3 | 0.615 | 4 |
| GBA + SMPD1 + MCOLN1 | 4.99 × 10⁻² | 3 | 0.317 | 4 | 0.404 | 5 |

*Corrected for covariates.
**Indicates number of markers included in the test.
¹Risk variants are variants more frequent among cases than controls; whereas, variants are considered protective when they are more frequent in controls than cases.
²No protective variants were observed.

TABLE 5
Gene wise SKAT analysis of LSD variants with MAF <0.05 in White subjects only

| | LBD vs. CTRL | | ADLBV vs. CTRL | | AD vs. CTRL | |
|---|---|---|---|---|---|---|
| | (n = 56 vs. 20) | | (n = 59 vs. 20) | | (n = 61 vs. 20) | |
| Sample size | | Marker | | Marker | | Marker |
| Gene | P value | (n)** | P value | (n)** | P value | (n)** |

All variants

| | | | | | | |
|---|---|---|---|---|---|---|
| GBA | 1.18 × 10⁻² | 10 | 0.306 | 7 | 0.795 | 4 |
| SMPD1 | 0.702 | 9 | 0.443 | 10 | 0.693 | 7 |
| HEXA | 0.912 | 7 | 0.770 | 9 | 0.177 | 5 |
| MCOLN1 | 0.194 | 4 | 0.129 | 8 | 0.117 | 5 |
| GBA + SMPD1 | 3.54 × 10⁻² | 19 | 0.205 | 17 | 0.853 | 11 |
| GBA + SMPD1 + MCOLN1 | 2.61 × 10⁻² | 23 | 9.97 × 10⁻² | 25 | 0.560 | 16 |

Risk variants¹

| | | | | | | |
|---|---|---|---|---|---|---|
| GBA | 1.02 × 10⁻² | 10 | 0.126 | 6 | 0.543 | 3 |
| SMPD1 | 0.276 | 8 | 0.027 | 7 | 0.149 | 4 |
| HEXA | 0.209 | 4 | 6.55 × 10⁻² | 6 | 0.539 | 2 |
| MCOLN1 | 0.189 | 4 | 7.19 × 10⁻² | 7 | 5.86 × 10⁻² | 4 |
| GBA + SMPD1 | 7.30 × 10⁻³ | 18 | 6.60 × 10⁻³ | 13 | 0.113 | 7 |
| GBA + SMPD1 + MCOLN1 | 6.30 × 10⁻³ | 22 | 2.70 × 10⁻³ | 20 | 3.22 × 10⁻² | 11 |

Protective variants¹

| | | | | | | |
|---|---|---|---|---|---|---|
| GBA | —² | | 0.743 | 1 | 0.651 | 1 |
| SMPD1 | 3.29 × 10⁻² | 1 | 0.682 | 3 | 0.563 | 3 |
| HEXA | 0.517 | 3 | 0.606 | 3 | 1.93 × 10⁻² | 3 |
| MCOLN1 | —² | | 0.794 | 1 | 0.826 | 1 |
| GBA + SMPD1 | 3.29 × 10⁻² | 1 | 0.772 | 4 | 0.432 | 4 |
| GBA + SMPD1 + MCOLN1 | 3.29 × 10⁻² | 1 | 0.822 | 5 | 0.579 | 5 |

*Corrected for covariates.
**Indicates number of markers included in the test.
¹Risk variants are variants more frequent among cases than controls; whereas, variants are considered protective when they are more frequent in controls than cases.

### Additive Effect of Multiple Variants in GBA, SMPD1 and MCOLN1

In exploratory analyses logistic regression analysis was also used to determine whether multiple variants in the same disease pathway are associated with disease pathology in an additive manner after adjusting for age and sex as covariates. Strong associations (p range:0.03−3.8×10⁻⁵) were also observed for LBD, ADLBV, and AD cases with multiple variants in GBA+SMPD1 or GBA+SMPD1+MCOLN1 (Table 3).

### GCase Activity is Decreased in LBD GBA Mutation Carriers Compared to LBD Non-Carriers

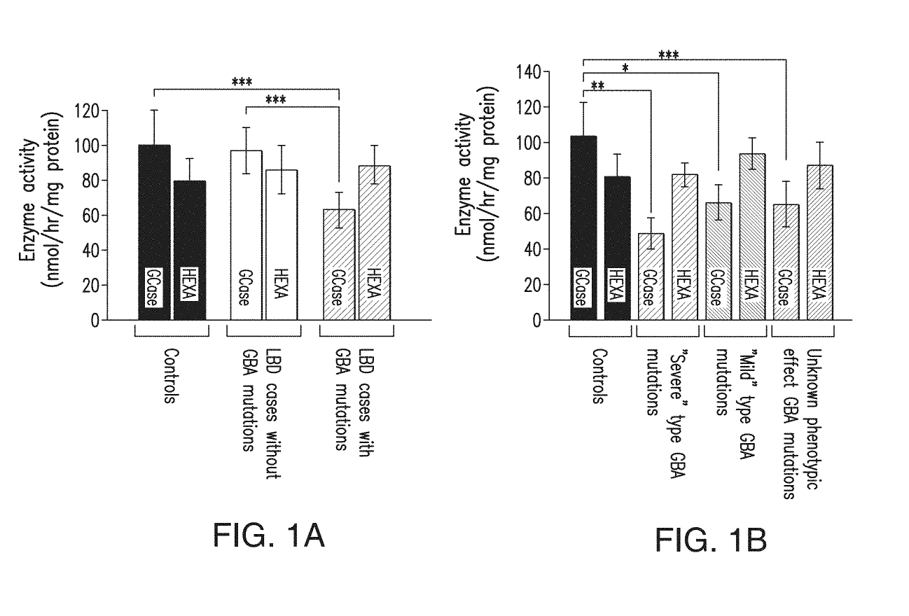

To determine whether carrier GBA mutation status was associated with reduced enzymatic activity (haploinsufficiency) the inventor assayed GCase activity in a subset of autopsy samples (n=64). GCase activity was measured in LBD brains from GBA mutation carriers (n=16), LBD brains without GBA mutations (n=18) and control brains (n=30) (FIG. 1) from the following brain regions Cerebellum, BA4 and BA9 and ScxV.

The enzyme activity of a second lysosomal hydrolase, α-hexosaminidase was also assayed to demonstrate specificity of decreased activity of GCase. Overall, the mean levels of GCase activity (p<0.001) and the β-glucocerebrosidase: α-hexosaminidase ratio (p<0.001) were significantly lower in GBA mutation carriers compared to non-carriers (FIG. 1). The inventor also observed significant differential enzyme activity of GCase or for the β-glucocerebrosidase: α-hexosaminidase ratio in subjects carrying GBA mutations classified phenotypically (as in Gaucher disease) as ‘severe’ type (e.g. 84insGG, L444P) (p<0.01) compared to subjects carrying GBA mutations classified phenotypically as ‘mild’ type (e.g. N370S, R496H) (p<0.05) or of unknown phenotypic effect (E326K, T369M) (p<0.001) compared to controls (FIG. 1). Lastly, the inventor examined the relation between GCase activity in subjects with a clinical diagnosis of dementia compared to cases without dementia. Overall, the mean levels of GCase activity (p=0.0021) and the β-glucocerebrosidase: α-hexosaminidase ratio (p=0.0014) were significantly lower in cases with dementia than in controls. The pattern of association between GBA mutation status and the GCase activity was comparable to the combined samples, suggesting that those with dementia and a neuropathological diagnosis of DLB are driving the association.

GBA mutation carriers show significant differences in lipid species and accumulation of ceramide and sphingolipids

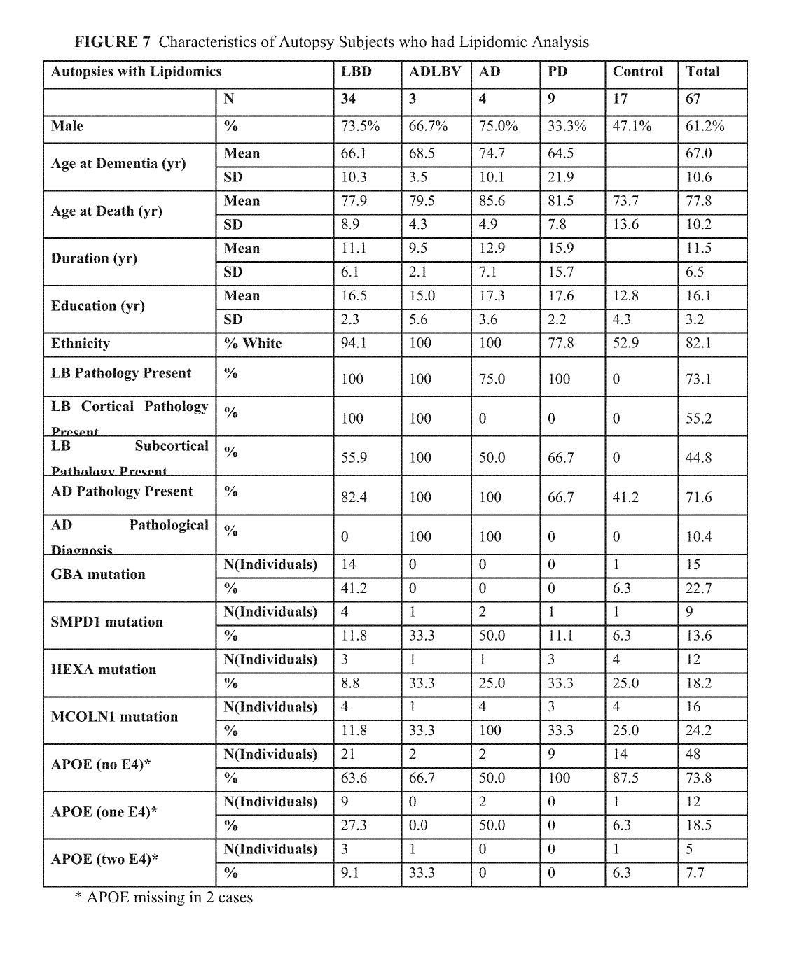

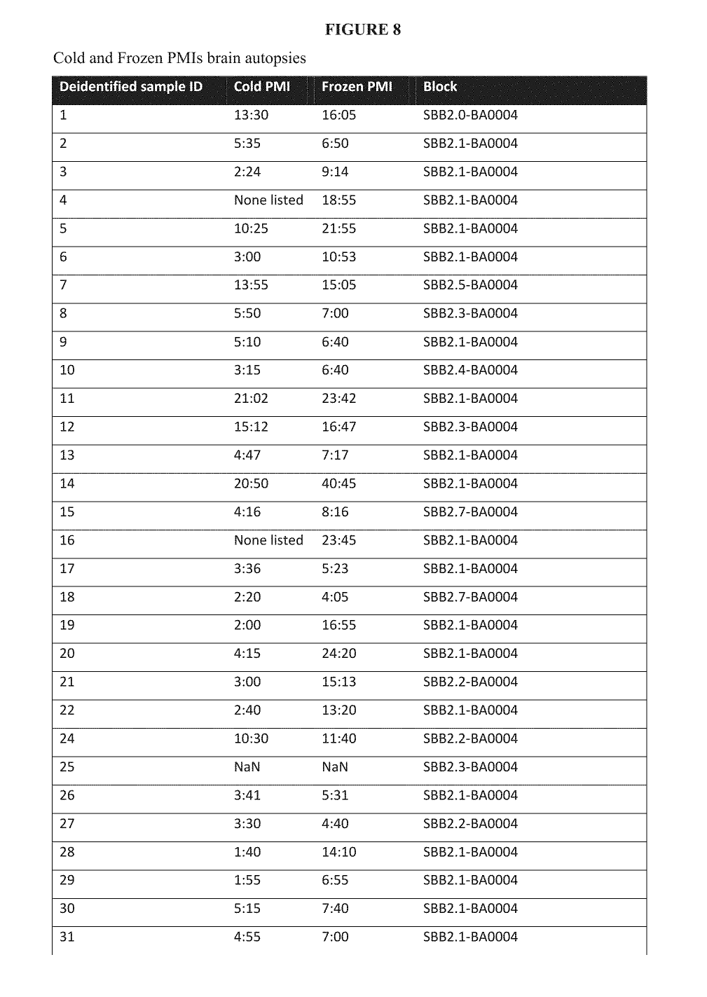

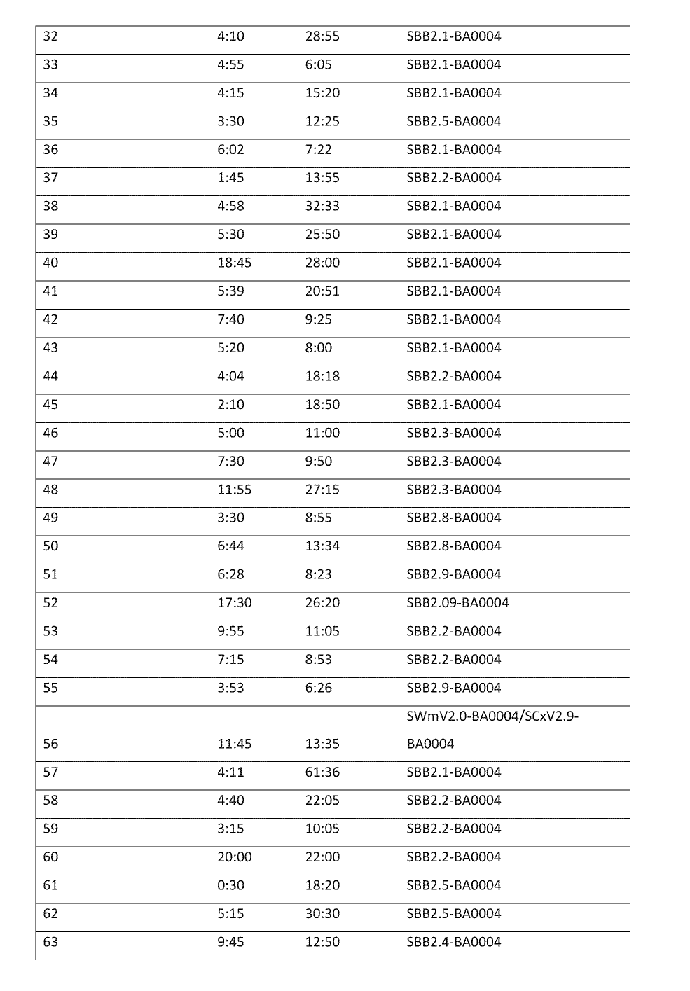

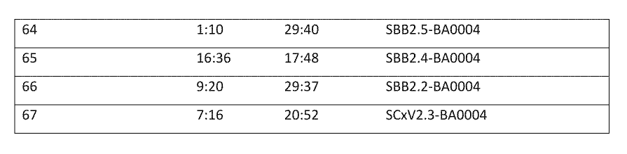

To determine the functional effect of reduced GCase activity in brains with LBs carrying GBA mutations compared to those without GBA mutations, AD, and control brains the inventor performed a lipidomic analysis in postmortem brain tissue (n=67) obtained from the primary motor cortex (BA4). The a priori hypothesis was that LBD GBA mutation carriers should show significant differences in ‘specific’ lipid species (substrate and product of GBA hydrolysis) and accumulation of ceramides and sphingolipids compared to those without GBA mutations, AD, and control brains. Characteristics of autopsy subjects with lipidomic analysis is shown in FIG. 7. The cold and frozen PMIs for autopsy tissue used in the analysis is shown in FIG. 8.

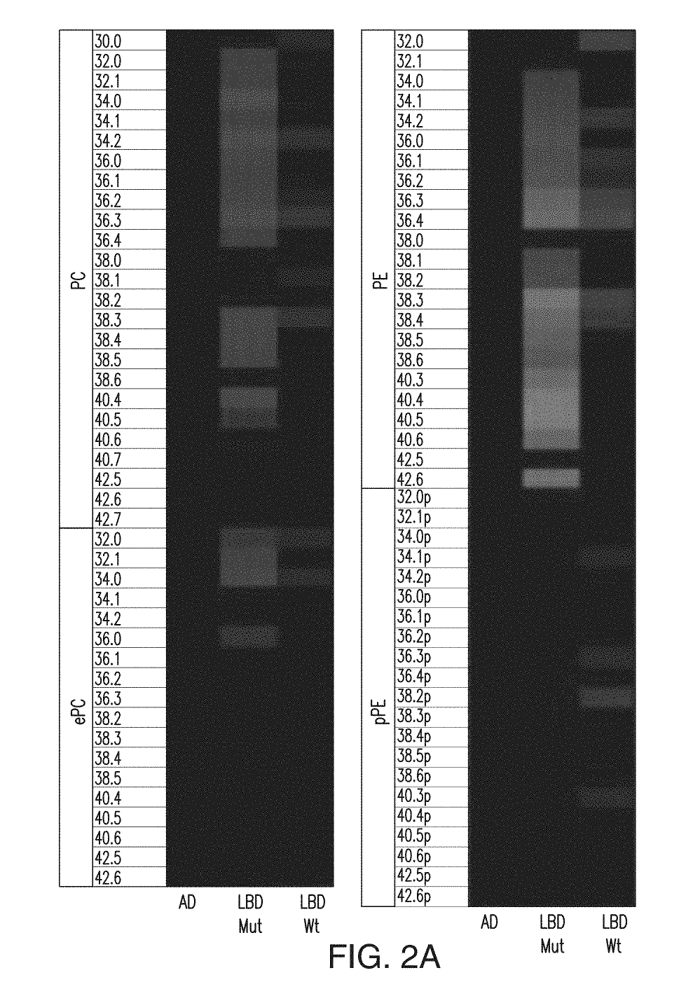

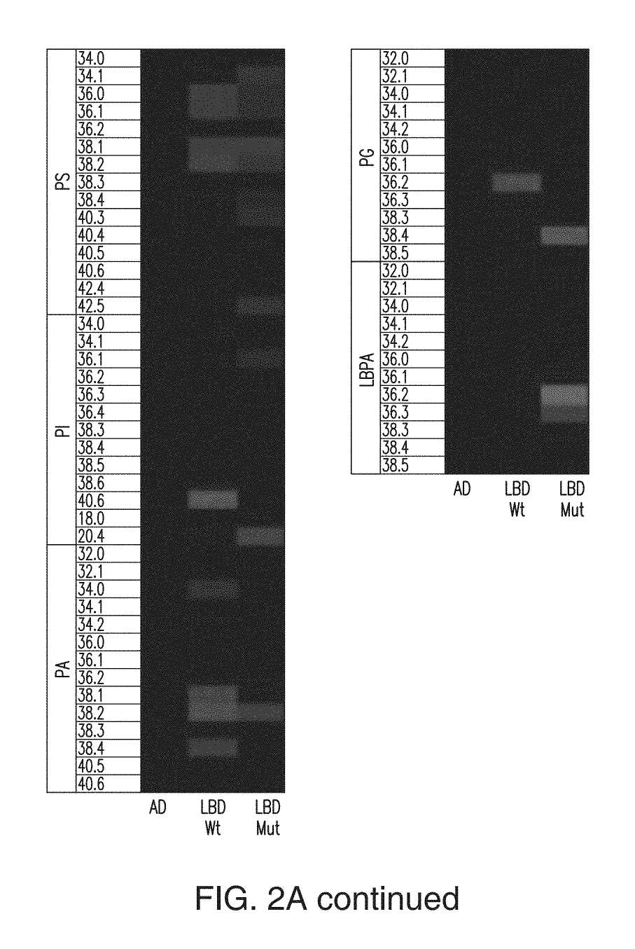

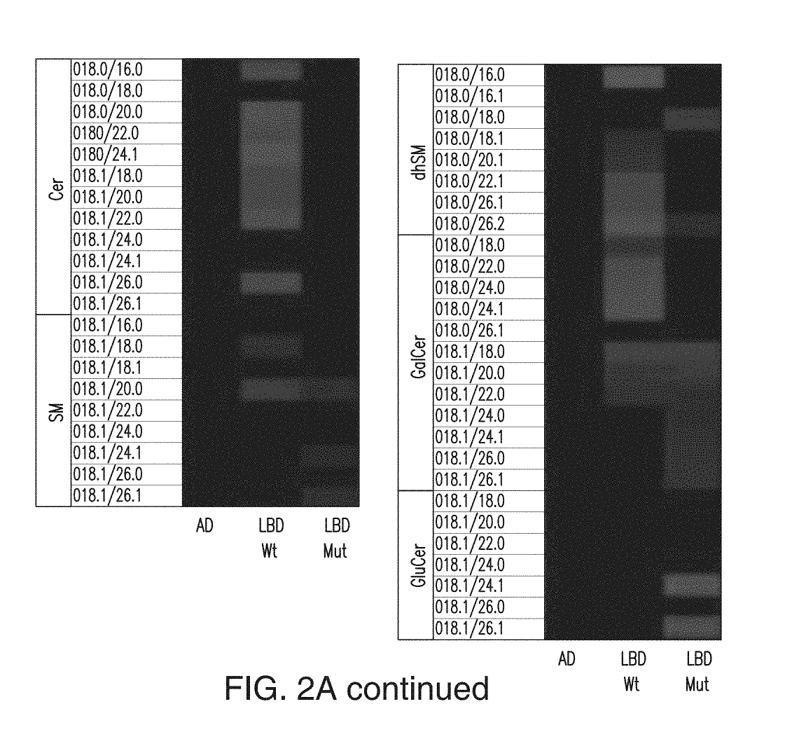

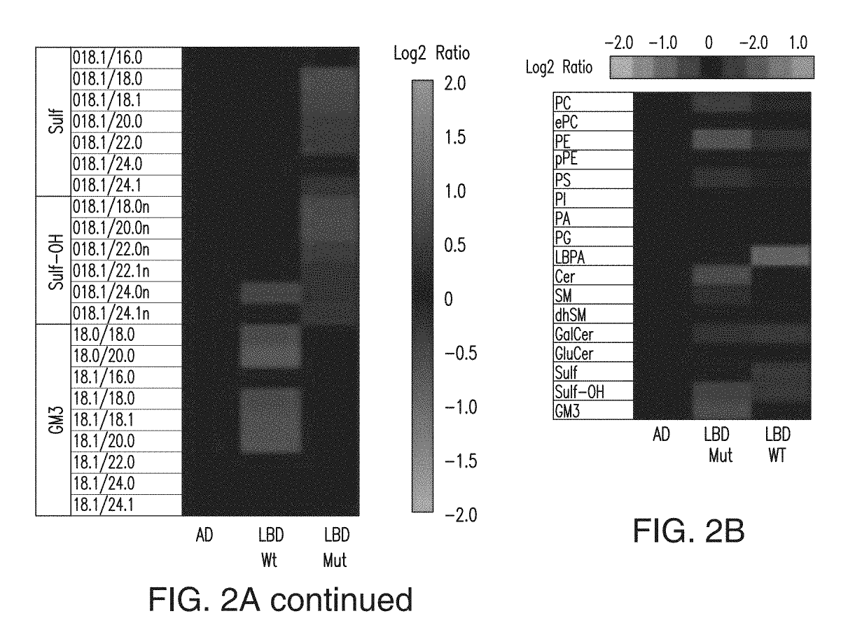

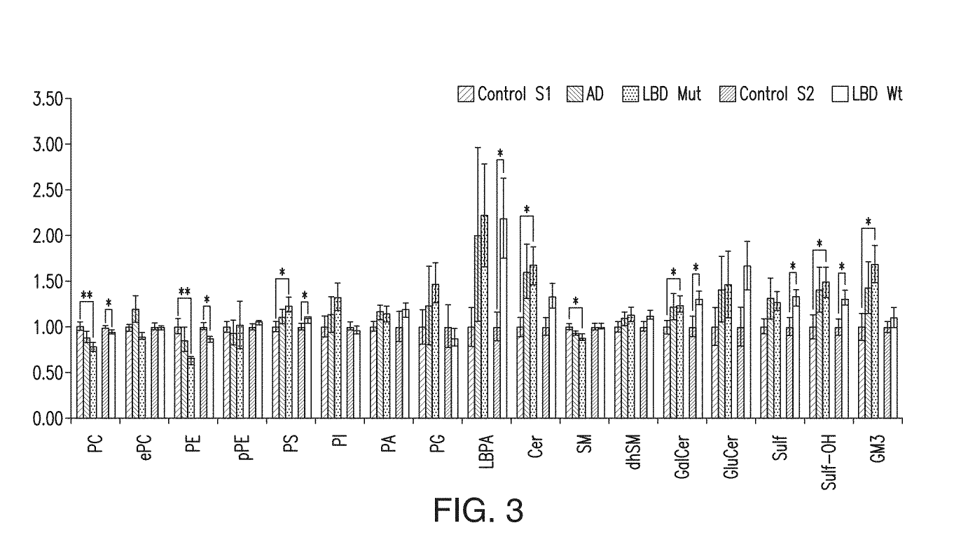

Several lipid classes were significantly altered in brains with LBs carrying GBA mutations compared to controls (P range: p<0.05-p<0.01) (FIG. 2 and FIG. 3) and this remained significant after using an false discovery rate (FDR) control to correct for multiple comparisons of lipids (q<0.05-q<0.01). Major phospholipid subclasses such as phosphatidylcholine (PC) and phosphatidylethanolamine (PE) were decreased while phosphatidylserine (PS) was increased. There were also striking changes in sphingolipid composition. A small but significant decline in the most abundant sphingolipid sphingomyelin (SM) was seen in the diseased tissue but was compensated by increased levels of select dihydrosphingomyelin (dhSM) species and total ceramide (Cer) levels. While there was a trend towards an increase in accumulation of the known GCase substrate, GluCer, the difference was not statistically significant (FIG. 2 and FIG. 3). However, the complex glycosphingolipid that is biosynthetically upstream of GluCer, GM3, is highly enriched in these tissues. In addition, there is also a significant accumulation of galactosylceramide (GalCer) and its biosynthetic derivative sulfatides containing hydroxy fatty acid (Sulf-OH).

In brains with LBs without GBA mutations, similar changes were observed in major phospholipids PC, PE and PS levels but to a lesser degree compared to those with GBA mutations, and in the sphingolipids GalCer, Sulf and Sulf-OH. Interestingly LBD brains with and without GBA mutations displayed an accumulation in lysobisphosphatidic acid LBPA (also known as bis(monoacylglycero)phosphate), a lipid that is specifically enriched in the late endosome and lysosome was observed. LBPA also showed a trend for increase in LBD brains with GBA mutations. To determine the specificity of the assay and also provide a reference point for lipid changes in the LBD tissue, the inventor also analyzed AD brains and found no significant changes. This is in contrast to a previous study [17], although different brain regions were analyzed (i.e., prefrontal cortex and entorhinal cortex).

Discussion In the current study the inventor performed a genetic analysis of four lysosomal storage disorder genes including GBA, HEXA, SMPD1, MCOLN1 in 231 brain autopsies from the New York Brain Bank at Columbia University. A biochemical analysis of GBA was also performed in a subset of brains. The results show that in addition to prior reported variants in GBA, variants in SMPD1 and MCOLN1 are also significantly associated with LB or ADLBV pathology. Additional gene-wise analyses for variants based on the SKAT algorithm also identified independent association of variants in GBA, SMPD1 and MCOLN1 that were significantly associated with LBD and ADLBV pathologies but not AD. Strong association and an additive effect of multiple variants in GBA+SMPD1 or GBA+SMPD1+MCOLN1 were also observed across all disease phenotypes analysed.

The importance of the lysosomal pathway in CNS function and LBD and PD is highlighted by the identification of genetic risk factors or rare variants/mutations in lysosomal genes in case-control association studies (GBA and NAGLU)[7,9-11], GWAS studies (LAMP3, SCARB2)[24,25] or linkage analysis and exome sequencing in PD families (ATP13A2, VPS35 (endolysosomal pathway)[26,27]. A large multisite study of brain autopsy samples from subjects with different forms of dementia identified GBA mutations in 7.6% (6/79) of pure DLB cases (OR, 7.6 [95% CI, 1.8-31.9]) compared to 3.6% (8/222) of ADLBV cases (OR, 4.6 [95% CI, 1.2-17.6]) [28].

In the study, multiple variants predicted to be deleterious or damaging in GBA, SMPD1 and MCOLN1 in autopsy samples were significantly associated with LB and ADLBV pathology. The inventor identified a total of 26 variants in autopsy samples that have been previously reported as mutations in lysosomal storage disorders. These causal mutations in lysosomal storage disorders are usually observed in the homozygous or compound heterozygous state whereas in the autopsy samples that the inventor examined these mutations were observed in the heterozygous state suggesting that haploinsufficiency of lysosomal genes may contribute to LB and ADLBV phenotype. Overall, ˜15% of all LBD autopsy samples also carried a variant in more than one lysosomal storage disease gene examined suggesting that ‘multiple hits’ in the same biochemical pathway, the lysosomal pathway, might increase risk for LBD.

The data also shows that GBA mutation status is associated with significantly reduced GCase activity and a neuropathological diagnosis of LBD suggesting that haploinsufficiency or partial enzyme activity leads to increase in α-synuclein levels and Lewy body pathology. A decrease in GCase activity has been reported previously in brain autopsies from patients with Type I Gaucher Disease and parkinsonism and more recently in brain autopsies from patients with PD that carry GBA mutations [4,29]. A decrease in GCase activity has also been reported in brain autopsies from patients with sporadic PD without GBA mutations, with the greatest reduction in the substantia nigra [29] and conflicting reports of decreased GCase activity in the frontal cortical regions [29, 30]. In LBD brain autopsy samples from the frontal cortex without GBA mutations the inventor did not observe a decrease in GCase activity and the findings are consistent with one published study [29]. These conflicting reports of a reduction in GCase activity in different brain regions from PD or LBD autopsy samples without GBA mutations may reflect different stages of disease progression, neuronal loss or α-synuclein accumulation.

The utility of lipidomics has previously been demonstrated as a means to understand dysregulation of lipid metabolism and generate novel insights linked to AD pathogenesis[17]. Applying similar methodologies to the analysis of the motor cortex region of LBD brains with and without GBA mutations, the inventor observed that there are significant alterations in both major phospholipid and sphingolipid subclasses compared to controls. In the case of LBD carrying GBA mutations, GluCer was not significantly accumulated as one might expect. This may be reconciled by the existence of non-lysosomal glucosylceramidase GBA2 that is significantly expressed in the brain and can compensate for the deficiency in GBA activity [31, 32]. Nevertheless, it is worth noting that in both cases of LBD with and without GBA mutation, there appears to be significant or a trend towards accumulation of other sphingolipid subclasses including Cer, GalCer, Sulf, Sulf-OH and GM3 and the unusual phospholipid LBPA. LBPA is enriched in late endosomes where it functions in biogenesis of multivesicular bodies [33] and also in lysosomes where it plays a role in stimulating the hydrolysis of membrane bound sphingolipids. The overall profile of both LBD with and without GBA mutation cases suggests a common theme of dysfunction occurring in the endolysosomal degradative pathway that ultimately lead to defects in lysosomal clearance of autophagosomes and an accumulation of α-synuclein in LBD.

Recent studies in AD suggest that disease pathogenesis may begin more than 20 years before the onset of dementia [34]. Similarly in LBD, non-motor symptoms may predate motor symptoms by decades suggesting pathophysiological changes before clinical onset [34-37]. To date, there are no effective biomarkers for LBD. The study suggests that combined genetic and lipidomic data may prove effective in disease risk prediction, biomarker development (CSF) and targeted therapeutic strategies

### References

1. McKeith I G, Perry E K, Perry R H. Report of the second dementia with Lewy body international workshop: diagnosis and treatment. Consortium on Dementia with Lewy Bodies. Neurology. 1999;53: 902-905.

2. McKeith I G. Clinical Lewy body syndromes. Ann N Y Acad Sci. 2000;920: 1-8.

3. McKeith I G. Spectrum of Parkinson's disease, Parkinson's dementia, and Lewy body dementia. Neurol Clin. 2000;18: 865-902.

4. Tayebi N, Walker J, Stubblefield B, Orvisky E, LaMarca M E, Wong K, et al. Gaucher disease with parkinsonian manifestations: does glucocerebrosidase deficiency contribute to a vulnerability to parkinsonism? Mol Genet Metab. 2003;79: 104-109.

5. Suzuki K, Iseki E, Togo T, Yamaguchi A, Katsuse O, Katsuyama K, et al. Neuronal and glial accumulation of alpha- and beta-synucleins in human lipidoses. Acta Neuropathol. 2007;114: 481-489.

6. Choi J H, Stubblefield B, Cookson M R, Goldin E, Velayati A, Tayebi N, et al. Aggregation of α-synuclein in brain samples from subjects with glucocerebrosidase mutations. Mol Genet Metab. 2011;104: 185-188. doi:10.1016/j .ymgme.2011.06.008

7. Winder-Rhodes S E, Garcia-Reitbock P, Ban M, Evans J R, Jacques T S, Kemppinen A, et al. Genetic and pathological links between Parkinson's disease and the lysosomal disorder Sanfilippo syndrome. Mov Disord. 2012;27: 312-315. doi: 10.1002/mds.24029

8. Cox T M, Cachon-Gonzalez M B. The cellular pathology of lysosomal diseases. J Pathol. 2012;226: 241-254. doi:10.1002/path.3021

9. Clark L N, Ross B M, Wang Y, Mejia-Santana H, Harris J, Louis E D, et al. Mutations in the glucocerebrosidase gene are associated with early-onset Parkinson disease. Neurology. 2007;69: 1270-1277.

10. Sidransky E, Nalls M A, Aasly J O, Aharon-Peretz J, Annesi G, Barbosa E R, et al. Multicenter analysis of glucocerebrosidase mutations in Parkinson's disease. N Engl J Med. 2009;361: 1651-1661. doi:10.1056/NEJMoa0901281

11. Clark L N, Kartsaklis L A, Wolf Gilbert R, Dorado B, Ross B M, Kisselev S, et al. Association of glucocerebrosidase mutations with dementia with lewy bodies. Arch Neuro1.2009;66: 578-583. doi:10.1001/archneuro1.2009.54

12. Gan-Or Z, Ozelius L J, Bar-Shira A, Saunders-Pullman R, Mirelman A, Kornreich R, et al. The p.L302P mutation in the lysosomal enzyme gene SMPD1 is a risk factor for Parkinson disease. Neurology. 2013;80: 1606-1610. doi: 10.1212/WNL.0b013e31828f180e

13. McKeith I G, Dickson D W, Lowe J, Emre M, O'Brien J T, Feldman H, et al. Diagnosis and management of dementia with Lewy bodies: third report of the DLB Consortium. Neurology. 2005;65: 1863-1872.

14. Mirra S S, Heyman A, McKeel D, Sumi S M, Crain B J, Brownlee L M, et al. The Consortium to Establish a Registry for Alzheimer's Disease (CERAD). Part II. Standardization of the neuropathologic assessment of Alzheimer's disease. Neurology. 1991;41: 479-486.

15. NACC Neuropathology (NP) Diagnosis Coding Guidebook. Seattle: University of Washington: National Alzheimer's Coordinating Center; 2005-2008.

16. Yang J, Lee S H, Goddard M E, Visscher P M. GCTA: a tool for genome-wide complex trait analysis. Am J Hum Genet. 2011;88: 76-82. doi: 10.1016/j.ajhg.2010.11.011

17. Chan R B, Oliveira T G, Cortes E P, Honig L S, Duff K E, Small S A, et al. Comparative lipidomic analysis of mouse and human brain with Alzheimer disease. J Biol Chem. 2012;287: 2678-2688. doi:10.1074/jbc.M111.274142

18. Chan R, Uchil P D, Jin J, Shui G, Ott D E, Mothes W, et al. Retroviruses human immunodeficiency virus and murine leukemia virus are enriched in phosphoinositides. J Virol. 2008;82: 11228-11238. doi:10.1128/JVI.00981-08

19. Wu M C, Lee S, Cai T, Li Y, Boehnke M, Lin X. Rare-variant association testing for sequencing data with the sequence kernel association test. Am J Hum Genet. 2011;89: 82-93. doi:10.1016/j.ajhg.2011.05.029

20. Hruska K S, LaMarca M E, Scott C R, Sidransky E. Gaucher disease: mutation and polymorphism spectrum in the glucocerebrosidase gene (GBA). Hum Mutat. 2008;29: 567-583. doi:10.1002/humu.20676

21. Irun P, Mallen M, Dominguez C, Rodriguez-Sureda V, Alvarez-Sala L A, Arslan N, et al. Identification of seven novel SMPD1 mutations causing Niemann-Pick disease types A and B. Clin Genet. 2013;84: 356-361. doi:10.1111/cge.12076

22. Simonaro C M, Desnick R J, McGovern M M, Wasserstein M P, Schuchman E H. The demographics and distribution of type B Niemann-Pick disease: novel mutations lead to new genotype/phenotype correlations. Am J Hum Genet. 2002;71: 1413-1419.

23. Triggs-Raine B, Richard M, Wasel N, Prence E M, Natowicz M R. Mutational analyses of Tay-Sachs disease: studies on Tay-Sachs carriers of French Canadian background living in New England. Am J Hum Genet. 1995;56: 870-879.

24. Nalls M A, Plagnol V, Hernandez D G, Sharma M, Sheerin U-M, Saad M, et al. Imputation of sequence variants for identification of genetic risks for Parkinson's disease: a meta-analysis of genome-wide association studies. Lancet. 2011; 377: 641-649. doi: 10.1016/S0140-6736(10)62345-8

25. Do C B, Tung J Y, Dorfman E, Kiefer A K, Drabant E M, Francke U, et al. Web-based genome-wide association study identifies two novel loci and a substantial genetic component for Parkinson's disease. PLoS Genet. 2011;7(6): e1002141 doi: 10.1371/journal.pgen.1002141

26. Di Fonzo A, Chien H F, Socal M, Giraudo S, Tassorelli C, Iliceto G, et al. ATP13A2 missense mutations in juvenile parkinsonism and young onset Parkinson disease. Neurology. 2007;68: 1557-1562.

27. Vilarino-Guell C, Wider C, Ross O A, Dachsel J C, Kachergus J M, Lincoln S J, et al. VPS35 mutations in Parkinson disease. Am J Hum Genet. 2011;89: 162-167. doi: 10.1016/j.ajhg.2011.06.001

28. Tsuang D, Leverenz J B, Lopez O L, Hamilton R L, Bennett D A, Schneider J A, et al. GBA mutations increase risk for Lewy body disease with and without Alzheimer disease pathology. Neurology. 2012;79: 1944-1950. doi: 10.1212/WNL.0b013e3182735e9a

29. Gegg M E, Burke D, Heales S J R, Cooper J M, Hardy J, Wood N W, et al. Glucocerebrosidase deficiency in substantia nigra of parkinson disease brains. Ann Neurol. 2012;72: 455-463. doi:10.1002/ana.23614

30. Murphy K E, Gysbers A M, Abbott S K, Tayebi N, Kim W S, Sidransky E, et al. Reduced glucocerebrosidase is associated with increased α-synuclein in sporadic Parkinson's disease. Brain. 2014;137: 834-848. doi:10.1093/brain/awt367

31. Boot R G, Verhoek M, Donker-Koopman W, Strijland A, van Marle J, Overkleeft H S, et al. Identification of the non-lysosomal glucosylceramidase as beta-glucosidase 2. J Biol Chem. 2007;282: 1305-1312.

32. Yildiz Y, Matern H, Thompson B, Allegood J C, Warren R L, Ramirez D M O, et al. Mutation of beta-glucosidase 2 causes glycolipid storage disease and impaired male fertility. J Clin Invest. 2006;116: 2985-2994.

33. Kobayashi T, Beuchat M H, Lindsay M, Frias S, Palmiter R D, Sakuraba H, et al. Late endosomal membranes rich in lysobisphosphatidic acid regulate cholesterol transport. Nat Cell Biol. 1999;1: 113-118.

34. Bateman R J, Xiong C, Benzinger T L S, Fagan A M, Goate A, Fox N C, et al. Clinical and biomarker changes in dominantly inherited Alzheimer's disease. N Engl J Med. 2012;367: 795-804. doi:10.1056/NEJMoa1202753

35. Braak H, Del Tredici K, Rub U, de Vos R A I, Jansen Steur E N H, Braak E. Staging of brain pathology related to sporadic Parkinson's disease. Neurobiol Aging. 2003;24: 197-211.

36. Hawkes C H. The prodromal phase of sporadic Parkinson's disease: does it exist and if so how long is it? Mov Disord. 2008;23: 1799-1807. doi:10.1002/mds.22242

37. Koller W C. When does Parkinson's disease begin? Neurology. 1992; 42: 27-31; discussion 41-28.

38. McKeith I G, Dickson D W, Lowe J, Emre M, O'Brien J T, et al. (2005) Diagnosis and management of dementia with Lewy bodies: third report of the DLB Consortium. Neurology 65: 1863-1872.

39. Mirra S S, Heyman A, McKeel D, Sumi S M, Crain B J, et al. (1991) The Consortium to Establish a Registry for Alzheimer's Disease (CERAD). Part II. Standardization of the neuropathologic assessment of Alzheimer's disease. Neurology 41: 479-486.

40. National Alzheimer's Coordinating Center. NACC Neuropathology (NP) Diagnosis Coding Guidebook. Seattle: University of Washington: (2005-2008).

41. Carmi S, Hui K Y, Kochav E, Liu X, Xue J, et al. (2014) Sequencing an Ashkenazi reference panel supports population-targeted personal genomics and illuminates Jewish and European origins. Nat Commun 5: 4835-4835.

42. González-Pérez A, López-Bigas N (2011) Improving the Assessment of the Outcome of Nonsynonymous SNVs with a Consensus Deleteriousness Score, Condel. The American Journal of Human Genetics 88: 440-449.

43. Takagi Y, Kriehuber E, Imokawa G, Elias P M, Holleran W M (1999) Beta-glucocerebrosidase activity in mammalian stratum corneum. J Lipid Res 40: 861-869.

44. Balducci C, Pierguidi L, Persichetti E, Parnetti L, Sbaragli M, et al. (2007) Lysosomal hydrolases in cerebrospinal fluid from subjects with Parkinson's disease. Mov Disord 22: 1481-1484.

45. Chan R B, Oliveira T G, Cortes E P, Honig L S, Duff K E, et al. (2012) Comparative lipidomic analysis of mouse and human brain with Alzheimer disease. J Biol Chem 287: 2678-2688.

46. Chan R, Uchil P D, Jin J, Shui G, Ott D E, et al. (2008) Retroviruses human immunodeficiency virus and murine leukemia virus are enriched in phosphoinositides. J Virol 82: 11228-11238.

### 6.2. Example 2:

### Role of MCOLN1 in Lewy Body Disease

Dementia with Lewy bodies (DLB) is one of the most common causes of dementia with a prevalence of 4.2 to 7.5% and incidence of 3.8% in newly diagnosed dementia patients. Clinically, the disease is characterized by cognitive decline, visual hallucinations and parkinsonism. Current treatment strategies for DLB are symptomatic which may be effective in the early stages of the disease but do not prevent disease progression. Identifying the genetic factors that contribute to DLB will lead to the ultimate goal of more effective treatments and disease modifying therapies. To date, there have been few genetic studies that have focused on DLB, which has been hindered by the mixed phenotype in families and complex inheritance pattern. In this proposal, the inventor will study a multigenerational Ashkenazi Jewish pedigree with dementia that carries a heterozygous MCOLN1 mutation by whole genome sequencing to dissect the contribution of genetic variation to dementia in this pedigree. The inventor believe this will be a powerful approach, and will lead to the identification of genetic risk factors contributing to dementia as well as providing possible insight into more effective treatments for MLIV in addition to DLB. α-Synuclein immunoreactivity, including LB, have been described as features seen in the neuropathology of several lysosomal storage disorders, including notably Gaucher Disease, but also Sandhoff disease, Tay Sachs Disease, and Sanfilippo syndrome. Characteristic inclusion or compound bodies composed predominantly of lipids and mucopolysaccharides-like material are also present in neurons. A goal of this proposal is to determine whether α-Synuclein, a protein that characteristically accumulates in Lewy bodies also accumulates in a Mucolipidosis Type IV mouse model.

The inventor proposes two Specific Aims:

AIM 1: To perform rare variant and gene-based linkage analysis in a multigenerational Ashkenazi Jewish pedigree with dementia: Pedigree-VAAST (pVAAST), a disease-gene identification tool for high throughput sequence data in pedigrees will be used to perform rare variant and gene-based linkage analysis in a multigenerational Ashkenazi Jewish pedigree with dementia to determine whether a previously identified heterozygous MCOLN1 mutation in family members contributes to dementia in this family. Whole genome sequencing will be performed in five family members that include parents and three offspring.

AIM 2: Neuropathological characterization of inclusion bodies in a Mucolipidosis Type IV (MLIV) mouse model. A mouse model for MLIV has been generated through knock-out of the Mcoln gene. One of the features of the mouse is enlarged late endosome/lysosomes (LELs) that accumulate storage material such as lipofuscin. To assess whether α-synuclein (a protein that characteristically accumulates in Lewy bodies) also accumulates in LELs, brain tissue from Mcoln-/−, Mcoln-/+and WT mice will be examined. In addition, the accumulation of Alzheimer's related proteins (beta amyloid and phosphorylated tau), and markers of inflammatory response will also be assessed.

Hypothesis: The inventor hypothesize that heterozygous MCOLN1 mutations may be a risk factor for Dementia with Lewy Bodies (DLB) and that the pre-synaptic neuronal protein α-Synuclein, that is linked genetically and neuropathologically to Lewy body disease (Parkinson's disease and DLB) and characteristically accumulates in Lewy bodies will accumulate in MLIV mice.

### Significance

Dementia with Lewy bodies (DLB) is one of the most common causes of dementia with a prevalence of 4.2 to 7.5% and incidence of 3.8% in newly diagnosed dementia patients.¹ Clinically, the disease is characterized by cognitive decline, visual hallucinations and parkinsonism. The cognitive symptoms of patients with DLB can fluctuate and the presence of parkinsonism is not required for a clinical diagnosis of DLB.²⁻⁴ In contrast to Alzheimer disease (AD) patients DLB patients have impairments in visuospatial skills, attention and executive functioning. Parkinsonism is often seen in patients with DLB and is associated with higher functional disability and is characterized by bilateral parkinsonism and a postural tremor. Neuropsychiatric features are common in DLB and can include visual hallucinations, depression, apathy and delusions.⁵ The visual hallucinations in DLB can occur earlier than usually observed in AD and within the first 5 years of a dementia diagnosis. Autonomic features such as orthostatic hypotension, constipation and urinary incontinence are commonly observed in DLB. The neuropathological hallmark of DLB is the widespread presence of inclusions commonly referred to as Lewy Bodies (LBs) and Lewy Neurites^{6,7} that are positive for the presynaptic neuronal protein α-Synuclein. However, pure LB pathology is only observed in 20-30% of patients with clinical DLB and most DLB brains also have low or intermediate AD pathology in the form of β-amyloid plaques (Aβ) and neurofibrillary tangles.^{8,9} Current treatment strategies for DLB are symptomatic which may be effective in the early stages of the disease but do not prevent disease progression. Identifying the genetic factors that contribute to DLB will lead to the ultimate goal of more effective treatments and disease modifying therapies.

Genetics of DLB: Family studies provide strong evidence for a genetic contribution to DLB with several studies reporting aggregation of DLB or a mixed phenotype of parkinsonism and dementia inherited in families.¹⁰⁻¹⁹ However, DLB is likely to be complex and the evidence to date suggests that the genetic etiology of DLB is likely to be influenced by some of the same risk factors for AD and PD in addition to other genetic risk factors. What is the evidence for a complex inheritance pattern?:1) DLB is usually a late onset disease, 2) lack of concordance in a twin study of DLB suggesting environmental factors may also play a role20 and 3) lack of gene identification in the only published linkage scan in a large three-generation Belgian family with prominent dementia and parkinsonism consistent with DLB.²¹ Although significant linkage to 2q35-q36 (Z=3.01 at D2S1242) was observed in the Belgian family, subsequent molecular genetic investigations including sequencing of genes in the candidate region and copy number variant analysis did not identify a pathogenic mutation or gene dosage mutation that co-segregated with DLB in the pedigree.²² The inventor and others have evaluated candidate genes for DLB based on familial genes and genetics risk factors identified in related neurodegenerative disorders. These genes and risk factors include PD genes (α-Synuclein (SNCA),^{23,11,15,19} Glucocerebrosidase (GBA),^{24,25} SCARB2²⁶ and AD genes (amyloid precursor protein gene (APP),²⁷ presenilin 1 (PSEN1),^{13,27} presenilin 2 gene (PSEN2)²⁷ and Apolipoprotein E (APOE) e4 allele).²⁶⁻²⁸ Collectively, these studies suggest that GBA and APOE represent the most significant risk factors for DLB identified so far. However, there have been few genetic studies that have focused on DLB, which has been hindered by the mixed phenotype in families and complex inheritance pattern. In this proposal, the inventor will study a multigenerational Ashkenazi Jewish pedigree with dementia that carries a heterozygous MCOLN1 mutation by whole genome sequencing to dissect the contribution of genetic variation to dementia in this pedigree. The inventor believe this will be a powerful approach, as the inventor outline below, and lead to the identification of genetic risk factors contributing to dementia as well as providing possible insight into more effective treatments for Mucolipidosis Type IV (MLIV) in addition to DLB.

Ashkenazi Jews (AJ) identified as Jewish individuals of Central- and Eastern European ancestry, form the largest genetic isolate in the United States. AJ demonstrate distinctive genetic characteristics²⁹ including a high prevalence of autosomal recessive diseases and a relatively high frequency of alleles that confer a strong risk of common diseases such as Parkinson's disease,³⁰ Crohn's disease³¹ and breast and ovarian cancer.³² The inventor and others have shown that AJ are a genetically distinct population close to other Jewish populations as well as to present day Middle Eastern and European populations.³³⁻⁴¹ The AJ population is much larger and/or experienced a more severe bottleneck than other founder populations such as Amish, Hutterites or Icelanders whose demographic histories have facilitated a number of genetic discoveries. ^{42,43}

Recently, the inventor reported a catalogue of 128 high coverage whole genome AJ sequences. Compared with a European reference panel, the AJ panel has 47% more novel and population-specific variants and the inventor have demonstrated that the AJ panel is necessary for interpretation and imputation of personal genomes and is eightfold more effective at filtering benign variants out of AJ clinical genomes.⁴⁴ Analysis of long chromosomal segments, which are abundant in AJ, confirms a recent severe bottleneck of ˜350 individuals which has facilitated cost-effective cataloguing of the vast majority of (prebottleneck) AJ variation, even when taking into account the large size of this population. What are the implications for this in studying complex disease? It suggests an increased power to detect rare alleles of large effect that drifted to higher frequencies during the bottleneck and for the first time the opportunity to determine the genetic architecture of complex disease that has not been possible previously in large outbred populations (E.g North America). Such an approach has been successful in other isolated populations.^{42,43} The inventor have previously demonstrated the power of this approach in the AJ population with the discovery of mutations in the GBA gene as a risk factor for Parkinson's disease (PD)⁴⁵⁻⁴⁷ and Dementia with Lewy bodies (DLB)²⁴ and a gene x gene interaction between LRRK2 and PARK16 in PD.⁴⁸ The own studies in the AJ population in addition to successes in other isolated populations serve as the motivation for the current study and to perform a genetic analysis of a multigenerational Ashkenazi Jewish pedigree with dementia that carries a heterozygous MCOLN1 mutation and to determine the contribution of genetic variation to dementia in this pedigree.

Lysosomal storage diseases (LSDs) are a group of metabolic disorders caused by genetic mutations in three classes of proteins:1) lysosomal hydrolases required for catabolic degradation, 2) lysosomal membrane proteins important for catabolite export or membrane trafficking and 3) non-lysosomal proteins that indirectly affect lysosomal function (reviewed in Platt 2014⁴⁹ and Samie and Xu 2014⁵⁰). The importance of the lysosome is highlighted by the large number of diseases that have been documented ranging from cancer to neurodegenerative disease, and the pathology in several tissues and organs. The classic feature of LSDs is the accumulation of undigested lipids in the lysosome leading to lysosomal dysfunction and cell death. More than 60 LSDs have been described which are multisystemic and clinically heterogeneous but often have a neurological involvement. A spectrum of neurological manifestations has been noted in patients with four of the most common lysosomal storage disorders in the AJ that include Gaucher disease (GD), Tay Sachs Disease (TSD), Niemann Pick Disease and Mucolipidosis type IV (MLIV) that can be classified based on age at onset and severity of the disease. The extent of variation of nervous system involvement and correlation with specific lysosomal gene mutations in patients with an intermediate or mild late-onset phenotype is currently unknown.⁴⁹ However, a spectrum of neurological manifestations has been noted in patients with mutations in each of these genes ranging from epilepsy, myoclonus, supranuclear gaze palsy, cerebellar ataxia, psychiatric symptoms, parkinsonism and dementia. ⁴⁹

Mucolipidosis Type IV: MLIV (OMIM 252650) is an autosomal recessive LSD associated with lysosomal inclusions (storage or compound bodies) in a variety of cell types including the nervous system. MLIV is progressive but is usually evident in the first year of life and presents with intellectual disability, corneal opacities and delayed developmental milestones. Ocular findings include retinal degeneration, myopia, strabismus and photophobia. Neurological symptoms include hypotonia and pyramidal tract signs. MLIV patients also have constitutive achlorhydria associated with secondary blood gastrin elevation and frequent malabsorption of iron from food. Mutations in mucolipin-1 (also known as TRPML1), a transmembrane protein of the transient receptor potential channel family, that is encoded by the MCOLN1 gene cause MLIV. Over 20 different MCOLN1 mutations have been described in MLIV patients. In the AJ, two founder mutations (IVS3-2 A>G and ˜6.5Kb deletion) account for 95% of cases. Currently, there is no specific therapy for MLIV.

Genetic Links between LSDs and Common Neurologic Disorders: Previously, the inventor have shown that heterozygous mutations in the GBA gene are a risk factor for Lewy Body Disorders including PD^{45,46,51-53} and DLB^{24,25} and the genetic link between GD and LBD is now widely accepted. Of note, the first genetic association between GBA and parkinsonism that the inventor reported in a pilot study, was in an AJ population. In a larger study, the inventor subsequently showed that the frequency of GBA mutations in AJ PD cases was more than double that of non-AJ PD demonstrating the power of identifying genetic risk factors in this population. In an international multicenter collaborative study in which the inventor participated with more than 5600 patients with PD that included both AJ and non-AJ subjects and an equal number of controls, demonstrated that overall among patients with PD, the odds ratio for carrying a GBA mutation was greater than 5. Mutations in GBA are also a significant risk factor for DLB^{24,25} confirming previous clinical observations that GBA-associated PD is characterized by an increased incidence of dementia.⁵³ In brain autopsies, the inventor showed that GBA mutation status was significantly associated with the presence of cortical LB (OR=6.48, 95% CI, 2.45-17.16, p<0.001) and a neuropathological diagnosis of DLB after adjusting for sex, age at death, and presence of APOE- 4.²⁵ Recently, other studies suggest that genetic variation in other LSDs may also contribute to LBD. A common SMPD1 L302P mutation has been reported as a risk factor for PD in the AJ population⁵⁴ and a genetic link between PD and the LSD Sanfilippo syndrome⁵⁵ has also been described. Herein the inventor present data that shows that in a large brain autopsy series variants in GBA, SMPD1 and MCOLN1 are associated with LB neuropathology. Neuropathology: α-Synuclein LB-like inclusion bodies are a feature of many LSDs: α-Synuclein immunoreactivity, including LB, have been described as features seen in the neuropathology of several lysosomal storage disorders, including notably GD^{56,57}, but also Sandhoff disease, TSD, and Sanfilippo syndrome.^{55,58,59} Subjects with GBA mutations who develop PD demonstrate on autopsy typical neuropathological hallmarks with post mortem α-synuclein post mortem inclusions and a-synuclein aggregates in neuronal cells. ⁵⁷. A significant decrease in Gcase activity has also been observed in both cerebrospinal fluid (CSF)^{60,61} and brain autopsy tissue^{62,63} from PD and DLB patients that carry GBA mutations in the heterozygous state. The own data support these findings. A more recent study, also observed an increased plasma oligomeric α-synuclein in patients with GD and several other LSDs. Neuropathology of Mucolipidosis Type IV: Mill findings in MLIV patients are characterized by widespread abnormal white matter, a dysplastic corpus callosum and cerebellar atrophy in older patients, and increased ferritin deposition in the thalamus and basal ganglia. Characteristic inclusion or compound bodies composed predominantly of lipids and mucopolysaccharides-like material are also present in neurons. A goal of this proposal is to determine whether α-Synuclein, a protein that characteristically accumulates in Lewy bodies also accumulates in Mcoln1-/− mice.

Discovery of DLB genes would have a major impact on the field. The inventor have carefully considered approaches to move the field forward, and the inventor believe that the approach described in this proposal is innovative for several reasons. First, the inventor have focused on one of the major impediments to previous genetic studies of DLB, which has been hindered by the mixed phenotype in families and complex inheritance pattern by performing whole genome sequencing to dissect the contribution of genetic variation to dementia in an AJ pedigree. Second, the inventor will perform state-of-the-art molecular genetic analyses, including WGS. By using WGS in this proposal the inventor will be able to confirm that coding variants were not missed because of poor coverage in WES data and this approach will also allow us to analyze both coding and non-coding variants in addition to indels and structural variants. Further, WGS from four members of a family (quartet⁶⁴; parents and discordant offspring) in addition to a most distantly related affected is a more powerful approach than WGS from fewer family members or sets of unrelated genomes because:1) it allows inheritance analyses that detect errors and identification of precise locations of recombination events. ⁶⁴2) it provides confident predictions of inheritance states and haplotypes. 3) it permits the detection of >70% of sequencing errors and thus reduces the search space for disease causing variants. ⁶⁴ These analyses would be far less powerful using exome data or sequencing of fewer family members. Third, the inventor will use novel analytic methods for linkage and analysis of rare variants to identify ET genes. Several methods (e.g., pVAAST) for analysis of WGS data and identification of rare variants in family based samples have recently been developed. By contrast, almost all previous studies that use NGS (WES) in families to identify disease genes employed a filter-based methodology, where variants identified in cases were checked for rarity (e.g., absent from SNP databases), potential functional impact (e.g., nonsynonymous variants), and sharing among affected (and possibly related) individuals. Although this approach was intuitive and reasonable, it had several limitations:1) failure to produce any measure of statistical uncertainty (e.g., gene-level p values), making it unfeasible to assess consistency with the null hypothesis; 2) no adjustment for background variation in each gene, thereby allowing large genes to rank high on the basis of their size alone; and 3) failure to account for expected levels of variant sharing among relatives of different types, which can affect the rank of the genes. Fourth, the inventor will perform an assessment of the impact of Mcoln1 deficiency on α-Synuclein accumulation in a mouse model. These studies have not previously been performed and could provide insight into more effective treatments for both MLIV and DLB.

In an R01 funded study (NS060113, Clark PI), the inventor investigated whether variants in other lysosomal storage disorder (LSD) genes also contribute to LBD disease pathogenesis. The inventor performed a genetic analysis of four LSD genes including GBA, HEXA, SMPD1, and MCOLN1 in 231 brain autopsies (Manuscript submitted in revision). Brain autopsies included neuropathologically defined LBD without Alzheimer Disease (AD) changes (n=59), AD without significant LB pathology (n=71), Alzheimer disease and lewy body variant (ADLBV) (n=68), and control brains without LB or AD neuropathology (n=33). Sequencing of HEXA, SMPD1, MCOLN1 and GBA followed by ‘gene wise’ genetic association analysis was performed. To determine the functional effect, a biochemical analysis of GBA in a subset of brains was also performed. GCase activity was measured in a subset of brain samples (n=64) that included LBD brains, with or without GBA mutations, and control brains. A lipidomic analysis was also performed in brain autopsies (n=67) and included LBD (n=34), ADLBV (n=3), AD (n=4), PD (n=9) and control brains (n=17), comparing GBA mutation carriers to non-carriers. In a ‘gene-wise’ analysis, variants in GBA, SMPD1 and MCOLN1 were significantly associated with LB pathology (p range:0.03-4.14 ×10⁻⁵). Overall, the mean levels of GCase activity were significantly lower in GBA mutation carriers compared to non-carriers (p<0.001). A significant increase and accumulation of several species for the lipid classes, ceramides and sphingolipids, was observed in LBD brains carrying GBA mutations compared to controls (p range: p<0.05-p<0.01). The study indicates that variants in GBA, SMPD1 and MCOLN1 are associated with LB pathology. Biochemical data comparing GBA mutation carrier to non-carriers support these findings, which have important implications for biomarker development and therapeutic strategies.

AIM 1-To Perform Rare Variant and Gene-Based Linkage Analysis in a Multigenerational Ashkenazi Jewish Pedigree with Dementia

Description of the Family: A multigenerational Ashkenazi Jewish pedigree with dementia that carries a previously identified heterozygous MCOLN1 mutation has agreed to participate in this research study. The pedigree was identified through the Division of Personalized Genomic Medicine at Columbia University Medical Center. WGS will be performed in five family members that include parents and three offspring.

WGS in the Pedigree: The inventor will generate >100Gb of sequence at >30X coverage per sample using the TruseqDNA nano kit and the Illumina Hiseq X ten platform. The capture and sequencing will be performed by the New York Genome Center.

Detection of Variants: The inventor have developed a pipeline for QC of FASTQ files, read alignment and mapping to the reference genome, and variant calling and filtering to generate a variant call format (VCF) file. QC of data will be performed using FASTQC (Babraham bioinformatics, UK) and alignment to the reference genome using the Burrows wheel aligner (BWA).⁶⁵ Variant calling and filtering will be performed using the genome analysis toolkit⁶⁶ (GATK and unified genotyper v1.6; Broad Institute) and annotation using ANNOVAR⁶⁷ and dbSNP, refseq and the 1000 genomes project. Data processing will be performed by a postdoctoral fellow in Dr. Clark's lab that has experience using these tools.

Single-nucleotide variant calling: SNV calling will be performed on all samples jointly using GATK unified genotyper v1.6. Calls will be filtered using GATK Variant Quality Score Recalibration and quality metrics will be evaluated. The following SNP filters will be applied including: Qual<30, Phred scale probability of polymorphism existing; QD<5.0, quality of depth of reads, HRun>5, homopolymer run; SB>0.10, strand bias and MQ0>=4 or MQ0/DP>0.1 for ambiguously mapped reads.

Indels and Structural Variants: The inventor will use a combination of 8 algorithms to generate a consensus of reliable indel and structural calls including GATK Unified Genotyper^{66,} Pindel⁶⁸, Breakdancer⁶⁹, CNVnator⁷⁰, FACADE⁷¹, MATE-CLEVER⁷², GenomeSTRiP⁷³ and SOAPdenovo⁷⁴. These algorithms are based on six approaches:1) gapped reads, 2) split reads, 3) read pairs, 4) read depth, 5) combined approaches and 6) de novo assembly. Each tool will be run and calls determined and filtered separately. The data will then be merged and further filtering applied to generate a dataset of reliable indel and structural variants. Variants will be divided into three groups based on size:1-20bp, 20-100bp and >100bp. The inventor have extensive previous experience with analysis of copy number and structural variation. ^{44,75}

Linkage and Rare Variant analysis: The inventor will perform a unified test of linkage and rare variant association and functional variant prediction as implemented in p-VAAST.⁷⁶ Unlike lod scores in traditional parametric linkage analysis, the lod score in pVAAST is designed for sequence data. Specifically, the statistical model assumes that dysfunctional variants influencing disease-susceptibility can be directly detected. As a result, the pVAAST lod score is in general more powerful than traditional linkage analysis with sequence data. pVAAST is built upon the composite likelihood ratio test (CLRTv) used in VAAST, but in addition, integrates linkage information (quantified by a lod score) as a separate log likelihood ratio in the pVAAST CLRT (CLRTp). pVAAST evaluates the significance of the CLRTp score using a combination of a randomization test and gene-drop simulation. pVAAST outperforms linkage and rare-variant association tests in simulations. The approach is robust to incomplete penetrance and locus heterogeneity and is applicable to a wide variety of genetic traits. The inventor have successfully implemented pVAAST in other genetic studies (Essential Tremor, R01 NS073872). The control genome dataset will consist of 1,057 exomes from the 1000 genomes project and 128 AJ genomes from the published dataset. Traits available for the AJ genome dataset include total modified mini mental state examination (mMMS) score, total unified Parkinson's disease rating scale (UPDRS) (Part II and Part III), family history of PD and AD in first degree relatives, depression, thought disorder and intellectual impairment.

Identification of Inherited and De Novo Variants: Inherited SNV, indel and structural variants in offspring will be determined by the presence of the variant in one or both parents. De novo SNV, indel and structural variants will be identified in offspring based on absence of the variant in both parents. The inventor will use a machine learning based classifier called forestDNM (RF-2). The method is designed to distinguish de novo variants form false positive de novo mutations that arose from errors in sequencing, alignment or variant calling.

Variant Annotation: SNVs will be functionally annotated using the variant annotation tool (VAT)⁷⁷ and snpEff⁷⁸ VAT provides annotation of both coding and non-coding variants whereas snpEff annotates and predicts the effects of coding variants on genes. Non-Synonymous SNVs will be annotated with in Silico prediction programs including PROVEAN⁷⁹, Mutation Taster⁸⁰, Polyphen⁸¹ and SIFT⁸². Annotation will also be performed using the ‘combined annotation dependent depletion score’ (CADD)⁸³. SNVs in OMIM and disease-causing SNVs in HGMD will be annotated. Gene Level annotation will be performed using the ‘residual variation intolerance score’ (RVIS)⁸⁴. Evolutionary conservation will be assessed and SNVs annotated with Genomic Evolutionary Rate Profiling (GERP) scores⁸⁵ . Indels will be annotated using indelMapper in VAT with GENCODE v16 annotations. Structural variants will be annotated using RefSeq.

Medical Annotation and Family Analysis: The inventor will examine the function of genes that carry rare variants predicted to be damaging or deleterious with documented neurodevelopmental, neurobehavioral or neurodegenerative phenotype in humans or in animal models (mouse, C.elegans, Drosophila, Zebrafish). For human annotation, the inventor will use published literature together with Genecards, the database for annotation, visualization and integrated discovery (DAVID) v6.7, the human phenotype ontology database (www.humanphenotype-ontology.org/), Phenotips, OMIM, HGMD, Orphanet and DECIPHER. For animal models the inventor will use published literature, Genecards, mammalian phenotype ontology, flybase, wormatlas, the zebrafish model organism database (zfin.org). For family analysis the inventor will assess the clinical phenotype of family members with and without the deleterious variant (s) for neuropsychiatric, dementia, parkinsonism, severity and duration. Clinical reassessment of the proband and family members will also be performed.

Classification as Known, Novel or Candidate Gene: Linked or associated variants will be classified according to the guidelines and recommendations of MacArthur et al (2014)86 for investigating causality of sequence variants in human disease. In assessing the evidence for candidate disease genes in monogenic disease families. The inventor will evaluate genes previously implicated with a similar phenotype (e.g., dementia, parkinsonism, neurodegenerative disease) based on literature reports, OMIM and HGMD, before exploring potential new genes.

### AIM 2-Neuropathological Characterization of Inclusion Bodies in a Mucolipidosis Type IV (MLIV) Mouse Model.

The inventor will perform an assessment of the impact of Mcoln1 deficiency on synuclein accumulation in a mouse model. The loss of Mcoln1 (TRPML1) results in enlarged late endosomes and lysosomes (LELs) and the accumulation of lysosomal storage materials such as lipofuscin in most cell types of MLIV patients⁸⁷ and

Mcoln knock-out mice⁸⁸. Using genetic knockout (KO) approaches, animal models of MLIV have been well established in mice⁸⁸, C. elegans⁸⁹, and Drosophila⁹⁰. The first murine model of Mcoln1 KO generated by Slaugenhaupt's group⁸⁸ displays clinical features of MLIV patients, Interestingly, progressive neurodegeneration phenotypes are prominent in Mcoln1 KO mice, which exhibit gait changes at an age of 3 months and gradually develop hind-limb paralysis and typically die at the age of 8-9 months⁸⁸. At the cellular level, MLIV-like dense granulomembranous storage bodies are observed in Mcoln1 KO neurons and glial cells. MLIV-like phenotypes are also observed in the knockout models of C. elegans and Drosophila. The cup-5 mutant worms exhibit decreased degradation of endocytosed proteins and accumulation of large vacuoles labeled with LEL markers, indicative of the defective endocytic trafficking⁹¹. Studies on the Drosophila model of MLIV demonstrate that progressive neuronal death in TRPML-null flies is likely due to impaired autophagy, which results in the accumulation of lysosomal lipofuscin and damaged mitochondria which is associated with high levels of apoptosis90. Additionally, the inefficient clearance of apoptotic neurons by TRPML-null glial cells has been proposed to aggravate cell death in neurons⁹⁰. The inventor therefore propose that one mechanism by which variants in Mcoln1 may cause DLB is through inefficient clearance of α-synuclein that could accumulate in LELs.

Mcoln1 is believed to channel iron ions across the endosomal/lysosomal membrane into the cell. The Mcoln1 KO mouse shows a reduction in cytosolic Fe²⁺ levels but an increase in intralysosomal Fe²⁺ levels⁹². Iron accumulation in Parkinson's disease has been reported since the 1920's and the interaction between iron and synuclein has been investigated at many levels from aggregation to transcription/translation and regulation (recently reviewed in Carboni and Lingor⁹³).

As a first step to explore how variants in Mcoln1 could be associated with DLB the inventor will examine whether deficiency in Mcoln1 causes synucleinopathy using the mouse model of MLIV developed by Dr. Slaugenhaupt. Dr. Slaugenhaupt has kindly agreed to supply us with Mcoln1 KO mice. To assess whether Mcoln-1 deficiency correlates with accumulated synuclein the inventor will immunolabel brain tissue from Mcoln-1 −/−, −/+and +/+(WT) mice with antibodies against murine α-synuclein (both phospho-independent and phosphorylated including S129). Synuclein accumulated in neurons and/or neurites will be assessed quantitatively. As the accumulation of aggregated proteins is often associated with gliosis, and glial cells appear to be deficient in their function in TRPML-1 null animals⁹⁰, the inventor will perform a preliminary screen for markers of inflammatory response such as the presence of reactive astrocytes (GFAP immunopositive) and activated microglia (CD-68 immunopositive for activated glia, Ibal for resting and activated glia). As mentioned previously, most DLB brains also have low or intermediate AD pathology in the form of β-amyloid plaques (Aβ) and neurofibrillary tangles.^{8,9} and TRPML-1 has been implicated in the intraneuronal clearance⁹⁴ of Aβ. The inventor will therefore examine the Mcoln deficient mice for accumulation of Aβ and phosphorylated tau. Murine AP can be visualized using antibody 4G8 and phosphorylated murine tau can be identified using antibodies relevant for AD such as AT8 (pS202/205) or PHF1 (pS396/404). While the inventor would expect Aβ and/or tau accumulation to be associated with the LELs as they are cleared through the endosomal/lysosomal system, the inventor will also examine the parenchyma for the presence of amyloid (especially diffuse amyloid deposits), and neurites and cell bodies for accumulated tau. Additional assessments can be performed by ELISA on frozen tissue if levels of Aβ or tau are thought to be low, or too diffuse to observe by IHC. In addition to pathological protein assessment, the sections will be labeled with LAMP1 to identify lysosomes. Ubiquitinylated proteins accumulate with defective autophagy therefore sections can be immunolabeled with anti-ubiquitin antibodies. Lipofuscin can be identified by autofluorescence (green) within a range of excitation wavelengths (for example, excitation at 480 nm). For high resolution colocalization studies, fluorescent proteins (for example lipofuscin and Alexa-tagged antibodies) can be visualized by confocal imaging. In general, four different brain regions will be assessed—cortex, hippocampus, brainstem and cerebellum. For histological examination the inventor will examine brain tissue from mice (equal numbers of male and female) at 2 different timepoints—three months of age when gait abnormalities are first seen and eight months of age when mice are severely affected. N=6 per genotype: Mcoln−/−, Mcoln+/− and WT.

### Reference

1. Vann Jones, S. A. & O'Brien, J. T. The prevalence and incidence of dementia with Lewy bodies: a systematic review of population and clinical studies. Psychological medicine 44, 673-683, (2014).

2. Auning, E. et al. Early and presenting symptoms of dementia with lewy bodies. Dementia and geriatric cognitive disorders 32, 202-208, (2011).

3. Walker, M. P. et al. The Clinician Assessment of Fluctuation and the One Day Fluctuation Assessment Scale. Two methods to assess fluctuating confusion in dementia. The British journal of psychiatry:the journal of mental science 177, 252-256 (2000).

4. Lee, D. R., Taylor, J. P. & Thomas, A. J. Assessment of cognitive fluctuation in dementia: a systematic review of the literature. International journal of geriatric psychiatry 27, 989-998, (2012).

5. Hamilton, J. M. et al. Early visuospatial deficits predict the occurrence of visual hallucinations in autopsy-confirmed dementia with Lewy bodies. The American journal of geriatric psychiatry:official journal of the American Association for Geriatric Psychiatry 20, 773-781, (2012).

6. McKeith, I. G., Perry, E. K. & Perry, R. H. Report of the second dementia with

Lewy body international workshop: diagnosis and treatment. Consortium on Dementia with Lewy Bodies. Neurology 53, 902-905 (1999).

7. McKeith, I. G. et al. Diagnosis and management of dementia with Lewy bodies: third report of the DLB Consortium. Neurology 65, 1863-1872, (2005).

8. McKeith, I. G. Spectrum of Parkinson's disease, Parkinson's dementia, and Lewy body dementia. Neurologic clinics 18, 865-902 (2000).

9. McKeith, I. G. Clinical Lewy body syndromes. Annals of the New York Academy of Sciences 920, 1-8 (2000).

10. Nervi, A. et al. Familial aggregation of dementia with Lewy bodies. Archives of neurology 68, 90-93, (2011).

11. Waters, C. H. & Miller, C. A. Autosomal dominant Lewy body parkinsonism in a four-generation family.

Annals of neurology 35, 59-64, (1994).

12 Denson, M. A. et al. Familial parkinsonism, dementia, and Lewy body disease: study of family G. Annals of neurology 42, 638-643, (1997).

13. Ishikawa, A. et al. A mutant PSEN1 causes dementia with Lewy bodies and variant Alzheimer's disease. Annals of neurology 57, 429-434, (2005).

14. Ohara, K. et al. Familial dementia with Lewy bodies (DLB). Clinical neuropathology 18, 232-239 (1999).

15. Spira, P. J., Sharpe, D. M., Halliday, G., Cavanagh, J. & Nicholson, G. A. Clinical and pathological features of a Parkinsonian syndrome in a family with an Ala53Thr alphα-synuclein mutation. Annals of neurology 49, 313-319 (2001).

16. Brett, F. M., Henson, C. & Staunton, H. Familial diffuse Lewy body disease, eye movement abnormalities, and distribution of pathology. Archives of neurology 59, 464-467 (2002).

17. Tsuang, D. W. et al. Familial dementia with lewy bodies: a clinical and neuropathological study of 2 families. Archives of neurology 59, 1622-1630 (2002).

18. Tsuang, D. W., DiGiacomo, L. & Bird, T. D. Familial occurrence of dementia with Lewy bodies. The American journal of geriatric psychiatry:official journal of the American Association for Geriatric Psychiatry 12, 179-188 (2004).

19. Zarranz, J. J. et al. The new mutation, E46K, of alphα-synuclein causes Parkinson and Lewy body dementia. Annals of neurology 55, 164-173, (2004).

20. Wang, C. S. et al. Twin pairs discordant for neuropathologically confirmed Lewy body dementia. Journal of neurology, neurosurgery, and psychiatry 80, 562-565, (2009).

21. Bogaerts, V. et al. A novel locus for dementia with Lewy bodies: a clinically and genetically heterogeneous disorder. Brain:a journal of neurology 130, 2277-2291, (2007).

22. Meeus, B. et al. Comprehensive genetic and mutation analysis of familial dementia with Lewy bodies linked to 2q35-q36. Journal of Alzheimer's disease:JAD 20, 197-205, (2010).

23. Golbe, L. I., Di Iorio, G., Bonavita, V., Miller, D. C. & Duvoisin, R. C. A large kindred with autosomal dominant Parkinson's disease. Annals of neurology 27, 276-282, (1990).

24. Clark, L. N. et al. Association of glucocerebrosidase mutations with dementia with lewy bodies. Archives of neurology 66, 578-583, (2009).

25. Nalls, M. A. et al. A multicenter study of glucocerebrosidase mutations in dementia with Lewy bodies. JAMA neurology 70, 727-735, (2013).

26. Bras, J. et al. Genetic analysis implicates APOE, SNCA and suggests lysosomal dysfunction in theetiology of dementia with Lewy bodies. Human molecular genetics, (2014).

27. Meeus, B. et al. DLB and PDD: a role for mutations in dementia and Parkinson disease genes? Neurobiology of aging 33, 629 e625-629 e618, (2012).

28. Kobayashi, S. et al. Apolipoprotein E4 frequencies in a Japanese population with Alzheimer's disease and dementia with Lewy bodies. PloS one 6, e18569, (2011).

29. Ostrer, H. & Skorecki, K. The population genetics of the Jewish people. Human genetics 132, 119-127, (2013).

30. Ozelius, L. J. et al. LRRK2 G2019S as a cause of Parkinson's disease in Ashkenazi Jews. The New England journal of medicine 354, 424-425, (2006).

31. Kenny, E. E. et al. A genome-wide scan of Ashkenazi Jewish Crohn's disease suggests novel susceptibility loci. PLoS genetics 8, e1002559, (2012).

32. Struewing, J. P. et al. The risk of cancer associated with specific mutations of BRCA1 and BRCA2 among Ashkenazi Jews. The New England journal of medicine 336, 1401-1408, (1997).

33. Atzmon, G. et al. Abraham's children in the genome era: major Jewish diaspora populations comprise distinct genetic clusters with shared Middle Eastern Ancestry. American journal of human genetics 86, 850-859 (2010).

34. Bray, S. M. et al. Signatures of founder effects, admixture, and selection in the Ashkenazi Jewish population. Proceedings of the National Academy of Sciences of the United States of America 107, 16222-16227, (2010).

35. Need, A. C., Kasperaviciute, D., Cirulli, E. T. & Goldstein, D. B. A genome-wide genetic signature of Jewish ancestry perfectly separates individuals with and without full Jewish ancestry in a large random sample of European Americans. Genome biology 10, R7, (2009).

36. Behar, D. M. et al. The genome-wide structure of the Jewish people. Nature 466, 238-242, (2010).

37. Kopelman, N. M. et al. Genomic microsatellites identify shared Jewish ancestry intermediate between Middle Eastern and European populations. BMC genetics 10, 80, (2009).

38. Guha, S. et al. Implications for health and disease in the genetic signature of the Ashkenazi Jewish population. Genome biology 13, R2, (2012).

39. Gusev, A. et al. The architecture of long-range haplotypes shared within and across populations. Molecular biology and evolution 29, 473-486, (2012). 40. Olshen, A. B. et al. Analysis of genetic variation in Ashkenazi Jews by high density SNP genotyping. BMC genetics 9, 14, (2008).

41. Palamara, P. F., Lencz, T., Darvasi, A. & Pe'er, I. Length distributions of identity by descent reveal finescale demographic history. American journal of human genetics 91, 809-822, (2012).

42. Helgason, A., Yngvadottir, B., Hrafnkelsson, B., Gulcher, J. & Stefansson, K. An Icelandic example of the impact of population structure on association studies. Nature genetics 37, 90-95, (2005).

43. Styrkarsdottir, U. et al. Nonsense mutation in the LGR4 gene is associated with several human diseases and other traits. Nature 497, 517-520, (2013).

44. Carmi, S. et al. Sequencing an Ashkenazi reference panel supports population-targeted personal genomics and illuminates Jewish and European origins. Nature communications 5, 4835, (2014).

45. Clark, L. N. et al. Pilot association study of the beta-glucocerebrosidase N370S allele and Parkinson's disease in subjects of Jewish ethnicity. Movement disorders:official journal of the Movement Disorder Society 20, 100-103, (2005).

46. Clark, L. N. et al. Mutations in the glucocerebrosidase gene are associated with early-onset Parkinson disease. Neurology 69, 1270-1277, (2007).

47. Vacic, V. et al. Genome-wide mapping of IBD segments in an Ashkenazi PD cohort identifies associated haplotypes. Human molecular genetics 23, 4693-4702, (2014).

48. MacLeod, D. A. et al. RAB7L1 interacts with LRRK2 to modify intraneuronal protein sorting and Parkinson's disease risk. Neuron 77, 425-439, (2013).

49. Platt, F. M. Sphingolipid lysosomal storage disorders. Nature 510, 68-75, (2014).

50. Samie, M. A. & Xu, H. Lysosomal exocytosis and lipid storage disorders. Journal of lipid research 55, 995-1009, (2014).

51. Sidransky, E. et al. Multicenter analysis of glucocerebrosidase mutations in Parkinson's disease. The New England journal of medicine 361, 1651-1661, (2009).

52. Alcalay, R. N. et al. Frequency of known mutations in early-onset Parkinson disease: implication for genetic counseling: the consortium on risk for early onset Parkinson disease study. Archives of neurology 67, 1116-1122, (2010).

53. Alcalay, R. N. et al. Cognitive performance of GBA mutation carriers with early-onset PD: the COREPD study. Neurology 78, 1434-1440, (2012).

54. Gan-Or, Z. et al. The p.L302P mutation in the lysosomal enzyme gene

SMPD1 is a risk factor for Parkinson disease. Neurology 80, 1606-1610, (2013).

55. Winder-Rhodes, S. E. et al. Genetic and pathological links between Parkinson's disease and the lysosomal disorder Sanfilippo syndrome. Movement disorders:official journal of the Movement Disorder Society 27, 312-315, (2012).

56. Tayebi, N. et al. Gaucher disease with parkinsonian manifestations: does glucocerebrosidase deficiency contribute to a vulnerability to parkinsonism? Molecular genetics and metabolism 79, 104- 109 (2003).

57. Choi, J. H. et al. Aggregation of alphα-synuclein in brain samples from subjects with glucocerebrosidase mutations. Molecular genetics and metabolism 104, 185-188, (2011).

58. Suzuki, K. et al. Neuronal and glial accumulation of alpha- and beta-synucleins in human lipidoses. Acta neuropathologica 114, 481-489, (2007).

59. Cox, T. M. & Cachon-Gonzalez, M. B. The cellular pathology of lysosomal diseases. The Journal of pathology 226, 241-254, (2012).

60. Balducci, C. et al. Lysosomal hydrolases in cerebrospinal fluid from subjects with Parkinson's disease. Movement disorders:official journal of the Movement Disorder Society 22, 1481-1484, (2007).

61. Parnetti, L. et al. Cerebrospinal fluid beta-glucocerebrosidase activity is reduced in Dementia with Lewy Bodies. Neurobiology of disease 34, 484-486, (2009).

62. Gegg, M. E. et al. Glucocerebrosidase deficiency in substantia nigra of parkinson disease brains. Annals of neurology 72, 455-463, (2012).

63. Murphy, K. E. et al. Reduced glucocerebrosidase is associated with increased alpha-synuclein in sporadic Parkinson's disease. Brain:a journal of neurology 137, 834-848, (2014).

64. Roach, J. C. et al. Analysis of genetic inheritance in a family quartet by whole-genome sequencing. Science 328, 636-639, (2010).

65. Li, H. & Durbin, R. Fast and accurate short read alignment with Burrows-Wheeler transform. Bioinformatics 25, 1754-1760, (2009).

66. McKenna, A. et al. The Genome Analysis Toolkit: a MapReduce framework for analyzing nextgeneration DNA sequencing data. Genome research 20, 1297-1303, (2010).

67. Wang, K., Li, M. & Hakonarson, H. ANNOVAR: functional annotation of genetic variants from highthroughput sequencing data. Nucleic acids research 38, e164, (2010).

68. Ye, K., Schulz, M. H., Long, Q., Apweiler, R. & Ning, Z. Pindel: a pattern growth approach to detect break points of large deletions and medium sized insertions from paired-end short reads. Bioinformatics 25, 2865-2871, (2009).

69. Chen, K. et al. BreakDancer: an algorithm for high-resolution mapping of genomic structural variation. Nature methods 6, 677-681, (2009).

70. Abyzov, A., Urban, A. E., Snyder, M. & Gerstein, M. CNVnator: an approach to discover, genotype, and characterize typical and atypical CNVs from family and population genome sequencing. Genome research 21, 974-984, (2011).

71. Coe, B. P., Chari, R., MacAulay, C. & Lam, W. L. FACADE: a fast and sensitive algorithm for the segmentation and calling of high resolution array CGH data. Nucleic acids research 38, e157, (2010).

72. Medvedev, P., Fiume, M., Dzamba, M., Smith, T. & Brudno, M. Detecting copy number variation with mated short reads. Genome research 20, 1613-1622, (2010).

73. Handsaker, R. E., Korn, J. M., Nemesh, J. & McCarroll, S. A. Discovery and genotyping of genome structural polymorphism by sequencing on a population scale. Nature genetics 43, 269-276, (2011).

74. Luo, R. et al. SOAPdenovo2: an empirically improved memory-efficient short-read de novo assembler. GigaScience 1, 18, (2012).

75. Liu, X. et al. Increased Rate of Sporadic and Recurrent Rare Genic Copy Number Variants in Parkinson's Disease Among Ashkenazi Jews. Molecular genetics & genomic medicine 1, 142-154, (2013).

76. Hu, H. et al. A unified test of linkage analysis and rare-variant association for analysis of pedigree sequence data. Nature biotechnology 32, 663-669, (2014).

77. Habegger, L. et al. VAT: a computational framework to functionally annotate variants in personal genomes within a cloud-computing environment. Bioinformatics 28, 2267-2269, (2012).

78. Cingolani, P. et al. A program for annotating and predicting the effects of single nucleotide polymorphisms, SnpEff: SNPs in the genome of Drosophila melanogaster strain w1118; iso-2; iso-3. Fly 6, 80-92, (2012).

79. Choi, Y., Sims, G. E., Murphy, S., Miller, J. R. & Chan, A. P. Predicting the functional effect of amino acid substitutions and indels. PloS one 7, e46688, (2012).

80. Schwarz, J. M., Cooper, D. N., Schuelke, M. & Seelow, D. MutationTaster2: mutation prediction for the deep-sequencing age. Nature methods 11, 361-362, (2014).

81. Adzhubei, I., Jordan, D. M. & Sunyaev, S. R. Predicting functional effect of human missense mutations using PolyPhen-2. Current protocols in human genetics/editorial board, Jonathan L. Haines ... [et al.] Chapter 7, Unit? 20, (2013).

82. Kumar, P., Henikoff, S. & Ng, P. C. Predicting the effects of coding non-synonymous variants on protein function using the SIFT algorithm. Nature protocols 4, 1073-1081, (2009).

83. Kircher, M. et al. A general framework for estimating the relative pathogenicity of human genetic variants. Nature genetics 46, 310-315, (2014).

84. Petrovski, S., Wang, Q., Heinzen, E. L., Allen, A. S. & Goldstein, D. B. Genic intolerance to functional variation and the interpretation of personal genomes. PLoS genetics 9, e1003709, (2013).

85. Cooper, G. M. et al. Distribution and intensity of constraint in mammalian genomic sequence. Genome research 15, 901-913, (2005).

86. MacArthur, D. G. et al. Guidelines for investigating causality of sequence variants in human disease. Nature 508, 469-476, (2014).

87. Slaugenhaupt, S. A. The molecular basis of mucolipidosis type IV. Current molecular medicine 2, 445-450 (2002).

88. Venugopal, B. et al. Neurologic, gastric, and opthalmologic pathologies in a murine model of mucolipidosis type IV. American journal of human genetics 81, 1070-1083, (2007).

89. Sun, T., Wang, X., Lu, Q., Ren, H. & Zhang, H. CUP-5, the C. elegans ortholog of the mammalian lysosomal channel protein MLN1/TRPML1, is required for proteolytic degradation in autolysosomes. Autophagy 7, 1308-1315, (2011).

90. Venkatachalam, K. et al. Motor deficit in a Drosophila model of mucolipidosis type IV due to defective clearance of apoptotic cells. Cell 135, 838-851, (2008).

91. Fares, H. & Greenwald, I. Regulation of endocytosis by CUP-5, the Caenorhabditis elegans mucolipin-1 homolog. Nature genetics 28, 64-68, (2001).

92. Dong, X. P. et al. The type IV mucolipidosis-associated protein TRPML1 is an endolysosomal iron release channel. Nature 455, 992-996, (2008).

93. Carboni, E. & Lingor, P. Insights on the interaction of alphα-synuclein and metals in the pathophysiology of Parkinson's disease. Metallomics:integrated biometal science, (2015).

94. Bae, M. et al. Activation of TRPML1 clears intraneuronal Abeta in preclinical models of HIV infection. The Journal of neuroscience:the official journal of the Society for Neuroscience 34, 11485-11503, (2014).

95. Duff, K. et al. Increased amyloid-beta42(43) in brains of mice expressing mutant presenilin 1. Nature 383, 710-713, (1996).

96. Wu, J. W. et al. Small misfolded Tau species are internalized via bulk endocytosis and anterogradely and retrogradely transported in neurons. The Journal of biological chemistry 288, 1856-1870, (2013).

### 6.3 Example 3:

### Glucocerebrosidase Mutations and Dementia with Lewy Bodies

### Introduction

A range of applications and methodologies are used to identify variation in both DNA sequence and gene expression levels in patient populations to determine genetic contribution to disease pathogenesis. These include: SNP analysis and case-control association studies, copy number variation discovery (CNV) and analysis, genome wide association studies (GWAS), fine mapping and gene identification, resequencing and mutation screening, whole genome expression profiling and methylation analysis.

Glucocerebrosidase Mutations are Associated with Dementia with Lewy Bodies in Autopsied Brains

GBA genotyping on a large sample of living ADRC (Alzheimer's Disease Research Center) subjects is proposed. The inventor hypothesize that subjects with GBA mutations will be have proportionally greater clinical manifestations of DLB, and pathological findings of DLB on autopsy.

### Enzyme Activity in Autopsy Tissue

In a subset of 64 autopsy samples from the New York Brain Bank, which included 34 subjects with Lewy body pathology and 30 ‘control’ subjects without Lewy body or Alzheimer disease pathology the inventor measured β-glucocerebrosidase activity to determine whether reduced enzymatic activity (haploinsufficiency) is associated with GBA mutation status and a neuropathological diagnosis of DLB. β-Glucocerebrosidase enzyme activity was measured in cerebellar brain tissue extracts from 64 subjects using the standard 4-methylumbelliferyl-β-D-glucopyranoside assay. The inventor also determined the enzyme activity of a second lysosomal hydrolase, α-hexosaminidase, to demonstrate specificity of decreased activity of β-glucocerebrosidase. α-hexosaminidase activity was determined in the same autopsy samples using the standard assay 4-methylumbelliferyl-2-acetamido-2-deoxy-β-D-glucopyranoside (4MUGlcNAc). β-Glucocerebrosidase enzyme activity for a subset of subjects is show in Table 6. Overall, the mean levels of β-glucocerebrosidase enzyme activity (p=0.0021) and the β-glucocerebrosidase: β-hexosaminidase ratio (p=0.0014) were significantly lower in cases with dementia than in controls, while the mean level of β- hexosaminidase activity (p=0.9051) was not. In GBA mutation carriers β-Glucocerebrosidase activity was substantially reduced when compared to non-GBA Lewy body cases. The inventor did not observe differential enzyme activity of β-glucocerebrosidase or for the β-glucocerebrosidase: β-hexosaminidase ratio in subjects carrying GBA mutations classified as ‘severe’ type (e.g. 84insGG, L444P) compared to subjects carrying GBA mutations classified as ‘mild’ type (e.g. N370S, R496H). Lastly, the inventor examined the relation between β-glucocerebrosidase activity in demented vs. non-demented subjects. The pattern of association between GBA mutation status and the β-glucocerebrosidase activity was comparable to the combined samples, suggesting that those with dementia are driving the association.

TABLE 6
Example of subjects with GBA mutations and Enzyme Activity

| | | | Initial | | | GBA Enzyme |
|---|---|---|---|---|---|---|
| Subject | | | Clinical | | Pathologic | Activity |
| Number | GBA Genotype | AAO | Presentation | Dementia | Findings | (% control) |
| 1 | T369M/+ | 72 | memory problems | Yes | LBV-AD | 35.1% |
| 2 | N370S/+ | 58 | parkinsonism | Yes | DLB | 42.7% |
| 3 | D409H/+ | 71 | memory problems | Yes | DLB | 31.2% |
| 4 | L444P/+ | 55 | parkinsonism | Yes | DLB | 49.4% |
| 5 | N370S/+ | 74 | parkinsonism | Yes | DLB | 65.6% |
| 6 | W184R/+ | 53 | parkinsonism | Yes | DLB | 64.2% |
| 7 | R496H/+ | 74 | parkinsonism | None | DLB | 91.7% |
| 8 | E326K + N188R + | 65 | hallucinations | Yes | DLB | 51.4% |
| | S196P + V191G | | | | | |
| 9 | E326K/+ | 41 | parkinsonism | Yes | DLB | 70.4% |
| 10 | E326K/+ | 58 | parkinsonism | None | PD | 72.3% |
| 11 | 84insGG/+ | 66 | memory problems | Yes | DLB | 65.5% |
| 12 | N370S/+ | 62 | parkinsonism | Yes | DLB | 64.5% |

ADRC-Clinical Core Patients and Controls Currently Available to this Project.

All UDS subjects who do not yet have blood banked are being actively recruited for blood donation. Blood samples are processed by the ADRC Genetics Core (ADRC-GC), and both DNA and plasma are banked. Presently there are 332 DNA samples banked. It is planned there will be at least 600 DNA samples available from the blood obtained by the Clinical Core and banked by the ADRC-GC. The Clinical Core plans to have at least 800 DNA samples banked, including a larger numbers of controls than presently available. The present set of 332 DNA samples includes 244 (73%) persons with dementia clinically diagnosed with Alzheimer's Disease (65%), other dementias (8%), cognitive impairment not meeting criteria for dementia (57 persons, 17%), and normal controls (32 persons, 10%). Average age is 75.6 ±9.6 yr, 54.2% are female. Ethnoracial distribution is 75.2% white, 4.5% African-American, 1.3% Asian-American, 13.8% Hispanic, and 5.1% other. Once DNA is isolated, all samples are genotyped for APOE. Tables 10A and 10B show the characteristics of the subjects in the UDS who are controls, cognitively impaired but not demented (‘MCI’) , and AD and DLB patients. Overall, few patients meet criteria for clinically probable DLB as primary diagnosis, but many patients have core symptoms typical of DLB. Since pathologically, as many as 35% of brains have DLB features, the clinical “diagnosis” is undercounting the disorder. Thus the analysis is mostly predicated on the item by item features rather than the diagnosis.

TABLE 7A
Data on Current ADRC Clinical Core Subjects in UDS cohort

| Status | N | Age | Sex: % F | Education | APOE- [symbol] 4 | Hallucinations | Parkinsonism | Fluctuations |
|---|---|---|---|---|---|---|---|---|
| Control | 60 | 75.3 ± 9.8 | 72% | 15.1 ± 3.6 | 40.0% | 0% | 2.0% | 0% |
| MCI | 110 | 75.4 ± 9.6 | 64% | 13.0 ± 5.3 | 34.3% | 1.8% | 2.9% | 4.8% |
| AD | 422 | 78.6 ± 9.7 | 62% | 13.0 ± 4.9 | 54.6% | 9.2% | 8.1% | 10.3% |
| DLB | 21 | 78.8 ± 9.3 | 39% | 14.2 ± 5.8 | 25.0% | 38.1% | 46.4% | 40.0% |
| Other | 56 | 65.8 ± 10.3 | 40% | 14.4 ± 4.2 | 31.6% | 7.1% | 20.9% | 25.0% |

[symbol]

indicates data missing or illegible when filed

TABLE 7B
Data on Current ADRC Clinical Core Subjects in UDS cohort for whom DNA is available.

| Status | N | Age | Sex: % F | Education | APOE- [symbol] 4 | Hallucinations | Parkinsonism | Fluctuations |
|---|---|---|---|---|---|---|---|---|
| Control | 32 | 72.4 ± 11.0 | 75% | 16.3 ± 3.4 | 59.4% | 0% | 3.1% | 0% |
| MCI | 57 | 70.4 ± 9.5 | 60% | 13.6 ± 5.0 | 38.6% | 0% | 5.3% | 0% |
| AD | 217 | 77.9 ± 8.7 | 49% | 12.7 ± 4.9 | 47.5% | 12.7% | 11.1% | 16.7% |
| DLB | 10 | 75.7 ± 5.6 | 33% | 15.4 ± 4.4 | 40.0% | 16.7% | 50.0% | 0% |
| Other | 16 | 69.5 ± 8.9 | 56% | 14.9 ± 2.9 | 56.2% | 8.3% | 25.0% | 0% |

[symbol]

indicates data missing or illegible when filed

### 6.4 Example 4:

### Glucocerebrosidase Mutations and Dementia with Lewy Bodies

### Introduction

It is hypothesized that inherited GBA mutations are a potentially useful clinical marker for the presence of cortical Lewy bodies and may be a genetic risk factor for the development of DLB.

Other preliminary data suggested that the functional impact of carrying a GBA mutation might be haploinsufficiency with reduced activity of β-glucocerebrosidase in autopsy tissue from DLB patients with this mutation. Reduced glucocerebrosidase activity may result in abnormal trafficking of alphasynuclein, thus resulting in Lewy body formation and perhaps the syndrome of DLB.

Genotyping on a large sample of living ADRC subjects is proposed to determine if subjects with GBA mutations will have relatively more clinical manifestations of DLB, and ultimately pathological findings DLB at autopsy. The specific aims the inventor propose are designed to show that GBA mutations are associated with the clinical and neuropathological diagnosis of DLB.

### Results

Determination of the clinical characteristics of persons with GBA mutations in the ADRC referral sample population, and comparison of the phenotypes of these individuals to those of non-GBA mutation carriers.

The GBA gene in a total of 592 subjects in the ADRC referral population was sequenced. GBA Mutations were found in 6.9% (41/592) of all subjects (Table 8). These subjects included 13 different mutations (one compound heterozygote). Classifying subjects by known phenotypic effect (in terms of Gaucher's disease) of the mutation, as in the previously published studies, revealed 2 silent (11191, Q226Q), 11 mild (all N370S), 3 severe (2 L444P and 1 R463C), 0 null, and 25 of unknown phenotypic effect (14 K(−27)R, 6 E326K, 1 R120Q, 1 W184R, 1 V460L, 1 D443N, 1 g.1367C>T). Overall, 12/592 (2.0%) of tested subjects, or 12/41 (29%) of subjects with a GBA mutation, had either a ‘mild’ or ‘severe’ mutation (wherein ‘mild’ and ‘severe’ is in reference to Gaucher disease phenotype). Among the 13 different GBA mutations found in the 41 individuals, 8 were missense mutations, 2 were silent mutations (synonymous substitutions), and none insertions/deletions. Novel mutations not previously reported (Hruska et al 2008) were present in 2 individuals. The most common mutations among ADRC subjects were N370S (9 individuals) and K(−27)R (14 individuals).

TABLE 8
GBA Mutations identified in 592 ADRC subjects

| | | Allele | | |
|---|---|---|---|---|
| #subjects | Mutation(s) | name | Exon | Type |
| 13 | g.1864A > G c.38A > G p.Lys13Arg | K(-27)R | 2 | unknown |
| 1 | g.1864A > G c.38A > G p.Lys13Arg + | K(-27)R + | 2.11 | unknown |
| | g.7549A > C c.1584A > C p.Ile528Ile | I528I | | |
| 11 | g.6728A > G c.1226A > G p.Asn409Ser | N370S | 9 | mild |
| 6 | g.6195G > A c.1093G > A p.Glu365Lys | E326K | 8 | unknown |
| 2 | g.7319T > C c.1448T > C p.Leu483Pro | L444P | 10 | severe |
| 1 | g.3940C > T c.474C > T p.Ile158Ile | I1191 | 5 | silent |
| 1 | g.4343T > C c.667T > C p.Trp223Arg | W184R | 6 | unknown |
| 1 | g.5026C > T c.795C > T p.Gln265Gln | Q226Q | 7 | silent |
| 1 | g.7314G > A c.1443G > A p.Asp482Asn | D443N | 10 | novel |
| 1 | g.7375C > T c.1504C > T p.Arg502Cys | R463C | 10 | severe |
| 1 | g.7366G > C c.1495G > C p.Val1499Leu | V460L | 10 | unknown |
| 1 | g.3942G > A c.476G > A p.Arg159Gln | R120Q | 5 | unknown |
| 1 | g.1367C > T | — | | novel |

Clinical correlation comparing GBA mutation carriers to non-carriers reveals that analyzing the demographic characteristics of the 41 GBA mutation-carriers versus the 551 non-carriers, the inventor found no difference in gender distribution, but a significant effect of ethnicity (Table 9). Overall, 53.7% (n=22) of mutation carriers are white non-Hispanic and the most common mutation observed is N370S (n=11). A total of 21.9% (n=9) of mutation carriers are black non-Hispanic and 17.1% (n=7) of mutation carriers are Hispanic subjects. Many of the black non-Hispanic and Hispanic subjects (87.5% =14/16) carried the variant K(−27)R, an sequence variant located in the leader portion of the enzyme. Grouped by genotype, the 17 individuals with E326K and N370S genotypes, were all non-Hispanic whites, but the 14 individuals with the K(−27)R genotype, were all non-white (8 non-Hispanic Black, 6 Hispanic). The remaining 5 genotypes were split among non- Hispanic whites (5) and non-whites (5). Thus, while it has been known that the E326K and N370S genotypes are more frequent among whites, in particular Ashkenazi Jewish individuals, the results suggest that the K(−27R) genotype may be a marker of possibly African origin, since many of the Carribean Hispanics have admixed African origins. This mutation is in the 39-residue signal peptide, removed from the mature enzyme during processing. The frequency of the K(−27)R variant reported in the exome variant server (NHLBI exome sequencing project (ESP)) confirms that it is a rare variant in European Americans (MAF=0.043%; 7020 chromosomes) and more common in African Americans (MAF=6.98%; 3738 chromosomes). Although polyphen prediction suggests K(−27)R is a benign variant, it may alter cytoplasmic transport or act as a modifier with other GBA mutations. This variant has been previously reported in a Brazilian patient with Type I Gaucher disease (N370S/W184R/K(−27)R) (Rozenberg et al 2006).

Genotype-phenotype correlations have been performed using the 592 subjects sequenced to date (Table 10). Comparing the 41 GBA mutation carriers to non-carriers, there was no significant difference in proportion with dementia, with Lewy Body phenotype diagnosis, or with UPDRS score or core Lewy Body symptoms such as hallucinations, etc. However, when only the 11 GBA mutation carriers with known Gaucher phenotype were considered, and compared to non-carriers, there was still no significant difference in the proportion demented, but there were possible trends towards a larger proportion of cases with Lewy Body phenotype diagnosis among the carriers, and toward greater UPDRS score and hallucinations (not shown in table) in the carriers.

TABLE 9
GBA mutation frequency by ethnicity

| GBA | | White, | Black, | | |
|---|---|---|---|---|---|
| mutation | TOTAL | non-Hisp | non-Hisp | Hispanic | Other |
| K(-27)R | 14 | 0 | 8 | 6 | 0 |
| N370S | 11 | 11 | 0 | 0 | 0 |
| E326K | 6 | 6 | 0 | 0 | 0 |
| Others | 10 | 5 | 1 | 1 | 3 |

| GBA | | White, | Non- |
|---|---|---|---|
| mutation | TOTAL | non-Hisp | White |
| K(-27)R | 14 | 0 | 14 |
| Other Mutations | 27 | 22 | 5 |

Chi-square: p < 0.000001

TABLE 10
Clinical characteristics of GBA mutation carriers versus non-carriers

| Characteristic | No GBA mutation | | GBA Mutation | | p-value | GBA (mild/sev) Mut | | P-value |
|---|---|---|---|---|---|---|---|---|
| Subjects/deaths/autopsies | 513/79/44 | | 41/4/2 | | ns | 13/2/2 | | |
| Dementia: n(%) | 282 | (51.5%) | 21 | (51.2%) | ns | 6 | (42.9%) | >.1 |
| APOE4+**: n/N(%) | 154/378 | (40.7%) | 9/22 | (40.9%) | ns | 3/10 | (30.0%) | >.1 |
| Lewy Body Phenotype*: n(%) | 19 | (3.5%) | 2 | (4.9%) | ns | 2 | (14.3%) | 0.09 |
| UPDRS**: mean total ± SD (N) | 7.87 ± 14.1 | (518) | 6.71 ± 12.2 | (38) | ns | 10.7 ± 16.4 | (13) | >.1 |
| Sex: n male (%) | 237 | (43.2%) | 15 | (36.6%) | ns | 8 | (57.1%) | >.1 |

*Lewy body phenotype = consensus primary diagnosis of LBD or concomitant diagnosis of PD
**Not all cases have UPDRS or yet have APOE genotype determined (N for UPDRS and APOE are listed).

### 6.5 Example 5:

### SMPD1 Mutations, sphingomyelinase Activity and α-Synuclein Accumulation in Parkinson's Disease

### Summary

SMPD1 was sequenced in two cohorts (New-York and Montreal) with a total of 1075 Parkinson's Disease (PD) patients and 975 controls. Acid sphingomyelinase (ASMase) activity was measured in dried blood spots by a mass-spectrometry-based assay in the New-York cohort (550 patients, 284 controls). In two cellular models (HeLa cells and BE(2)-M17 cells), SiRNA SMPD1 knockdown was performed, and α-synuclein quantity was estimated.

SMPD1 mutations were more common in PD (n=525) versus controls (n=691) in the Montreal cohort (5.3% versus 2.9%, p=0.037), driven by the p.A487V mutation (1.5% versus. 0.14%, p=0.0065). This mutation was found in additional three patients and one control in the New-York cohort (combined frequency 1.0% versus 0.2%, OR=5.03, 95%C1.11-22.75, p=0.024). In the New-York cohort, there was no significant differences in ASMase activity between PD and control (4.64 ±1.68 versus 4.62 ±1.64, p=0.84), but among PD cases, reduced ASMase activity was associated with an earlier age—at-onset of PD, with 3.5-5.8 years earlier onset in the lowest quartile vs. the highest quartile of ASMase activity (p=0.01-0.001 in all comparisons). Knockdown of SMPD1 resulted in increase of α-synuclein in both HeLa cells and BE(2)-M17 cells.

The results support an association between SMPD1 and its product

ASMase with PD, possibly because of α-synuclein accumulation in presence of low ASMase activity. Further genetic and functional studies are necessary to better elucidate the role of SMPD1 in PD and other synucleinopathies.

### Methods

### Study Populations

Basic demographic characteristics of the two cohorts that were analyzed in the current study are described in Table 11. The study population included 1075 PD patients and 975 controls, including 525 patients and 691 controls collected at the Montreal Neurological Institute, Montreal, Canada, and 550 patients and 284 controls recruited at Columbia University, N.Y., USA. All patients and controls were unrelated. The cohort from Montreal (termed hereafter MTL cohort) was composed of French- Canadian PD patients and controls that were recruited in Quebec, Canada, and French PD patients and controls that were recruited in Montpellier, France. The French-Canadian/French control was composed of two control groups, elderly controls (n=105, average age at enrollment of 61.5 ±7.6 years) and young controls (n=576, average age at enrollment of 34.6 ±6.4 years, data on age was missing for 11 individuals). Since there was no significant difference in the frequency of SMPD1 mutations between the elderly and young controls, these two groups could be analyzed as a single control population. Detailed description of the recruitment and demographics of the N.Y. cohort, also called “Spot” study, was previously published.^{3,}

²⁰ All patients were diagnosed according to the UK brain bank criteria, except that patients who had family history of PD were not excluded from the study. All participants signed informed consent forms prior to their enrollment into the study, and the institutional review boards approved the study protocols.

TABLE 11
Basic demographic variables of the study populations

| | MTL | | | NY | | |
|---|---|---|---|---|---|---|
| | Patients | Controls | p^{a} | Patients | Controls | p^{a} |
| Number | 525 | 691 | — | 550 | 284 | — |
| Men, % | 334 (63.6%) | 274 (39.7%) | <0.05 | 355 (64.5%) | 100 (35.2%) | <0.05 |
| Age at | 65.7 ± 10.9 | 38.7 ± 11.8 | <0.05 | 65.9 ± 10.9 | 64.9 ± 9.7 | NS |
| Reported AJ | 0 (0%) | 0 (0%) | — | 240 (43.6%) | 116 (40.1%) | NS |

MTL, samples collected in Montreal from Quebec and Montpellier, France;
NY, New-York;
SD, standard deviation;
NS, non-significant
^{a}p value was calculated using student's t-test for the continuous variables and Fisher's exact test for the categorical variables.

### Sequencing and Analysis of SMPD1 Variants

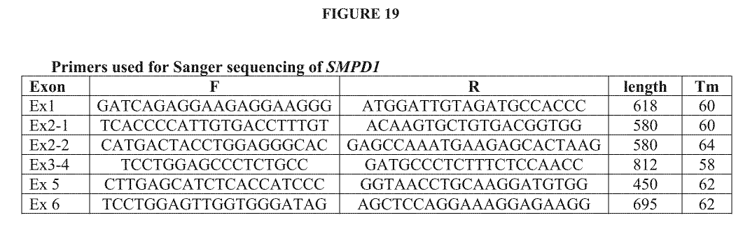

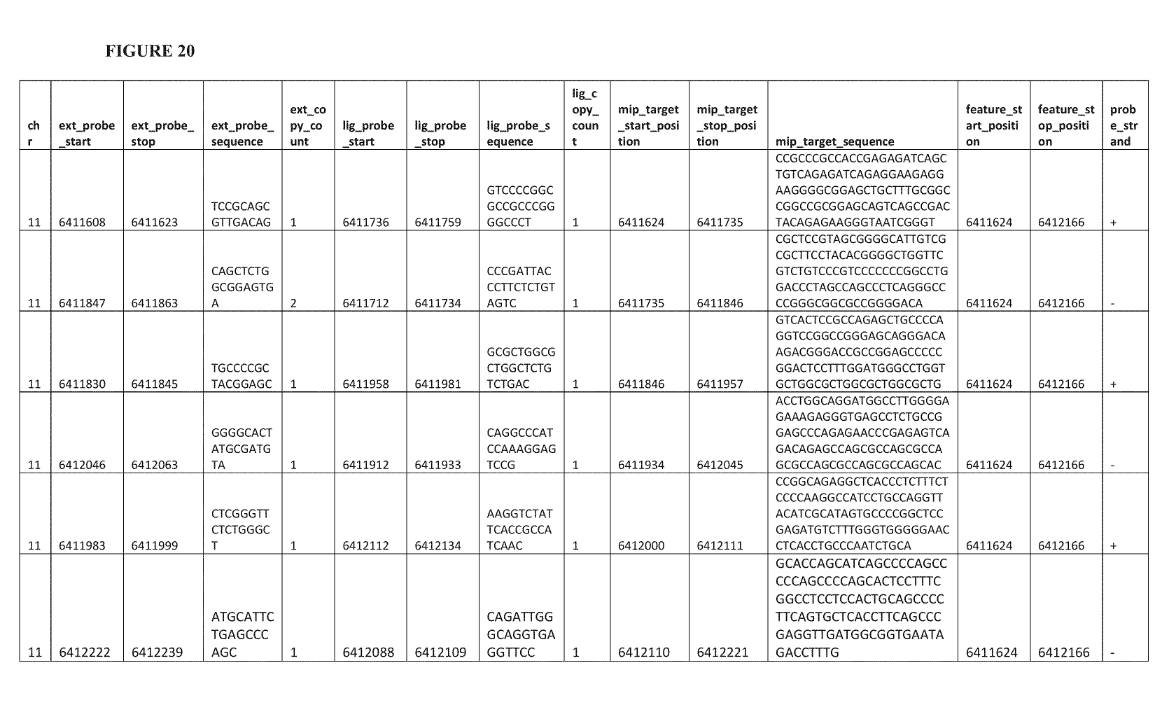

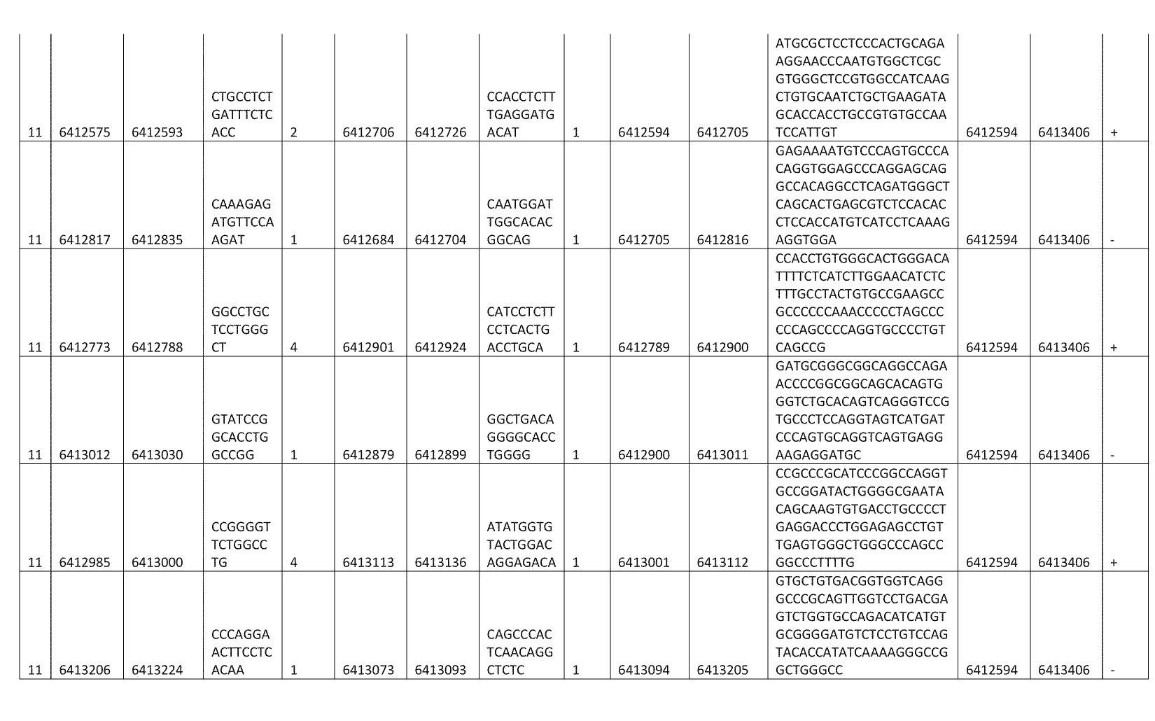

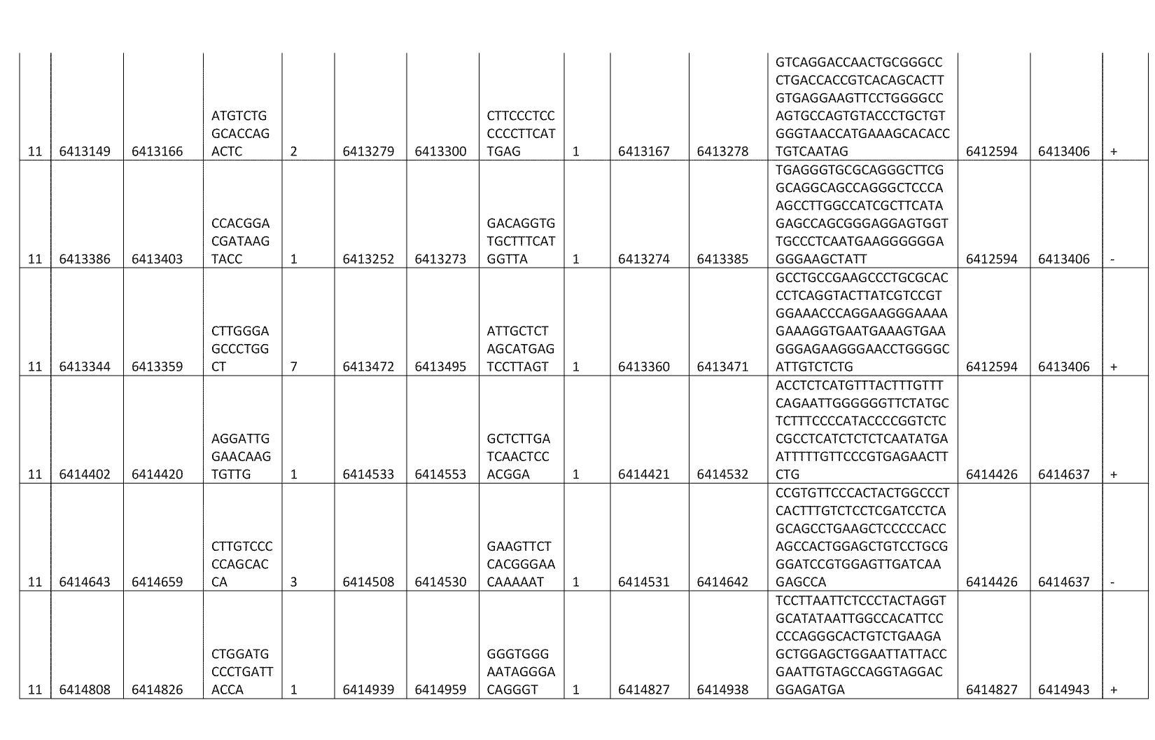

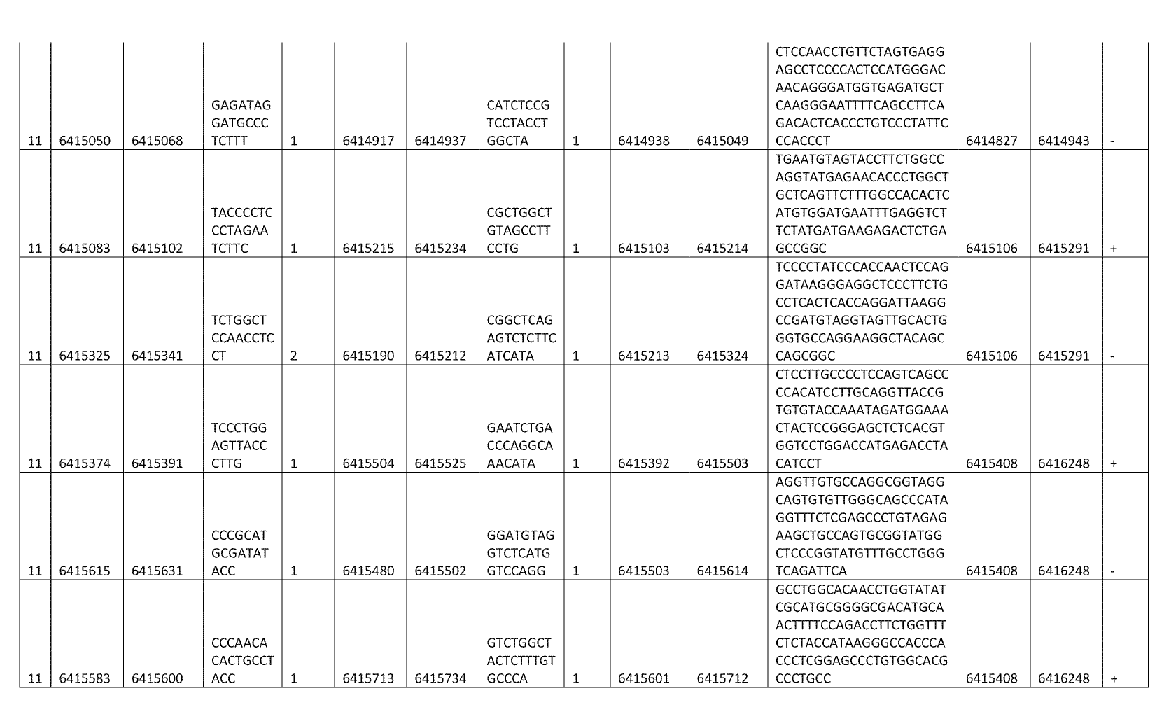

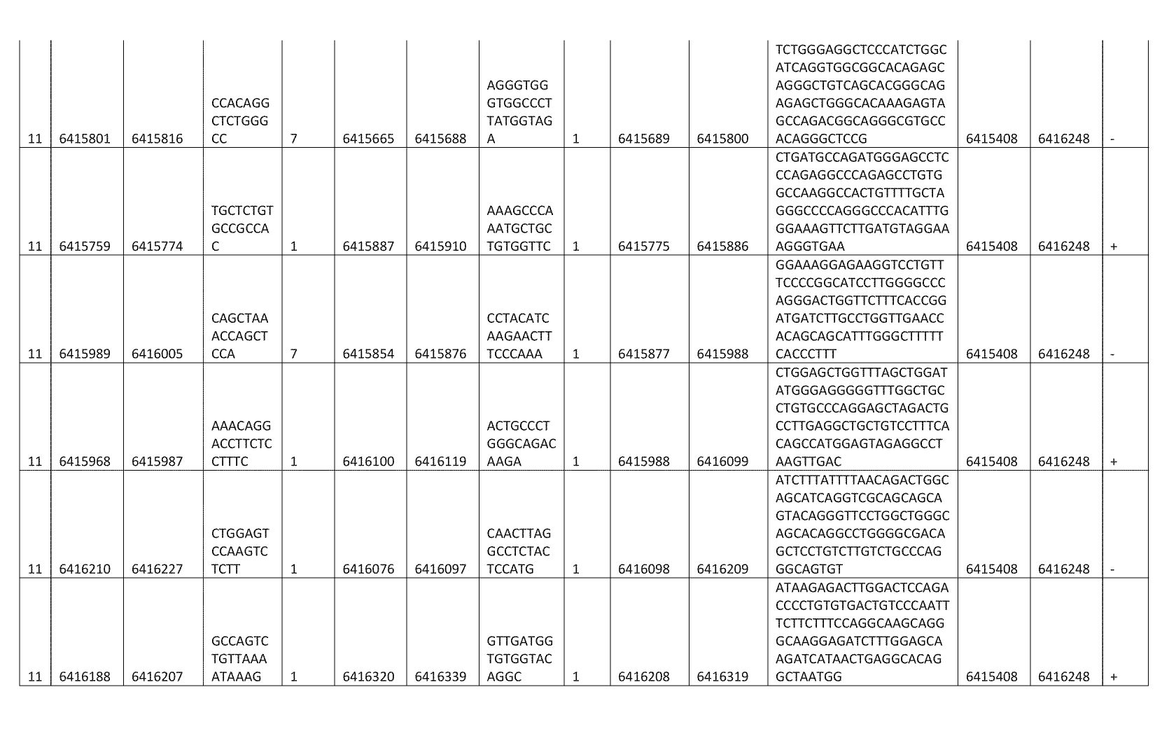

DNA was extracted using standard salting out protocol. In the N.Y. cohort, sequencing was done using Sanger sequencing (FIG. 19 details the primers used for PCR amplification and sequencing), PCRs were performed using the AmpliTaq Gold DNA Polymerase (Applied Biosystems, Foster City, Calif.) and the amplicons were sequenced at the Genome Quebec Innovation Centre (Montreal, Quebec, Canada) using a 3730XL DNAnalyzer (Applied Biosystems, Calif.). The results were analysed using the Genalys V3.3b software. In the MTL cohort, targeted next generation sequencing of the entire coding region of SMPD1 was performed. To capture SMPD1, the inventor used molecular inversion probes (MIPs) as was previously described.²¹ In brief, probes were designed (detailed in FIG. 20) with the following principles for each MW:1) Resides on the opposite strand as the previous MW, 2) Predicted to be a high performer, 3) Has minimal overlap with the previous MIP and 4) The targeting arms were adapted to the presence of known common SNPs. The MIPs were phosphorylated at a concentration of 0.1 picomoles/μl using T4 Polynucleotide Kinase and 10X T4 DNA Ligase Buffer (New England Biolabs, Ipswich, Mass.) at 37° C. for 45 minutes followed by 65° C. for 20 minutes. The MIPs were then diluted to 0.01 picomoles/μl, and the probes were hybridized to 100 ng of genomic DNA at 95° C. for 10 minutes followed by 60° C. for 24 hours. Gap-filling and ligation were done using Ampligase DNA ligase (Illumina, San Diego, Calif.), Ampligase 10X Buffer (Illumina), Hemo Kleen Taq (New England Biolabs) and dNTPs. To remove the linear DNA, exonucleases I and III (New England Biolabs) were used at 37° C. for 45 minutes and 95° C. for 2 minutes. The library was amplified by PCR using 2X iProof (Biorad, Hercules, Calif.), Sybr Green (Invitrogen, Carlsbad, Calif.) and indexed reverse PCR primers at 98° C. for 30 seconds, followed by 18 cycles of 10 seconds at 98° C., 30 seconds at 60° C., and 30 seconds at 72° C., and one final step at 72° C. for 2 minutes. Finally, the samples were purified with Agencourt AMPure XP (Beckman Coulter, Montreal, Canada) purification beads. The library was sequenced with the Illumina HiSeq 2500 platform at the McGill University and Genome Québec Innovation Centre. Sequence processing, alignment and variant calling were done using Burrows-Wheeler Aligner, (BWA) ²² the Broad Institute

Genome Analysis Toolkit (GATK v.2.6.4)²³ and ANNOVAR.²⁴ Data on the frequency of each SMPD1 variant was extracted from public databases including Exome Aggregation Consortium (ExAC), Cambridge, Mass. (http://exac.broadinstitute.org), 1000 Genomes Proj ect (www.1000genomes.org), NHLBI GO Exome Sequencing Project—Exome Variant Server Ontp://evs.gs.washington.edu/EVS/) and dbSNP132. Online prediction tools were used to assess deleteriousness (SIFT,²⁵ PolyPhen2,²⁶ and GERP++²⁷). Only variants with high coverage and quality were included in the analysis. The p.A487V variant was confirmed using Sanger sequencing as described above.

Acid sphingomyelinase Enzymatic Activity

Dried blood spots were obtained as previously described. ^{28, 29} In brief, blood samples were collected in a 10 cc EDTA tube. Seventy five microliters of blood was “spotted” on each of five circles on a filter paper (Whatman 903 protein savor card, St. Louis, Mich.) and dried at room temperature for at least 4 hours. Absorbent filter paper was then stored in a sealed plastic bag with desiccants and a humidity indicator in a −20° C. freezer and later shipped to the laboratories at room temperature. Upon receipt, the samples were stored at −80° C. before analysis.

ASMase activity was measured using a previously published protocol as part of a multiplex assay together with four additional lysosomal enzymes.³⁰ In summary, ASMase was extracted from a 3.2 mm-diameter punch from a dried blood spot sample in 70 μl of 20 mM sodium phosphate buffer (pH 7.1) on a 96-well plate. Ten μl of the dried blood spot extract was added to 15 μl of ASMase substrate/internal standard mixtures (The Center for Disease Control and Prevention, Atlanta, GA), 0.33 mmol/L C6-sphingomyelin and 6.67 μmol/L C4-ceramide in a 0.93 mol/L Sodium Acetate Trihydrate (Mallinckrodt, St. Louis, Mich.) +0.60 mmol/L Zinc Chloride (Sigma, St. Louis, Mich.), pH 5.7 buffer with sodium taurocholate (1.0 g/L, Sigma St. Louis, Mich.). The substrate has previously been selected because it does not exist in human blood, and due to the fact it has a similar structure to the smallest natural ASMase substrate. Sealed plates were incubated on an orbital shaker at 37° C. for 20 hours. Reactions were quenched with 100 μl of organic solution (ethyl acetate:methanol, 1:1) following liquid-liquid and solid phase extractions. The samples were dried under nitrogen, sealed and stored at −20° C. Prior to tandem mass spectrometry (MS/MS) analysis, plates were thawed and reconstituted with 200 μl of a solvent mixture (80:20 acetonitrile:water containing 0.2% formic acid).

All analyses were monitored on an API 4000 triple quadrupole mass spectrometer (ABSciex, Framingham, Mass., USA) by selected ion monitoring mode (Multiple Reaction Monitoring, MRM). The enzyme activity of each sample was calculated from the ion abundance ratio of product to internal standard as measured by the mass spectrometer. Background activity of a blank filter paper was subtracted from the dried blood spot activity. Activity was expressed as micromoles of product per liter of whole blood per hour (μmol/l/h). Two quality-control (QC) samples with previously established activity levels for each enzyme and disease positive samples were included in each plate for QC. All Genzyme scientists were blinded to Parkinson's and genetic status.

### Cell Cultures, SiRNAs and Cell Transfection

HeLa and BE(2)-M17 cells were obtained from ATCC and were maintained at 37° C. and 5% CO2. HeLa cells were cultured in Dulbecco's modified Eagle medium (DMEM, Wisent Inc.) supplemented with 10% heat-inactivated fetal bovine serum (FBS), 2 mM glutamine and 100 units/ml penicillin (Wisent Inc.). BE(2)-M17 cells were cultured in a medium containing 50% of Eagle's Minimum Essential Medium (EMEM) (Wisent Inc.), 50% of HAM-F12 (Wisent Inc.) supplemented with 10% heat-inactivated fetal bovine serum (FBS), 2 mM glutamine and 100 units/ml penicillin (Wisent Inc.). A human siRNA “ASM/1” (Life technologies, siRNA ID: s13167) was used to knockdown SMPD. The Silencer® Select Negative Control No. 1 siRNA “NT/1” (4390843, Life Technologies) was used as non-targeting siRNA. Another siRNA targeting SMPD1 “ASM/2” (QIAGEN, Hs_SMPD1_5 FlexiTube siRNA, Cat. No.: SI03096121) was used for replication. AllStars Negative Control siRNA (QIAGEN, Cat. No.: SI03650318) was used as non-targeting siRNA (NT/2) for ASM/2. The siRNA oligos used to knockdown SMPD1 was as follow: “ASM/1: 5′-UCACAGCACUUGUGAGGAAtt-3′” and “ASM/2: 5′-CTGCTGTGGGTAACCTAGAAA-3′”. For siRNA transfections, cells were passed into plates containing the siRNA (final concentration of 10 nM), HiPerFect (Qiagen), and DMEM (Wisent Inc.) and incubated for 48 h. siRNA transfections were repeated and at 48 h post-second transfection, cells were lysed for western blotting.

### Immunoblotting and α-Synuclein Accumulation

Cells were lysed using RIPA lysis buffer (20 mM Tris-HCl, pH 7.4, 0.1% SDS, 150 mM NaCl and 1% Nonidet P-40) with a mixture of protease inhibitors (aprotinin, leupeptin, benzamidine, PMSF). After adding Laemmli buffer with DTT to the samples, they were boiled for 10 min and separated by SDS-PAGE. Then separated samples were transferred to a nitrocellulose membrane. For α-synuclein, the membrane was incubated with phosphate-buffered senile (PBS) containing 0.4% paraformaldehyde (PFA) for 30 min as described previously.³¹ Membranes were blocked with 5% skim milk in PBS containing 0.1% Tween-20 (PBS-T) and incubated overnight at 4 ° C. with primary antibody in PBS-T containing 1% milk. As primary antibody, rabbit polyclonal anti-ASM antibody (PA5-30177,Thermofisher Scientific) and mouse anti-a-synunclein antibody (clone 42/α-Synuclein, BD Transduction Laboratories) were used at a dilution 1:1000, and mouse monoclonal anti-actin antibody (MAB1501, Millipore) was used at a dilution 1:40000. Membranes were washed with PBS-T containing 1% milk three times for 5 min and then incubated with a secondary antibody, horseradish peroxidase-conjugated anti-mouse IgG or anti-rabbit IgG antibody (Jackson) at a 1:10000 dilution in PBS-T containing 1% milk for 1 hour at room temperature.³¹ Finally membranes were washed three times with PBST³¹ and were developed using ECL substrate (PerkinElmer Life Sciences). Densitometric quantification was performed using Image Studio Lite.

### Statistical Analysis

Data are presented in the text and tables as percentages for categorical variables and as average ±standard deviation (SD) for continuous variables. To examine the association between all rare SMPD1 variants and PD in each site (MTL and N.Y.), Fisher's exact test, followed by binary logistic regression with age and gender as covariates were used. To examine the association of specific SMPD1 variants with PD, only Fisher's exact test could be used due to the rarity of these variants.

As above, ASMase activity was available in the N.Y. cohort. The association between ASMase activity and PD status was tested using Student T Test, and logistic regression adjusting for gender and age. To examine the association of ASMase activity with AAO the several tests were performed. Linear regression models with the AAO as the dependent variable and gender and ASMase activity as independent variables were performed. Since age at enrollment (AAE) and AAO are co-linear (i.e. patients with earlier AAO are also enrolled at earlier age to the study), disease duration was used rather than AAE as a covariate. The regression models were repeated excluding early-onset PD cases and those with LRRK2 G2019S or GBA mutations. The cohort was further divided to quartiles based on ASMase activity in the control population, and ANOVA was performed to examine the association between ASMase activity quartiles and demographics and disease characteristics (including AAO). To determine the association between specific SMPD1 variants and enzymatic activity the non- parametric Mann-Whitney tests were used due to the small number of individuals with a specific variant. Lastly ASMase activity between LRRK2 G2019S and GBA mutations carriers and non-carriers was compared. SPSS V.21 was used for all the statistical analyses.

### Meta-Analysis

For the meta-analysis, the R package Metafor was used as previously described.⁴ Six studies, including 5 that were previously published¹³⁻¹⁷ and the current study were included in the meta-analysis. In one study,¹⁵ a very common variant, the Leu-Ala (Val) repeat in exon 1 of SMPD1, was associated with PD, but it could not be included in the analysis due to its high frequency and the resulting heterogeneity of the model.

### Results

Association of SMPD1 Mutations with Parkinson Disease; Possible Role for the p.A487V Mutation

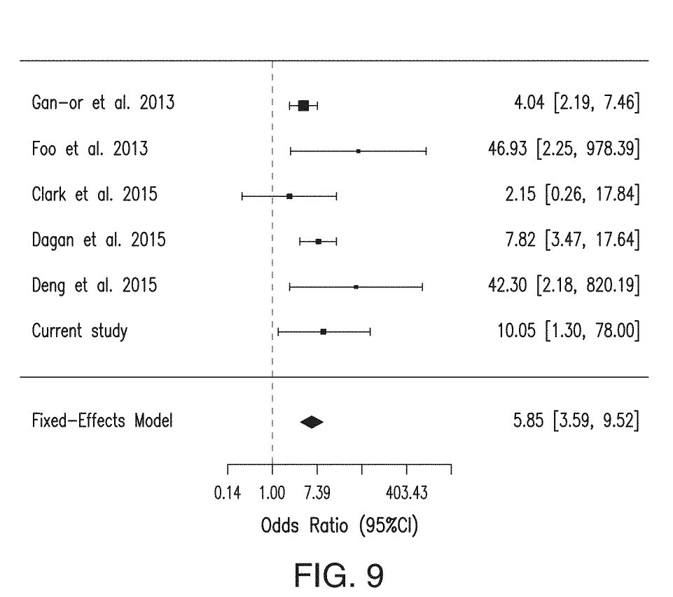

In the MTL cohort, a total of 29 different SMPD1 variants that affect the coding sequence or alternative splicing were identified (Table 12). Among patients, 5.3% (n=28) carried a rare SMPD1 variant, compared to 2.9% (n=20) among controls (OR=1.88, 95% CI 1.05-3.39, p=0.037). After adjustment for age and sex, this association was not significant, which is likely due to the patients with SMPD1 variants, who had a trend towards an earlier AAE (Mann-Whitney U test, p=0.079). Since data on age at onset for this cohort was not available, it could not be determined if the earlier AAE is due to earlier AAO. However, the association was mainly driven by a single variant, p.A487V, found in 8 (1.5%) patients and in one (0.14%) control (OR=10.68, 95%CI 1.3-85.6, p=0.0065). In the N.Y. cohort, the p.A487V variant was found in 3 patients (0.54%) and in one control (0.35%, p=1.0). Combining the two cohorts, the p.A487V was found in 11 (1.0%) patients and 2 (0.2%) controls (OR=5.03, 95%CI 1.11-22.75, p=0.024). The frequency of rare SMPD1 variants in the N.Y. cohort was not significantly different between patients and controls, 3.8% versus 5.3%, p=0.34. The frequency of rare variants that had reduced activity in patients and controls were further compared. Although these variants (p.R291H, p.P331A, p.R378H, p.A487V, p.G492S, p.R498L, p.E517V, p.G530A, fs.c1829delCGG, p.R610H) were more frequent in patients (n=12, 2.2%) than in controls (n=4, 1.4%), these differences did not reach statistical significance. To further estimate the effect of SMPD1 variants on risk for synucleinopathies, a meta-analysis was conducted of 6 available studies, including the current study. Five of the studies compared PD patients and control and one study compared pathologically-confirmed Lewy-body disease patients with controls. The forest plot of the analysis is presented in FIG. 9, with an OR of 5.8 (95% CI 3.6-9.5, p<0.0001, Tarone's Test for Heterogeneity p=0.0935).

TABLE 12
SMPD1 variants in PD patients and controls in the MTL cohort

| | | Patients | | Controls | | |
|---|---|---|---|---|---|---|
| Variant | Pathogenicity^{a} | (n = 525) | | (n = 691) | | p |
| p.E108K | Unknown | 0 | (0.0%) | 1 | (0.1%) | NA |
| p.V114M | Probably pathogenic | 1 | (0.2%) | 0 | (0.0%) | NA |
| p.I127V | Unknown | 1 | (0.2%) | 0 | (0.0%) | NA |
| p.E141D | Unknown | 1 | (0.2%) | 0 | (0.0%) | NA |
| p.C159R | Probably pathogenic | 1 | (0.2%) | 0 | (0.0%) | NA |
| p.D225Y | Unknown | 1 | (0.2%) | 0 | (0.0%) | NA |
| p.C228R | Probably pathogenic | 0 | (0.0%) | 1 | (0.1%) | NA |
| p.G247S | Probably pathogenic | 0 | (0.0%) | 1 | (0.1%) | NA |
| p.K251R | Unknown | 1 | (0.2%) | 0 | (0.0%) | NA |
| p.D253A | Unknown | 1 | (0.2%) | 0 | (0.0%) | NA |
| p.Q289X | Probably pathogenic | 1 | (0.2%) | 0 | (0.0%) | NA |
| p.R291H | Probably pathogenic | 3 | (0.6%) | 3 | (0.4%) | p > 0.0 |
| p.R296Q | Probably pathogenic | 1 | (0.2%) | 0 | (0.0%) | NA |
| p.P325A | Probably pathogenic | 0 | (0.0%) | 1 | (0.1%) | NA |
| p.R341H | Unknown | 0 | (0.0%) | 1 | (0.1%) | NA |
| p.L379F | Unknown | 2 | (0.4%) | 1 | (0.1%) | p > 0.0 |
| p.R418Q | Unknown | 1 | (0.2%) | 0 | (0.0%) | NA |
| IVS3 + 2T > C | Probably pathogenic | 1 | (0.2%) | 0 | (0.0%) | NA |
| p.K435R | Unknown | 0 | (0.0%) | 1 | (0.1%) | NA |
| p.W437C | Probably pathogenic | 1 | (0.2%) | 0 | (0.0%) | NA |
| p.V462M | Unknown | 0 | (0.0%) | 1 | (0.1%) | NA |
| p.A487V | Unknown^{b} | 8 | (1.5%) | 1 | (0.1%) | p = 0.00 |
| p.G492S | Probably pathogenic | 1 | (0.2%) | 3 | (0.4%) | p > 0.0 |
| p.G504X | Probably pathogenic | 0 | (0.0%) | 1 | (0.1%) | NA |
| p.G508R | Not pathogenic | 205 | (39.0%) | 249 | (36.0%) | p > 0.0 |
| p.E517V | Probably pathogenic | 1 | (0.2%) | 2 | (0.3%) | p > 0.0 |
| p.E543X | Probably pathogenic | 1 | (0.2%) | 0 | (0.0%) | NA |
| p.V559I | Unknown | 0 | (0.0%) | 1 | (0.1%) | NA |
| p.M566T | Unknown | 0 | (0.0%) | 1 | (0.1%) | NA |
| Total rare | | 28 | (5.3%) | 20 | (2.9%) | p = 0.0 |
| Total | | 12 | (2.3%) | 12 | (1.7%) | p > 0.05 |
| “probably | | | | | | |

NA, not applicable
^{a}Based on its association with Neimann-Pick type A or B, using a recently published database of pathogenic mutations that was recently published,¹⁸ or if it results in a stop codon.

ASMase Enzymatic Activity is Associated with Specific SMPD1 Variants and with Age at Onset of PD

Among control, there was no association between age and gender and ASMase activity (p=0.11 for age, p=0.46 for gender). There was no difference between the average enzymatic activity in patients and controls (4.64 μmol/l/h ±1.68 μmol/l/h and 4.62 μmol/l/h ±1.64 μmol/l/h, respectively, p=0.84). Similarly, there was no difference after excluding carriers of SMPD1 mutations (4.66 μmol/l/h ±1.66 μmol/l/h and 4.60 μmol/l/h ±1.59 μmol/l/h, respectively, p=0.66). However, among PD cases, reduced ASMase enzymatic activity was associated with an earlier AAO. In a linear regression model with AAO as the dependent variable, and gender, disease duration and enzymatic activity as co-variates, ASMase activity was strongly associated with earlier AAO (B=1.072, β=0.155, 95% CI for B 0.55- 1.60, p<0.001, i.e., for each 1 μmol/l/h reduction age at onset was younger by 1.1 year). The inventor further divided the cohort to quartiles based on the enzymatic activity in controls (Table 14). First, the entire cohort was analyzed, followed by analysis of late-onset PD only (AAO≧50 years old), and further exclusion of carriers of GBA and LRRK2 mutations that can also affect the AAO. In all analyses, there was a gradual decrease in AAO with the decrease of enzymatic activity, with significant differences of 3.5-5.8 years between the first and last quartiles in the different analyses (Table 14, p<0.01 for all comparisons). Other demographics and disease characteristics were not different among the quartiles (Table 15) among PD patients with late-onset PD who do not carry GBA or LRRK2 mutations. Similar results were obtained (i.e., only AAO was significantly different between the quartiles) when early onset PD and GBA and LRRK2 mutation carriers were included. The LRRK2 p.G2019S and GBA mutations were not associated with ASMase activity (data not shown).

TABLE 13
SMPD1 variants and their enzymatic activity
in PD patients and controls in the NY cohort

| | | | | | | p value, |
|---|---|---|---|---|---|---|
| | | | | | ASMase | comparing |
| | | | | | activity^{a} | ASMase activity |
| | Patients | | Controls | | (combined PD | in carriers vs. |
| Variant | (n = 550) | | (n = 284) | | and controls) | non-carriers^{b} |
| Rare variants | | | | | | |
| p.Q19R | 4 | (0.7%) | 2 | (0.7%) | 6.31 ± 2.67 | 0.13 |
| p.V114M^{c} | 2 | (0.4%) | 0 | (0%) | 5.04 ± 1.45 | 0.59 |
| p.P187S | 1 | (0.2%) | 3 | (1.1%) | 5.70 ± 1.43 | 0.13 |
| p.G269S | 0 | (0%) | 1 | (0.4%) | 4.59 | NA |
| p.R291H^{c} | 1 | (0.2%) | 0 | (0%) | 3.45 | NA |
| p.P331A | 1 | (0.2%) | 0 | (0%) | 2.76 | NA |
| p.R378H^{c} | 0 | (0%) | 1 | (0.4%) | 2.22 | NA |
| p.R389C | 1 | (0.2%) | 1 | (0.4%) | 7.51 ± 3.03 | 0.08 |
| p.W393G^{c} | 3 | (0.5%) | 3 | (1.1%) | 5.14 ± 1.77 | 0.54 |
| p.A487V | 3 | (0.5%) | 1 | (0.4%) | 2.85 ± 0.59 | 0.01 |
| p.G492S^{c} | 1 | (0.2%) | 0 | (0%) | 2.16 | NA |
| p.R498L^{c} | 2 | (0.4%) | 0 | (0%) | 3.32 ± 1.20 | 0.20 |
| p.E517V^{c} | 1 | (0.2%) | 2 | (0.7%) | 2.38 ± 0.47 | 0.007 |
| p.G530A | 1 | (0.2%) | 0 | (0%) | 3.00 | NA |
| c.1829delCGG | 1 | (0.2%) | 0 | (0%) | 2.14 | NA |
| p.M613I | 0 | (0%) | 1 | (0.4%) | 7.61 | NA |
| p.R610H | 1 | (0.2%) | 0 | (0%) | 2.14 | NA |
| Common variants | | | | | | |
| p.G508R | G/G 345 | (62.7%) | G/G 196 | (66.9%) | 4.96 ± 1.73 | |
| | G/R 186 | (33.8%) | G/R 80 | (30.3%) | 4.09 ± 1.37 | |
| | R/R 19 | (3.5%) | R/R 8 | (2.8%) | 3.56 ± 1.18 | p < 0.001 d |
| Non-carriers^{e} | 529 | (96.2%) | 269 | (94.7%) | 4.64 ± 1.64 | |

ASMase, acid sphingomyelinase
^{a}The activity of ASMase is measured in μmol/l/h units
^{b}Mann-Whitney test
^{c}A pathogenic variant that may lead to Neimann-Pick type A or B, based on a recently published database of pathogenic mutations.¹⁸
d Both ANOVA and the non-parametric Kruskal-Wallis tests were performed, in both p < 0.001
^{e}Non-carriers of rare SMPD1 mutations, not including the p.G508R variant

TABLE 14
ASMase activity quartiles^{a} and age at onset of PD.

| | Quartile 1 | Quartile 2 | Quartile 3 | Quartile 4 | p value b |
|---|---|---|---|---|---|
| ASMase activity range | <3.485 | 3.485-4.46 | 4.46-5.64 | >5.64 | |
| μmol/l/h) | | | | | |

All patients

| | | | | | |
|---|---|---|---|---|---|
| N | 134 | 148 | 146 | 120 | |
| Average AAO ± SD | 58.6 ± 10.3 | 58.2 ± 12.2 | 58.2 ± 12.4 | 62.2 ± 11.0 | p = 0.01 |

All patients with late-onset PD (AAO ≧ 50)

| | | | | | |
|---|---|---|---|---|---|
| N | 115 | 117 | 111 | 104 | |
| Average AAO ± SD | 61.5 ± 7.2 | 63.0 ± 7.5 | 63.5 ± 8.3 | 65.0 ± 8.8 | p = 0.01 |

All patients excluding GBA and LRRK2 mutation carriers

| | | | | | |
|---|---|---|---|---|---|
| N | 109 | 119 | 113 | 89 | |
| Average AAO ± SD | 57.4 ± 10.4 | 58.2 ± 12.5 | 58.1 ± 12.9 | 63.2 ± 11.2 | p = 0.003 |

All patients with late-onset PD, excluding GBA and LRRK2 mutation carriers

| | | | | | |
|---|---|---|---|---|---|
| N | 91 | 93 | 85 | 80 | |
| Average AAO ± SD | 60.7 ± 7.1 | 63.4 ± 7.6 | 64.0 ± 8.2 | 65.5 ± 9.1 | p = 0.001 |

ASMase, acid sphingomyelinase; N, number; AAO, age at onset; SD, standard deviation.
^{a}Quartiles were determined according to ASMase activity in controls, however nearly identical results were calculated when the quartiles were determined according to patients only, or patients and controls combined.
b Analysis of variance (ANOVA).

TABLE 15
ASMase activity quartiles^{a} and clinical characteristics of late
onset PD patients, non- carriers of GBA or LRRK2 mutations

| | Quartile 1 | Quartile 2 | Quartile 3 | Quartile 4 | p value b |
|---|---|---|---|---|---|
| N | 91 | 93 | 85 | 80 | |
| ASMase activity mean ± SD, | 2.84 ± 0.42 | 4.00 ± 0.29 | 5.02 ± 0.35 | 6.99 ± 1.56 | |
| ASMase activity range, μmol/l/h | 1.86-3.48 | 3.49-4.44 | 4.47-5.61 | 5.65-13.5 | |
| Women, % | 41.8% | 34.4% | 34.1% | 32.5% | p = 0.58 |
| Age at enrolment mean ± SD, | 66.0 ± 7.9 | 68.3 ± 7.7 | 70.1 ± 8.4 | 72.1 ± 8.7 | p < 0.001 |
| Age at PD onset mean ± SD, years | 60.7 ± 7.1 | 63.4 ± 7.6 | 64.0 ± 8.2 | 65.5 ± 9.1 | p = 0.001 |
| 1^{st} degree relative with PD. % | 12.1% | 15.1% | 22.4% | 11.3% | p = 0.17 |
| Disease duration ± SD, years | 5.4 ± 4.6 | 4.9 ± 4.4 | 6.1 ± 5.2 | 6.7 ± 5.2 | p = 0.09 |
| Levodopa equivalent daily dose | 493 ± 433 | 463 ± 426 | 434 ± 364 | 506 ± 393 | p = 0.66 |
| mean ± SD, mg | | | | | |
| UPDRS III mean ± SD | 19.2 ± 10.5 | 16.8 ± 10.1 | 17.9 ± 9.1 | 18.8 ± 10.6 | p = 0.40 |
| Montreal cognitive assessment, | 26.0 ± 2.8 | 25.0 ± 3.2 | 25.3 ± 3.8 | 24.8 ± 4.5 | p = 0.10 |

N, number; ASMase, sphingomyelinase; SD, standard deviation; AAE, age at enrolment; AAO, age at onset.
^{a}Quartiles were determined according to ASMase activity in controls, however similar results were calculated when the quartiles were determined according to patients only, or patients and controls combined.
b p values were calculated by chi-square test for categorical variables and Analysis of variance (ANOVA) for continuous variables. Bonferroni correction for multiple comparisons set the cut-off for statistical significance on p < 0.00625

### Knockdown of SMPD1 in Cellular Models Leads to Accumulation of α-Synuclein

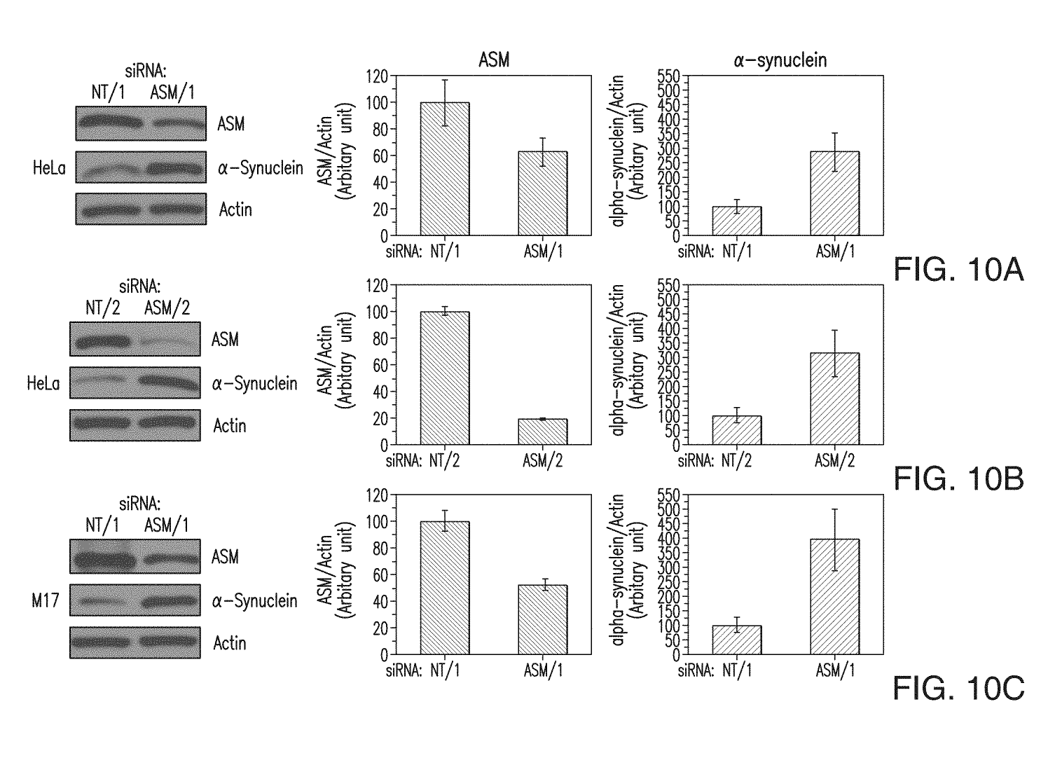

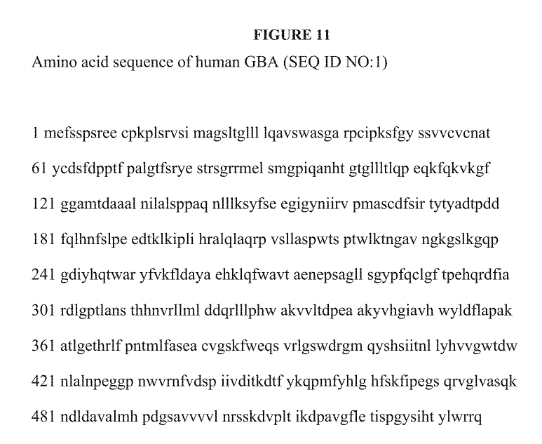

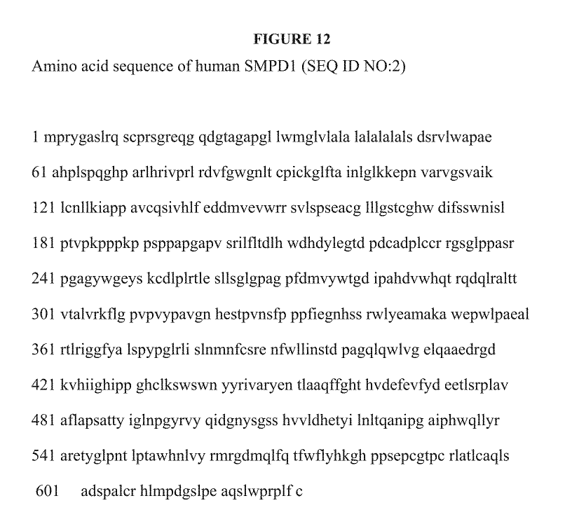

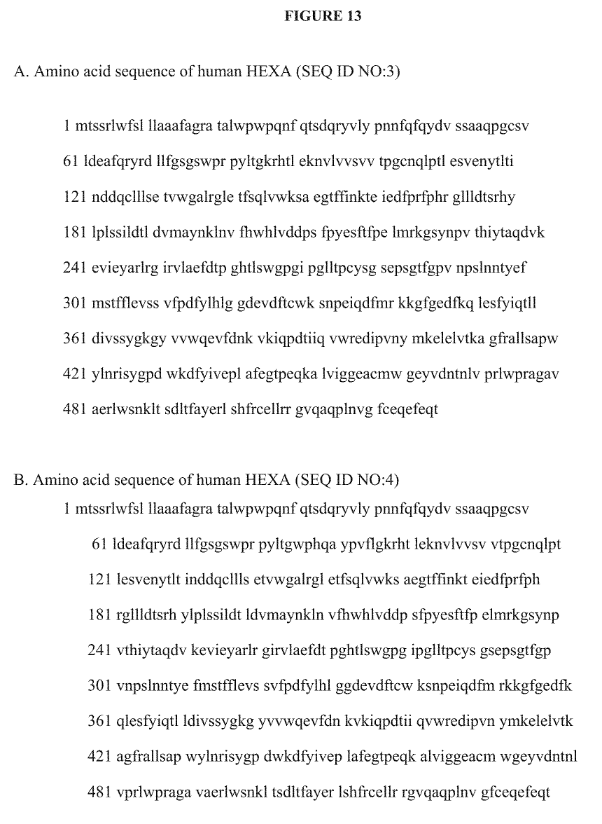

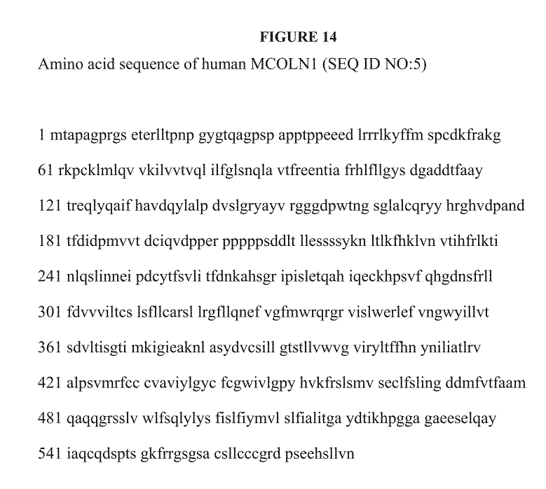

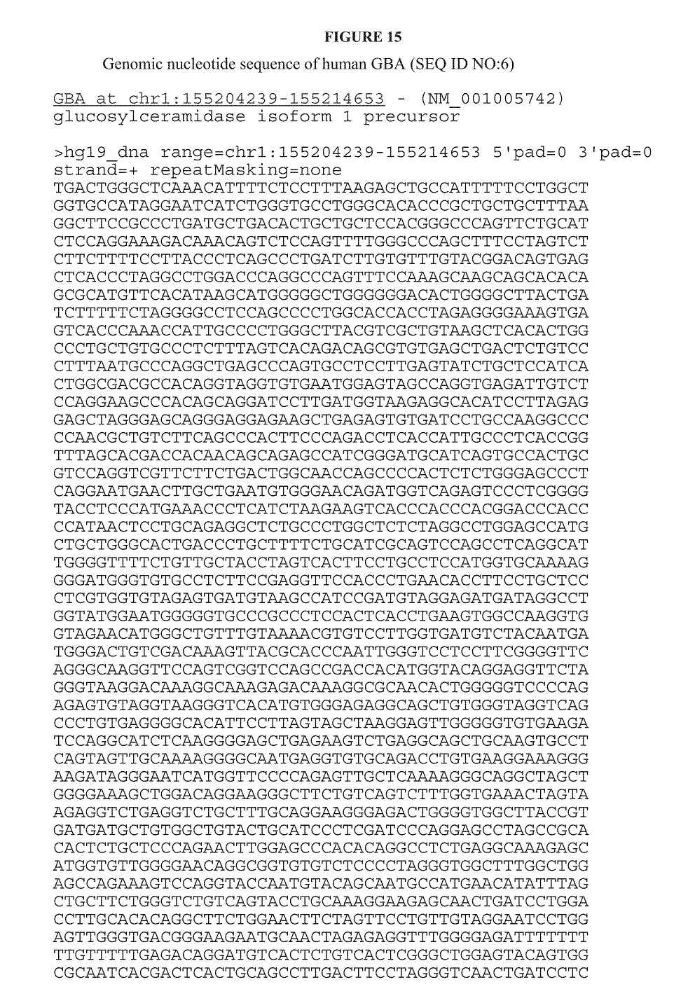

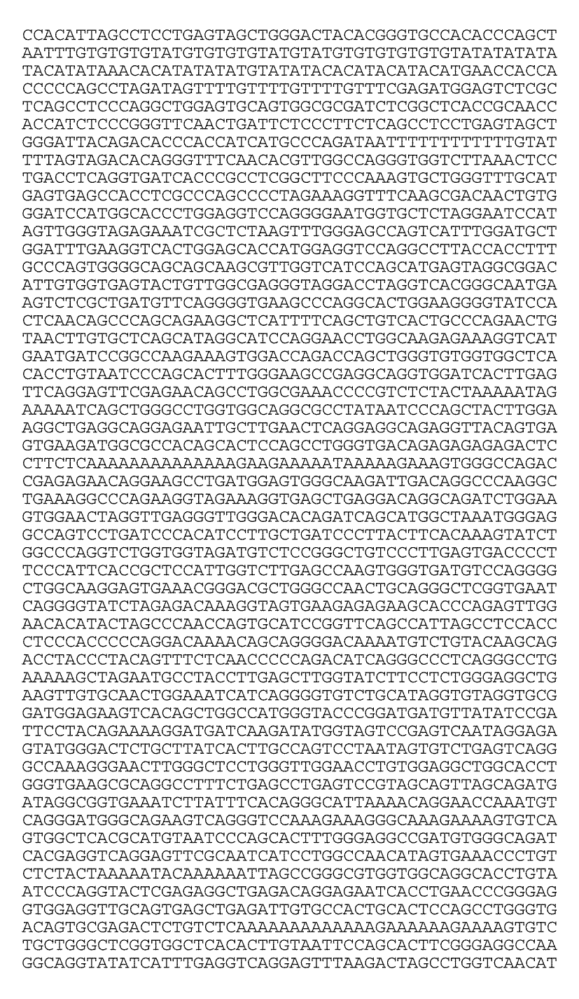

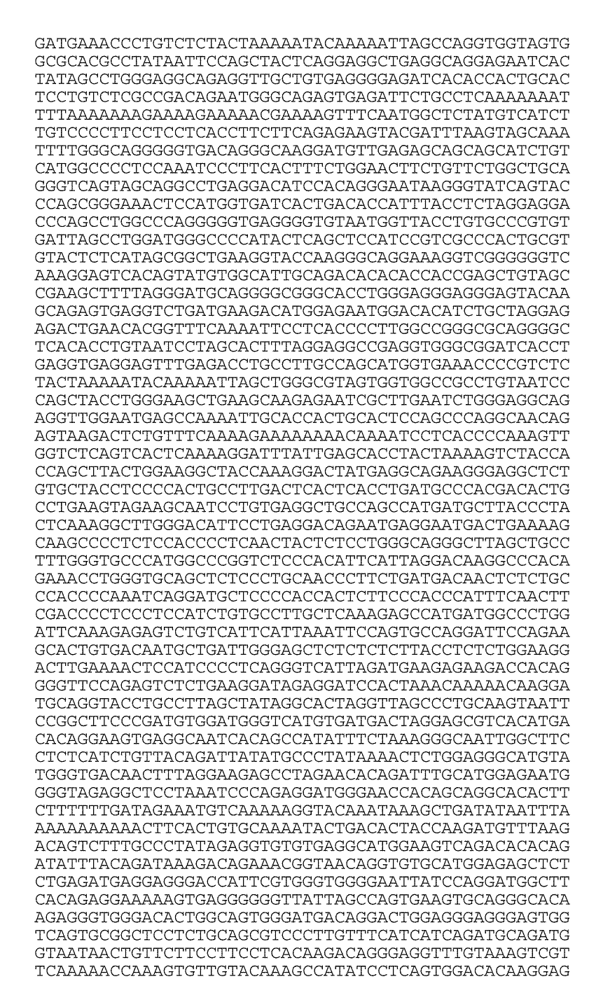

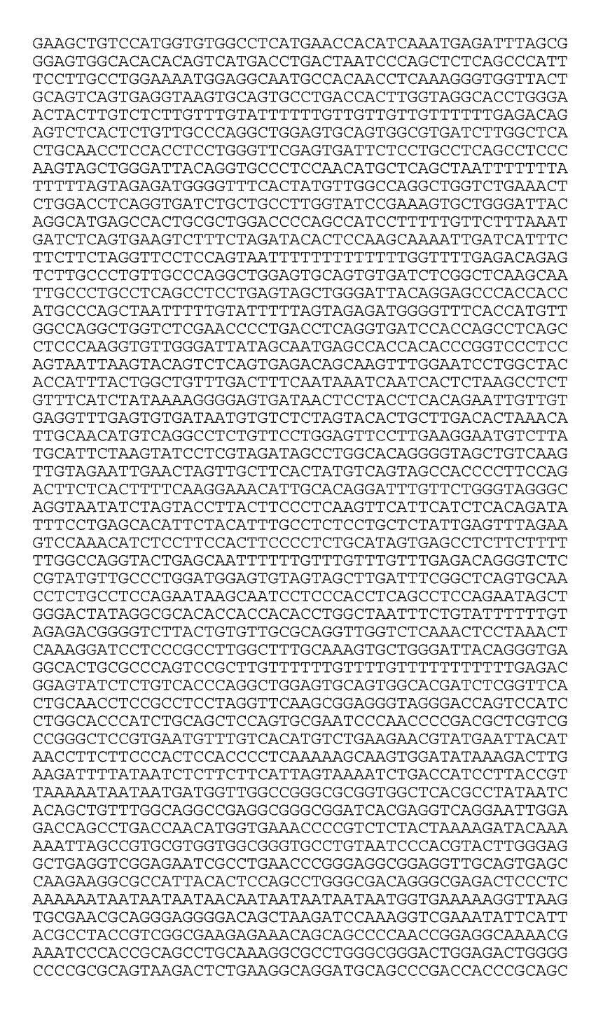

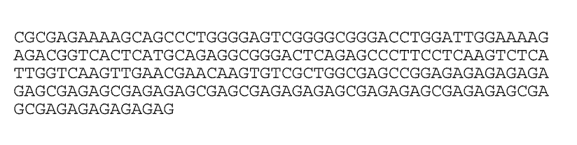

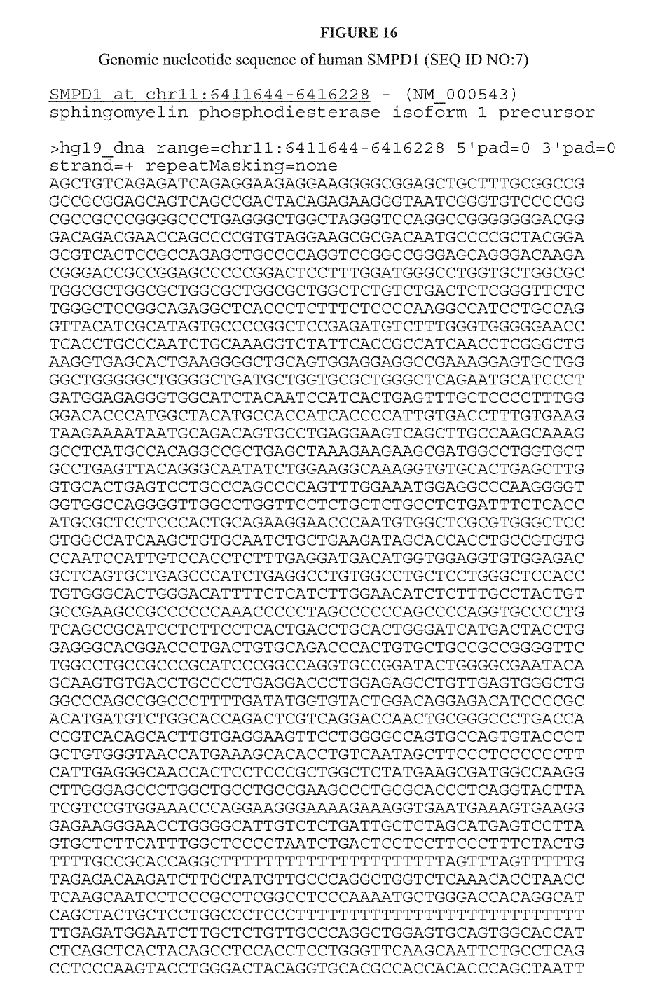

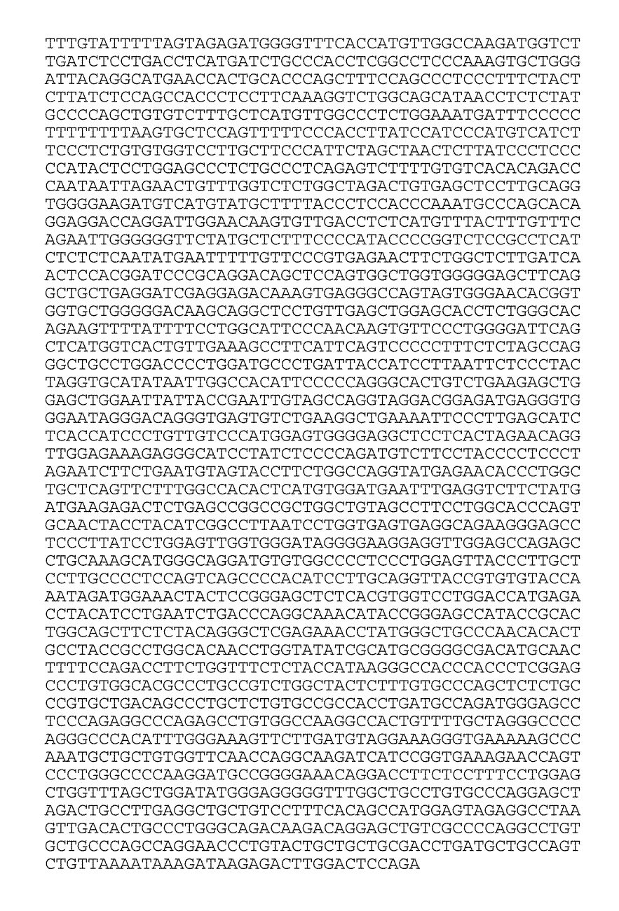

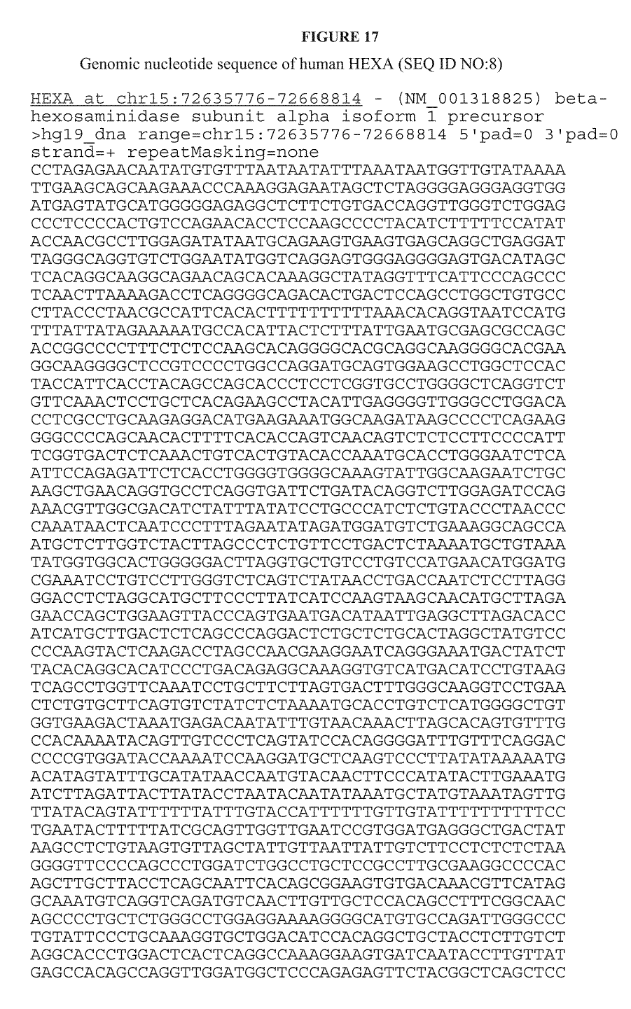

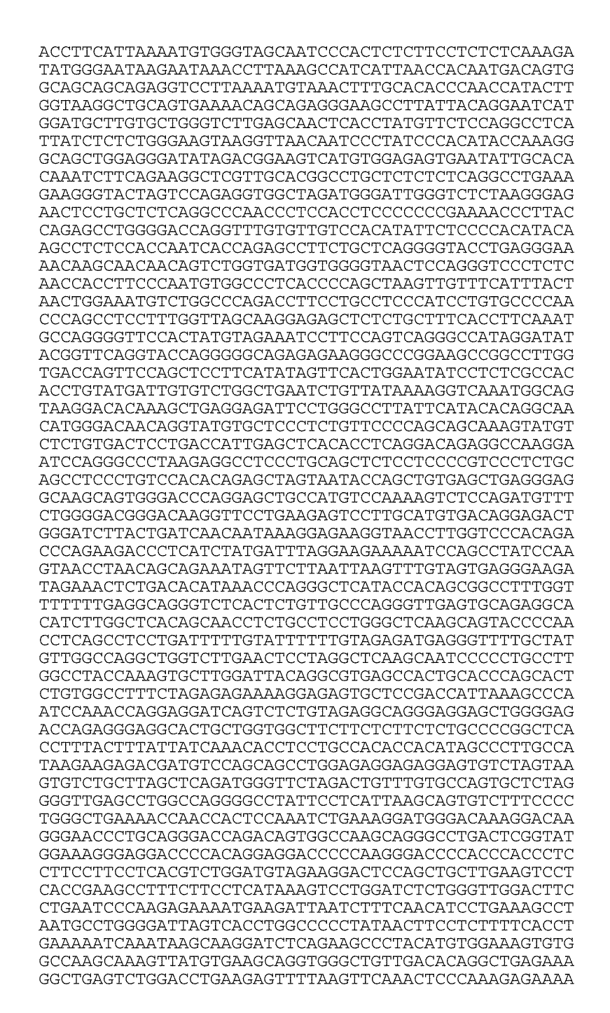

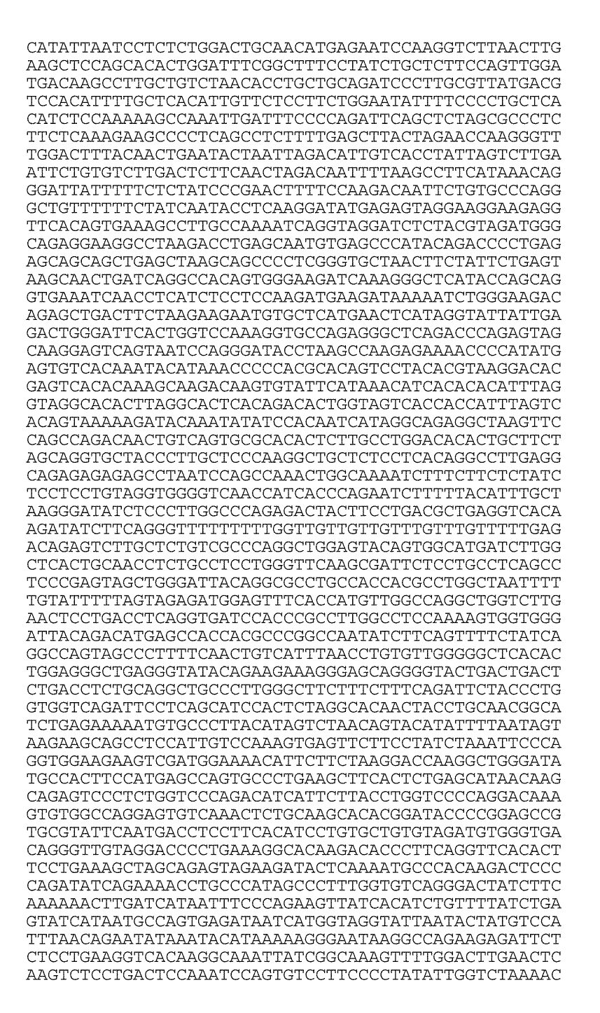

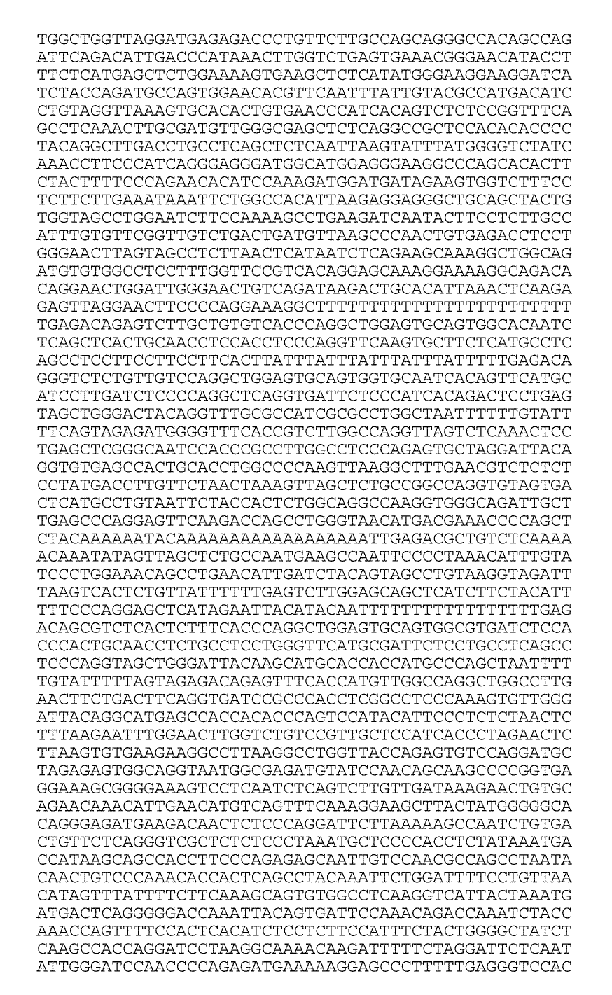

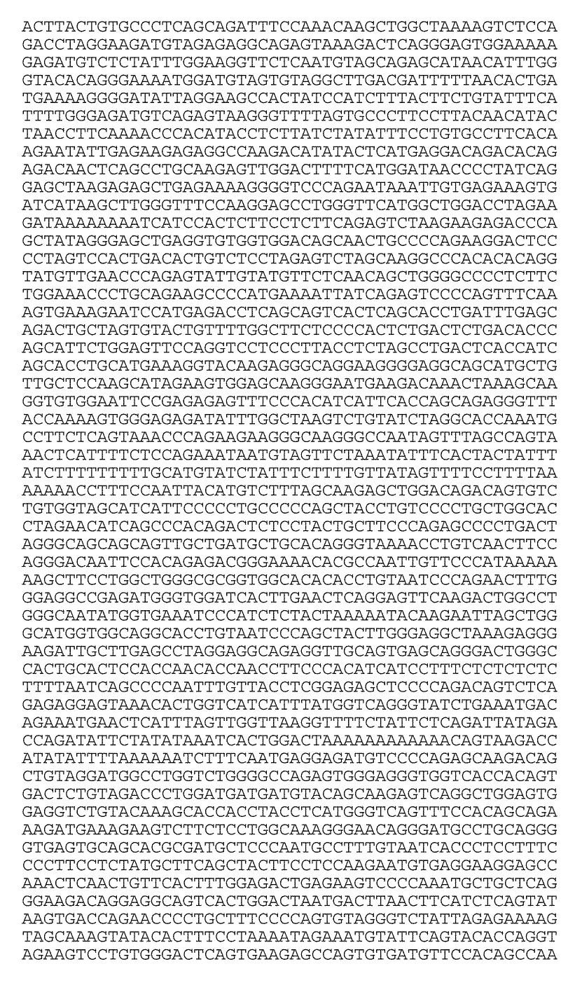

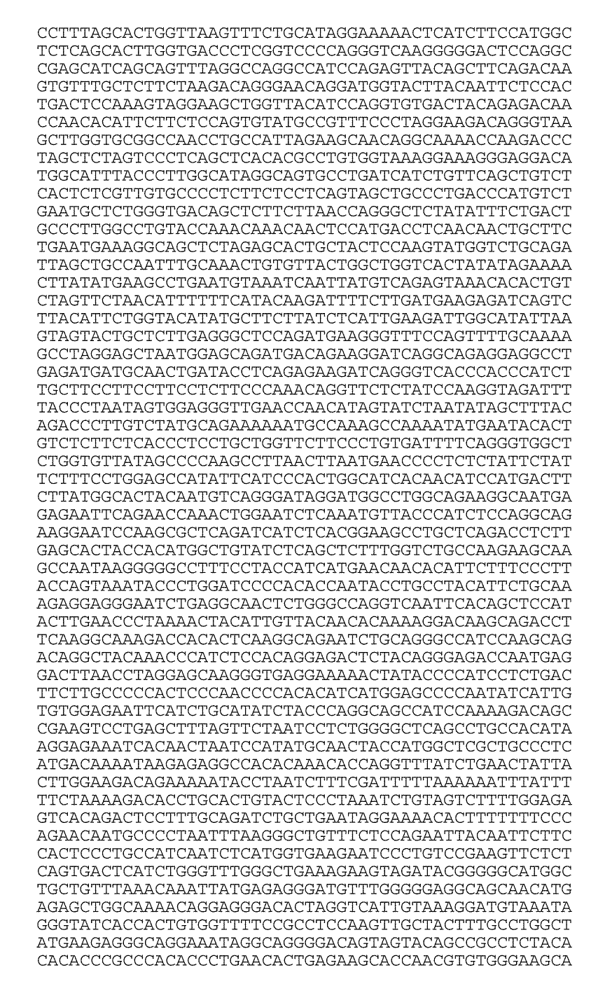

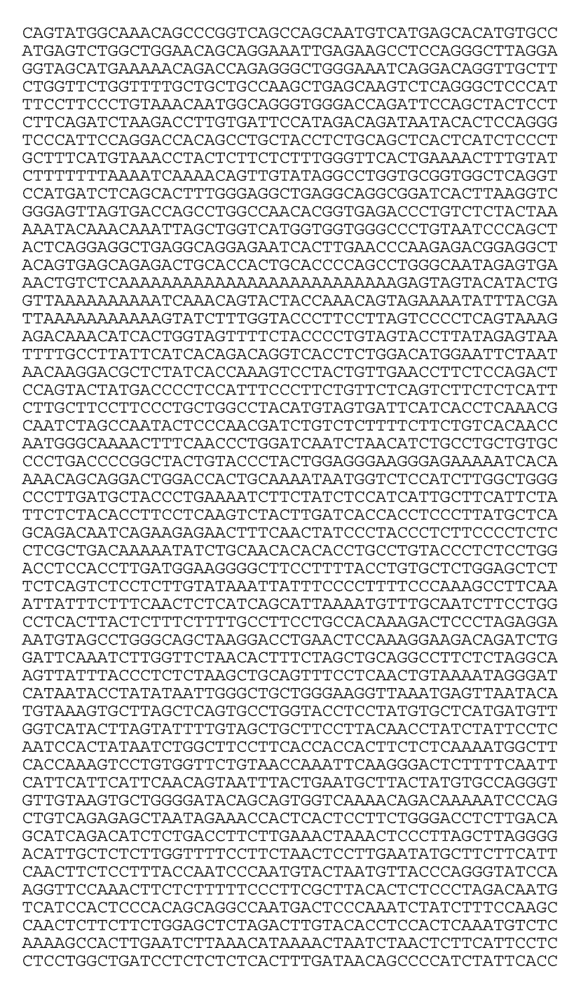

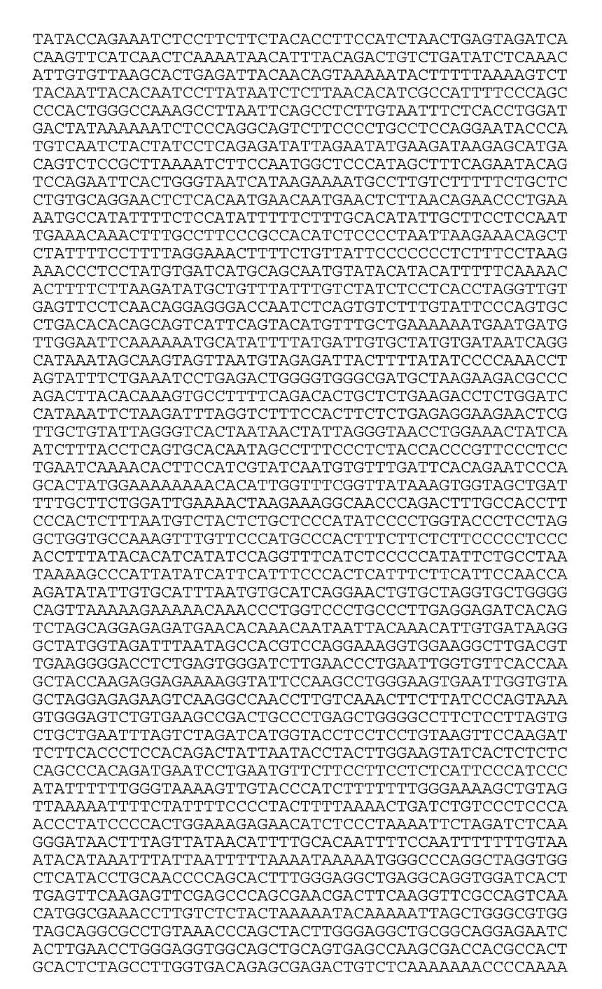

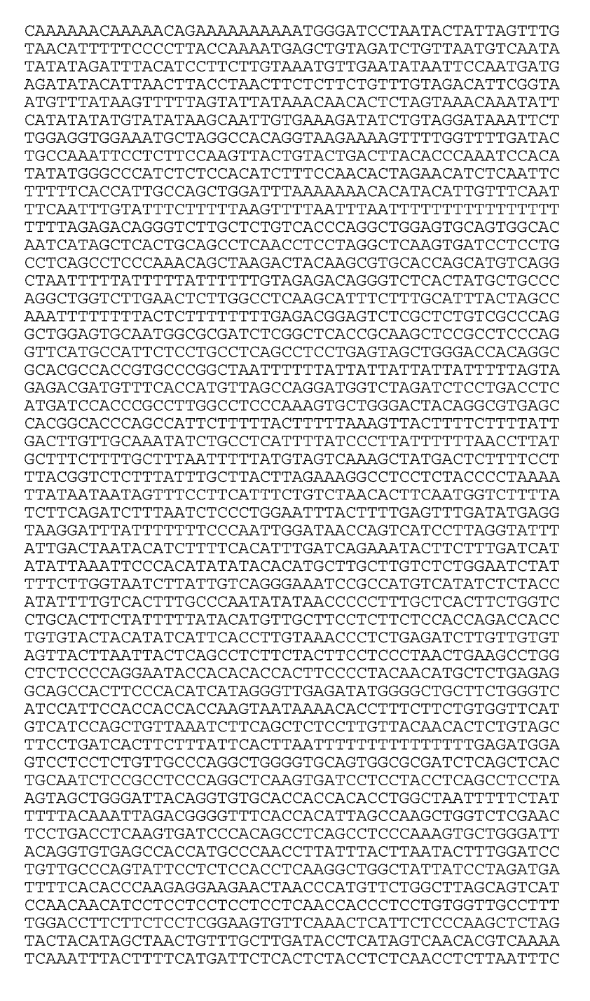

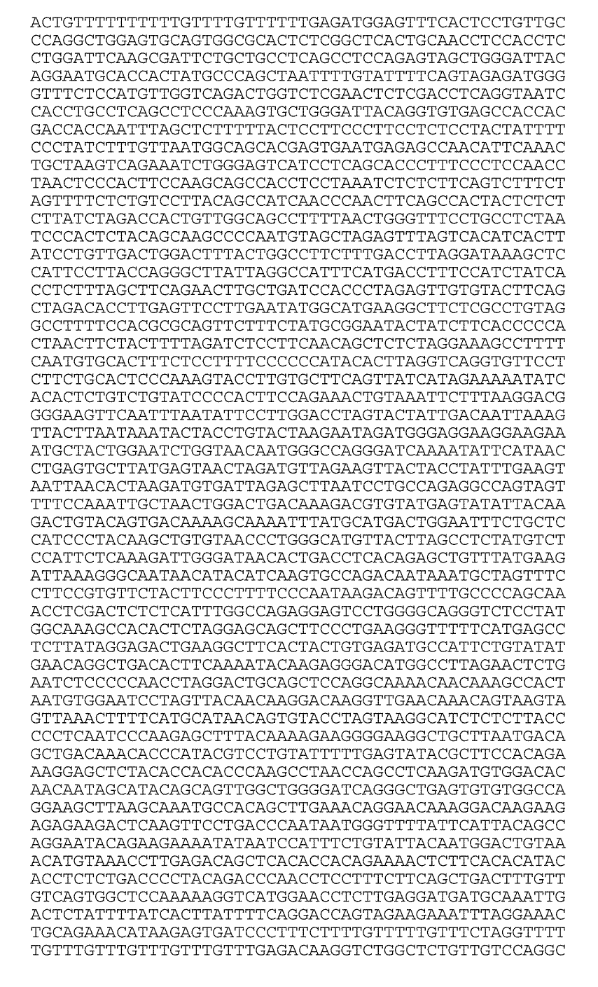

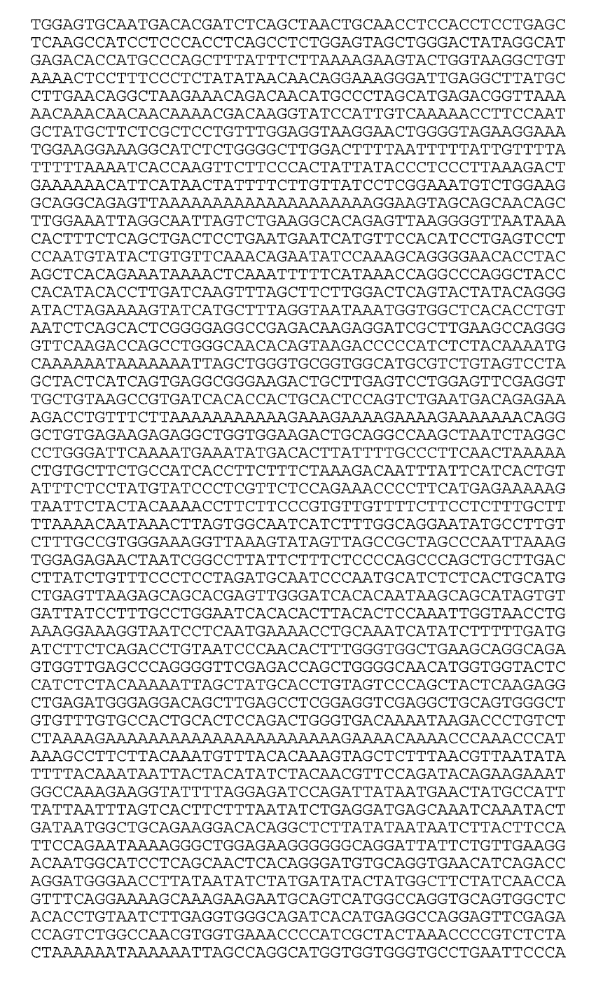

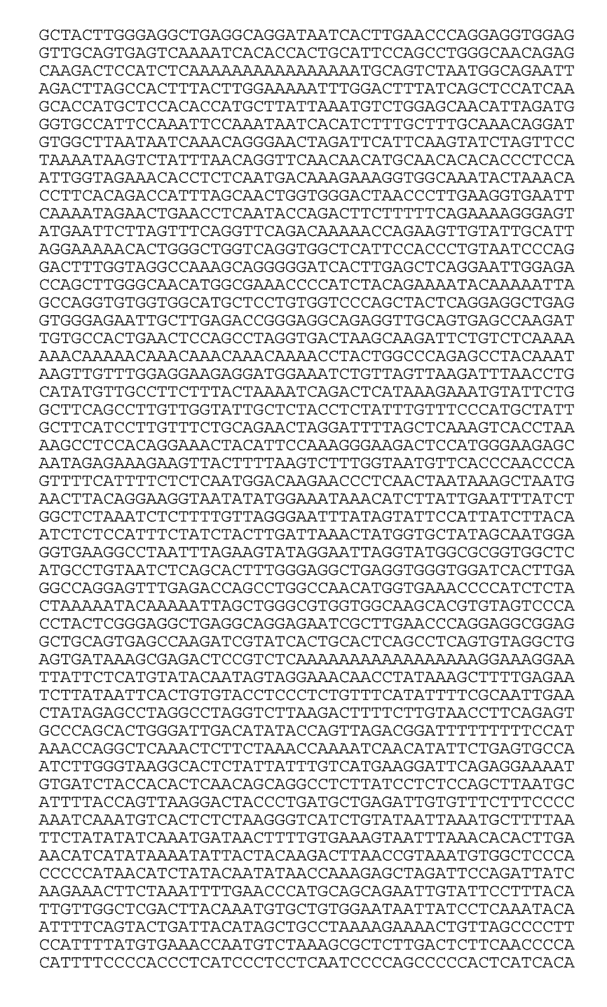

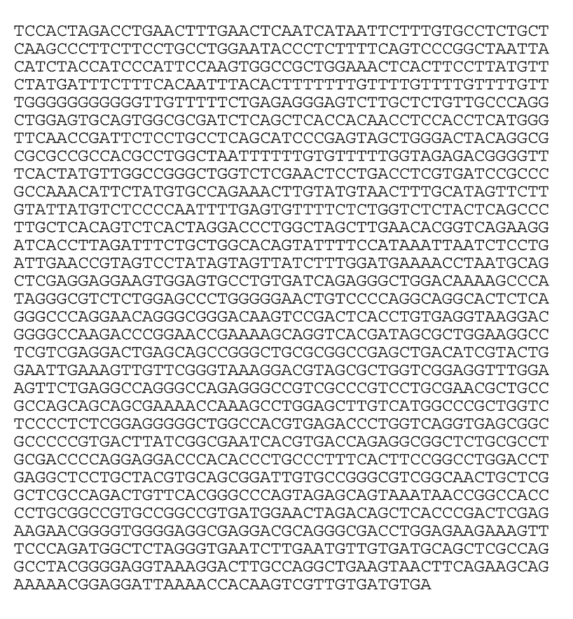

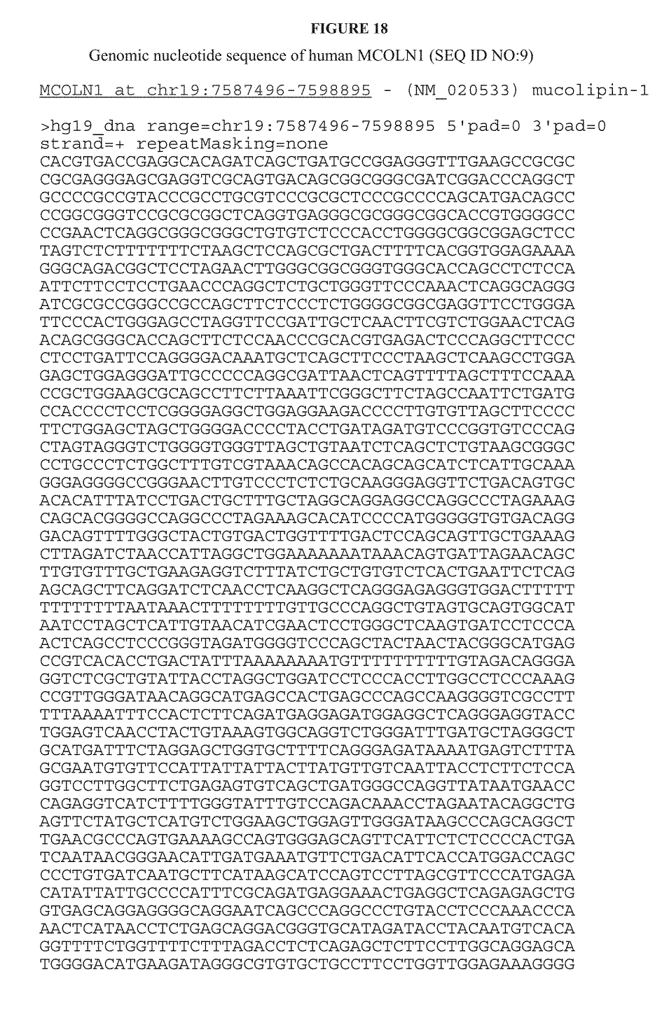

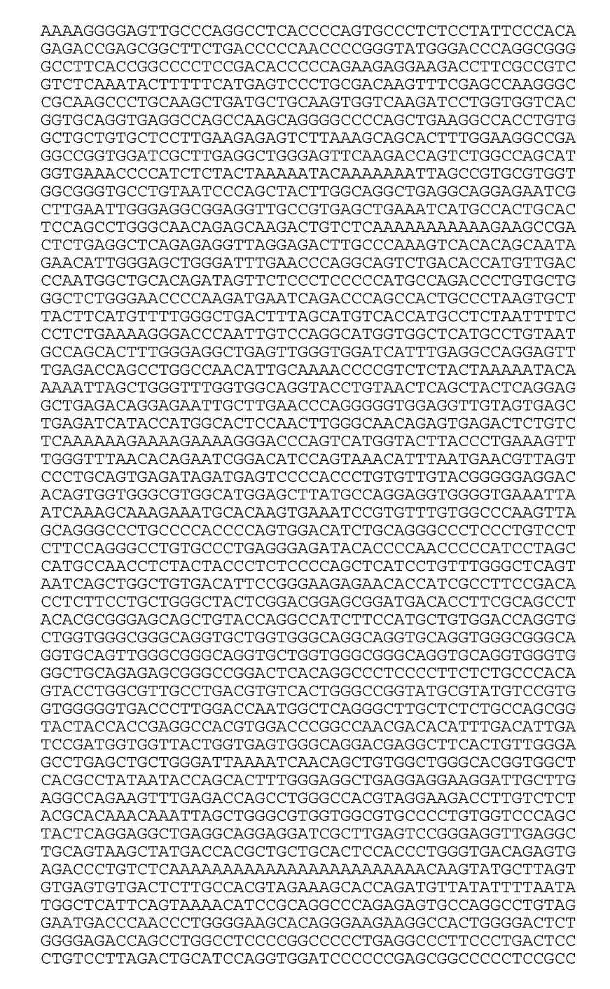

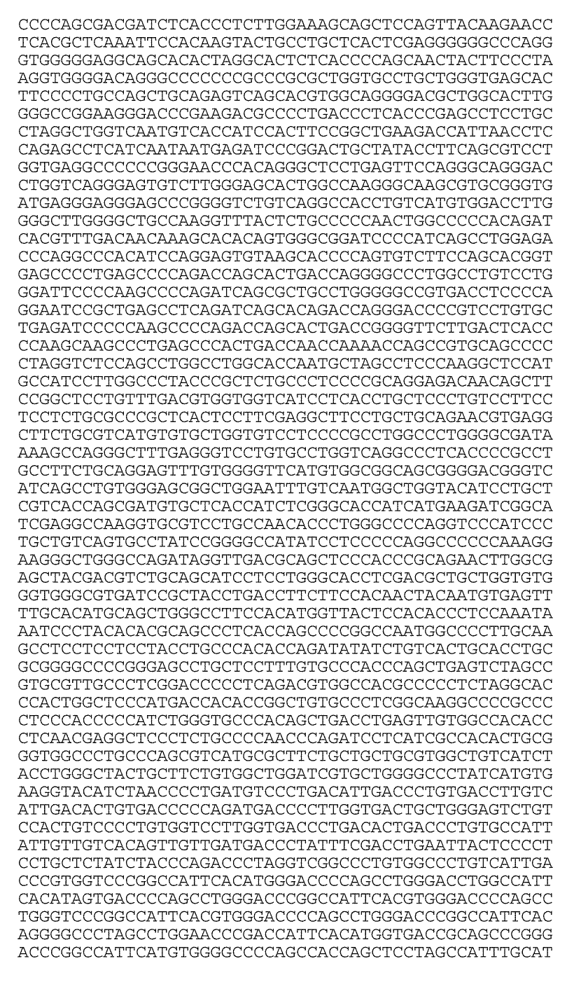

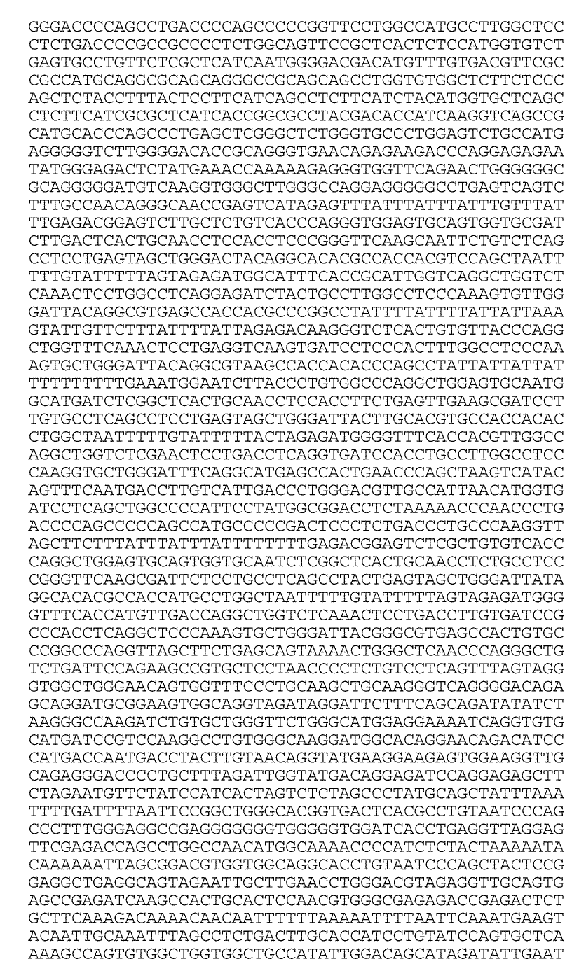

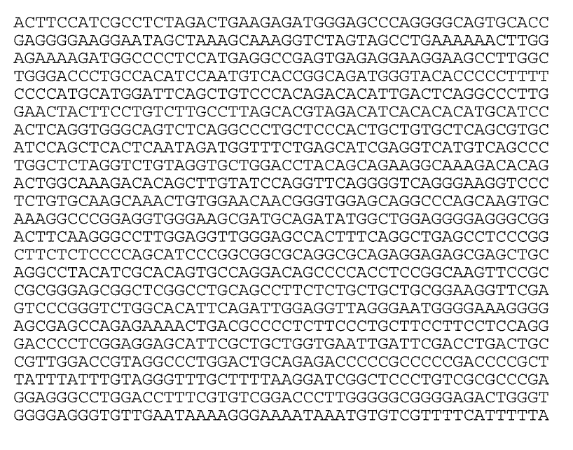

Two cellular models, HeLa cells and the dopaminergic cell line M17, were used to examine the effects of knockdown of SMPD1 on α-synuclein accumulation. Two different SiRNAs were used (see methods), and each was repeated twice. In both models, SiRNA knockdown led to reduction in quantity of ASMase, and to an average increase of about 3-fold of α-synuclein quantity (FIG. 10).

### Conclusion

The current study shows that specific SMPD1 variants are associated with PD, and that low ASMase activity is associated with earlier AAO of PD. The study also shows that the SMPD1 knockdown in two cellular models resulted in reduced ASMase levels, which may lead to α-synuclein accumulation.

### References

1. Gan-Or Z, Dion P A, Rouleau G A. Genetic perspective on the role of the autophagy-lysosome pathway in Parkinson disease. Autophagy. 2015;11(9):1443-57.
2. Cuervo A M, Stefanis L, Fredenburg R, Lansbury P T, Sulzer D. Impaired degradation of mutant alphα-synuclein by chaperone-mediated autophagy. Science. 2004 Aug 27;305(5688):1292-5.
3. Alcalay R N, Levy O A, Waters C C, et al. Glucocerebrosidase activity in Parkinson's disease with and without GBA mutations. Brain. 2015 Sep;138(Pt 9):2648-58.
4. Gan-Or Z, Amshalom I, Kilarski L L, et al. Differential effects of severe vs mild GBA mutations on Parkinson disease. Neurology. 2015 Mar 3;84(9):880-7.
5. Lesage S, Anheim M, Condroyer C, et al. Large-scale screening of the Gaucher's disease- related glucocerebrosidase gene in Europeans with Parkinson's disease. Hum Mol Genet. 2011 Jan 1;20(1):202-10.
6. Neumann J, Bras J, Deas E, et al. Glucocerebrosidase mutations in clinical and pathologically proven Parkinson's disease. Brain. 2009 Jul;132(Pt 7):1783-94.
7. Sidransky E, Nalls M A, Aasly J O, et al. Multicenter analysis of glucocerebrosidase mutations in Parkinson's disease. N Engl J Med. 2009 Oct 22;361(17):1651-61.
8. Mata I F, Samii A, Schneer S H, et al. Glucocerebrosidase gene mutations: a risk factor for Lewy body disorders. Arch Neurol. 2008 Mar;65(3):379-82.
9. Tsuang D, Leverenz J B, Lopez O L, et al. GBA mutations increase risk for Lewy body disease with and without Alzheimer disease pathology. Neurology. 2012 Nov 6;79(19):1944-50.
10. Mitsui J, Matsukawa T, Sasaki H, et al. Variants associated with Gaucher disease in multiple system atrophy. Ann Clin Transl Neurol. 2015 Apr;2(4):417-26.
11. Gan-Or Z, Mirelman A, Postuma R B, et al. GBA mutations are associated with Rapid Eye Movement Sleep Behavior Disorder. Ann Clin Transl Neurol. 2015 Sep;2(9):941-5.
12. Gegg M E, Burke D, Heales S J, et al. Glucocerebrosidase deficiency in substantia nigra of parkinson disease brains. Ann Neurol. 2012 Sep;72(3):455-63.
13. Clark L N, Chan R, Cheng R, et al. Gene-wise association of variants in four lysosomal storage disorder genes in neuropathologically confirmed Lewy body disease. PLoS One. 2015;10(5):e0125204.
14. Dagan E, Schlesinger I, Ayoub M, et al. The contribution of Niemann-Pick SMPD1 mutations to Parkinson disease in Ashkenazi Jews. Parkinsonism Relat Disord. 2015 Sep;21(9):1067-71.
15. Deng S, Deng X, Song Z, et al. Systematic Genetic Analysis of the SMPD1 Gene in Chinese Patients with Parkinson's Disease. Mol Neurobiol. 2015 Sep 16.
16. Foo J N, Liany H, Bei J X, et al. Rare lysosomal enzyme gene SMPD1 variant (p.R591C) associates with Parkinson's disease. Neurobiol Aging. 2013 Dec;34(12):2890 e13-5.
17. Gan-Or Z, Ozelius L J, Bar-Shira A, et al. The p.L302P mutation in the lysosomal enzyme gene SMPD1 is a risk factor for Parkinson disease. Neurology. 2013 Apr 23;80(17):1606-10.
18. Zampieri S, Filocamo M, Pianta A, et al. SMPD1 Mutation Update: Database and Comprehensive Analysis of Published and Novel Variants. Hum Mutat. 2015 Oct 26.
19. Schuchman EH, Wasserstein MP. Types A and B Niemann-Pick disease. Best Pract Res Clin Endocrinol Metab. 2015 Mar;29(2):237-47.
20. Sakanaka K, Waters C H, Levy O A, et al. Knowledge of and interest in genetic results among Parkinson disease patients and caregivers. J Genet Couns. 2014 Feb;23(1):114-20.
21. O'Roak B J, Vives L, Fu W, et al. Multiplex targeted sequencing identifies recurrently mutated genes in autism spectrum disorders. Science. 2012 Dec 21;338(6114):1619-22 .
22. Li H, Durbin R. Fast and accurate short read alignment with Burrows-Wheeler transform. Bioinformatics. 2009 Jul 15;25(14):1754-60.
23. McKenna A, Hanna M, Banks E, et al. The Genome Analysis Toolkit: a MapReduce framework for analyzing next-generation DNA sequencing data. Genome Res. 2010 Sep;20(9):1297-303.
24. Wang K, Li M, Hakonarson H. ANNOVAR: functional annotation of genetic variants from high-throughput sequencing data. Nucleic Acids Res. 2010 Sep;38(16):e164.
25. Kumar P, Henikoff S, Ng PC. Predicting the effects of coding non-synonymous variants on protein function using the SIFT algorithm. Nat Protoc. 2009;4(7):1073-81.
26. Adzhubei IA, Schmidt S, Peshkin L, et al. A method and server for predicting damaging missense mutations. Nat Methods. 2010 Apr;7(4):248-9.
27. Davydov EV, Goode DL, Sirota M, Cooper GM, Sidow A, Batzoglou S. Identifying a high fraction of the human genome to be under selective constraint using GERP++. PLoS Comput Biol. 2010;6(12):e1001025.
28. Olivova P, Cullen E, Titlow M, et al. An improved high-throughput dried blood spot screening method for Gaucher disease. Clin Chim Acta. 2008 Dec;398(1-2):163-4.
29. Reuser A J, Verheijen F W, Bali D, et al. The use of dried blood spot samples in the diagnosis of lysosomal storage disorders--current status and perspectives. Mol Genet Metab. 2011 Sep- Oct;104(1-2):144-8.Zhang X K, Elbin C S, Chuang W L, et al. Multiplex enzyme assay screening of dried blood spots for lysosomal storage disorders by using tandem mass spectrometry. Clin Chem. 2008 Oct;54(10):1725-8.
30. Lee B R, Kamitani T. Improved immunodetection of endogenous alpha-synuclein. PLoS One. 2011;6(8):e23939.
31. Martinez-Vicente M, Sovak G, Cuervo A M. Protein degradation and aging. Exp Gerontol. 2005 Aug-Sep;40(8-9):622-33.
32. Rhein C, Naumann J, Muhle C, et al. The Acid Sphingomyelinase Sequence Variant p.A487V Is Not Associated With Decreased Levels of Enzymatic Activity. JIMD Rep. 2013;8:1-6.
33. Pankratz N, Beecham G W, DeStefano A L, et al. Meta-analysis of Parkinson's disease: identification of a novel locus, RIT2. Ann Neurol. 2012 Mar;71(3):370-84.

Various publications are cited herein, the contents of which are hereby incorporated by reference in their entireties.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps. Patents, patent applications, publications, product descriptions and protocols are cited throughout this application the disclosures of which are incorporated herein by reference in their entireties for all purposes.

We claim:

1. A method of determining whether a subject is at risk of having Lewy body disease, the method comprising obtaining a biological sample from a subject and determining the presence of one or more variants in a gene selected from the group consisting of GBA, SMPD1, HEXA, MCOLN1, and combinations thereof, in the biological sample, wherein the presence of the one or more variants indicates that the subject is at risk of having Lewy body disease.

2. The method of claim 1, wherein the GBA variant comprises one or more of:
a single nucleotide polymorphism (SNP) rs114099990;
a mutation in the GBA genomic (g.) or cDNA (c.) nucleic acid sequence selected from the group consisting of g.1864A>G, c.38A>G, g.7549A>C, c.1584A>C, g.3940C>T, c.474C>T, g.5026C>T, c.795C>T, g.7314G>A, c.1443G>A, g. 7366G>C, c.1495G>C, g.3942G>A, c.476G>A, g.1367C>T, and combinations thereof; and
a mutation in a GBA amino acid sequence selected from the group consisting of p.Lys13Arg, p.Asp482Asn, p.Va1499Leu, p.Arg159Gln, and combinations thereof.

3. The method of claim 1, wherein the SMPD1 variant comprises one or more of:
a single nucleotide polymorphism (SNP) selected from the group consisting of rs144465428, rs1050228, rs71056748, rs7951904, rs1050239, rs8164, rs72896268, rs2723669, rs142178073, rs144873307, rs142787001 and combinations thereof;
a c.1829delCGG mutation in the SMPD1 cDNA nucleic acid sequence; and
a mutation in an SMPD1 amino acid sequence selected from the group consisting of p.Q19R, p.V36A, p.Leu49_Ser50insAL, p.Leu49_Ser50insALAL, p.D212D, p.E358K, p.G508R, p.R542L, p.V3011, p.M33I, p.G492S, p.E517V, p.R418Q, p.R291H, p.A487V, p.P331A, p.R378H, p.R498L, p.G530A, p.R610H, and combinations thereof

4. The method of claim 1, wherein the HEXA variant comprises one or more of:
a single nucleotide polymorphism (SNP) selected from the group consisting of rs2302449, rs387906309, rs73440586, rs117513345, rs1800428, rs121907970, rs117160567, rs2288259, rs1800431, rs121907954, rs117160567, rs10220917, and combinations thereof;
a 1277_1278insTATC mutation in the HEXA genomic nucleic acid sequence, a c.672+30T>G mutation in the HEXA cDNA sequence, or combinations thereof; and
a mutation in a HEXA amino acid sequence selected from the group consisting of p.Y4271, p.R247W, p.I436V, p.G269S, p.V192I, and combinations thereof.

5. The method of claim 1, wherein the MCOLN1 variant comprises one or more of:
a single nucleotide polymorphism (SNP) selected from the group consisting of rs45513896, rs145706318, rs73003348, rs2305889, rs139922988, rs145386883, rs686796, rs113261161, rs61736600, rs612862, rs142259322, rs147754092, and combinations thereof; and
a mutation in an MCOLN1 amino acid sequence selected from the group consisting of p.P197S, p.T261M, p.S257R, p.A138V, and combinations thereof

6. The method of claim 1, wherein at least two variants are present, wherein one of the at least two variants is in GBA, and the other variant is in SMPD1.

7. The method of claim 1, wherein at least three variants are present, wherein a first variant is in GBA, a second variant is in SMPD1, and a third variant is in MCOLN1.

8. The method of claim 1, wherein the one or more variants is a mutation that causes a lysosomal storage disorder when present in a subject.

9. The method of claim 1, wherein the one or more variants comprise a variant in GBA, and wherein GCase activity is decreased in the subject compared to a subject without LBD, or a reference level determined from one or more subjects without LBD.

10. The method of claim 1, wherein the subject has a decreased β-glucocerebrosidase: α-hexosaminidase protein activity ratio compared to a subject without LBD, or a reference level determined from one or more subjects without LBD.

11. The method of claim 1, wherein the subject comprising the one or more variant further comprises an altered lipid profile.

12. The method of claim 11, wherein the level of one or more of phosphatidylcholine, sphingolipid sphingomyelin and phosphatidylethanolamine is decreased in the subject compared to a subject without LBD, or a reference level determined from one or more subjects without LBD.

13. The method of claim 11, wherein the level of one or more of dihydrosphingomyelin, ceramide glycosphingolipid, phosphatidylserine and galactosylceramide is increased in the subject compared to a subject without LBD, or a reference level determined from one or more subjects without LBD.

14. The method of claim 1, wherein a Minor Allele Frequency of the one or more variants is less than 0.05.

15. The method of claim 1, wherein the subject is human.

16. The method of claim 1, wherein the biological sample is selected from the group consisting of a brain sample, a blood sample, and a cerebral spinal fluid sample.

17. The method of claim 1, wherein the presence of the variant is detected by in situ hybridization.

18. A method of preventing or treating Lewy body disease in a subject, comprising:
(a) determining the presence of one or more variants in a biological sample obtained from the subject, wherein the one or more variants are in a gene selected from the group consisting of GBA, SMPD1, HEXA, MCOLN1, and combinations thereof; and
(b) if the one or more variants are present in the biological sample, treating the subject with a Lewy body disease therapy.

19. The method of claim 18, wherein the Lewy body disease therapy comprises one or more of:
gene therapy;
administering a nucleic acid encoding a protein with one or more of GBA, SMPD1, HEXA, and MCOLN1 protein activity; and
protein replacement therapy comprising administering a protein with one or more of GBA, SMPD1, HEXA, and MCOLN1 protein activity to the subject.

20. A kit for determining whether a subject is at risk of having Lewy body disease, comprising reagents for detecting the presence of one or more variants in a gene selected from the group consisting of GBA, SMPD1, HEXA, MCOLN1, and combinations thereof, in a biological sample from a subject.

21. The kit of claim 20, wherein the reagent comprises one or more of:
a plurality of nucleic acid probes that specifically hybridize to a nucleic acid comprising the one or more variants; and
an antibody or antigen-binding fragment thereof, that specifically binds to a protein encoded by a nucleic acid comprising the one or more variants.
